(19) United States
(12) Patent Application Publication
(10) Pub. No.: US 2022/0334123 A1
(43) Pub. Date: Oct. 20, 2022

(54) SYSTEMS FOR PROTEIN CORONA ANALYSIS

(71) Applicant: Seer, Inc., Redwood City, CA (US)

(72) Inventors: Omid FAROKHZAD, Waban, MA (US); Asim SIDDIQUI, San Francisco, CA (US); Margaret DONOVAN, San Francisco, CA (US); John E. BLUME, Bellingham, WA (US); Craig STOLARCZYK, San Mateo, CA (US)

(21) Appl. No.: 17/733,876

(22) Filed: Apr. 29, 2022

Related U.S. Application Data
(63) Continuation of application No. PCT/US2020/058422, filed on Oct. 30, 2020.
(60) Provisional application No. 62/946,899, filed on Dec. 11, 2019.
(60) Provisional application No. 62/945,030, filed on Dec. 6, 2019.
(60) Provisional application No. 62/929,847, filed on Nov. 2, 2019.

(51) Int. Cl. G01N 33/68; G01N 33/543; G16B 40/20; G16B 5/00

(52) U.S. Cl. CPC G01N 33/6845; G01N 33/54313; G01N 33/6848; G16B 40/20; G16B 5/00

(57) ABSTRACT

Described herein are methods and systems for identifying protein-protein interactions using particle panels and protein corona formation. Also disclosed herein are systems and methods for enrichment analysis between protein annotations and particle biophysicochemical properties.

### CROSS-REFERENCE TO RELATED APPLICATIONS

The present application is a bypass continuation of International Application No. PCT/US2020/058422, filed on Oct. 30, 2020, which claims priority to and benefit from U.S. Provisional Application Nos. 62/929,847 filed Nov. 2, 2019; 62/945,030 filed Dec. 6, 2019; and 62/946,899 filed Dec. 11, 2019, the entire contents of each of which are herein incorporated by reference.

### BACKGROUND

Changes in protein-protein interactions may be indicative of biological changes or disease processes.

### SUMMARY

Disclosed herein are systems and methods for analyzing protein-particle interactions and protein-protein interactions. Interactions between biological molecules and particles and protein-protein interactions on particles may provide insights on protein-protein interactions across biological samples.

The present disclosure provides methods, compositions, and particles for assaying for proteins. In some aspects, the present disclosure provides methods of assaying a protein-protein interaction in a sample, comprising: (a) obtaining data comprising biomolecule information for a plurality of distinct biomolecule coronas from the sample, wherein the plurality of distinct biomolecule coronas correspond to a plurality of distinct particle types, wherein the plurality of distinct particle types comprises a first particle type; (b) detecting at least a primary protein and a secondary protein in a biomolecule corona of a first particle type from the data, and (c) identifying the protein-protein interaction by measuring the primary protein associated with the first particle type and the secondary protein associated with the first particle type, wherein the secondary protein is more strongly associated with the primary protein than the first particle type, thereby indicating a presence of the protein-protein interaction between the primary protein and secondary protein.

In some embodiments, the measuring comprises detecting associations of at least (i) the primary protein and the first particle type, (ii) the secondary protein and the first particle type, and (iii) the primary protein and the secondary protein, wherein the secondary protein has a greater association with the first protein than with the first particle type. In some embodiments, the method comprises detecting that the secondary protein is more strongly associated with the primary protein than the first particle type. In some embodiments, the measuring comprises quantifying the primary protein associated with the first particle type and the second protein associated with the first particle type.

In some embodiments, the data further comprises biomolecule information from a plurality of samples assaying using the plurality of distinct particle types. In some embodiments, the sample comprises a plurality of samples, each sample of the plurality assayed using one or more distinct particle types of the plurality of distinct particle types. In some embodiments, the plurality of samples comprise different total particle concentrations of the plurality of distinct particle types. In some embodiments, the plurality of samples comprise total particle concentrations between 100 fM and 100 nM. In some embodiments, the plurality of samples comprise a sample comprising a total particle concentration of between 1 pM and 100 pM and a sample comprising a total particle concentration of between 500 pM and 10 nM.

In some embodiments, the plurality of samples comprises samples comprising differences in a condition. In some embodiments, the condition comprises pH, osmolarity, ionic strength, conductivity, dielectric constant, viscosity, reduction potential, or any combination thereof. In some embodiments, the plurality of samples comprises a sample comprising a pH of between 5 and 7 and a sample comprising a pH of between 7.5 and 9.5.

In some embodiments, the identifying comprises determining a relationship between the protein-protein interaction and the condition. In some embodiments, the relationship comprises a pKa. In some embodiments, the identifying comprises determining whether the primary protein and the secondary protein occupy different layers in the biomolecule corona from among the plurality of distinct biomolecule coronas associated with the first particle type or the second particle type.

In some embodiments, the method further comprises determining that the secondary protein is more strongly associated with the primary protein than the first particle type, which determining comprises calibrating the data of (a) against a protein-protein interaction map. In some embodiments, the protein-protein interaction map comprises distances calculated at least in part from: (i) biochemical pathways; or (ii) protein-protein interactions.

In some embodiments, the detecting comprises measuring abundances of the primary protein and the secondary protein in the at least a subset of biomolecule coronas from among the plurality of biomolecule coronas. In some embodiments, the identifying comprises measuring a relationship between the abundances of the primary protein and the secondary protein in the at least the subset of biomolecule coronas from among the plurality of biomolecule coronas. In some embodiments, the identifying further comprises measuring the primary protein and the secondary protein associated with a second particle type.

In some embodiments, the assaying further comprises: determining a between-particle score based on a first signal detected upon binding of the primary protein to the particle type of the plurality of distinct particle types and a second signal detected upon binding of the first protein to a second particle type of the plurality of distinct particle types, and determining a same-particle score based on the first signal detected upon binding of the primary protein to the particle type and a third signal detected upon binding of the secondary protein to the particle type. In some embodiments, the assaying further comprises identifying the protein-protein interaction between the primary protein and the secondary protein when the same-particle score is greater than the between-particle score.

In some embodiments, the first signal, the second signal, and the third signal, the between-particle score, the same-particle score, or any combination thereof are used as training data for a machine learning algorithm. In some embodiments, the machine learning algorithm generates a trained classifier based on the training data. In some embodiments, the trained classifier identifies the protein-protein interactions in an experimental sample.

In some embodiments, the method further comprising identifying a biological state in the sample by identifying the presence or absence of the protein-protein interaction in the sample from the subject using the trained classifier. In some embodiments, the machine learning algorithm comprises weighting from a protein-protein interaction map or a biochemical pathway map.

In some embodiments, the method comprises determining a plurality of same-particle scores. In some embodiments, the method comprises identifying the protein-protein interaction between the primary protein and the secondary protein based on the plurality of same-particle scores. In some embodiments, the method comprises identifying the protein-protein interaction between the primary protein and the secondary protein based on the plurality of same-particle scores. In some embodiments, the between-particle score is less than about 0.24. In some embodiments, the same-particle score is greater than about 0.54.

In some embodiments, the plurality of same-particle scores comprises same particle scores corresponding to different samples from among a plurality of samples. In some embodiments, the plurality of samples comprises samples comprising different types of particles. In some embodiments, the plurality of samples comprises samples comprising different total particle concentrations. In some embodiments, the plurality of samples comprises samples comprising different conditions.

In some embodiments, the method comprises determining a plurality of same protein scores. In some embodiments, the method further comprises determining that the primary protein or the secondary protein is more strongly associated with the first particle type or the second particle type. In some embodiments, the method further comprises determining that the secondary protein is more strongly associated with the primary protein or a particle type from among the first particle type and the second particle type. In some embodiments, the determining the same particle-score comprises determining that the primary protein and the secondary protein occupy different layers of a biomolecule corona from among the plurality of the distinct biomolecule coronas.

In some embodiments, the plurality of distinct biomolecule coronas comprises a nucleic acid, a small molecule, a protein, a lipid, a polysaccharide, or any combination thereof. In some embodiments, the plurality of distinct biomolecule coronas comprises a protein pair whose concentrations differ by at least 6 orders of magnitude in the sample. In some embodiments, the plurality of distinct biomolecule coronas comprises a protein pair whose concentrations differ by at least 8 orders of magnitude in the sample. In some embodiments, the plurality of distinct biomolecule coronas comprises a protein pair whose concentrations differ by at least 10 orders of magnitude in the sample. In some embodiments, the biomolecule information comprises proteomic data for the plurality of distinct biomolecule coronas.

In some embodiments, the protein-protein interaction comprises hydrogen bonds, Van der Waals forces, or ionic bonds. In some embodiments, the protein-protein interaction comprises a contact surface between the primary protein and secondary protein of at least 500 Å². In some embodiments, the protein-protein interaction comprises a contact surface between the primary protein and secondary protein of at least 1000 Å². In some embodiments, the protein-protein interaction comprises a contact surface between the primary protein and secondary protein of at least 1500 Å².

In some embodiments, the identifying comprises determining a conformation, a post-translational modification, substrate binding, cofactor binding, or damage to the primary protein or the secondary protein. In some embodiments, the post-translational modification comprises cleavage, N-terminal extension, glycosylation, iodination, acetylation, degradation, acylation, biotinylation, amidation, alkylation, methylation, terminal amino acid cyclization, adenylation, ADP-ribosylation, sulfonation, prenylation, hydroxylation, decarboxylation, glutamylation, glycosylation, isoprenylation, lipoylation, phosphopantetheinylation, phosphorylation, and sulfation, or any combination thereof.

In some embodiments, the plurality of distinct particle types comprises at least 3 particle types. In some embodiments, the plurality of distinct particle types comprises at least 5 particle types. In some embodiments, the plurality of distinct particle types differ from each other by one or more physicochemical properties. In some embodiments, the one or more physicochemical properties are selected from the group consisting of: composition, size, surface charge, hydrophobicity, hydrophilicity, surface functionality, surface topography, surface curvature, shape, and any combination thereof. In some embodiments, the surface functionality comprises a small molecule functionalization. In some embodiments, the small molecule functionalization comprises an amine functionalization, a carboxylate functionalization, a monosaccharide functionalization, an oligosaccharide functionalization, a phosphate sugar functionalization, a sulfate sugar functionalization, an alcohol functionalization, a ether functionalization, an ester functionalization, an amide functionalization, a carbonate functionalization, a carbamate functionalization, a urea functionalization, a benzyl functionalization, a phenyl functionalization, a phenol functionalization, an aniline functionalization, an imidazole functionalization, an indole functionalization, a fluoride functionalization, a chloride functionalization, a bromide functionalization, a sulfide functionalization, a nitro functionalization, a thiol functionalization, a nitrogenous base functionalization, an aminopropyl functionalization, a boronic acid functionalization, an N-succinimidyl ester functionalization, a PEG functionalization, a methyl ether functionalization, a triethoxylpropylaminosilane functionalization, a silicon alkoxide functionalization, a phenol-formaldehyde functionalization, an organosilane functionalization, an ethylene glycol functionalization, a PCP functionalization, a citrate functionalization, a lipoic acid functionalization, or any combination thereof. In some embodiments, the small molecule functionalization comprises a silica functionalized particle, an amine functionalized particle, a silicon alkoxide functionalized particle, a polystyrene functionalized particle, and a saccharide functionalized particle. In some embodiments, the small molecule functionalization comprises an amine functionalization, a phosphate sugar functionalization, a carboxylate functionalization, a silica functionalization, an organosilane functionalization, or any combination thereof. In some embodiments, the small molecule functionalization comprises a silica functionalization, an ethylene glycol functionalization, and an amine functionalization, or any combination thereof.

In some embodiments, the surface functionality comprises one or more macromolecular functionalization. In some embodiments, the one or more macromolecular functionalization comprises a macromolecule attached to the surface of the particle, and wherein the macromolecule comprises a protein-functionalization, a polysaccharide functionalization, or any combination thereof. In some embodiments, the macromolecule is attached to the surface of the particle by a flexible linker. In some embodiments, the flexible linker comprises a length of at least 4 nanometers (nm). In some embodiments, the macromolecule is attached to the surface of the particle by a rigid linker. In some embodiments, the rigid linker comprises a length of at least 2 nm. In some embodiments, the macromolecule comprises dextran. In some embodiments, the macromolecule comprises a protein. In some embodiments, the macromolecular functionalization comprises a plurality of ubiquitin molecules bound to the particle. In some embodiments, the macromolecular functionalization comprises a plurality of ubiquitin molecules bound to the particle in a plurality of orientations or through a C-termini.

In some embodiments, the plurality of distinct particle types comprises one or more small molecule functionalized particle and one or more macromolecular functionalized particle. In some embodiments, the plurality of distinct particle types comprises one or more positively charged particle and one or more negatively charged particle. In some embodiments, the plurality of distinct particle types further comprises one or more neutral particle. In some embodiments, the plurality of distinct particle types comprises at least one positively charged particle and at least one neutral particle. In some embodiments, the plurality of distinct particle types comprises at least one negatively charged particle and at least one neutral particle.

In some embodiments, the biomolecule corona of the plurality of distinct biomolecule coronas comprises: (i) a primary biomolecule corona comprising a first layer of proteins directly binding to a surface of a particle type of the plurality of particle types; and (ii) a secondary biomolecule corona comprising a second layer of proteins that bind to proteins in the primary corona; and wherein identifying the protein-protein interaction comprises identifying an interaction between the primary protein in the primary biomolecule corona and the secondary protein in the secondary biomolecule corona. In some embodiments, the biomolecule information distinguishes the primary and secondary biomolecule coronas. In some embodiments, the detecting further comprises detecting a protein class.

In some embodiments, the protein class comprises a protein class selected from among the group consisting of protease inhibitors, disulfide bond containing proteins, sterol metabolism proteins, innate immunity proteins, serine protease inhibitors, inflammatory response proteins, lipid metabolism proteins, glycoproteins, disease mutation proteins, age-related macular degeneration-related proteins, atherosclerosis proteins, very low density lipoproteins (VLDL), nucleus proteins, serine proteases, zinc proteins, hydroxylases, isopeptide bond proteins, transmembrane helix proteins, phosphoproteins, secreted proteins, membrane proteins, cytoskeletal proteins, myopathy proteins, proteins with serine protease homology, transmembrane beta stain proteins, antioxidant proteins, protein synthesis inhibitor, non-syndromic deafness proteins, congenital dyserythropoietic proteins, mental retardation related proteins, corneal dystrophy proteins, RNA editing proteins, Alzheimer's related proteins, copper proteins, hemoglobin-binding proteins, actin-binding proteins, deafness related proteins, hereditary hemolytic anemia proteins, cytolysis proteins, heme proteins, eibrinolysis proteins, hyperlipidemia proteins, amyloid proteins, amyloidosis related proteins, pyrrolidone carboxylic acid proteins, high density lipid (HDL) proteins, signal proteins, blood coagulation proteins, glycated proteins, adaptive immunity proteins, muscle proteins, chaperone proteins, ribonucleoproteins, nucleosome core proteins, chromosomal proteins, mRNA splicing proteins, ER-Golgi transport proteins, complement activation lectin pathway proteins, autocatalytic cleavage proteins, Ubl conjugation proteins, SH2 domain proteins, coated pit proteins, tissue remodeling proteins, mRNA processing proteins, spliceosome proteins, citrullinated proteins, RNA-binding proteins, Ribosomal proteins, EGF-like domain proteins, sulfated proteins, complement alternate pathway proteins, immunity proteins, meostasis proteins, oxidized proteins, immunoglobulins, oxygen transport proteins, thioester bond containing proteins, bence-j ones protein, thrombophilia related proteins, membrane attack complex proteins, integrins, vasoactive proteins, sialic acid proteins, iron proteins, acute phase proteins, hypotensive agent proteins, mineral balance proteins, systemic lupus erthyematosus proteins, chromophore-containing proteins, bait region proteins, atrial septal defect related proteins, airport syndrome proteins, pyruvate enzymes, aortic aneurysm related proteins, hemolytic uremic syndrome related proteins, lipid degradation related proteins, ATP-binding proteins, polymorphism proteins, stress response proteins, repeat proteins, acetylated proteins, transmembrane proteins, methylated proteins, cytoplasmic proteins, calcium binding proteins, post-virus interaction proteins, complement pathway proteins, cell adhesion proteins, cholesterol metabolism proteins, heparin-binding proteins, immunoglobulin domain proteins, lipid transport proteins, steroid metabolism proteins, and transport proteins, or any combination thereof. In some embodiments, the protein class comprises a plurality of proteins comprising a common function, common biological localization, common cofactor, common structural motif, common PTM, common biological state.

In some embodiments, the identifying the protein-protein interaction comprises identifying a biological state. In some embodiments, the identifying the protein-protein interaction comprises identifying a signal transduction pathway associated with the biological state. In some embodiments, the biological state is a phenotype. In some embodiments, the phenotype is a healthy biological state. In some embodiments, the phenotype is a disease biological state. In some embodiments, the identifying the disease biological state comprises identifying the stage of the disease biological state. In some embodiments, the stage the disease biological state is an early or pre-onset stage.

In some embodiments, the plurality of distinct biomolecule coronas are formed by contacting the sample with the plurality of distinct particle types. In some embodiments, the method comprises generating the plurality of distinct biomolecule coronas by separating a plurality of particle types from the sample. In some embodiments, the method comprises contacting the sample with the plurality of particle types prior to the generating.

In some embodiments, the method comprises generating the data by assaying the sample, wherein assaying comprises performing one or more assays selected from the group consisting of: a biomolecule corona assay, a particle enrichment assay, an affinity binding assay, a mass spectrometric assay, an isoelectric focusing assay, a chromatographic assay, a salting out assay, a gradient centrifugation assay, or any combination thereof. In some embodiments, the assay comprises a mass spectrometric assay.

In various aspects provided herein are kits for performing the methods of the present disclosure. In some embodiments, a kit comprises the first particle type and the second particle type, wherein the first particle type and second particle type are one or more particle types selected from the group consisting of micelles, liposomes, iron oxide particles, silver particles, gold particles, palladium particles, quantum dots, platinum particles, titanium particles, silica particles, metal or inorganic oxide particles, synthetic polymer particles, copolymer particles, terpolymer particles, polymeric particles with metal cores, polymeric particles with metal oxide cores, polystyrene sulfonate particles, polyethylene oxide particles, polyoxyethylene glycol particles, polyethylene imine particles, polylactic acid particles, polycaprolactone particles, polyglycolic acid particles, poly(lactide-co-glycolide polymer particles, cellulose ether polymer particles, polyvinylpyrrolidone particles, polyvinyl acetate particles, polyvinylpyrrolidone-vinyl acetate copolymer particles, polyvinyl alcohol particles, acrylate particles, polyacrylic acid particles, crotonic acid copolymer particles, polyethlene phosphonate particles, polyalkylene particles, carboxy vinyl polymer particles, sodium alginate particles, carrageenan particles, xanthan gum particles, gum acacia particles, Arabic gum particles, guar gum particles, pullulan particles, agar particles, chitin particles, chitosan particles, pectin particles, karaya tum particles, locust bean gum particles, maltodextrin particles, amylose particles, corn starch particles, potato starch particles, rice starch particles, tapioca starch particles, pea starch particles, sweet potato starch particles, barley starch particles, wheat starch particles, hydroxypropylated high amylose starch particles, dextrin particles, levan particles, elsinan particles, gluten particles, collagen particles, whey protein isolate particles, casein particles, milk protein particles, soy protein particles, keratin particles, polyethylene particles, polycarbonate particles, polyanhydride particles, polyhydroxyacid particles, polypropylfumerate particles, polycaprolactone particles, polyamine particles, polyacetal particles, polyether particles, polyester particles, poly(orthoester) particles, polycyanoacrylate particles, polyurethane particles, polyphosphazene particles, polyacrylate particles, polymethacrylate particles, polycyanoacrylate particles, polyurea particles, polyamine particles, polystyrene particles, poly(lysine) particles, chitosan particles, dextran particles, poly(acrylamide) particles, derivatized poly(acrylamide) particles, gelatin particles, starch particles, chitosan particles, dextran particles, gelatin particles, starch particles, poly-β-amino-ester particles, poly(amido amine) particles, poly lactic-co-glycolic acid particles, polyanhydride particles, bioreducible polymer particles, and 2-(3-aminopropylamino)ethanol particles, protein functionalized particles, ubiquitin functionalized particles, polysaccharide coated particles, dextran functionalized particles, or any combination thereof. In some embodiments, the first particle type and the second particle type are one or more particle types selected from the group consisting of carboxylate (Citrate) superparamagnetic iron oxide nanoparticle (SPION), a phenol-formaldehyde coated SPION, a silica-coated SPION, a polystyrene coated SPION, a carboxylated poly(styrene-co-methacrylic acid) coated SPION, a N-(3-Trimethoxysilylpropyl)diethylenetriamine coated SPION, a poly(N-(3-(dimethylamino)propyl) methacrylamide) (PDMAPMA)-coated SPION, a 1,2,4,5-Benzenetetracarboxylic acid coated SPION, a poly(Vinylbenzyltrimethylammonium chloride) (PVBTMAC) coated SPION, a carboxylate, PAA coated SPION, a poly(oligo(ethylene glycol) methyl ether methacrylate) (POEGMA)-coated SPION, a carboxylate microparticle, a polystyrene carboxyl functionalized particle, a carboxylic acid coated particle, a silica particle, a carboxylic acid particle, an amino surface particle, a silica amino functionalized particle, a Jeffamine surface particle, a polystyrene particle, a particle coated with a dextran based coating of about 0.13 μm in diameter, or a silica silanol coated particle. In some embodiments, the first particle type and the second particle type are one or more particle types selected from the group consisting of silica-coated particles, N-(3-Trimethoxysilylpropyl)diethylenetriamine coated particles, poly(N-(3-(dimethylamino)propyl) methacrylamide) (PDMAPMA)-coated particles, phosphate-sugar functionalized polystyrene particles, amine functionalized polystyrene particles, polystyrene carboxyl functionalized particles, ubiquitin functionalized polystyrene particles, dextran coated particles, or any combination thereof, wherein one or more of the particles optionally comprises a paramagnetic or superparamagnetic core material. In some embodiments, wherein the first particle type and the second particle type are one or more particle types selected from the group consisting of silica particles, poly(acrylamide) particles, polyethylene glycol particles, or any combination thereof, wherein one or more of the particles optionally comprises a paramagnetic or superparamagnetic core material. In some embodiments, the first particle type and the second particle type comprises a macromolecular functionalized particle and a small molecule functionalized particle.

In some embodiments, a kit comprises a resuspension buffer. In some embodiments, a kit comprises a digestion buffer. In some embodiments, a kit comprises a denaturation buffer. In some embodiments, a kit comprises comprising a lysis buffer. In some embodiments, a kit comprises comprises a substrate, wherein the substrate comprises a plurality of partitions, and wherein, of the plurality of partitions, a first partition comprises the first particle type and a second partition comprises the second particle type. In some embodiments, a substrate comprises a multi-well plate.

Various aspects of the present disclosure provide methods for using a kit disclosed herein to detect a protein-protein interaction in a sample, comprising: (i) adding a sample to at least a subset of the plurality of partitions, (ii) adding a buffer to said at least said subset of the plurality of partitions, thereby generating mass spectrometric samples, (iii) performing mass spectrometric analysis on at least a subset of the mass spectrometric samples, thereby generating mass spectrometric data, and (iv) identifying a protein-protein interaction based on the mass spectrometric data. In some embodiments, the protein-protein interaction is identified no more than 7 hours after (i). In some embodiments, the protein-protein interaction is identified no more than 6 hours after (i). In some embodiments, the protein-protein interaction is identified no more than 5 hours after (i). In some embodiments, the protein-protein interaction is identified no more than 4 hours after (i). In some embodiments, the protein-protein interaction is identified no more than 3 hours after (i). In some embodiments, the protein-protein interaction is identified no more than 2 hours after (i).

Aspects of the present disclosure provide a capture particle comprising: a first physicochemical property selected from the group consisting of a magnetic core, a polystyrene core, a metal core, a gold core, a metal oxide core, an iron oxide core, a polymeric core, and a silica core; a second physicochemical property selected from the group consisting of a carboxylated surface, an amino surface, a silica surface, a polymer surface, a phosphate sugar functionalized surface, a phenol functionalized surface, a citrate functionalized surface, a Jeffamine surface, and a silica silanol surface; and a bait molecule. In some embodiments, the bait molecule comprises ubiquitin, a ubiquitin-like protein, or a fragment thereof. In some embodiments, the ubiquitin, the ubiquitin like protein, or the fragment thereof is linked to the particle through an amine of the ubiquitin, the ubiquitin like protein, or the fragment thereof. In some embodiments, the amine is a random amine of the ubiquitin or the fragment thereof. In some embodiments, the ubiquitin, the ubiquitin-like protein, or the fragment thereof is linked to the particle through a C-terminal carboxylate of the ubiquitin, the ubiquitin-like protein, or the fragment thereof. In some embodiments, the bait molecule comprises a plurality of ubiquitin, ubiquitin-like proteins, fragments of ubiquitin like proteins, or a combination thereof. In some embodiments, the bait molecule comprises dextran. In some embodiments, no more than 10% of the surface of the particle is covered by the bait molecule. In some embodiments, no more than 20% of the surface of the particle is covered by the bait molecule. In some embodiments, no more than 30% of the surface of the particle is covered by the bait molecule. In some embodiments, no more than 40% of the surface of the particle is covered by the bait molecule. In some embodiments, no more than 50% of the surface of the particle is covered by the bait molecule. In some embodiments, no more than 60% of the surface of the particle is covered by the bait molecule. In some embodiments, no more than 70% of the surface of the particle is covered by the bait molecule. In some embodiments, no more than 80% of the surface of the particle is covered by the bait molecule. In some embodiments, the bait molecule binds a protein selected from the group consisting of: a ubiquitinated protein, an RNA splicing protein, an mRNA splicing protein, an ER-Golgi transport protein, a tissue remodeling protein, a complement activation lectin pathway protein, a coated pit protein, an SH2 domain protein, a chaperone, a ribosomal protein, a ribonucleoprotein, an RNA-binding protein, a nucleosome core protein, a citrullinated protein, a spliceosome protein, or any combination thereof.

Various aspects of the present disclosure provide a method of assaying for a target protein in a sample using a capture particle, comprising contacting a sample comprising the target protein with a capture particle. In some embodiments, the target protein is a ubiquitinated protein, an RNA splicing protein, an mRNA splicing protein, an ER-Golgi transport protein, a tissue remodeling protein, a complement activation lectin pathway protein, a coated pit protein, an SH2 domain protein, a chaperone, a ribosomal protein, a ribonucleoprotein, an RNA-binding protein, a nucleosome core protein, a citrullinated protein, a spliceosome protein, or any combination thereof. In some embodiments, the assaying imparts a measurable conformational change in the target protein. In some embodiments, the relative abundance of the target protein on the capture particle is greater than the relative abundance of the protein in the sample. In some embodiments, the relative abundance of the target protein on the capture particle is greater than for a control capture particle lacking the bait molecule and comprising a similar size and composition as the capture particle.

Various aspects of the present disclosure provide a method of assaying a protein-protein interaction in a sample, the method comprising: contacting a sample with a capture particle, wherein upon contacting the sample with the capture particle, a first protein in the sample binds the bait molecule and wherein upon binding the bait molecule, the first protein undergoes a conformational change; assaying for a second protein, wherein the second protein binds the first protein upon the first protein undergoing a conformational change. In some embodiments, the second protein is unbound from the first protein in the absence of the capture particle.

Various aspects of the present disclosure provide a method of identifying a drug targeting pathway in a sample, the method comprising: obtaining proteins that interact with (i) a first particle type and (ii) a second particle type by separating a plurality of particle types comprising the first particle type and the second particle type from the sample, wherein a surface of the first particle type in the plurality of particles types comprises a bait molecule, and wherein the proteins comprise: a primary protein that directly interacts with the bait molecule of the first particle type; and a secondary protein that indirectly interacts with the bait molecule of the first particle type by binding the first protein; assaying the proteins to identify the presence or absence of a protein-protein interaction indicative of the drug targeting pathway. In some embodiments, the bait molecule comprises ubiquitin or dextran. In some embodiments, prior to the obtaining, the method comprises contacting the sample with the plurality of particle types.

In some embodiments, the assaying further comprises: determining a between-particle score based on a first signal detected upon binding of the primary protein to the first particle type and a second signal detected upon binding of the primary protein to the second particle type, and determining a same-particle score based on the first signal and a third signal detected upon binding of the secondary protein to the first particle type. In some embodiments, the method comprises identifying the protein-protein interaction between the primary protein and the secondary protein when the same-particle score is greater than the between particle score. In some embodiments, the method comprises identifying a protein-bait molecule interaction between the primary protein and the bait molecule when the between-particle score is greater than a predetermined threshold. In some embodiments, the method comprises generating a protein-protein interaction map comprising at least 10, at least 100, at least 500, or at least 1000 proteins indicative of the drug targeting pathway. In some embodiments, the method comprises identifying at least at least 2 protein-bait interactions, at least 5 protein-bait interactions, at least 10 protein-bait interactions, at least 25 protein-bait interactions, at least 50 protein-bait interactions, at least 100 protein-bait interactions, or at least 1000 protein-bait interactions.

In some embodiments, the method further comprises comparing the protein-protein interaction to a reference protein-protein interaction. In some embodiments, the reference protein-protein interaction is from a protein-protein interaction database. In some embodiments, the reference protein-protein interaction is present in a sample lacking a disease phenotype. In some embodiments, the reference protein-protein interaction is present in a sample obtained from a subject having or suspected of having a disease phenotype. In some embodiments, the reference protein-protein interaction is detected by enzyme-linked immunosorbent assay (ELISA), immunofluorescence, yeast-hybrid, size exclusion chromatography, surface plasmon resonance, or any combination thereof.

In some embodiments, the drug targeting pathway is a signal transduction pathway. In some embodiments, the drug targeting pathway is implicated in a disease biological state. In some embodiments, the disease biological state is cancer. In some embodiments, the disease biological state is a neurological disease. In some embodiments, the neurological disease is Alzheimer's disease.

In some embodiments, a method provides for identifying a state of a target protein associated with a drug targeting pathway, and further comprises: assaying the proteins to measure an amount of the target protein; and identifying the state of the target protein based on the measured amount of the target protein. In some embodiments, the first particle type directly binds to the target protein in a first state and the first particle type indirectly binds to the target protein in a second state. In some embodiments, a surface of the second particle type comprises the bait molecule. In some embodiments, a surface of the second particle type comprises a second bait molecule. In some embodiments, the first particle type directly or indirectly binds to the target protein in a first state and the second particle type directly or indirectly binds to the target protein in a second state.

In some embodiments, a surface of the first particle type comprises a first bait molecule in a first conformation and a surface of the second particle type comprises the first bait molecule in a second conformation; and the proteins comprise: a first set of proteins that interact with the first particle type; and a second set of proteins that interact with the second particle type, wherein the first set of proteins and the second set of proteins are different in (i) protein content or (ii) concentration of a protein. In some embodiments, obtaining the first set of proteins and obtaining the second set of proteins is concurrent.

In some embodiments, the first signal is detected upon binding of a primary protein in the first set of proteins to the first particle type; the second signal is detected upon binding of the primary protein in the first set of proteins to the second particle type; and the third signal is detected upon binding of a secondary protein in the second set of proteins to the first particle type. In some embodiments, the method comprises identifying a protein-protein interaction between the first protein and the second protein when the same-particle score is greater than the between-particle score. In some embodiments, the same-particle score is at least 1, 1.5, 2, 2.5, 3, or 3.5 standard deviations above the mean same-particle score for the sample. In some embodiments, a method comprises identifying a protein-bait molecule interaction between the primary protein and the bait molecule when the between-particle score is greater than about 0.6. In some embodiments, the between-particle score is greater than about 0.7. In some embodiments, the between-particle score is greater than about 0.85.

In some embodiments, a method comprises generating a primary protein-bait interaction map comprising at least 10, at least 100, at least 500, or at least 1000 proteins indicative of protein-bait interactions in the first conformation and a secondary protein-bait interaction map comprising at least 10, at least 100, at least 500, or at least 1000 proteins indicative of protein-bait interactions in the second conformation. In some embodiments, the bait molecule is a small molecule. In some embodiments, the bait molecule is a protein. In some embodiments, the small molecule or the protein is a therapeutic agent.

In some embodiments, a method comprises contacting 2 or more, 3 or more, 4 or more, 5 or more, 10 or more, 100 or more, at 500 or more, or 1000 or more samples with the plurality of distinct particle types. In some embodiments, the 2 or more, 3 or more, 4 or more, 5 or more, 10 or more, 100 or more, at 500 or more, or 1000 or more samples are derived from a single volume of a biological sample. In some embodiments, one or more sample(s) of the 2 or more, 3 or more, 4 or more, 5 or more, 10 or more, 100 or more, at 500 or more, or 1000 or more samples are labeled with a sample-specific tag. In some embodiments, the sample-specific tag is a mass tag. In some embodiments, the plurality of particle types comprises 2 or more, 3 or more, 4 or more, 5 or more, 6 or more, 7 or more, 8 or more, 9 or ore, 10 or more, 20 or more, 30 or more, 40 or more, 50 or more, or 100 or more particle types. In some embodiments, the identifying is completed in at most 1 hour. In some embodiments, the identifying is completed in at most 50 minutes. In some embodiments, the identifying is completed in at most 40 minutes. In some embodiments, the identifying is completed in at most 30 minutes. In some embodiments, the identifying is completed in at most 20 minutes. In some embodiments, the identifying is completed in at most 10 minutes. In some embodiments, the sample has a volume of less than 1 mL, less than 0.9 mL, less than 0.8 mL, less than 0.7 mL, less than 0.6 mL, less than 0.5 mL, less than 0.1 mL, less than 0.05 mL, or less than 0.01 mL. In some embodiments, a method comprises generating a protein-protein interaction map comprising at least 10, at least 100, at least 500, or at least 1000 proteins.

In some embodiments, a method further comprising identifying one or more protein-protein interactions, 2 or more protein-protein interactions, 5 or more protein-protein interactions, 10 or more protein-protein interactions, 25 or more protein-protein interactions, 50 or more protein-protein interactions, 100 or more protein-protein interactions, or 1000 or more protein-protein interactions. In some embodiments, a method further comprising identifying 10 or more, 100 or more, 500 or more, or 1000 or more non-interacting proteins. In some embodiments, the first particle type differs from the second particle type in the plurality of particle types by a physicochemical property. In some embodiments, a method comprises generating a database of the first signal, the second signal, the third signal, the first particle type, the second particle type, the first protein, the second protein, the between-particle score, the same-particle score, the protein-protein interaction, the biological state, the drug targeting pathway, or any combination thereof. In some embodiments, a method comprises outputting a report of the first signal, the second signal, the third signal, the first particle type, the second particle type, the first protein, the second protein, the between-particle score, the same-particle score, the protein-protein interaction, the biological state, the drug targeting pathway, or any combination thereof.

Various aspects of the present disclosure provide a system comprising: computer memory comprising data comprising biomolecule information for a plurality of distinct biomolecule coronas from a sample, wherein the plurality of distinct biomolecule coronas corresponds to a plurality of distinct particle types, wherein the plurality of distinct particle types comprises a first particle type; a computer in communication with the computer memory, wherein the computer comprises a computer processor and computer readable medium comprising machine-executable code that, upon execution by the one or more computer processors, implements a method comprising: (i) receiving the data from the computer memory; (ii) from the data, detecting at least a primary protein and a secondary protein in a biomolecule corona of a first particle type; and (iii) identifying the protein-protein interaction by measuring the association of the primary protein with the first particle type, the association of the secondary protein with the first particle type, and the association of the primary protein with the secondary protein, wherein the association of the primary protein with the secondary protein is greater than the association of the secondary protein with the first particle type, thereby indicating a presence of the protein-protein interaction between the primary protein and secondary protein.v In some embodiments, (ii) is repeated for at least a subset of the plurality of distinct biomolecule coronas prior to (iii). In some embodiments, said at least said subset of distinct biomolecule coronas is associated with multiple particle types from among the plurality of distinct particle types. In some embodiments, the measuring comprises identifying a variance in an association of (iii) across said at least said subset of distinct biomolecule coronas. In some embodiments, (ii) and (iii) are repeated for a plurality of distinct pairs of primary and secondary proteins. In some embodiments, the identifying comprises distinguishing the association of the primary protein with the secondary protein from the association of the primary protein with a third protein.

In some embodiments, the associations in (iii) comprise scores, wherein the scores are based on correlations. In some embodiments, the score of the primary protein with the secondary protein is at least 0.5 greater than the score of the secondary protein with the first particle type. In some embodiments, the score of the primary protein with the secondary protein is at least 0.68 greater than the score of the secondary protein with the first particle type. In some embodiments, the score of the primary protein with the secondary protein is at least 0.8 greater than the score of the secondary protein with the first particle type. In some embodiments, the score calculated based on Pearson value or correlation.

In some embodiments, the detecting of (ii) comprises identifying an abundance of the primary protein and an abundance of the secondary protein in the biomolecule corona. In some embodiments, (iii) further comprises calibrating an association of (iii) with a weighted algorithm or a machine learning algorithm. In some embodiments, the machine learning algorithm comprises weighting from a protein-protein interaction map or a biochemical pathway map. In some embodiments, (ii) further comprises detecting a protein class in the biomolecule corona of the first protein type. In some embodiments, (iii) further comprises modifying an association from among the associations of (iii) based on the protein class detected in (ii). In some embodiments, the measuring comprises a factorization or a decomposition of the data. In some embodiments, an association from (iii) comprises a calibration with a weighting factor from the factorization or the decomposition of the data. In some embodiments, the system detects a biological state based on the protein-protein interaction between the primary protein and the secondary protein. In some embodiments, the data is transmitted to the computer memory over a communication network.

In some embodiments, the system identifies a particle functionalization to increase or decrease a putative abundance of the protein-protein interaction detected in an additional set of biomolecule information based on the identified protein-protein interaction.

Various aspects of the present disclosure provide a method for assaying proteins, comprising: identifying a target protein or target protein cluster based on an identified protein-protein interaction; and selecting or functionalizing a particle type based on the identified target protein or target protein cluster.

Various aspects of the present disclosure provide a method for designing a particle to assay for a protein-protein interaction, comprising: identifying a target protein cluster of interest, wherein the target protein cluster comprises a plurality of proteins; and functionalizing the particle to bind the plurality of proteins with an affinity of no greater than 10 μM. In some embodiments, a method of designing a particle to assay for a protein-protein interaction comprises adding the particle to a particle panel, and determining that the particle generates a same protein score of less than 0.5 for at least a subset of proteins from among the plurality of proteins. In some embodiments, the same protein score is less than 0.4. In some embodiments, the same protein score is less than 0.3. In some embodiments, the same protein score is less than 0.2. In some embodiments, the same protein score is less than 0.1. In some embodiments, the same protein score is less than 0. In some embodiments, the same protein score is less than −0.1. In some embodiments, the same protein score is less than −0.2. In some embodiments, the same protein score is less than −0.3. In some embodiments, the same protein score comprises a Pearson correlation value. In some embodiments, the identifying comprises determining that fewer than 10% of the proteins from among the target protein cluster of interest comprises a protein-protein interaction within a protein-protein interaction database. In some embodiments, the identifying comprises determining that fewer than 4% of the proteins from among the target protein cluster of interest comprises a protein-protein interaction within the protein-protein interaction database. In some embodiments, the identifying comprises determining that fewer than 1% of the proteins from among the target protein cluster of interest comprises a protein-protein interaction within the protein-protein interaction database. In some embodiments, the functionalizing comprises a macromolecular surface functionalization. In some embodiments, the macromolecular functionalization comprises a ubiquitin or ubiquitin-like protein. In some embodiments, the particle binds the plurality of proteins with an affinity of no greater than 100 μM. In some embodiments, the particle binds the plurality of proteins with an affinity of no greater than 1 mM.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings (also “Figure” and “FIG.” herein) of which:

FIG. 1 shows several examples of particle types and several ways the particle surfaces can be functionalized. In some cases, the particles may be nanoparticles.

FIG. 2 shows the separation of superparamagnetic iron oxide nanoparticles (SPIONs) from the remaining solution. As illustrated in the left photo, the SPIONs are dispersed in solution, seen as a dark, opaque solution in a glass vial, prior to or concurrent with application of a magnet to the side of the vial. Within 30 seconds of applying a magnet to the side of the vial, the SPIONs are separated from the solution, as illustrated by accumulation of dark particles next to the magnet and an increase in solution transparency in the photo on the right. Upon shaking the separated solution shown in the right image, the particles return to the dispersed state shown in the left image within 5 seconds. The SPIONs have a fast response.

FIG. 3 shows the concentration responses for spiked proteins as compared to the controls. The spikes change with concentration. Endogenous protein controls did not change with concentration. FIG. 3 shows data from spike recovery experiments of CRP. The protein was spiked at 4 levels: 2×, 5×, 10×, and 100λ. HX-42 (SP-006) (left) and HX-97 (right, same as SP-007) were used.

FIG. 4A-B illustrate a schematic of the formation of particle protein corona (FIG. 4A), and an embodiment of the present disclosure, the Proteograph platform workflow, based on multi-particle type protein corona approach and mass spectrometry for plasma proteome analysis (FIG. 4B). FIG. 4A show three distinct particle types (depicted in the center of the figure, with the top, middle, and bottom spheres representing the three distinct particle types), each different from the other by at least one physicochemical property, which leads to the formation of different protein corona compositions on the particle surfaces. FIG. 4B shows the corona analysis workflow with Proteograph, which includes: (1) particle-plasma incubation and protein corona formation; (2) particle protein corona purification by a magnet; (3) digestion of corona proteins; and (4) liquid chromatography mass spectrometry analysis (LC-MS). In this context, each plasma-NP well is a sample for a total of 96 samples per plate.

FIG. 5 illustrates characterization of the three superparamagnetic iron oxide nanoparticles (SPIONs) shown in the left-most first column, which from top to bottom, are: silica-coated SPION (SP-003), poly(N-(3-(dimethylamino)propyl) methacrylamide) (PDMAPMA)-coated SPION (SP-007), and poly(oligo(ethylene glycol) methyl ether methacrylate) (POEGMA)-coated SPION (SP-011), by the following methods: scanning electron microscopy (SEM, second columns of images), dynamic light scattering (DLS, third column of graphs), transmission electron microscopy (TEM, fourth column of images), high-resolution transmission electron microscopy (HRTEM, fifth column), and X-ray photoelectron spectroscopy (XPS, sixth column), respectively. DLS shows three replicates of each particle type. The HRTEM pictures were recorded at the surface of individual SP-003, SP-007, and SP-011 particle types, respectively, and the arrow points to the region of amorphous SiO2 (top HRTEM image) coating and amorphous SiO2/polymer coatings (middle and bottom HRTEM images) on the particle surface.

FIG. 6 shows the dynamic range for proteins observed on neat plasma vs. SP-003, SP-007, and SP-011 particles by comparison to a compiled database from Keshishian et al. (Mol Cell Proteomics. 2015 September; 14(9):2375-93. doi: 10.1074/mcp.M114.046813. Epub 2015 Feb. 27.)(top panel).

FIG. 7 shows a correlation of the maximum intensities of particle corona proteins vs. plasma proteins to the published concentration of the same proteins.

FIG. 8 shows the reproducibility of particle corona intensities for each particle type (SP-003, SP-007, and SP-011) as demonstrated by three replicates using the same plasma sample.

FIG. 9A shows a schematic for synthesis of SPION core.

FIG. 9B shows a schematic for synthesis of silica-coated SPION (SP-003).

FIG. 9C shows a schematic for synthesis of vinyl group functionalized SPION.

FIG. 9D shows a schematic for synthesis of poly(N-(3-(dimethylamino)propyl) methacrylamide) (PDMAPMA)-coated SPION (SP-007).

FIG. 9E shows a schematic for synthesis of poly(oligo(ethylene glycol) methyl ether methacrylate) (POEGMA)-coated SPION (SP-011).

FIG. 10 shows the linearity of measurements for C-reactive proteins (CRP) on the SP-007 nanoparticles in a spike-recovery experiment for four different peptides.

FIG. 11 shows the linearity of measurement for peptide features of Angiogenin in a spike-recovery experiment.

FIG. 12 shows the linearity of measurement for peptide features of S10A8 in a spike-recovery experiment.

FIG. 13 shows the linearity of measurement for peptide features of S10A9 in a spike-recovery experiment.

FIG. 14 shows the linearity of measurement for peptide features of C-reactive protein (CRP) in a spike-recovery experiment.

FIG. 15 shows matching and coverage of a particle panel of the 10 distinct particle types to a 5,304-plasma protein database of MS intensities. The ranked intensities for the database proteins are shown in the top panel (“Database”), the intensities for proteins from simple plasma MS evaluation are shown in the second panel (“Plasma”) and the intensities for the optimal 10-particle type panel are shown in the remaining panels. The most intense protein is in the upper left corner of the panel, and the least intense protein is in the lower right corner of the panel. The plasma protein intensities database is from Keshishian et al. (2015). Multiplexed, Quantitative Workflow for Sensitive Biomarker Discovery in Plasma Yields Novel Candidates for Early Myocardial Injury. Molecular & Cellular Proteomics, 14(9), 2375-2393.

FIG. 16 shows coverage of protein-protein interaction map by proteins detected by the nanoparticles for A) proteins known to be present in plasma and B) all proteins. Differences in shading indicate differences in the abundances of proteins identified on the nanoparticles.

FIG. 17 shows distribution of the number of peptides used to define each of the 2,009 protein groups as measured for the optimized 10 NP panel across the 16 subject plasma samples. The peptide counts include the razor and unique peptides as defined within an associated MaxQuant proteinGroups.txt file. 84% of the 2,009 protein groups included more than one razor and/or unique peptide to define the group.

FIG. 18 shows count of the number of protein groups (1% protein false discovery rate (FDR) from MaxQuant) as measured across the optimized 10-NP panel and across the 16 subject plasma samples (sampleID). A total of 2,009 protein groups were defined by the LCMS data processed through MaxQuant, with 84% being defined by more than one razor and/or unique peptide sequence.

FIG. 19 shows significantly enriched annotations from A) Gene Ontology Cellular Component (GOCC), B) Gene Ontology Biological Process (GOBP), C) Protein families (Pfam), D) Kyoto Encyclopedia of Genes and Genomes (KEGG) comparing one NP corona versus all others (difference of median protein group abundance) in a 1D annotation enrichment. The following thresholds were applied: annotation group size >10, B.H. FDR <5% for at least one corona. Hierarchical clustering is based on the 1D score. The 1D score ranges from −1 to 1, dark shading indicates depletion, light shading indicates enrichment.

FIG. 20A and FIG. 20B show schematics illustrating a method to identify protein-protein interactions (PPIs) present in biomolecule corona. FIG. 20A shows a protein (dark gray small ovals 2005) that binds directly to two particle types with distinct physicochemical properties (“P1” and “P2”). Because the protein binds directly to both particle types, the measured protein intensity is well correlated on both particle types across multiple samples. Protein intensity across different samples (e.g., a protein intensity pattern) for each particle type is depicted by the jagged line to the right of each particle. FIG. 20B shows a first protein (dark gray small ovals 2005) that binds directly to a first particle type (“P1”) and binds indirectly to a second particle type (“P2”). The first protein 2005 binds to the second particle type P2 through protein-protein interactions with a second protein (lighter gray small oval 2010). Because the first protein binds to the second particle type through the second protein, the protein intensities of the first protein and the second protein on the second particle type are well correlated across multiple samples. Since the first protein binds directly to the first particle type but indirectly to the second particle type, the first protein intensity is not well correlated on the first particle type and the second particle type across multiple samples. Protein intensity across different samples for each protein on particle type is depicted by the jagged line to the right of each protein and particle type.

FIG. 21 shows distributions of protein correlations across multiple subject samples for two different particle types (P39 and P65). The top plot shows correlations of identified proteins across 288 samples between the two particle types. The bottom plot shows pairwise correlations for all protein parings on each of the two particle types. Protein pairings which showed high correlation within the two particle types (indicated by the box on the right side of the bottom plot) and where one of protein of the pair showed low correlation between the two particle types (indicated by the box on the left side of the top plot) were identified as potential protein-protein interactions.

FIG. 22 shows a plot of the protein-protein interaction candidates identified in FIG. 21. The x-axis of each plot shows the correlation of the identified proteins between the two particle types (as plotted in the top panel of FIG. 21), and the y-axis of each plot shows the pairwise correlation between the protein-protein interaction candidates (as plotted in the bottom panel of FIG. 21) on either the P39 particle type (left plot) or the P65 particle type (right plot). Interactions falling in the zone of high correlation (>0.5) on the y-axis and the zone of loose correlation on the x-axis (<|0.5|), identified by the boxed regions, correspond to potential protein-protein interactions.

FIG. 23 shows a plot of the protein-protein interaction candidates identified in FIG. 21 and plotted in FIG. 22. The x-axis of each plot shows the average of the correlation of a protein between two particles and the pairwise correlation of two proteins interaction candidates on the same particle type (P39, left plot, or P65, right plot). The y-axis shows the difference between the pairwise correlation of two proteins as interaction candidates on the same particle type and the correlation of a protein between two particles. Protein pairs with high difference between correlations, denoted by boxes, represent protein pairs with high potential for protein-protein interactions.

FIG. 24 shows a table of correlation values for potential protein-protein interaction pairs identified from the data plotted in FIG. 21-FIG. 23. Initial correlation values (“Corr_I”) indicate the correlation between the protein intensity of the initial protein (“Initial”) on the P39 and P65 particle types. Anchor correlation values (“Corr_A”) indicate the correlation between the protein intensity of the initial protein and the anchor protein (“Anchor”) on the same particle type (“Particle”). The protein-protein interaction score from the String database is provided where applicable.

FIG. 25 shows a schematic of a protein corona analysis assay, also referred to as a Proteograph assay, performed on a biofluid.

FIG. 26 shows a schematic of a protein corona analysis assay, also referred to as a Proteograph assay, to identify protein fingerprints on multiple particle types (e.g., “biosensors”).

FIG. 27 shows a schematic of primary proteins, secondary proteins, tertiary proteins, and so on, interacting with a particle. Primary proteins are proteins which are aggregated primarily through their direct interactions with the particle surface. Secondary proteins are proteins which are aggregated primarily through their interactions with primary proteins. Tertiary proteins are proteins which are aggregated primarily through their interactions with secondary proteins. Additional protein layers may also form.

FIG. 28 shows protein-protein interaction maps of biological and physical protein-protein interactions from the STRING public database (string-db.org). Protein-protein interaction maps were colored by whether or not a protein is identified in a corona of either a P-033 particle type (left plot) or a S-064 particle type (right plot). Proteins that were identified in the particle corona are lightly shaded, and proteins that were not identified in the particle corona are darkly shaded. Patterns present in each protein-protein interaction map indicated that the patterns are different for each particle type and that the patterns are non-random, indicating that there is a relationship between the proteins present in the protein corona and the underlying biology represented by the protein-protein interaction map.

FIG. 29 shows a table of probabilities that a particle sampled the observed number of proteins from that group based on particle type, shown in columns, and protein cluster, shown in rows. Cell shading depicts whether the protein cluster is over represented or under represented on the given particle type. Light shading indicates that the protein cluster was underrepresented. Dark shading indicates that the protein cluster was over represented. Moderate shading can indicate that the identification of the protein cluster was commensurate with random sampling. Some clusters are consistently over or under represented across particles. Some clusters show differential behavior across particles.

FIG. 30A-D show hub proteins (FIG. 30A and FIG. 30C) and protein domains (FIG. 30B and FIG. 30D) common to many proteins in each of two under represented protein clusters, cluster 17 (FIG. 30A and FIG. 30B) and cluster 18 (FIG. 30C and FIG. 30D).

FIG. 31 shows a schematic illustrating a method to determine both primary and secondary proteins using protein corona analysis. Secondary proteins in a protein corona may be removed biochemically while primary proteins remain attached to the particle. With a diverse set of particles and a sufficient number of protein coronas, protein-protein interactions may be identified. If protein B is only observed as a secondary protein when protein A is present as a primary protein (or vice-versa), then a protein-protein interaction between protein A and protein B is identified.

FIG. 32 shows a computer system that is programmed or otherwise configured to implement methods provided herein.

FIG. 33 shows the number of protein groups identified on 9 different types of particles following collection from human plasma.

FIG. 34A-J show the human plasma abundances of proteins collected onto 9 different types of proteins from human plasma. Panel A provides an overlay of protein abundance data for all 9 particle types. Panels B-J individually show the human plasma abundances for ubiquitin functionalized particles (Panel B), dextran functionalized particles (Panel C), cis-ubiquitin functionalized particles (Panel D), polystyrene carboxyl functionalized particles (Panel E), poly(N-(3-(dimethylamino)propyl) methacrylamide) (PDMAPMA)-coated SPIONs (Panel F), Silica-coated SPIONs (Panel G), phosphate sugar functionalized particles (Panel H), amine functionalized (Panel I), and N-(3-Trimethoxysilylpropyl)diethylenetriamine coated SPIONs (Panel J). Panels B-J provide vertical lines indicating the 25^{th} percentile, 50^{th} percentile, and 75^{th} percentile proteins identified on each particle in terms of their human plasma abundances.

FIG. 34K shows the number and relative concentrations of protein groups collected on each of the 9 types of particles overviewed in FIG. 34A-J.

FIG. 35A shows the total mass of protein collected on each of 9 particle types upon contacting human plasma.

FIG. 35B displays the number of protein groups collected on each of the 9 particle types from FIG. 35A as a function of the mass of the total mass of protein collected.

FIG. 36A provides an UpSet plot summarizing the shared types of protein groups identified on the 9 particle types provided in FIG. 35A.

FIG. 36B is shows the number of identified protein groups that are unique to ubiquitin functionalized (S-164-001) and dextran functionalized (P-073) particles as well as the number of protein groups common both particle types (650).

FIG. 37A illustrates the degrees of correlation among identified protein groups between the 9 particle types from FIG. 35A.

FIG. 37B provides a principle component analysis plot for the protein groups collected on the particle types from FIG. 35A.

FIG. 38A shows the Pearson correlations for protein groups collected on ubiquitin functionalized particles (S-164-001) and dextran functionalized particles (P-073-010 and P-073-011).

FIG. 38B provides false discovery rate (FDR) adjusted p-values for 100 plasma protein classes observed on ubiquitin functionalized and dextran coated particles. FIG. 38C-D highlight specific portions of FIG. 38B.

FIG. 38E provides p-values for the protein classes collected on the dextran and ubiquitin functionalized particle of FIG. 38A.

FIG. 38F illustrates the number of protein groups identified on the ubiquitin functionalized and dextran functionalized particles of FIG. 38A.

FIG. 38G provides a principle component analysis plot for the multiple plasma assay replicates performed with the particles of FIG. 38A.

FIG. 38H provides false discovery rate (FDR) adjusted p-values for about 100 plasma protein classes observed on the particles of FIG. 35A.

FIG. 39A shows Jaccard indices for the proteins identified on the particles of FIG. 35A across multiple human plasma assays.

FIG. 39B provides Jaccard index comparisons for the proteins identified in separate assays on the particles of FIG. 35A.

FIG. 40A provides the proportions of platelet markers among proteins collected on the particles of FIG. 35A.

FIG. 40B shows the platelet indices from FIG. 40A plotted as a function of the number of protein groups identified on each particle.

FIG. 41A shows the distribution of mass spectrometric signal intensities for non-ubiquitin associated (‘Background’) proteins identified on dextran functionalized particles (P-073-010 & P-073-011), ubiquitin functionalized particles (S-164-001), and on a particle panel comprising 6 small molecule functionalized particles (V1.1_panel).

FIG. 41B shows the distribution of mass spectrometric signal intensities corresponding to ubiquitin-associated proteins identified on the particles of FIG. 41A.

FIG. 41C provides the human plasma concentrations of the ubiquitin-associated proteins identified on the particles of FIG. 41A.

FIG. 42A-G display the intensities of mass spectrometric features corresponding to five separate ubiquitin hub proteins collected on dextran functionalized particles (P-073-010 & P-073-011), ubiquitin functionalized particles (S-164-001 & S-164-002), and cis-ubiquitin functionalized particles (S-163-001 & S-163-002) and on a particle panel comprising 6 small molecule functionalized particles (V1.1 panel).

FIG. 43A illustrates a method for modifying a particle panel (V1.1) by replacing a particle type with a macromolecular functionalized particle.

FIG. 43B summarizes the protein group counts collected from human plasma onto the particle panels generated from the method outlined in FIG. 43A.

FIG. 44 illustrates a method for designing a macromolecular functionalized particle.

FIG. 45 shows the protein counts (number of proteins identified from corona analysis) for panel sizes ranging from 1 particle type to 12 particle types.

FIG. 46 illustrates a method for identifying a protein-protein interaction with biomolecule corona data.

FIG. 47 provides protein-protein interaction maps generated from the STRING PPI database using proteins detected in samples from 276 subjects. Dots represent individual proteins, with lighter shading representing higher abundance. Panel A corresponds to samples from healthy patients. Panel B corresponds to samples from patients with early stage non-small cell lung cancer (NSCLC). Panel C corresponds to samples from patients with late stage NSCLC.

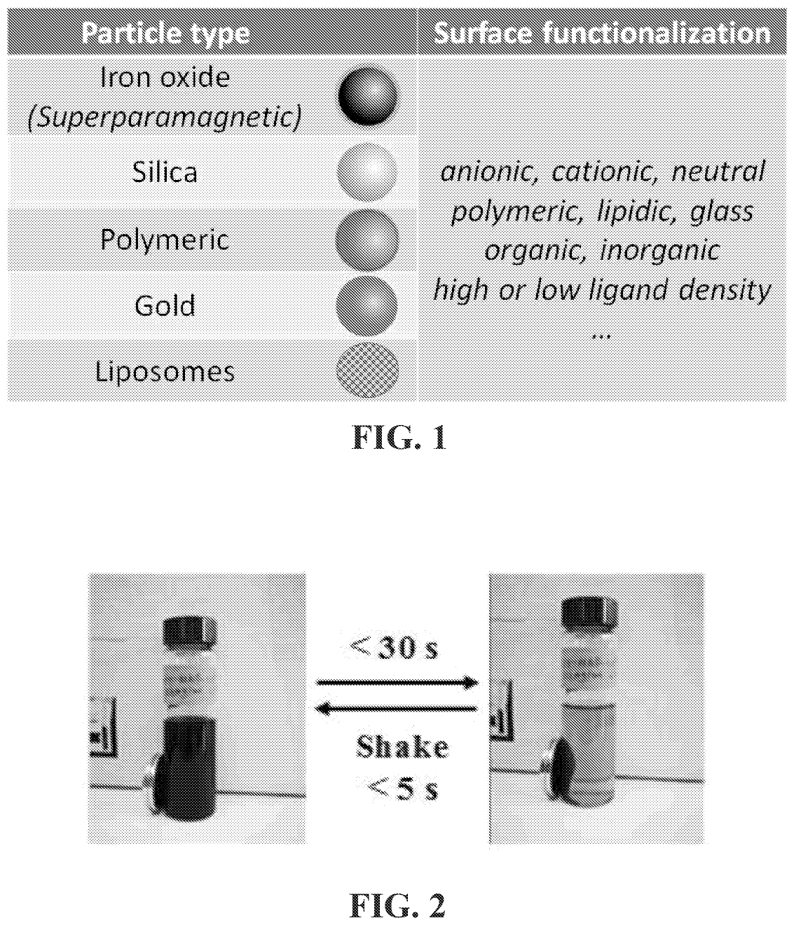

### DETAILED DESCRIPTION

Disclosed herein are methods and systems for identifying protein-protein interactions using particle panels and biomolecule corona formation. Also disclosed herein are systems and methods for one-dimensional (1D) enrichment analysis between protein annotations and particle physicochemical properties. Interactions within particle corona may reveal correlations by 1D enrichment analysis between protein annotations and particle biophysicochemical properties. There may be specific relationships at the particle biological surface.

The methods described herein may be used to identify protein-protein interactions (PPIs), for example in a biological sample. Protein-protein interactions constitute a deep layer of the complex human proteome. Based solely on sequence, it is estimated that the human proteome comprises more than 10⁶ unique proteins. Post-translational modifications (PTMs) augment this diversity, potentially increasing the number of unique human proteins beyond 10⁷. However, structure and chemical functionalization alone can be insufficient for predicting or assessing protein activity, as functional interactions between proteins themselves (e.g., protein-protein interactions) can be a major determinant of protein behavior. Thus, identifying protein-protein interactions can be essential for identifying a biological state, such as a metabolic state or disease.

Nonetheless, identifying protein-protein interactions has remained a major challenge in the field of diagnostics. Assaying for protein-protein interactions is typically slow, user-intensive, and narrowly focused. Many assays, such as pull down and co-immunoprecipitation, scan for interactions by a selected type of protein, rather than between any pair of proteins within a sample. Furthermore, such assays typically lack the ability to determine whether a protein-protein interaction is present within a cell or organism, and thus have limited diagnostic utility.

Disclosed herein are rapid and facile methods for identifying potential pluralities of protein-protein interactions in a biological sample. A protein-protein interaction (PPI) may comprise direct or indirect interactions between two or more proteins. An interaction may comprise hydrogen bonds, Van der Waals forces, ionic bonds, polar interactions, salt bridges, substrate co-complexation, leucine zippers, complementary surface structures, hydrophobic interactions, or a combination thereof. A protein-protein interaction may be identified by correlating protein intensities (e.g., intensities identified by mass spectrometry) measured in two or more samples across particle types and within particle types. Protein corona analysis may be performed on two or more samples using a particle panel comprising two or more particle types. Protein identities and intensities may be determined for proteins present in the biomolecule corona corresponding to a particular sample and a particular particle type.

A biomolecule corona may include nucleic acids, small molecules, proteins, lipids, polysaccharides, or any combination thereof, adsorbed to the surface of a particle form a sample in which the particle is incubated. nucleic acid, a small molecule, a protein, a lipid, a polysaccharide, or any combination thereof.

A biomolecule corona may comprise a primary corona and a secondary corona. A primary corona may comprise proteins that directly interact with the surface of the particle. A secondary corona may comprise proteins that indirectly interact with the surface of the particle, for example by binding to proteins in the primary corona. A protein may be identified in two or more samples on a single particle type. The protein intensity measured on the single particle type across the two or more samples may be used to generate a protein intensity pattern corresponding to the protein and the particle type.

A protein-protein interaction may be identified by contacting two or more samples with two or more particle types. For example, a protein-protein interaction may be identified by contacting a sample with 2 to 5 particle types. A protein-protein interaction may be identified by contacting a sample with 3 to 5 particle types. A protein-protein interaction may be identified by contacting a sample with 4 to 6 particle types. A protein-protein interaction may be identified by contacting a sample with 4 to 8 particle types. A protein-protein interaction may be identified by contacting a sample with 5 to 8 particle types. A protein-protein interaction may be identified by contacting a sample with 6 to 8 particle types. A protein-protein interaction may be identified by contacting a sample with 6 to 12 particle types. A protein-protein interaction may be identified by contacting a sample with 8 to 12 particle types. A protein-protein interaction may be identified by contacting a sample with 10 to 15 particle types.

In some embodiments, the two or more particle types may be contacted to at least about 5, at least about 10, at least about 15, at least about 20, at least about 25, at least about 50, at least about 100, at least about 150, at least about 200, at least about 250, at least about 300, at least about 350, at least about 400, at least about 450, at least about 500, at least about 550, at least about 600, at least about 650, at least about 700, at least about 750, at least about 800, at least about 850, at least about 900, at least about 950, at least about 1000, at least about 1500, at least about 2000, at least about 2500, at least about 3000, at least about 3500, at least about 4000, at least about 4500, at least about 5000 samples. The samples may be in a single sample volume.

A sample may be labeled with a sample-specific tag (e.g., a sample-specific mass tag). Two or more samples labeled with sample-specific mass tags may be assayed using protein corona analysis with mass spectrometry to identify protein-protein interactions present in the two or more samples. The two or more samples are contacted with at least about 2, at least about 3, at least about 4, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20, at least about 25, at least about 30, at least about 35, at least about 40, at least about 45, at least about 50, at least about 60, at least about 70, at least about 80, at least about 90, at least about 100, at least about 110, at least about 120, at least about 130, at least about 140, at least about 150, or at least about 200 particle types. The two or more particle types may comprise a particle type provided in TABLES 1, 7, 9, 10, 11, or 17.

Protein intensity patterns may be generated for two or more protein-particle type combinations. For example, a first protein pattern may be generated for a first protein on a first particle type. A second protein pattern may be generated for the first protein on a second particle type. A third protein pattern may be generated for a second protein on the second particle type. A fourth protein pattern may be generated for the second protein on the first particle type. A protein intensity pattern may be generated for at least about 3, at least about 4, at least about 5, at least about 10, at least about 20, at least about 30, at least about 40, at least about 50, at least about 60, at least about 70, at least about 80, at least about 90, at least about 100, at least about 200, at least about 300, at least about 400, at least about 500, at least about 600, at least about 700, at least about 800, at least about 900, at least about 1000, at least about 1100, at least about 1200, at least about 1300, at least about 1400, at least about 1500, at least about 1600, at least about 1700, at least about 1800, at least about 1900, at least about 2000, at least about 2500, at least about 3000, at least about 3500, at least about 4000, at least about 4500, or at least about 5000 protein-particle type combinations.

### Protein-Protein Interactions

A correlation between two protein intensity patterns may be measured to determine a likelihood of a protein-protein interaction.

An identified protein-protein interaction may be a solution-phase protein-protein interaction, an on-particle protein-protein interaction, or a combination thereof. A protein-protein interaction may comprise hydrogen bonding, Van der Waals, ionic, exchange, hydrophobic, salt bridge-mediated, covalent, or entropic driving forces. A protein-protein interaction may comprise at least 2, at least 3, at least 4, at least 5, at least 6, at least 8, at least 10, at least 12 or more proteins. A protein-protein interaction may indicate the presence of a protein aggregate, such as an alpha-synuclein aggregate. A protein-protein interaction may comprise a denatured or partially denatured protein.

A protein-protein interaction may occur in solution, on a particle, or both. In many cases, a protein-protein interaction strength changes minimally upon binding particle binding by the interacting proteins. Accordingly, a protein-protein interaction may drive the binding of a protein to a particle. For example, the second protein may have a greater affinity for the first protein in the primary corona of a particle than for the particle, itself, and associate more strongly with the particle when the first protein is present in a sample. In such cases, the protein-protein interaction between the first and second proteins may be detected by identifying that the association between the first and second proteins is greater than either or both of the associations of the first and second proteins to the particle.

Furthermore, a protein may alter its binding to a particle upon conversion from a first state to a second state. The change in states may comprise a change in conformation. The change in states may comprise a post-translational modification (e.g., glycosylation or prenylation, or phosphorylation). The change in states may comprise a change in substrate or cofactor binding. A protein may directly bind to a particle (e.g., occupy a primary corona) when in a first state and indirectly bind (e.g., occupy a secondary corona) when in a second state. Such a change in binding may be measured, and thus used to distinguish the state of the protein. Furthermore, the change in binding may affect protein-protein interaction formation between the protein and a second protein present in the sample. Thus, detection of a protein-protein interaction may identify a protein's state.

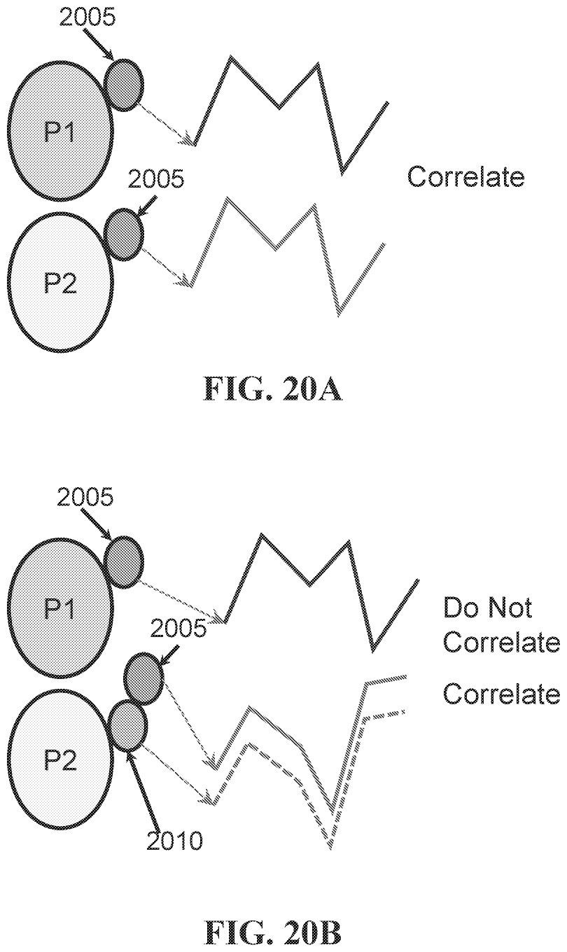

An association or correlation between two protein intensity patterns may be measured to determine a likelihood of a direct interaction between a protein and a particle type. As illustrated schematically in FIG. 20A and FIG. 20B, the presence or absence of a protein-protein interaction between a first protein and a second protein may be identified by measuring (1) a “same protein” score or correlation between a first protein 2005 intensity pattern of the first protein on a first particle type and second protein intensity pattern of the first protein on a second particle type and (2) a “same particle” score or correlation between the first protein intensity pattern of the first protein on the first particle type and a third protein intensity pattern of the second protein 2010 on the first particle type. In some cases, a protein-protein interaction may be identified between the first protein 2005 and the second protein 2010 if the same protein correlation is low and the same particle correlation is high.

A protein-protein interaction may be identified between the first protein and the second protein by a same particle score or correlation. The identification may comprise determining that a same particle score or correlation is greater than the same particle scores or correlations for other protein pairs on the same particle. For example, a protein-protein interaction may be identified by a same particle score comprising a Pearson correlation and 2.5 standard deviations higher than the mean same particle score for protein pairs identified from a sample. A protein-protein interaction may be identified between the first protein and the second protein by a plurality of same particle scores above a predefined cutoff determined by measuring same particle scores for known protein-protein interactions.

Strength of the protein-protein interaction may be quantified from the same particle correlation or score, the same protein correlation or score, or a combination of the same particle and same protein correlation(s) or score(s). Quantifying the strength of the protein-protein interaction may comprise quantifying the thermodynamics of the first protein binding to the second protein, or may comprise quantifying an upper or lower bound for the thermodynamics of the first protein binding to the second protein.

A protein-protein interaction may comprise a hub protein. A hub protein may be a protein which comprises a protein-protein interaction with a plurality of different proteins. For instance, a hub protein may comprise protein-protein interactions with 2 or more different proteins. A hub protein may comprise protein-protein interactions with 3 or more different proteins. A hub protein may comprise protein-protein interactions with 4 or more different proteins. A hub protein may comprise protein-protein interactions with 5 or more different proteins. A hub protein may comprise protein-protein interactions with 6 or more different proteins. A hub protein may comprise protein-protein interactions with 10 or more different proteins. A hub protein may comprise protein-protein interactions with 15 or more different proteins. A hub protein may comprise protein-protein interactions with 30 or more different proteins. A hub protein may comprise protein-protein interactions with 50 or more proteins. A hub protein may comprise a protein-protein interaction with a structural motif (e.g., a zinc finger) common to a group or class of proteins. The plurality of proteins bound by many hub proteins comprise a common physical or structural characteristic, such as a particular post-translational modification (e.g., a glycosylation pattern) or a particular tertiary structural motif. Thus, hub proteins can be useful in identifying clusters of proteins capable of forming protein-protein interactions. Identification of a hub protein may elucidate a large number of protein-protein interactions. A hub protein, once identified, may be used as a bait molecule or as a macromolecular functionalization on a particle to collect a set of proteins that form protein-protein interactions with the hub protein.

A same protein score may be based on a same protein correlation. A same particle score may be based on a same particle correlation.

A protein-protein interaction may be identified between a first protein and a second protein if a same protein correlation is no more than about 0.6, no more than about 0.58, no more than about 0.56, no more than about 0.55, no more than about 0.54, no more than about 0.52, no more than about 0.5, no more than about 0.48, no more than about 0.46, no more than about 0.45, no more than about 0.44, no more than about 0.42, no more than about 0.4, no more than about 0.38, no more than about 0.36, no more than about 0.35, no more than about 0.34, no more than about 0.32, no more than about 0.3, no more than about 0.28, no more than about 0.26, no more than about 0.25, no more than about 0.24, no more than about 0.22, no more than about 0.2, no more than about 0.18, no more than about 0.16, no more than about 0.15, no more than about 0.14, no more than about 0.12, or no more than about 0.1. A protein-protein interaction may be identified between a first protein and a second protein if a same particle correlation is at least about 0.4, at least about 0.42, at least about 0.44, at least about 0.45, at least about 0.46, at least about 0.48, at least about 0.5, at least about 0.52, at least about 0.54, at least about 0.55, at least about 0.56, at least about 0.58, at least about 0.6, at least about 0.62, at least about 0.64, at least about 0.65, at least about 0.66, at least about 0.68, at least about 0.7, at least about 0.72, at least about 0.74, at least about 0.75, at least about 0.76, at least about 0.78, at least about 0.8, at least about 0.82, at least about 0.84, at least about 0.85, at least about 0.86, at least about 0.88, at least about 0.9, at least about 0.92, at least about 0.94, at least about 0.95, at least about 0.96, at least about 0.98, or about 1.

A protein-protein interaction may be identified by comparing same protein and same particle correlations for two or more protein pairings. The two or more protein parings may be identified randomly. Same protein and same particle correlations may be compared for at least about 2, at least about 3, at least about 4, at least about 5, at least about 10, at least about 15, at least about 20, at least about 25, at least about 50, at least about 100, at least about 150, at least about 200, at least about 250, at least about 300, at least about 350, at least about 400, at least about 450, at least about 500, at least about 550, at least about 600, at least about 650, at least about 700, at least about 750, at least about 800, at least about 850, at least about 900, at least about 950, at least about 1000, at least about 1500, at least about 2000, at least about 2500, at least about 3000, at least about 3500, at least about 4000, at least about 4500, or at least about 5000 protein pairings.

The methods provided herein to identify a protein-protein interaction may be completed in no more than about 450 minutes, no more than about 420 minutes, no more than about 390 minutes, no more than about 360 minutes, no more than about 330 minutes, no more than about 330 minutes, no more than about 300 minutes, no more than about 270 minutes, no more than about 240 minutes, no more than about 210 minutes, 180 minutes, no more than about 160 minutes, no more than about 140 minutes, no more than about 120 minutes, no more than about 110 minutes, no more than about 100 minutes, no more than about 90 minutes, no more than about 80 minutes, no more than about 70 minutes, no more than about 60 minutes, no more than about 55 minutes, no more than about 50 minutes, no more than about 45 minutes, no more than about 40 minutes, no more than about 35 minutes, no more than about 30 minutes, no more than about 25 minutes, no more than about 20 minutes, no more than about 15 minutes, no more than about 10 minutes, or no more than about 5 minutes.

An advantage of the methods and compositions of the present disclosure is the ability to analyze small sample volumes. The methods described herein may be performed using a sample volume of no more than about 0.01 mL, no more than about 0.02 mL, no more than about 0.03 mL, no more than about 0.05 mL, 0.1 mL, no more than about 0.2 mL, no more than about 0.3 mL, no more than about 0.4 mL, no more than about 0.5 mL, no more than about 0.6 mL, no more than about 0.7 mL, no more than about 0.8 mL, no more than about 0.9 mL, no more than about 1 mL, no more than about 1.1 mL, no more than about 1.2 mL, no more than about 1.3 mL, no more than about 1.4 mL, no more than about 1.5 mL, no more than about 1.6 mL, no more than about 1.7 mL, no more than about 1.8 mL, no more than about 1.9 mL, no more than about 2 mL, no more than about 2.1 mL, no more than about 2.2 mL, no more than about 2.3 mL, no more than about 2.4 mL, or no more than about 2.5 mL. The sample may be a biological sample. Particles may be suspended in the solution, or the sample may be mixed with a solution or suspension comprising particles. The sample may be mixed in a ratio of at least a 20:1, at least a 15:1, at least a 12:1, at least a 10:1, at least an 8:1, at least a 5:1, at least a 4:1, at least a 3:1, at least a 2:1, at least a 3:2, at least a 1:1, at least a 2:3, at least a 1:2, at least a 1:3, at least a 1:4, at least a 1:5, at least a 1:8, at least a 1:10, at least a 1:12, at least a 1:15, or at least a 1:20 with a solution or suspension comprising particles. The sample may be mixed in a ratio of at most a 20:1, at most a 15:1, at most a 12:1, at most a 10:1, at most an 8:1, at most a 5:1, at most a 4:1, at most a 3:1, at most a 2:1, at most a 3:2, at most a 1:1, at most a 2:3, at most a 1:2, at most a 1:3, at most a 1:4, at most a 1:5, at most a 1:8, at most a 1:10, at most a 1:12, at most a 1:15, or at most a 1:20 with a solution or suspension comprising particles. For example, a 10 μL portion of a sample may be mixed with 50 μL of a suspension comprising particles.

The methods provided herein may identify a plurality of protein-protein interactions in a biological sample. Many analysis methods are limited to identifying protein-protein interactions between an elected protein (e.g., a protein immobilized within a column) and proteins in a purified sample. In this sense, other methods for detecting protein-protein interactions may be biased, as identification of a protein-protein interaction depends on the initial election of a selected protein. Methods of the present disclosure can identify protein-protein interactions between any proteins (e.g., between any 2 or 3 proteins) in a sample. The methods of the present disclosure are unbiased in that protein-protein interactions are not identified merely based on an initially elected protein. Thus, the methods of the present disclosure are well suited for identifying new protein-protein interactions that were not previously known, and for identifying protein-protein interactions that are pertinent to native intracellular and intra-organismal conditions (i.e., identifying a protein-protein interaction that is present within the organism from which a biological sample was obtained). Analysis of biomolecule corona data may identify 1-3 protein-protein interactions in a biological sample. Analysis of biomolecule corona data may identify at least 2 protein-protein interactions in a biological sample Analysis of biomolecule corona data may identify at least 3 protein-protein interactions in a biological sample. Analysis of biomolecule corona data may identify at least 5 protein-protein interactions in a biological sample. Analysis of biomolecule corona data may identify at least 8 protein-protein interactions in a biological sample. Analysis of biomolecule corona data may identify at least 10 protein-protein interactions in a biological sample. Analysis of biomolecule corona data may identify at least 15 protein-protein interactions in a biological sample. Analysis of biomolecule corona data may identify at least 20 protein-protein interactions in a biological sample. Analysis of biomolecule corona data may identify at least 30 protein-protein interactions in a biological sample. Analysis of biomolecule corona data may identify at least 50 protein-protein interactions in a biological sample.

A protein-protein interaction may be specific to a sample type. For example, a protein-protein interaction may be identified in a first sample type but not in a second sample type. In some instances, the presence or absence of a protein-protein interaction may depend on a biological state of a sample. Identification of a protein-protein interaction may be used to determine a biological state. A protein-protein interaction may be associated with a biological state using an analysis method. The analysis method may weight a datapoint (e.g., an identified protein or protein group) based on an identified protein-protein interaction. Furthermore, the analysis method may utilize a protein-protein interaction as a datapoint (e.g., comparable to the presence or abundance of a particular protein). A protein-protein interaction datapoint may comprise a weight, such as a same particle score or a Pearson correlation. Accordingly, two protein-protein interactions identified in a sample may provide differently weighted contributions to the identification of a biological state. An analysis method may cluster data based on an identified protein-protein interaction. As a non-limiting example, two cancer states may be distinguished by the identification of a protein-protein interaction. For example, a number of protein-protein interactions in the polo-like kinase 1 (PLK1) signaling pathway can be specific to late stage colon cancer. Thus, an analysis method could first identify colon cancer from biomolecule corona data, and then determine the stage of the colon cancer by identifying at least one protein-protein interaction from among the biomolecule corona data.

The biological state may be a disease state. A disease state may be cancer or a neurological disease state (e.g., Alzheimer's disease). The biological state may be a healthy state. For example, a protein-protein interaction may present in biological samples from subjects with cancer, and the protein-protein interaction may not be present in biological samples from subjects without cancer, or a protein-protein interaction may present in biological samples from subjects without cancer, and the protein-protein interaction may not be present in biological samples from subjects with cancer. A biological state may comprise a phenotype. A protein-protein interaction that has been identified to correspond to a biological state, for example using the protein corona analysis methods disclosed herein, may be used to identify a biological state of a sample corresponding to an unknown biological state. For example, a protein-protein interaction that has been identified as corresponding to cancer may be used to determine whether a subject has cancer by detecting the presence or absence of the protein-protein interaction in a biological sample from the subject. In some instances, a protein-protein interaction present in a biological sample may be compared to a reference protein-protein interaction (e.g., a protein-protein interaction identified by ELISA, immunofluorescence, yeast-hybrid, size exclusion chromatography, surface plasmon resonance, or any combination thereof

Disease States

The methods, compositions, and systems described herein can be used to determine a disease state, and/or prognose or diagnose a disease or disorder. The diseases or disorders contemplated include, but are not limited to, for example, cancer, cardiovascular disease, endocrine disease, inflammatory disease, a neurological disease and the like.

The methods, compositions, and systems described herein can be used to determine, prognose, and/or diagnose a cancer disease state. The term “cancer” is meant to encompass any cancer, neoplastic and preneoplastic disease that is characterized by abnormal growth of cells, including tumors and benign growths. Cancer may, for example, be lung cancer, pancreatic cancer, or skin cancer. In many cases, the methods, compositions and systems described herein are not only able to diagnose cancer (e.g. determine if a subject (a) does not have cancer, (b) is in a pre-cancer development stage, (c) is in early stage of cancer, (d) is in a late stage of cancer) but are able to determine the type of cancer.

The methods, compositions, and systems of the present disclosure can additionally be used to detect other cancers, such as acute lymphoblastic leukemia (ALL); acute myeloid leukemia (AML); cancer in adolescents; adrenocortical carcinoma; childhood adrenocortical carcinoma; unusual cancers of childhood; AIDS-related cancers; kaposi sarcoma (soft tissue sarcoma); AIDS-related lymphoma (lymphoma); primary cns lymphoma (lymphoma); anal cancer; appendix cancer—see gastrointestinal carcinoid tumors; astrocytomas, childhood (brain cancer); atypical teratoid/rhabdoid tumor, childhood, central nervous system (brain cancer); basal cell carcinoma of the skin—see skin cancer; bile duct cancer; bladder cancer; childhood bladder cancer; bone cancer (includes ewing sarcoma and osteosarcoma and malignant fibrous histiocytoma); brain tumors; breast cancer; childhood breast cancer; bronchial tumors, childhood; burkitt lymphoma—see non-hodgkin lymphoma; carcinoid tumor (gastrointestinal); childhood carcinoid tumors; carcinoma of unknown primary; childhood carcinoma of unknown primary; cardiac (heart) tumors, childhood; central nervous system; atypical teratoid/rhabdoid tumor, childhood (brain cancer); embryonal tumors, childhood (brain cancer); germ cell tumor, childhood (brain cancer); primary cns lymphoma; cervical cancer; childhood cervical cancer; childhood cancers; cancers of childhood, unusual; cholangiocarcinoma—see bile duct cancer; chordoma, childhood; chronic lymphocytic leukemia (CLL); chronic myelogenous leukemia (CML); chronic myeloproliferative neoplasms; colorectal cancer; childhood colorectal cancer; craniopharyngioma, childhood (brain cancer); cutaneous t-cell lymphoma—see lymphoma (mycosis fungoides and sèzary syndrome); ductal carcinoma in situ (DCIS)—see breast cancer; embryonal tumors, central nervous system, childhood (brain cancer); endometrial cancer (uterine cancer); ependymoma, childhood (brain cancer); esophageal cancer; childhood esophageal cancer; esthesioneuroblastoma (head and neck cancer); ewing sarcoma (bone cancer); extracranial germ cell tumor, childhood; extragonadal germ cell tumor; eye cancer; childhood intraocular melanoma; intraocular melanoma; retinoblastoma; fallopian tube cancer; fibrous histiocytoma of bone, malignant, and osteosarcoma; gallbladder cancer; gastric (stomach) cancer; childhood gastric (stomach) cancer; gastrointestinal carcinoid tumor; gastrointestinal stromal tumors (GIST) (soft tissue sarcoma); childhood gastrointestinal stromal tumors; germ cell tumors; childhood central nervous system germ cell tumors (brain cancer); childhood extracranial germ cell tumors; extragonadal germ cell tumors; ovarian germ cell tumors; testicular cancer; gestational trophoblastic disease; hairy cell leukemia; head and neck cancer; heart tumors, childhood; hepatocellular (liver) cancer; histiocytosis, langerhans cell; hodgkin lymphoma; hypopharyngeal cancer (head and neck cancer); intraocular melanoma; childhood intraocular melanoma; islet cell tumors, pancreatic neuroendocrine tumors; kaposi sarcoma (soft tissue sarcoma); kidney (renal cell) cancer; langerhans cell histiocytosis; laryngeal cancer (head and neck cancer); leukemia; lip and oral cavity cancer (head and neck cancer); liver cancer; lung cancer (non-small cell and small cell); childhood lung cancer; lymphoma; male breast cancer; malignant fibrous histiocytoma of bone and osteosarcoma; melanoma; childhood melanoma; melanoma, intraocular (eye); childhood intraocular melanoma; merkel cell carcinoma (skin cancer); mesothelioma, malignant; childhood mesothelioma; metastatic cancer; metastatic squamous neck cancer with occult primary (head and neck cancer); midline tract carcinoma with nut gene changes; mouth cancer (head and neck cancer); multiple endocrine neoplasia syndromes; multiple myeloma/plasma cell neoplasms; mycosis fungoides (lymphoma); myelodysplastic syndromes, myelodysplastic/myeloproliferative neoplasms; myelogenous leukemia, chronic (cml); myeloid leukemia, acute (aml); myeloproliferative neoplasms, chronic; nasal cavity and paranasal sinus cancer (head and neck cancer); nasopharyngeal cancer (head and neck cancer); neuroblastoma; non-hodgkin lymphoma; non-small cell lung cancer; oral cancer, lip and oral cavity cancer and oropharyngeal cancer (head and neck cancer); osteosarcoma and malignant fibrous histiocytoma of bone; ovarian cancer; childhood ovarian cancer; pancreatic cancer; childhood pancreatic cancer; pancreatic neuroendocrine tumors (islet cell tumors); papillomatosis (childhood laryngeal); paraganglioma; childhood paraganglioma; paranasal sinus and nasal cavity cancer (head and neck cancer); parathyroid cancer; penile cancer; pharyngeal cancer (head and neck cancer); pheochromocytoma; childhood pheochromocytoma; pituitary tumor; plasma cell neoplasm/multiple myeloma; pleuropulmonary blastoma; pregnancy and breast cancer; primary central nervous system (CNS) lymphoma; primary peritoneal cancer; prostate cancer; rectal cancer; recurrent cancer; renal cell (kidney) cancer; retinoblastoma; rhabdomyosarcoma, childhood (soft tissue sarcoma); salivary gland cancer (head and neck cancer); sarcoma; childhood rhabdomyosarcoma (soft tissue sarcoma); childhood vascular tumors (soft tissue sarcoma); ewing sarcoma (bone cancer); kaposi sarcoma (soft tissue sarcoma); osteosarcoma (bone cancer); soft tissue sarcoma; uterine sarcoma; sèzary syndrome (lymphoma); skin cancer; childhood skin cancer; small cell lung cancer; small intestine cancer; soft tissue sarcoma; squamous cell carcinoma of the skin—see skin cancer; squamous neck cancer with occult primary, metastatic (head and neck cancer); stomach (gastric) cancer; childhood stomach (gastric) cancer; t-cell lymphoma, cutaneous—see lymphoma (mycosis fungoides and sèzary syndrome); testicular cancer; childhood testicular cancer; throat cancer (head and neck cancer); nasopharyngeal cancer; oropharyngeal cancer; hypopharyngeal cancer; thymoma and thymic carcinoma; thyroid cancer; transitional cell cancer of the renal pelvis and ureter (kidney (renal cell) cancer); carcinoma of unknown primary; childhood cancer of unknown primary; unusual cancers of childhood; ureter and renal pelvis, transitional cell cancer (kidney (renal cell) cancer; urethral cancer; uterine cancer, endometrial; uterine sarcoma; vaginal cancer; childhood vaginal cancer; vascular tumors (soft tissue sarcoma); vulvar cancer; wilms tumor and other childhood kidney tumors; or cancer in young adults.

The methods, compositions, and systems of the present disclosure may be used to detect a cardiovascular disease state. As used herein, the terms “cardiovascular disease” (CVD) or “cardiovascular disorder” are used to classify numerous conditions affecting the heart, heart valves, and vasculature (e.g., veins and arteries) of the body and encompasses diseases and conditions including, but not limited to atherosclerosis, myocardial infarction, acute coronary syndrome, angina, congestive heart failure, aortic aneurysm, aortic dissection, iliac or femoral aneurysm, pulmonary embolism, atrial fibrillation, stroke, transient ischemic attack, systolic dysfunction, diastolic dysfunction, myocarditis, atrial tachycardia, ventricular fibrillation, endocarditis, peripheral vascular disease, and coronary artery disease (CAD). Further, the term cardiovascular disease refers to conditions in subjects that ultimately have a cardiovascular event or cardiovascular complication, referring to the manifestation of an adverse condition in a subject brought on by cardiovascular disease, such as sudden cardiac death or acute coronary syndrome, including, but not limited to, myocardial infarction, unstable angina, aneurysm, stroke, heart failure, non-fatal myocardial infarction, stroke, angina pectoris, transient ischemic attacks, aortic aneurysm, aortic dissection, cardiomyopathy, abnormal cardiac catheterization, abnormal cardiac imaging, stent or graft revascularization, risk of experiencing an abnormal stress test, risk of experiencing abnormal myocardial perfusion, and death.

As used herein, the ability to detect, diagnose or prognose cardiovascular disease, for example, atherosclerosis, can include determining if the patient is in a pre-stage of cardiovascular disease, has developed early, moderate or severe forms of cardiovascular disease, or has suffered one or more cardiovascular event or complication associated with cardiovascular disease.

Atherosclerosis (also known as arteriosclerotic vascular disease or ASVD) is a cardiovascular disease in which an artery-wall thickens as a result of invasion and accumulation and deposition of arterial plaques containing white blood cells on the innermost layer of the walls of arteries resulting in the narrowing and hardening of the arteries. The arterial plaque is an accumulation of macrophage cells or debris, and contains lipids (cholesterol and fatty acids), calcium and a variable amount of fibrous connective tissue. Diseases associated with atherosclerosis include, but are not limited to, atherothrombosis, coronary heart disease, deep venous thrombosis, carotid artery disease, angina pectoris, peripheral arterial disease, chronic kidney disease, acute coronary syndrome, vascular stenosis, myocardial infarction, aneurysm or stroke. In one embodiment the automated apparatuses, compositions, and methods of the present disclosure may distinguish the different stages of atherosclerosis, including, but not limited to, the different degrees of stenosis in a subject.

In some cases, the disease or disorder detected by the methods, compositions, or systems of the present disclosure is an endocrine disease. The term “endocrine disease” is used to refer to a disorder associated with dysregulation of endocrine system of a subject. Endocrine diseases may result from a gland producing too much or too little of an endocrine hormone causing a hormonal imbalance, or due to the development of lesions (such as nodules or tumors) in the endocrine system, which may or may not affect hormone levels. Suitable endocrine diseases able to be treated include, but are not limited to, e.g., Acromegaly, Addison's Disease, Adrenal Cancer, Adrenal Disorders, Anaplastic Thyroid Cancer, Cushing's Syndrome, De Quervain's Thyroiditis, Diabetes, Follicular Thyroid Cancer, Gestational Diabetes, Goiters, Graves' Disease, Growth Disorders, Growth Hormone Deficiency, Hashimoto's Thyroiditis, Hurthle Cell Thyroid Cancer, Hyperglycemia, Hyperparathyroidism, Hyperthyroidism, Hypoglycemia, Hypoparathyroidism, Hypothyroidism, Low Testosterone, Medullary Thyroid Cancer, MEN 1, MEN 2A, MEN 2B, Menopause, Metabolic Syndrome, Obesity, Osteoporosis, Papillary Thyroid Cancer, Parathyroid Diseases, Pheochromocytoma, Pituitary Disorders, Pituitary Tumors, Polycystic Ovary Syndrome, Prediabetes, Silent, Thyroiditis, Thyroid Cancer, Thyroid Diseases, Thyroid Nodules, Thyroiditis, Turner Syndrome, Type 1 Diabetes, Type 2 Diabetes, and the like.

In some cases, the disease or disorder detected by methods, compositions, or systems of the present disclosure is an inflammatory disease. As referred to herein, inflammatory disease refers to a disease caused by uncontrolled inflammation in the body of a subject. Inflammation is a biological response of the subject to a harmful stimulus which may be external or internal such as pathogens, necrosed cells and tissues, irritants etc. However, when the inflammatory response becomes abnormal, it results in self-tissue injury and may lead to various diseases and disorders. Inflammatory diseases can include, but are not limited to, asthma, glomerulonephritis, inflammatory bowel disease, rheumatoid arthritis, hypersensitivities, pelvic inflammatory disease, autoimmune diseases, arthritis; necrotizing enterocolitis (NEC), gastroenteritis, pelvic inflammatory disease (PID), emphysema, pleurisy, pyelitis, pharyngitis, angina, acne vulgaris, urinary tract infection, appendicitis, bursitis, colitis, cystitis, dermatitis, phlebitis, rhinitis, tendonitis, tonsillitis, vasculitis, autoimmune diseases; celiac disease; chronic prostatitis, hypersensitivities, reperfusion injury; sarcoidosis, transplant rejection, vasculitis, interstitial cystitis, hay fever, periodontitis, atherosclerosis, psoriasis, ankylosing spondylitis, juvenile idiopathic arthritis, Behcet's disease, spondyloarthritis, uveitis, systemic lupus erythematosus, and cancer. For example, the arthritis includes rheumatoid arthritis, psoriatic arthritis, osteoarthritis or juvenile idiopathic arthritis, and the like.

The methods, compositions, and systems of the present disclosure may detect a neurological disease state. Neurological disorders or neurological diseases are used interchangeably and refer to diseases of the brain, spine and the nerves that connect them. Neurological diseases include, but are not limited to, brain tumors, epilepsy, Parkinson's disease, Alzheimer's disease, ALS, arteriovenous malformation, cerebrovascular disease, brain aneurysms, epilepsy, multiple sclerosis, Peripheral Neuropathy, Post-Herpetic Neuralgia, stroke, frontotemporal dementia, demyelinating disease (including but are not limited to, multiple sclerosis, Devic's disease (i.e. neuromyelitis optica), central pontine myelinolysis, progressive multifocal leukoencephalopathy, leukodystrophies, Guillain-Barre syndrome, progressing inflammatory neuropathy, Charcot-Marie-Tooth disease, chronic inflammatory demyelinating polyneuropathy, and anti-MAG peripheral neuropathy) and the like. Neurological disorders also include immune-mediated neurological disorders (IMNDs), which include diseases with at least one component of the immune system reacts against host proteins present in the central or peripheral nervous system and contributes to disease pathology. IMNDs may include, but are not limited to, demyelinating disease, paraneoplastic neurological syndromes, immune-mediated encephalomyelitis, immune-mediated autonomic neuropathy, myasthenia gravis, autoantibody-associated encephalopathy, and acute disseminated encephalomyelitis.

Methods, systems, and/or apparatuses of the present disclosure may be able to accurately distinguish between patients with or without Alzheimer's disease. These may also be able to detect patients who are pre-symptomatic and may develop Alzheimer's disease several years after the screening. This provides advantages of being able to treat a disease at a very early stage, even before development of the disease.

The methods, compositions, and systems of the present disclosure can detect a pre-disease stage of a disease or disorder. A pre-disease stage is a stage at which the patient has not developed any signs or symptoms of the disease. A pre-cancerous stage would be a stage in which cancer or tumor or cancerous cells have not be identified within the subject. A pre-neurological disease stage would be a stage in which a person has not developed one or more symptom of the neurological disease. The ability to diagnose a disease before one or more sign or symptom of the disease is present allows for close monitoring of the subject and the ability to treat the disease at a very early stage, increasing the prospect of being able to halt progression or reduce the severity of the disease.

The methods, compositions, and systems of the present disclosure may detect the early stages of a disease or disorder. Early stages of the disease can refer to when the first signs or symptoms of a disease may manifest within a subject. The early stage of a disease may be a stage at which there are no outward signs or symptoms. For example, in Alzheimer's disease an early stage may be a pre-Alzheimer's stage in which no symptoms are detected yet the patient will develop Alzheimer's months or years later.

Identifying a disease in either pre-disease development or in the early states may often lead to a higher likelihood for a positive outcome for the patient. For example, diagnosing cancer at an early stage (stage 0 or stage 1) can increase the likelihood of survival by over 80%. Stage 0 cancer can describe a cancer before it has begun to spread to nearby tissues. This stage of cancer is often highly curable, usually by removing the entire tumor with surgery. Stage 1 cancer may usually be a small cancer or tumor that has not grown deeply into nearby tissue and has not spread to lymph nodes or other parts of the body.

In some cases, the methods, compositions, and systems of the present disclosure are able to detect intermediate stages of the disease. Intermediate states of the disease describe stages of the disease that have passed the first signs and symptoms and the patient is experiencing one or more symptom of the disease. For example, for cancer, stage II or III cancers are considered intermediate stages, indicating larger cancers or tumors that have grown more deeply into nearby tissue. In some instances, stage II or III cancers may have also spread to lymph nodes but not to other parts of the body.

Further, the methods, compositions, and systems of the present disclosure may be able to detect late or advanced stages of the disease. Late or advanced stages of the disease may also be called “severe” or “advanced” and usually indicates that the subject is suffering from multiple symptoms and effects of the disease. For example, severe stage cancer includes stage IV, where the cancer has spread to other organs or parts of the body and is sometimes referred to as advanced or metastatic cancer.

The methods of the present disclosure can include processing the biomolecule corona data of a sample against a collection of biomolecule corona datasets representative of a plurality of diseases and/or a plurality of disease states to determine if the sample indicates a disease and/or disease state. For example, samples can be collected from a population of subjects over time. Once the subjects develop a disease or disorder, the present disclosure allows for the ability to characterize and detect the changes in biomolecule fingerprints over time in the subject by computationally analyzing the biomolecule fingerprint of the sample from the same subject before they have developed a disease to the biomolecule fingerprint of the subject after they have developed the disease. Samples can also be taken from cohorts of patients who all develop the same disease, allowing for analysis and characterization of the biomolecule fingerprints that are associated with the different stages of the disease for these patients (e.g. from pre-disease to disease states).

In some cases, the methods, compositions, and systems of the present disclosure are able to distinguish not only between different types of diseases, but also between the different stages of the disease (e.g. early stages of cancer). This can comprise distinguishing healthy subjects from pre-disease state subjects. The pre-disease state may be stage 0 or stage 1 cancer, a neurodegenerative disease, dementia, a coronary disease, a kidney disease, a cardiovascular disease (e.g., coronary artery disease), diabetes, or a liver disease. Distinguishing between different stages of the disease can comprise distinguishing between two stages of a cancer (e.g., stage 0 vs stage 1 or stage 1 vs stage 3).

### Protein Analysis

A protein-protein interaction may be indicative of a state of a protein. A protein-protein interaction or the lack of a protein-protein interaction may indicate that a protein is in a particular conformation, has a post-translational modification, has a cofactor or substrate bound, has damage (e.g., oxidative damage), or has a particular oxidation state (e.g., a 4 electron reduced multi-copper oxidase). In such cases, a protein-protein interaction may only occur when one or more proteins is in a particular state.

One or more of a protein intensity pattern, a same protein correlation (e.g., a Pearson correlation value or a Spearman correlation value above a threshold such as 0.6 or 0.85), a same particle correlation (e.g., a standard deviation above a threshold such as 1.5 or 2), a protein pairing, or a protein-protein interaction may be used as training data for a machine learning algorithm. The machine learning algorithm may generate a trained classifier based on the training data. In some cases, the trained classifier may be used to identify a protein-protein interaction in an experimental sample.

In some cases, a protein-protein interaction may be indicative of a drug targeting pathway. The drug targeting pathway may be a signal transduction pathway. The drug targeting pathway may be associated with a disease state. A protein-protein interaction indicative of a drug targeting pathway may be identified by identifying protein-protein interactions using a particle type comprising a bait molecule. The particle may be surface modified with the bait molecule. A bait molecule may be a drug, a therapeutic agent, a small molecule, a peptide, or a protein. A bait molecule may interact with a protein in a specific conformation.

A bait molecule modified particle of the present disclosure may be used to assay for a protein in a sample, such as a complex biological sample. For example, the bait molecule may be a small molecule that is directly conjugated to the surface of the particle or passively adsorbed to the surface of the particle. The small molecule may be conjugated to the surface of the particle after synthesis of the particle or, alternatively, may be incorporated into the process of synthesizing the particle. A particle bearing a small molecule bait can be used for specific proteins of interest in a sample. One or more proteins from the sample may specifically bind the bait molecule.

In one example, a bait molecule modified particle bearing a small molecule may specifically bind a first protein from the sample. Said first protein may undergo a conformation change upon binding to the bait molecule. Upon undergoing said conformational change, the first protein may additionally bind a second protein from the sample. In some aspects, said first protein and said second protein thereby may only interact in the presence of a particle bearing the bait molecule. In other aspects, said first protein and said second protein may still bind in solution even in the absence of the particle. A bait molecule may comprise a macromolecule such as a peptide (e.g., an antibody, receptor protein, or fragment thereof), a peptoid, a polysaccharide (e.g., an alginate), or a nucleic acid (e.g., an aptamer).

A protein-protein interaction may be indicative of a drug targeting pathway if the protein-protein interaction is present in a biomolecule corona formed on a particle comprising a bait molecule (e.g., a drug). A bait molecule may be chosen to interrogate for a particular drug targeting pathway. For example, an unreactive analogue of a substrate of interest may be used as a bait molecule to assay for enzymes with an affinity for the substrate. Analogously, a signaling tag may be used as a bait molecule to assay for members of signaling pathways involving the tag. A bait molecule may comprise ubiquitin. A bait molecule may comprise dextran.

In other applications, the bait molecule modified particle may be used to probe or identify a particular protein-protein interaction indicative of a drug targeting pathway. Identifying a protein-protein interaction indicative of a drug targeting pathway may comprise contacting a sample (e.g., a biological sample) with one or more particle types, wherein one or more particle types comprise a bait molecule. A protein intensity pattern may be generated using the protein corona analysis methods described herein. One or more same protein correlations, one or more same particle correlations, or a combination thereof may be measured using two or more protein intensity patterns, as described herein. The same protein correlation, the same particle correlation, or both may be used to identify a protein-protein interaction corresponding to a drug targeting pathway. In some instances, identifying a protein-bait molecule interaction may comprise identifying a same protein score above a predetermined cutoff. In some instances, identifying a protein-bait molecule interaction may comprise identifying a same protein score of at least 0.5. In some instances, identifying a protein-bait molecule interaction may comprise identifying a same protein score of at least 0.6. In some instances, identifying a protein-bait molecule interaction may comprise identifying a same protein score of at least 0.7. In some instances, identifying a protein-bait molecule interaction may comprise identifying a same protein score of at least 0.8. In some instances, identifying a protein-bait molecule interaction may comprise identifying a same protein score of at least 0.9. In some instances, identifying a protein-bait molecule interaction may comprise identifying a same protein score of at least 0.95. In some instances, identifying a protein-bait molecule interaction may comprise identifying a same protein score of at least 0.98.

A protein-protein interaction map may cluster proteins based on their physiological functions, form of expression or activity regulation, structures, physiological localization, role in metabolic pathways, drug and agonist responsiveness, substrate type(s), cofactor type(s), or any combination therein. A protein-protein interaction map may comprise pairwise scores between proteins corresponding to their degree of similarity. For example, a protein-protein interaction generated from identified metabolic pathways may provide a high pairwise score for two proteins that participate in the same metabolic pathway, and low pairwise scores for two proteins that serve disparate physiological roles.

A protein-protein interaction map may be generated comprising two or more protein-protein interactions corresponding to the drug targeting pathway. The protein-protein interaction map may comprise at least about 2, at least 3, at least 4, at least 5, at least about 10, at least about 15, at least about 20, at least about 25, at least about 50, at least about 100, at least about 150, at least about 200, at least about 250, at least about 300, at least about 350, at least about 400, at least about 450, at least about 500, at least about 550, at least about 600, at least about 650, at least about 700, at least about 750, at least about 800, at least about 850, at least about 900, at least about 950, at least about 1000, at least about 1500, at least about 2000, at least about 2500, at least about 3000, at least about 3500, at least about 4000, at least about 4500, or at least about 5000 proteins indicative of the drug targeting pathway. A protein-protein interaction map may comprise at least 10, at least 100, at least 500, or at least 1000 non-interacting proteins. A protein-protein interaction map may comprise at least 2 protein-protein interactions, at least 5 protein-protein interactions, at least 10 protein-protein interactions, at least 25 protein-protein interactions, at least 50 protein-protein interactions, at least 100 protein-protein interactions, or at least 1000 protein-protein interactions.

A protein-protein interaction map may be used to calibrate protein-protein interaction analysis. A protein-protein interaction map may provide variable weighting coefficients (e.g., based on pairwise scores from the protein-protein interaction map) for same particle scores. For example, an analysis method may lower a same particle score for a pair of proteins with divergent metabolic roles and subcellular localizations, and raise a same particle score for a pair of proteins known to participate in the same metabolic pathway and be co-expressed by a single type of cell. Thus, identifying a protein-protein interaction may comprise calibrating a protein-protein association with a protein-protein interaction map. For example, a method of the present disclosure may comprise obtaining data comprising biomolecule information for a plurality of distinct biomolecule coronas from the sample, detecting at least a primary protein and a secondary protein in a biomolecule corona of a first particle type from the data, measuring the primary protein associated with the first particle type and the secondary protein associated with the first particle type, determining an association between the primary and secondary proteins, and calibrating the association between the primary and secondary proteins with a protein-protein interaction map.

The particle panels disclosed herein can be used to identifying a number of proteins, peptides, protein groups, or protein-protein interactions using a protein corona analysis (also referred to as “Proteograph”) workflow described herein. Protein corona analysis may comprise contacting a sample to distinct particle types (e.g., a particle panel), forming biomolecule corona on the distinct particle types, and identifying the biomolecules in the biomolecule corona (e.g., by mass spectrometry). Feature intensities, as disclosed herein, refers to the intensity of a discrete spike (“feature”) seen on a plot of mass to charge ratio versus intensity from a mass spectrometry run of a sample. These features can correspond to variably ionized fragments of peptides and/or proteins. Using the data analysis methods described herein, feature intensities can be sorted into protein groups. Protein groups refer to two or more proteins that are identified by a shared peptide sequence. Alternatively, a protein group can refer to one protein that is identified using a unique identifying sequence. For example, if in a sample, a peptide sequence is assayed that is shared between two proteins (Protein 1: XYZZX and Protein 2: XYZYZ), a protein group could be the “XYZ protein group” having two members (protein 1 and protein 2). Alternatively, if the peptide sequence is unique to a single protein (Protein 1), a protein group could be the “ZZX” protein group having one member (Protein 1). Each protein group can be supported by more than one peptide sequence. Protein detected or identified according to the instant disclosure can refer to a distinct protein detected in the sample (e.g., distinct relative other proteins detected using mass spectrometry). Thus, analysis of proteins present in distinct coronas corresponding to the distinct particle types in a particle panel yields a high number of feature intensities. This number decreases as feature intensities are processed into distinct peptides, further decreases as distinct peptides are processed into distinct proteins, and further decreases as peptides are grouped into protein groups (two or more proteins that share a distinct peptide sequence).

### Particle Types

Particle types consistent with the methods disclosed herein can be made from various materials. For example, particle materials consistent with the present disclosure include metals, polymers, magnetic materials, and lipids. Magnetic particles may be iron oxide particles. Examples of metal materials include any one of or any combination of gold, silver, copper, nickel, cobalt, palladium, platinum, iridium, osmium, rhodium, ruthenium, rhenium, vanadium, chromium, manganese, niobium, molybdenum, tungsten, tantalum, iron and cadmium, or any other material described in U.S. Pat. No. 7,749,299. A particle consistent with the compositions and methods disclosed herein may be a superparamagnetic iron oxide nanoparticle (SPION).

Examples of polymers include any one of or any combination of polyethylenes, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, or polyamines, a polyalkylene glycol (e.g., polyethylene glycol (PEG)), a polyester (e.g., poly(lactide-co-glycolide) (PLGA), polylactic acid, or polycaprolactone), or a copolymer of two or more polymers, such as a copolymer of a polyalkylene glycol (e.g., PEG) and a polyester (e.g., PLGA). The polymer may comprise a lipid-terminated polyalkylene glycol and a polyester, or any other material disclosed in U.S. Pat. No. 9,549,901.

Examples of lipids that can be used to form the particles of the present disclosure include cationic, anionic, and neutrally charged lipids. For example, particles can be made of any one of or any combination of dioleoylphosphatidylglycerol (DOPG), diacylphosphatidylcholine, diacylphosphatidylethanolamine, ceramide, sphingomyelin, cephalin, cholesterol, cerebrosides and diacylglycerols, dioleoylphosphatidylcholine (DOPC), dimyristoylphosphatidylcholine (DMPC), and dioleoylphosphatidylserine (DOPS), phosphatidylglycerol, cardiolipin, diacylphosphatidylserine, diacylphosphatidic acid, N-dodecanoyl phosphatidylethanolamines, N-succinyl phosphatidylethanolamines, N-glutarylphosphatidylethanolamines, lysylphosphatidylglycerols, palmitoyloleyolphosphatidylglycerol (POPG), lecithin, lysolecithin, phosphatidylethanolamine, lysophosphatidylethanolamine, dioleoylphosphatidylethanolamine (DOPE), dipalmitoyl phosphatidyl ethanolamine (DPPE), dimyristoylphosphoethanolamine (DMPE), distearoyl-phosphatidyl-ethanolamine (DSPE), palmitoyloleoyl-phosphatidylethanolamine (POPE) palmitoyloleoylphosphatidylcholine (POPC), egg phosphatidylcholine (EPC), distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), palmitoyloleyolphosphatidylglycerol (POPG), 16-O-monomethyl PE, 16-O-dimethyl PE, 18-1-trans PE, palmitoyloleoyl-phosphatidylethanolamine (POPE), 1-stearoyl-2-oleoyl-phosphatidyethanolamine (SOPE), phosphatidylserine, phosphatidylinositol, sphingomyelin, cephalin, cardiolipin, phosphatidic acid, cerebrosides, dicetylphosphate, and cholesterol, or any other material listed in U.S. Pat. No. 9,445,994, which is incorporated herein by reference in its entirety.

Examples of particles of the present disclosure are provided in TABLE 1.

TABLE 1
Example particles of the present disclosure

| Batch No. | Type | Particle ID | Description |
|---|---|---|---|
| S-001-001 | HX-13 | SP-001 | Carboxylate (Citrate) superparamagnetic iron oxide NPs |
| | | | (SPION) |
| S-002-001 | HX-19 | SP-002 | Phenol-formaldehyde coated SPION |
| S-003-001 | HX-20 | SP-003 | Silica-coated superparamagnetic iron oxide NPs |
| | | | (SPION) |
| S-004-001 | HX-31 | SP-004 | Polystyrene coated SPION |
| S-005-001 | HX-38 | SP-005 | Carboxylated Poly(styrene-co-methacrylic acid), P(St- |
| | | | co-MAA) coated SPION |
| S-006-001 | HX-42 | SP-006 | N-(3-Trimethoxysilylpropyl)diethylenetriamine coated |
| | | | SPION |
| S-007-001 | HX-56 | SP-007 | poly(N-(3-(dimethylamino)propyl) methacrylamide) |
| | | | (PDMAPMA)-coated SPION |
| S-008-001 | HX-57 | SP-008 | 1,2,4,5-Benzenetetracarboxylic acid coated SPION |
| S-009-001 | HX-58 | SP-009 | PVBTMAC coated |
| | | | poly(vinylbenzyltrimethylammonium chloride) |
| | | | (PVBTMAC) coated SPION |
| S-010-001 | HX-59 | SP-010 | Carboxylate, PAA coated SPION |
| S-011-001 | HX-86 | SP-011 | poly(oligo(ethylene glycol) methyl ether methacrylate) |
| | | | (POEGMA)-coated SPION |
| S-163-001 | | S-163 | Cis-ubiquitin-functionalized |
| S-164-001 | | S-164 | Ubiquitin-functionalized |
| P-033-001 | P33 | SP-333 | Carboxylate functionalized 1 μm magnetic |
| | | | microparticle, surfactant free |
| P-039-003 | P39 | SP-339 | Polystyrene carboxyl functionalized |
| P-041-001 | P41 | SP-341 | Carboxylic acid |
| P-047-001 | P47 | SP-365 | Silica |
| P-048-001 | P48 | SP-348 | Carboxylic acid, 150 nm |
| P-053-001 | P53 | SP-353 | Amino surface microparticle, 0.4-0.6 μm |
| P-056-001 | P56 | SP-356 | Silica amino functionalized microparticle, 0.1-0.39 μm |
| P-063-001 | P63 | SP-363 | Jeffamine surface, 0.1-0.39 μm |
| P-064-001 | P64 | SP-364 | Polystyrene microparticle, 2.0-2.9 μm |
| P-065-001 | P65 | SP-365 | Silica |
| P-069-001 | P69 | SP-369 | Carboxylated Original coating, 50 nm |
| P-073-001 | P73 | SP-373 | Dextran based coating, 0.13 μm |
| P-074-001 | P74 | SP-374 | Silica Silanol coated with lower acidity |

A particle of the present disclosure may be synthesized, or a particle of the present disclosure may be purchased from a commercial vendor. For example, particles consistent with the present disclosure may be purchased from commercial vendors including Sigma-Aldrich, Life Technologies, Fisher Biosciences, nanoComposix, Nanopartz, Spherotech, and other commercial vendors. In some cases, a particle of the present disclosure may be purchased from a commercial vendor and further modified, coated, or functionalized.

An example of a particle type of the present disclosure may be a carboxylate (Citrate) superparamagnetic iron oxide nanoparticle (SPION), a phenol-formaldehyde coated SPION, a silica-coated SPION, a polystyrene coated SPION, a carboxylated poly(styrene-co-methacrylic acid) coated SPION, a N-(3-Trimethoxysilylpropyl)diethylenetriamine coated SPION, a poly(N-(3-(dimethylamino)propyl) methacrylamide) (PDMAPMA)-coated SPION, a 1,2,4,5-Benzenetetracarboxylic acid coated SPION, a poly(Vinylbenzyltrimethylammonium chloride) (PVBTMAC) coated SPION, a carboxylate, PAA coated SPION, a poly(oligo(ethylene glycol) methyl ether methacrylate) (POEGMA)-coated SPION, a carboxylate microparticle, a polystyrene carboxyl functionalized particle, a carboxylic acid coated particle, a silica particle, a carboxylic acid particle of about 150 nm in diameter, an amino surface microparticle of about 0.4-0.6 μm in diameter, a silica amino functionalized microparticle of about 0.1-0.39 μm in diameter, a Jeffamine surface particle of about 0.1-0.39 μm in diameter, a polystyrene microparticle of about 2.0-2.9 μm in diameter, a silica particle, a carboxylated particle with an original coating of about 50 nm in diameter, a particle coated with a dextran based coating of about 0.13 μm in diameter, or a silica silanol coated particle with low acidity.

Particles that are consistent with the present disclosure can be made and used in methods of forming protein coronas after incubation in a biofluid at a wide range of sizes. In some cases, a particle of the present disclosure may be a nanoparticle. In some cases, a nanoparticle of the present disclosure may be from about 10 nm to about 1000 nm in diameter. For example, the nanoparticles disclosed herein can be at least 10 nm, at least 100 nm, at least 200 nm, at least 300 nm, at least 400 nm, at least 500 nm, at least 600 nm, at least 700 nm, at least 800 nm, at least 900 nm, from 10 nm to 50 nm, from 50 nm to 100 nm, from 100 nm to 150 nm, from 150 nm to 200 nm, from 200 nm to 250 nm, from 250 nm to 300 nm, from 300 nm to 350 nm, from 350 nm to 400 nm, from 400 nm to 450 nm, from 450 nm to 500 nm, from 500 nm to 550 nm, from 550 nm to 600 nm, from 600 nm to 650 nm, from 650 nm to 700 nm, from 700 nm to 750 nm, from 750 nm to 800 nm, from 800 nm to 850 nm, from 850 nm to 900 nm, from 100 nm to 300 nm, from 150 nm to 350 nm, from 200 nm to 400 nm, from 250 nm to 450 nm, from 300 nm to 500 nm, from 350 nm to 550 nm, from 400 nm to 600 nm, from 450 nm to 650 nm, from 500 nm to 700 nm, from 550 nm to 750 nm, from 600 nm to 800 nm, from 650 nm to 850 nm, from 700 nm to 900 nm, or from 10 nm to 900 nm in diameter. In some cases, a nanoparticle may be less than 1000 nm in diameter.

A particle of the present disclosure may be a microparticle. A microparticle may be a particle that is from about 1 μm to about 1000 μm in diameter. For example, the microparticles disclosed here can be at least 1 μm, at least 10 μm, at least 100 μm, at least 200 μm, at least 300 μm, at least 400 μm, at least 500 μm, at least 600 μm, at least 700 μm, at least 800 μm, at least 900 μm, from 10 μm to 50 μm, from 50 μm to 100 μm, from 100 μm to 150 μm, from 150 μm to 200 μm, from 200 μm to 250 μm, from 250 μm to 300 μm, from 300 μm to 350 μm, from 350 μm to 400 μm, from 400 μm to 450 μm, from 450 μm to 500 μm, from 500 μm to 550 μm, from 550 μm to 600 μm, from 600 μm to 650 μm, from 650 μm to 700 μm, from 700 μm to 750 μm, from 750 μm to 800 μm, from 800 μm to 850 μm, from 850 μm to 900 μm, from 100 μm to 300 μm, from 150 μm to 350 μm, from 200 μm to 400 μm, from 250 μm to 450 μm, from 300 μm to 500 μm, from 350 μm to 550 μm, from 400 μm to 600 μm, from 450 μm to 650 μm, from 500 μm to 700 μm, from 550 μm to 750 μm, from 600 μm to 800 μm, from 650 μm to 850 μm, from 700 μm to 900 μm, or from 10 μm to 900 μm in diameter. In some cases, a microparticle may be less than 1000 μm in diameter.

The ratio between surface area and mass can be a determinant of a particle's properties in the methods of the instant disclosure. For example, the number and types of biomolecules that a particle adsorbs from a solution may vary with the particle's surface area to mass ratio. The particles disclosed herein can have surface area to mass ratios of 3 to 30 cm²/mg, 5 to 50 cm²/mg, 10 to 60 cm²/mg, 15 to 70 cm²/mg, 20 to 80 cm²/mg, 30 to 100 cm²/mg, 35 to 120 cm²/mg, 40 to 130 cm²/mg, 45 to 150 cm²/mg, 50 to 160 cm²/mg, 60 to 180 cm²/mg, 70 to 200 cm²/mg, 80 to 220 cm²/mg, 90 to 240 cm²/mg, 100 to 270 cm²/mg, 120 to 300 cm²/mg, 200 to 500 cm²/mg, 10 to 300 cm²/mg, 1 to 3000 cm²/mg, 20 to 150 cm²/mg, 25 to 120 cm²/mg, or from 40 to 85 cm²/mg. Small particles (e.g., with diameters of 50 nm or less) can have higher surface area to mass ratios than large particles (e.g., with diameters of 200 nm or more). In some cases (e.g., for small particles), the particles can have surface area to mass ratios of 200 to 1000 cm²/mg, 500 to 2000 cm²/mg, 1000 to 4000 cm²/mg, 2000 to 8000 cm²/mg, or 4000 to 10000 cm²/mg. In some cases (e.g., for large particles), the particles can have surface area to mass ratios of 1 to 3 cm²/mg, 0.5 to 2 cm²/mg, 0.25 to 1.5 cm²/mg, or 0.1 to 1 cm²/mg.

In some cases, a plurality of particles (e.g., of a particle panel) used with the methods described herein may have a range of surface area to mass ratios. In some cases, the range of surface area to mass ratios for a plurality of particles is less than 100 cm²/mg, 80 cm²/mg, 60 cm²/mg, 40 cm²/mg, 20 cm²/mg, 10 cm²/mg, 5 cm²/mg, or 2 cm²/mg. In some cases, the surface area to mass ratios for a plurality of particles varies by no more than 40%, 30%, 20%, 10%, 5%, 3%, 2%, or 1% between the particles in the plurality. In some cases, the plurality of particles may comprise at least 2, 3, 4, 5, 6, 7, 8, 9, 10, 12, 15, 20, or more different types of particles.

In some cases, a plurality of particles (e.g., in a particle panel) may have a wider range of surface area to mass ratios. In some cases, the range of surface area to mass ratios for a plurality of particles is greater than 100 cm²/mg, 150 cm²/mg, 200 cm²/mg, 250 cm²/mg, 300 cm²/mg, 400 cm²/mg, 500 cm²/mg, 800 cm²/mg, 1000 cm²/mg, 1200 cm²/mg, 1500 cm²/mg, 2000 cm²/mg, 3000 cm²/mg, 5000 cm²/mg, 7500 cm²/mg, 10000 cm²/mg, or more. In some cases, the surface area to mass ratios for a plurality of particles (e.g., within a panel) can vary by more than 100%, 200%, 300%, 400%, 500%, 1000%, 10000% or more. In some cases, the plurality of particles with a wide range of surface area to mass ratios comprises at least 2, 3, 4, 5, 6, 7, 8, 9, 10, 12, 15, 20, or more different types of particles.

A particle may comprise a wide array of physical properties. A physical property of a particle may include composition, size, surface charge, hydrophobicity, hydrophilicity, surface functionality, surface topography, surface curvature, porosity, core material, shell material, shape, and any combination thereof.

A surface functionality may comprise a polymerizable functional group, a positively or negatively charged functional group, a zwitterionic functional group, an acidic or basic functional group, a polar functional group, or any combination thereof. A surface functionality may comprise carboxyl groups, hydroxyl groups, thiol groups, cyano groups, nitro groups, ammonium groups, alkyl groups, imidazolium groups, sulfonium groups, pyridinium groups, pyrrolidinium groups, phosphonium groups, aminopropyl groups, amine groups, boronic acid groups, N-succinimidyl ester groups, PEG groups, streptavidin, methyl ether groups, triethoxylpropylaminosilane groups, PCP groups, citrate groups, lipoic acid groups, BPEI groups, or any combination thereof. A particle from among the plurality of particles may be selected from the group consisting of: micelles, liposomes, iron oxide particles, silver particles, gold particles, palladium particles, quantum dots, platinum particles, titanium particles, silica particles, metal or inorganic oxide particles, synthetic polymer particles, copolymer particles, terpolymer particles, polymeric particles with metal cores, polymeric particles with metal oxide cores, polystyrene sulfonate particles, polyethylene oxide particles, polyoxyethylene glycol particles, polyethylene imine particles, polylactic acid particles, polycaprolactone particles, polyglycolic acid particles, poly(lactide-co-glycolide polymer particles, cellulose ether polymer particles, polyvinylpyrrolidone particles, polyvinyl acetate particles, polyvinylpyrrolidone-vinyl acetate copolymer particles, polyvinyl alcohol particles, acrylate particles, polyacrylic acid particles, crotonic acid copolymer particles, polyethlene phosphonate particles, polyalkylene particles, carboxy vinyl polymer particles, sodium alginate particles, carrageenan particles, xanthan gum particles, gum acacia particles, Arabic gum particles, guar gum particles, pullulan particles, agar particles, chitin particles, chitosan particles, pectin particles, karaya tum particles, locust bean gum particles, maltodextrin particles, amylose particles, corn starch particles, potato starch particles, rice starch particles, tapioca starch particles, pea starch particles, sweet potato starch particles, barley starch particles, wheat starch particles, hydroxypropylated high amylose starch particles, dextrin particles, levan particles, elsinan particles, gluten particles, collagen particles, whey protein isolate particles, casein particles, milk protein particles, soy protein particles, keratin particles, polyethylene particles, polycarbonate particles, polyanhydride particles, polyhydroxyacid particles, polypropylfumerate particles, polycaprolactone particles, polyamine particles, polyacetal particles, polyether particles, polyester particles, poly(orthoester) particles, polycyanoacrylate particles, polyurethane particles, polyphosphazene particles, polyacrylate particles, polymethacrylate particles, polycyanoacrylate particles, polyurea particles, polyamine particles, polystyrene particles, poly(lysine) particles, chitosan particles, dextran particles, poly(acrylamide) particles, derivatized poly(acrylamide) particles, gelatin particles, starch particles, chitosan particles, dextran particles, gelatin particles, starch particles, poly-(3-amino-ester particles, poly(amido amine) particles, poly lactic-co-glycolic acid particles, polyanhydride particles, bioreducible polymer particles, and 2-(3-aminopropylamino)ethanol particles, and any combination thereof.

Particles of the present disclosure may differ by one or more physicochemical property. The one or more physicochemical property is selected from the group consisting of: composition, size, surface charge, hydrophobicity, hydrophilicity, roughness, density surface functionality, surface topography, surface curvature, porosity, core material, shell material, shape, and any combination thereof. The surface functionality may comprise a macromolecular functionalization, a small molecule functionalization, or any combination thereof. A small molecule functionalization may comprise an aminopropyl functionalization, amine functionalization, boronic acid functionalization, carboxylic acid functionalization, alkyl group functionalization, N-succinimidyl ester functionalization, monosaccharide functionalization, phosphate sugar functionalization, sulfurylated sugar functionalization, ethylene glycol functionalization, streptavidin functionalization, methyl ether functionalization, trimethoxysilylpropyl functionalization, silica functionalization, triethoxylpropylaminosilane functionalization, thiol functionalization, PCP functionalization, citrate functionalization, lipoic acid functionalization, ethyleneimine functionalization. A particle panel may comprise a plurality of particles with a plurality of small molecule functionalizations selected from the group consisting of silica functionalization, trimethoxysilylpropyl functionalization, dimethylamino propyl functionalization, phosphate sugar functionalization, amine functionalization, and carboxyl functionalization.

A small molecule functionality may comprise a polar functional group. Non-limiting examples of polar functional groups comprise carboxyl group, a hydroxyl group, a thiol group, a cyano group, a nitro group, an ammonium group, an imidazolium group, a sulfonium group, a pyridinium group, a pyrrolidinium group, a phosphonium group or any combination thereof. In some embodiments, the functional group is an acidic functional group (e.g., sulfonic acid group, carboxyl group, and the like), a basic functional group (e.g., amino group, cyclic secondary amino group (such as pyrrolidyl group and piperidyl group), pyridyl group, imidazole group, guanidine group, etc.), a carbamoyl group, a hydroxyl group, an aldehyde group and the like.

A small molecule functionality may comprise an ionic or ionizable functional group. Non-limiting examples of ionic or ionizable functional groups comprise an ammonium group, an imidazolium group, a sulfonium group, a pyridinium group, a pyrrolidinium group, a phosphonium group.

A small molecule functionality may comprise a polymerizable functional group. Non-limiting examples of the polymerizable functional group include a vinyl group and a (meth)acrylic group. In some embodiments, the functional group is pyrrolidyl acrylate, acrylic acid, methacrylic acid, acrylamide, 2-(dimethylamino)ethyl methacrylate, hydroxyethyl methacrylate and the like.

A surface functionality may comprise a charge. For example, a particle can be functionalized to carry a net neutral surfacce charge, a net positive surface charge, a net negative surface charge, or a zwitterionic surface. Surface charge can be a determinant of the types of biomolecules collected on a particle. Accordingly, optimizing a particle panel may comprise selecting particles with different surface charges, which may not only increase the number of different proteins collected on a particle panel, but also increase the likelihood of detecting a protein-protein interaction. A particle panel may comprise a positively charged particle and a negatively charged particle. A particle panel may comprise a positively charged particle and a neutral particle. A particle panel may comprise a positively charged particle and a zwitterionic particle. A particle panel may comprise a neutral particle and a negatively charged particle. A particle panel may comprise a neutral particle and a zwitterionic particle. A particle panel may comprise a negative particle and a zwitterionic particle. A particle panel may comprise a positively charged particle, a negatively charged particle, and a neutral particle. A particle panel may comprise a positively charged particle, a negatively charged particle, and a zwitterionic particle. A particle panel may comprise a positively charged particle, a neutral particle, and a zwitterionic particle. A particle panel may comprise a negatively charged particle, a neutral particle, and a zwitterionic particle.

The present disclosure includes compositions (e.g., particle panels) and methods that comprise two or more particles differing in at least one physicochemical property. A composition or method of the present disclosure may comprise 3 to 6 particles differing in at least one physicochemical property. A composition or method of the present disclosure may comprise 4 to 8 particles differing in at least one physicochemical property. A composition or method of the present disclosure may comprise 4 to 10 particles differing in at least one physicochemical property. A composition or method of the present disclosure may comprise 5 to 12 particles differing in at least one physicochemical property. A composition or method of the present disclosure may comprise 6 to 14 particles differing in at least one physicochemical property. A composition or method of the present disclosure may comprise 8 to 15 particles differing in at least one physicochemical property. A composition or method of the present disclosure may comprise 10 to 20 particles differing in at least one physicochemical property. A composition or method of the present disclosure may comprise at least 2 distinct particle types, at least 3 distinct particle types, at least 4 distinct particle types, at least 5 distinct particle types, at least 6 distinct particle types, at least 7 distinct particle types, at least 8 distinct particle types, at least 9 distinct particle types, at least 10 distinct particle types, at least 11 distinct particle types, at least 12 distinct particle types, at least 13 distinct particle types, at least 14 distinct particle types, at least 15 distinct particle types, at least 20 distinct particle types, at least 25 particle types, or at least 30 distinct particle types.

Surface functionalities can influence the composition of a particle's biomolecule corona. Such surface functionalities can include small molecule functionalization or macromolecular functionalization.

A surface functionalization may comprise a small molecule functionalization, a macromolecular functionalization, or a combination of two or more such functionalizations. A macromolecular functionalization may comprise a biomacromolecule, such as a protein or a polynucleotide (e.g., a 100-mer DNA molecule). A macromolecular functionalization may be comprise a protein, polynucleotide, or polysaccharide, or may be comparable in size to any of the aforementioned classes of species. For example, a macromolecular functionalization may comprise a volume of at least 6 nm³, at least 8 nm³, at least 12 nm³, at least 15 nm³, at least 20 nm³, at least 30 nm³, at least 50 nm³, at least 80 nm³, at least 120 nm³, at least 180 nm³, at least 300 nm³, at least 500 nm³, at least 800 nm³, at least 1200 nm³, at least 1500 nm³, or at least 2000 nm³. A macromolecular functionalization may comprise a surface area of at least at least 15 nm², at least 20 nm², at least 25 nm², at least 40 nm², at least 80 nm², at least 150 nm², at least 300 nm², at least 500 nm², at least 800 nm², at least 1200 nm², or at least 1500 nm². A macromolecular functionalization may comprise a bait molecule.

A macromolecular functionalization may comprise a specific form of attachment to a particle. A macromolecule may be tethered to a particle via a linker. The linker may hold the macromolecule close to the particle, thereby restricting its motion and reorientation relative to the particle, or may extend the macromolecule away from the particle. The linker may be rigid (e.g., a polyolefin linker) or flexible (e.g., a nucleic acid linker). A linker may be no more than 0.5 nm in length, no more than 1 nm in length, no more than 1.5 nm in length, no more than 2 nm in length, no more than 3 nm in length, no more than 4 nm in length, no more than 5 nm in length, no more than 8 nm in length, or no more than 10 nm in length. A linker may be at least 1 nm in length, at least 2 nm in length, at least 3 nm in length, at least 4 nm in length, at least 5 nm in length, at least 8 nm in length, at least 12 nm in length, at least 15 nm in length, at least 20 nm in length, at least 25 nm in length, or at least 30 nm in length. As such, a surface functionalization on a particle may project beyond a primary corona associated with the particle. A surface functionalization may also be situated beneath or within a biomolecule corona that forms on the particle surface.

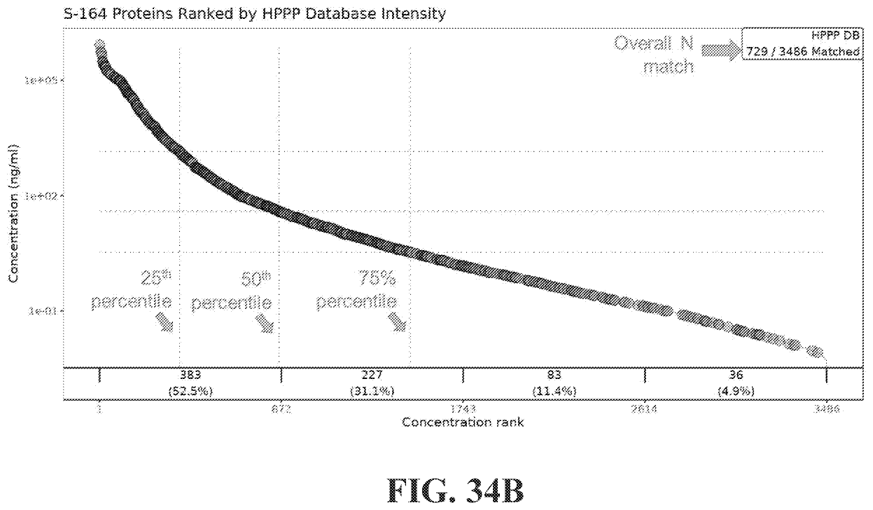

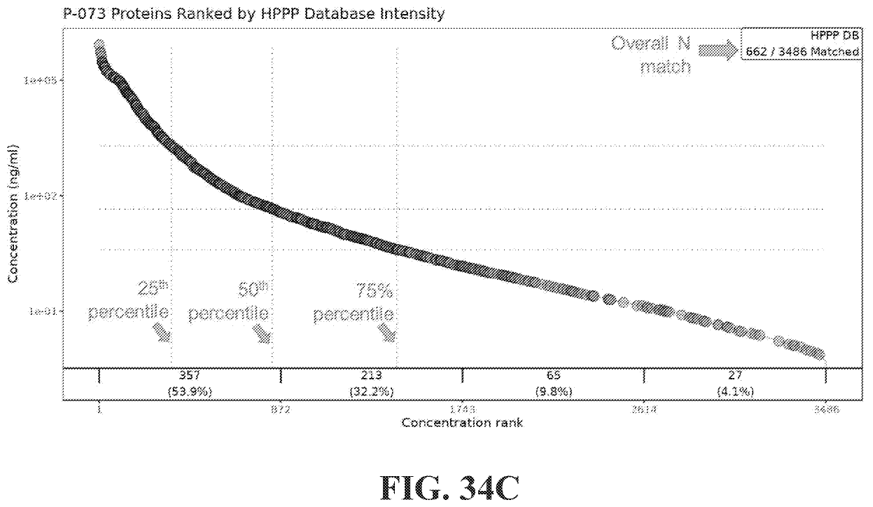

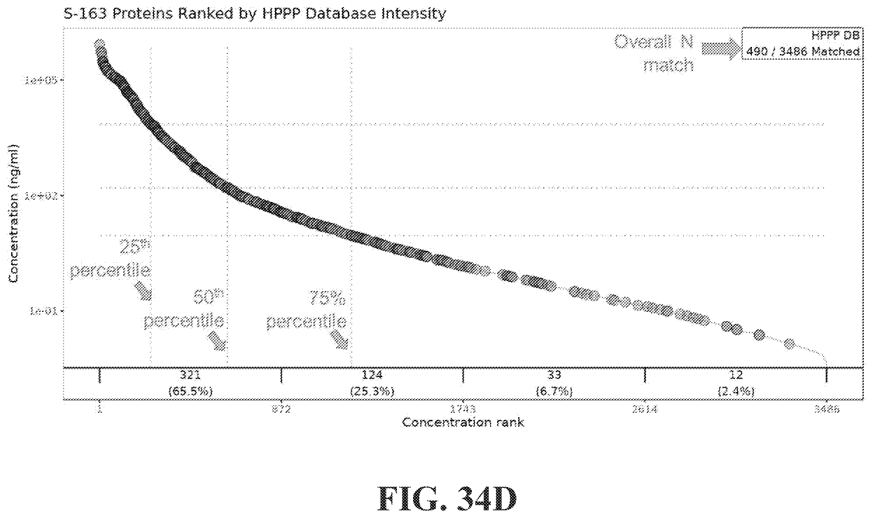

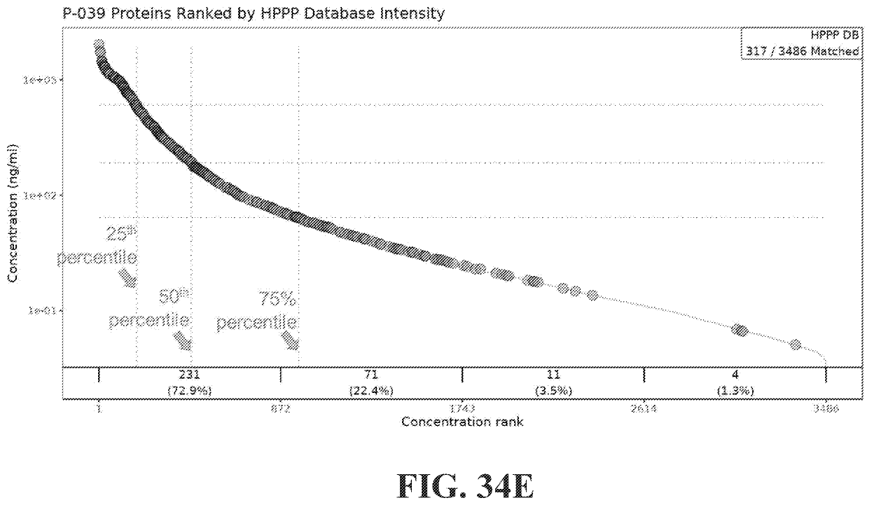

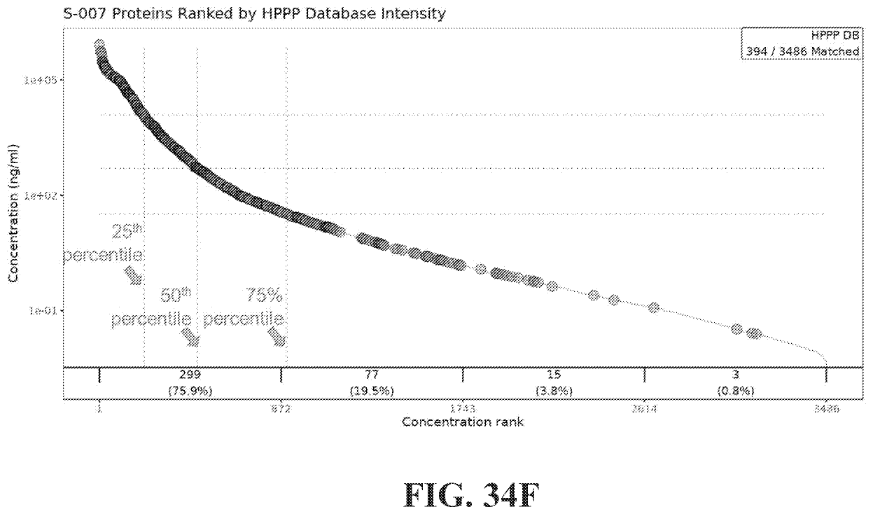

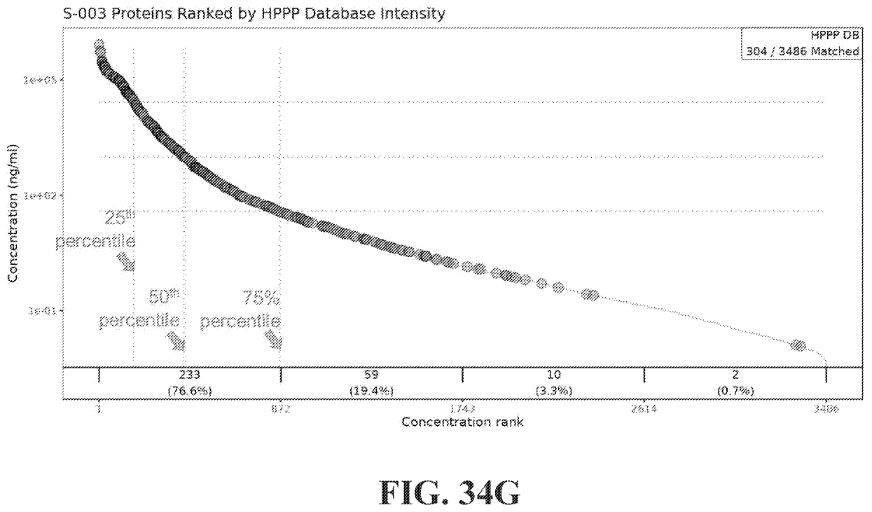

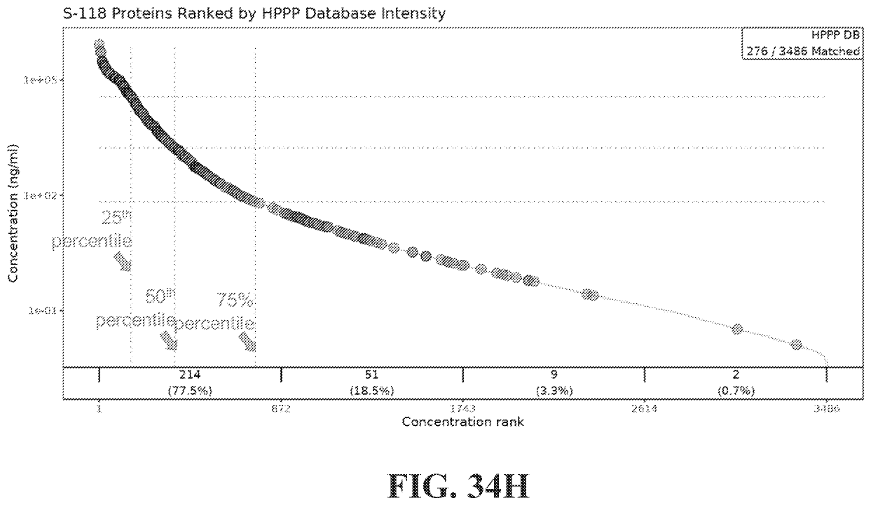

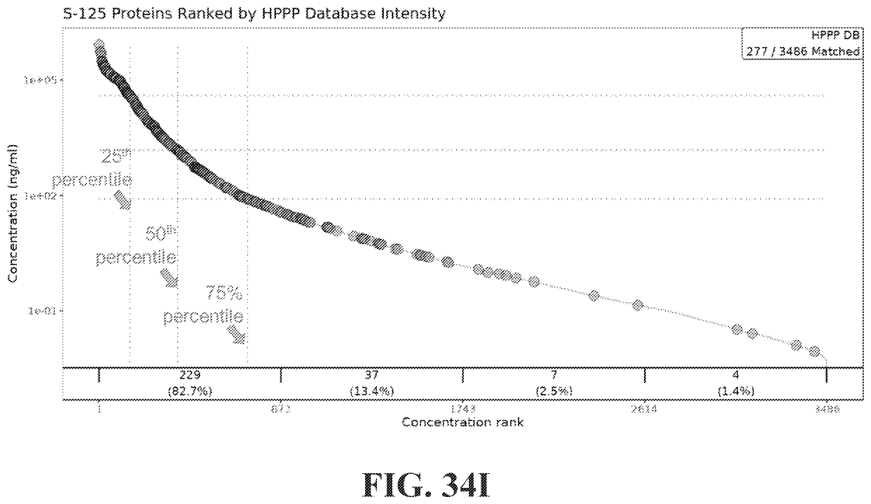

A macromolecule may be tethered at a specific location, such as a protein's C-terminus, or may be tethered at a number of possible sites. For example, the present disclosure provides cis-ubiquitin particles (S-163), which comprise activated ubiquitin covalently attached to linkers via its N-terminus, and ubiquitin particles (S-164), which comprise ubiquitin covalently attached to linkers via any of its surface exposed lysine residues. As can be seen in FIG. 34B and FIG. 34D, this difference in tethering can meaningfully change the number of proteins and types of proteins that form in the biomolecule corona of a particle.

A particle may comprise different degrees of coverage by a macromolecular functionalization. A particle may comprise a macromolecular functionalization that covers less than 5%, less than 10%, less than 20%, less than 30%, less than 40%, less than 50%, less than 60%, or less than 70% of its surface. For example, a particle with a surface area of 40000 nm² may comprise an average of 40 ubiquitin molecules on its surface, thereby covering about 9% of its surface. A particle may comprise at least 30%, at least 40%, at least 50%, at least 60%, at least 70%, at least 80%, at least 90%, at least 95%, or close to 100% surface coverage from a macromolecular functionalization. For example, a particle may comprise a dextran coating covering the entirety of its surface.

A macromolecular functionalized particle may collect a greater number of biomolecules (e.g., proteins) from a sample than a small molecule functionalized particle. This concept is illustrated in FIG. 34, which shows the number of plasma proteins collected on particles bearing macromolecular functionalizations (FIG. 34B-D) and on particles bearing small molecule functionalizations (FIG. 34E-K). In this example, the macromolecular functionalized particles not only collected more proteins from the plasma sample than did the small molecule functionalized particles, but also a higher proportion of low abundance proteins. At one extreme, the ubiquitin functionalized particles (S-164, FIG. 34B) collected more than 360 proteins with plasma abundances of less than 80 ng/ml, contrasting with the polystyrene carboxyl functionalized particles (P-039, FIG. 34E), which collected fewer than 80 of such proteins.

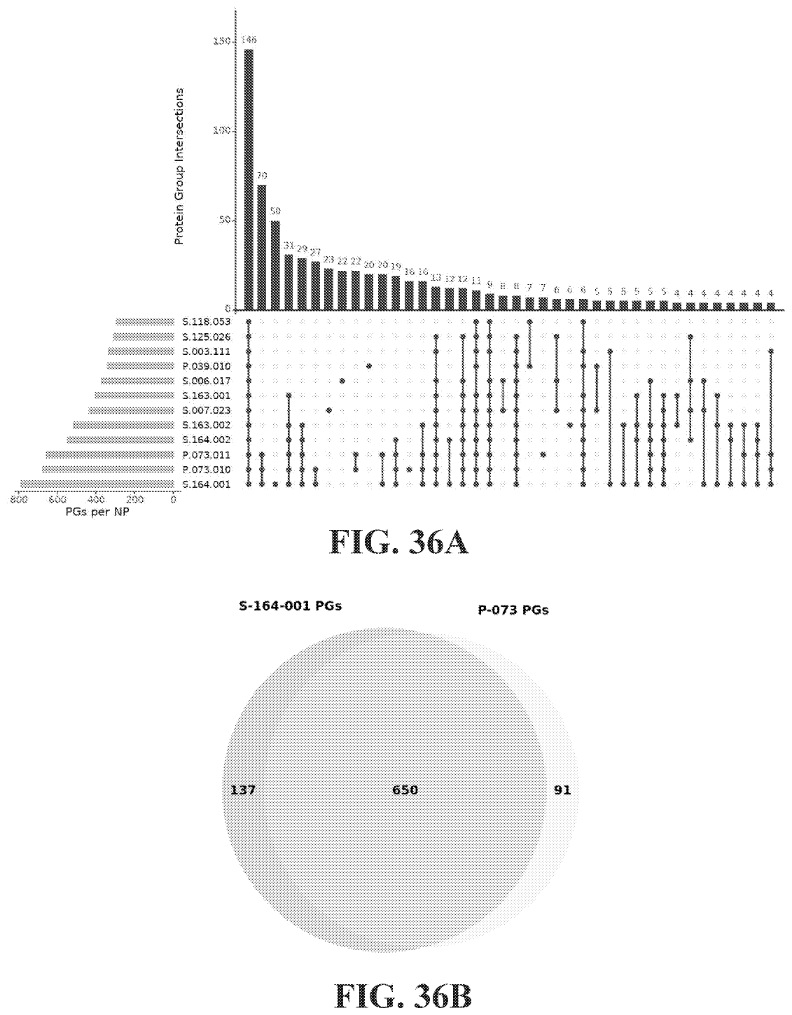

Furthermore, as is shown in FIG. 36A, the types of biomolecules (e.g., proteins) collected on small molecule functionalized and macromolecular functionalized particles can greatly differ. FIG. 36A summarizes the results of a protein corona assay in which 6 macromolecular functionalized particles (S-163-001, S-163-002, S-164-001, S-164-002, P-073-10, P-073-11) and 6 small molecule functionalized particles (S-118-053, S-125-026, S-003-111, P-039-010, S-006-001, and S-007-023) were independently contacted to human plasma. The 6 macromolecular functionalized particles collected more than 300 types of proteins not observed on the small molecule functionalized proteins, indicating that the macromolecular functionalized particles were able to profile a portion of the plasma sample that is inaccessible to small molecule functionalized particles.

A particle may comprise a single surface functionalization, such as a single type of protein, or a plurality of surface functionalizations, such as a plurality of different types of proteins. A particle may comprise a plurality of macromolecular functionalizations. For example, a particle may comprise 2, 3, 4, 5, 6, 8, 10, 15, 20, or 25 or more types of proteins as surface functionalizations. A particle may comprise a combination of macromolecular and small molecule surface functionalizations. For example, a particle may comprise a combination of ubiquitin (macromolecular) and phosphate sugar (small molecule) molecules linked to its surface. A plurality of surface functionalizations may be randomly or evenly distributed over a particle surface, or may be localized to particular regions of the particle.

A surface functionalization may comprise a high affinity for a particular biomolecule or class of biomolecules. For example, a small molecule surface functionalization may comprise a nonpolar moiety (such as an organosilane) that interacts strongly with nonpolar protein functional groups and alpha helices. Analogously, a macromolecular surface functionalization may comprise a peptide (e.g., an antibody) with a high affinity for a specific molecular target.

A macromolecular surface functionalization may comprise a peptide that does not have a high affinity for any of the biomolecules present in a sample. Such a peptide may comprise a binding affinity of no greater than 200 nM, of no greater than 500 nM, no greater than 1 no greater than 5 no greater than 10 no greater than 50 no greater than 100 no greater than 500 no greater than 1 mM, no greater than 5 mM, or no greater than 10 mM for any biomolecule within a particular sample, or for any biomolecule present at a concentration of at least 1 pM, at least 10 pM, at least 100 pM, at least 1 nM, at least 10 nM, at least 100 nM, or at least 1 μM within the sample. As is shown in FIG. 34B and FIG. 34D, which depict proteins absorbed from plasma samples onto ubiquitin functionalized particles, a particle comprising a low target affinity macromolecular functionalization can collect a greater number of biomolecules (e.g., proteins) from a sample than a particle bearing small molecule functionalizations, such as those shown in FIG. 34E-J. A particle may comprise a ubiquitin surface functionalization. A particle may comprise a dextran surface functionalization.

A particle may comprise a small molecule functionalization. A small molecule functionalization may comprise a mass of fewer than 600 Daltons, fewer than 500 Daltons, fewer than 400 Daltons, fewer than 300 Daltons, fewer than 200 Daltons, or fewer than 100 Daltons. A small molecule functionalization may comprise an ionizable moiety, such as a chemical group with a pKₐ or pK_{b} of less than 6 or 7. A small molecule functionalization may comprise a small organic molecule such as an alcohol (e.g., octanol), an amine, an alkane, an alkene, an alkyne, a heterocycle (e.g., a piperidinyl group), a heteroaromatic group, a thiol, a carboxylate, a carbonyl, an amide, an ester, a thioester, a carbonate, a thiocarbonate, a carbamate, a thiocarbamate, a urea, a thiourea, a halogen, a sulfate, a phosphate, a monosaccharide, a disaccharide, a lipid, or any combination thereof. For example, a small molecule functionalization may comprise a phosphate sugar, a sugar acid, or a sulfurylated sugar.

A particle of the present disclosure may be contacted with a biological sample (e.g., a biofluid) to form a biomolecule corona. The particle and biomolecule corona may be separated from the biological sample, for example by centrifugation, magnetic separation, filtration, or gravitational separation. The particle types and biomolecule corona may be separated from the biological sample using a number of separation techniques. Non-limiting examples of separation techniques include comprises magnetic separation, column-based separation, filtration, spin column-based separation, centrifugation, ultracentrifugation, density or gradient-based centrifugation, gravitational separation, or any combination thereof. A protein corona analysis may be performed on the separated particle and biomolecule corona. A protein corona analysis may comprise identifying one or more proteins in the biomolecule corona, for example by mass spectrometry. A single particle type (e.g., a particle of a type listed in TABLE 1) may be contacted to a biological sample. A plurality of particle types (e.g., a plurality of the particle types provided in TABLE 1) may be contacted to a biological sample. The plurality of particle types may be combined and contacted to the biological sample in a single sample volume. The plurality of particle types may be sequentially contacted to a biological sample and separated from the biological sample prior to contacting a subsequent particle type to the biological sample. Protein corona analysis of the biomolecule corona may compress the dynamic range of the analysis compared to a total protein analysis method.

The particles of the present disclosure may be used to serially interrogate a sample by incubating a first particle type with the sample to form a biomolecule corona on the first particle type, separating the first particle type, incubating a second particle type with the sample to form a biomolecule corona on the second particle type, separating the second particle type, and repeating the interrogating (by incubation with the sample) and the separating for any number of particle types. In some cases, the biomolecule corona on each particle type used for serial interrogation of a sample may be analyzed by protein corona analysis. The biomolecule content of the supernatant may be analyzed following serial interrogation with one or more particle types.

A particle type of the present disclosure may be used to serially interrogate a sample followed by corona analysis of proteins in the protein corona formed upon incubation of the particle type with the sample. Serial interrogation may be performed with two particle types in a round-by-round fashion. Serial interrogation may also include subsequent interrogation with additional particle times. A particle of the present disclosure may be used to deplete a sample prior to the above described method of serial interrogation. A particle type may be contacted to a sample to form biomolecule corona on a surface of the particle type, and the particle may be separated from the sample, thereby depleting the sample. This strategy may be used to deplete one or more proteins (e.g., one or more high abundance proteins) from a sample. The biomolecule content of the supernatant of a depleted sample may be analyzed. In some cases, the supernatant of the depleted sample may be used in any of the protein corona analysis methods disclosed herein.

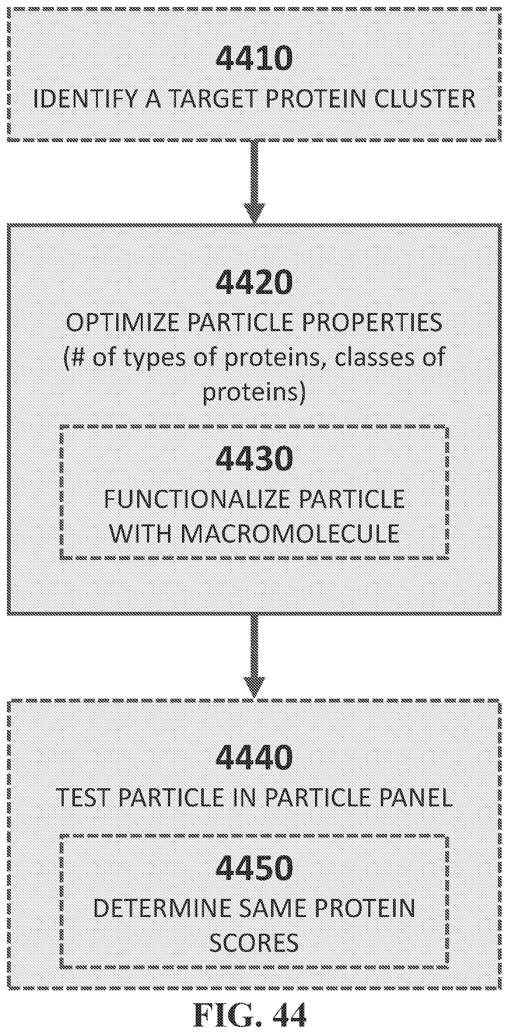

A particle may be designed to interrogate for protein-protein interactions among a particular class, type, or cluster (e.g., a collection of multiple protein classes or groups) of proteins. Much of the human and of other proteomes have been minimally queried, and may comprise underrepresented or unknown protein-protein interactions. Accordingly, a particle may be selected or designed to optimally to query for protein-protein interactions (summarized in FIG. 44).

As illustrated in FIG. 44, such a process may optionally comprise identifying a target protein group or cluster of interest 4410. The protein group or cluster may comprise fewer than expected protein-protein interactions (in comparison to e.g., what would be expected based on identified hub proteins and interactions listed in the string database). The protein group or cluster may reside within a portion of the proteome that is relevant to a number of biological states (e.g., diseases). The protein group or cluster may be related by a common structural motif, such as a tertiary structural feature or a post-translational modification (e.g., a particular glycosylation pattern or a post-translationally appended protein group, such as ubiquitin).

A particle may be optimized 4420 to identify protein-protein interactions. The protein-protein interactions targeted by a particle may be from among a target protein group or cluster or may be from a particular sample or sample type. A method for identifying a protein-protein interaction comprises identifying a stronger association between two proteins than between the proteins and the particle type(s) on which they were collected. Thus, a particle with a high affinity for proteins from a sample or from the target group or cluster may not be optimal for identifying protein-protein interactions, as the particle may generate strong associations with the proteins of interest. Therefore, a method for optimizing a particle for identifying protein-protein interactions may comprise designing the particle to have a moderate or low affinity for the proteins from the sample, target protein group, or cluster.

Optimizing a particle for identifying protein-protein interactions may optionally comprise functionalizing the particle with a macromolecule 4430 (i.e., a macromolecular functionalization) to enrich for particular protein-protein interactions. The macromolecular functionalization may be chosen to interact with a common feature among a target protein group or cluster, such as a common post-translational modification (e.g., a glycosylation pattern or a protein appendage such as ubiquitin). The macromolecular functionalization may be selected to enhance collection of a target protein group or cluster and to simultaneously generate moderate or weak associations with proteins from the target group or cluster.

For example, a particle may be functionalized with a macromolecule that comprises no greater than 10 mM binding affinity (e.g., by measured or predicted dissociation constant (K_{d})) for a subset of proteins from the target protein group or cluster. A particle may be functionalized with a macromolecule that comprises no greater than 1 mM binding affinity for a subset of proteins from the target protein group or cluster. A particle may be functionalized with a macromolecule that comprises no greater than 100 μM binding affinity for a subset of proteins from the target protein group or cluster. A particle may be functionalized with a macromolecule that comprises no greater than 50 μM binding affinity for a subset of proteins from the target protein group or cluster. A particle may be functionalized with a macromolecule that comprises no greater than 20 μM binding affinity for a subset of proteins from the target protein group or cluster. A particle may be functionalized with a macromolecule that comprises no greater than 10 binding affinity for a subset of proteins from the target protein group or cluster. A particle may be functionalized with a macromolecule that comprises no greater than 1 μM binding affinity for a subset of proteins from the target protein group or cluster. The subset of proteins from the target protein group or cluster may be representative set of 2 proteins, 3 proteins, 4 proteins, 5 proteins, 8 proteins, 10 proteins, or 15 proteins from among the protein group or cluster. The binding affinity may be binding affinity for a protein in a complex biological sample, or for a purified protein.

As an example, the present disclosure provides ubiquitin functionalized particles designed to interrogate protein-protein interactions among ubiquitinated proteins. Ubiquitinated proteins are a diverse cluster of proteins that span a wide range of important physiological functions, including in transcriptional and lysosomal recycling. Ubiquitin was chosen as a macromolecular functionalization in part because of its mM-range homodimerization affinity. Thus, the ubiquitin functionalized particles of the present disclosure comprise sufficiently high affinities for ubiquitinated proteins to enable their collection and identification, and sufficiently low affinities to allow protein-protein interactions to be identified from among ubiquitinated proteins.

Optionally, a macromolecular functionalized particle may be added to a particle panel 4440. A particle panel may comprise a plurality of particle types, and may provide for the particle types to be collectively or separately be contacted to a sample. For example, a particle panel may provide 5 types of particles as a powdered mixture. Alternatively, a particle panel may provide 5 types of particles in separate solutions disposed in separate partitions of a multi-well plate (e.g., a 96 well plate). A particle panel may be designed for breadth, for example by collecting a large number of different protein groups, or for depth, such as by collecting a large number of proteins from a particular protein class. The particle panel design process may comprise the addition of a macromolecular functionalized particle with either orthogonal or complementary protein collection relative to other particles present in the panel. Optimizing the particle may comprise determining same protein scores for at least a subset of proteins from the target protein group or cluster 4450 by comparing protein identifications of the optimized particle and the particles on the particle panel. Optimizing the particle may comprise determining that the same protein scores for the subset of proteins from the target protein group are no higher than 0.6, 0.5, 0.4, 0.3, or 0.2.

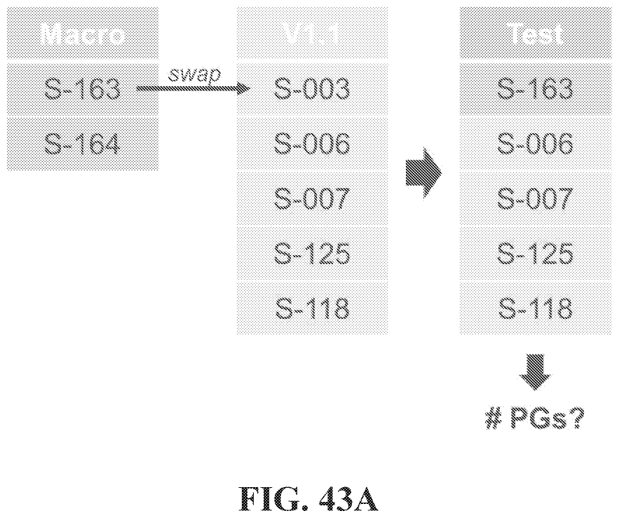

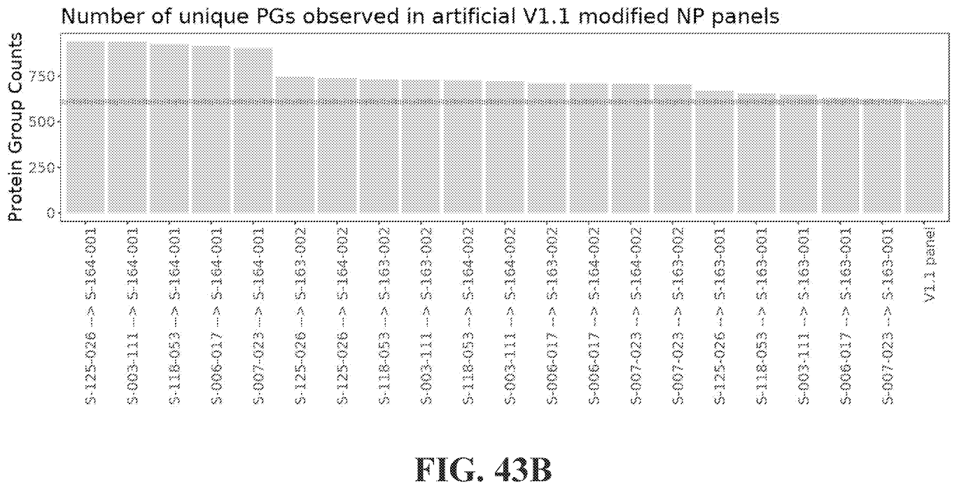

FIG. 43B provides an example of such a design process, and summarizes the protein group counts collected from plasma for particle panels comprising 4 small molecule functionalized particles selected from the group consisting of the types S-003, S-006, S-007, 5-118, and S-125 summarized in TABLE 17, and one macromolecular functionalized particle selected from the group consisting of the types S-163 and S-164 summarized in TABLE 17. As can be seen on the left of the plot, providing a macromolecular functionalized particle replacement for a small molecule functionalized particle resulted in protein group count increases of as much as 400.

### Particle Panels

The present disclosure provides compositions and methods of use thereof for assaying a sample for proteins. Compositions described herein include particle panels comprising one or more than one distinct particle types. Particle panels described herein can vary in the number of particle types and the diversity of particle types in a single panel. For example, particles in a panel may vary based on size, polydispersity, shape and morphology, surface charge, surface chemistry and functionalization, and base material. Panels may be incubated with a sample to be analyzed for proteins and protein concentrations. Proteins in the sample adsorb to the surface of the different particle types in the particle panel to form a protein corona. The exact protein and the concentration of protein that adsorbs to a certain particle type in the particle panel may depend on the composition, size, and surface charge of said particle type. Thus, each particle type in a panel may have different protein coronas due to adsorbing a different set of proteins, different concentrations of a particular protein, or a combination thereof. Each particle type in a panel may have mutually exclusive protein coronas or may have overlapping protein coronas. Overlapping protein coronas can overlap in protein identity, in protein concentration, or both. The present disclosure also provides methods for selecting a particle types for inclusion in a panel depending on the sample type. Particle types included in a panel may be a combination of particles that are optimized for removal of highly abundant proteins. Particle types also consistent for inclusion in a panel are those selected for adsorbing particular proteins of interest. The particles can be nanoparticles. The particles can be microparticles. The particles can be a combination of nanoparticles and microparticles.

The particle panels disclosed herein can be used to identify the number of distinct proteins disclosed herein, and/or any of the specific proteins disclosed herein, over a wide dynamic range. For example, the particle panels disclosed herein comprising distinct particle types, can enrich for proteins in a sample, which can be identified using the Proteograph workflow, over the entire dynamic range at which proteins are present in a sample (e.g., a plasma sample). In some cases, a particle panel including any number of distinct particle types disclosed herein, enriches and identifies proteins over a dynamic range of at least 2. In some cases, a particle panel including any number of distinct particle types disclosed herein, enriches and identifies proteins over a dynamic range of at least 3. In some cases, a particle panel including any number of distinct particle types disclosed herein, enriches and identifies proteins over a dynamic range of at least 4. In some cases, a particle panel including any number of distinct particle types disclosed herein, enriches and identifies proteins over a dynamic range of at least 5. In some cases, a particle panel including any number of distinct particle types disclosed herein, enriches and identifies proteins over a dynamic range of at least 6. In some cases, a particle panel including any number of distinct particle types disclosed herein, enriches and identifies proteins over a dynamic range of at least 7. In some cases, a particle panel including any number of distinct particle types disclosed herein, enriches and identifies proteins over a dynamic range of at least 8. In some cases, a particle panel including any number of distinct particle types disclosed herein, enriches and identifies proteins over a dynamic range of at least 9. In some cases, a particle panel including any number of distinct particle types disclosed herein, enriches and identifies proteins over a dynamic range of at least 10. In some cases, a particle panel including any number of distinct particle types disclosed herein, enriches and identifies proteins over a dynamic range of at least 11. In some cases, a particle panel including any number of distinct particle types disclosed herein, enriches and identifies proteins over a dynamic range of at least 12. In some cases, a particle panel including any number of distinct particle types disclosed herein, enriches and identifies proteins over a dynamic range of at least 13. In some cases, a particle panel including any number of distinct particle types disclosed herein, enriches and identifies proteins over a dynamic range of at least 14. In some cases, a particle panel including any number of distinct particle types disclosed herein, enriches and identifies proteins over a dynamic range of at least 15. In some cases, a particle panel including any number of distinct particle types disclosed herein, enriches and identifies proteins over a dynamic range of at least 20. In some cases, a particle panel including any number of distinct particle types disclosed herein, enriches and identifies proteins over a dynamic range of from 2 to 100. In some cases, a particle panel including any number of distinct particle types disclosed herein, enriches and identifies proteins over a dynamic range of from 2 to 20. In some cases, a particle panel including any number of distinct particle types disclosed herein, enriches and identifies proteins over a dynamic range of from 2 to 10. In some cases, a particle panel including any number of distinct particle types disclosed herein, enriches and identifies proteins over a dynamic range of from 2 to 5. In some cases, a particle panel including any number of distinct particle types disclosed herein, enriches and identifies proteins over a dynamic range of from 5 to 10.

A particle panel including any number of distinct particle types disclosed herein, enriches and identifies a single protein or protein group. In some cases, the single protein or protein group may comprise proteins having different post-translational modifications. For example, a first particle type in the particle panel may enrich a protein or protein group having a first post-translational modification, a second particle type in the particle panel may enrich the same protein or same protein group having a second post-translational modification, and a third particle type in the particle panel may enrich the same protein or same protein group lacking a post-translational modification. In some cases, the particle panel including any number of distinct particle types disclosed herein, enriches and identifies a single protein or protein group by binding different domains, sequences, or epitopes of the single protein or protein group. For example, a first particle type in the particle panel may enrich a protein or protein group by binding to a first domain of the protein or protein group, and a second particle type in the particle panel may enrich the same protein or same protein group by binding to a second domain of the protein or protein group.

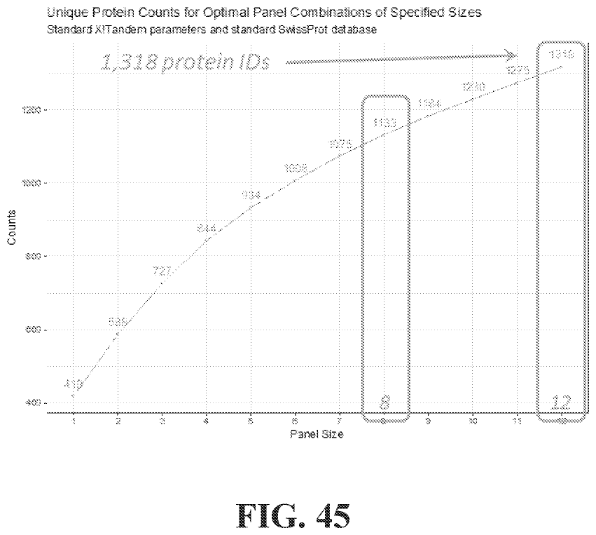

A particle panel can have more than one particle type. Increasing the number of particle types in a panel can be a method for increasing the number of proteins that can be identified in a given sample. An example of how increasing panel size may increase the number of identified proteins is shown in FIG. 45, in which a panel size of one particle type identified 419 different proteins, a panel size of two particle types identified 588 different proteins, a panel size of three particle types identified 727 different proteins, a panel size of four particle types identified 844 proteins, a panel size of five particle types identified 934 different proteins, a panel size of six particle types identified 1008 different proteins, a panel size of seven particle types identified 1075 different proteins, a panel size of eight particle types identified 1133 different proteins, a panel size of nine particle types identified 1184 different proteins, a panel size of 10 particle types identified 1230 different proteins, a panel size of 11 particle types identified 1275 different proteins, and a panel size of 12 particle types identified 1318 different proteins.

In some cases, a panel size of one particle type is capable of identifying 200 to 600 different proteins. In some cases, a panel size of two particle types is capable of identifying 300 to 700 different proteins. In some cases, a panel size of three particle types is capable of identifying 500 to 900 different proteins. In some cases, a panel size of four particle types is capable of different 600 to 1000 unique proteins. In some cases, a panel size of five particle types is capable of identifying 700 to 1100 different proteins. In some cases, a panel size of six particle types is capable of identifying 800 to 1200 different proteins. In some cases, a panel size of seven particle types is capable of identifying 850 to 1250 different proteins. In some cases, a panel size of eight particle types is capable of identifying 900 to 1300 different proteins. In some cases, a panel size of nine particle types is capable of identifying 950 to 1350 different proteins. In some cases, a panel size of 10 particle types is capable of identifying 1000 to 1400 different proteins. In some cases, a panel size of 11 particle types is capable of identifying 1050 to 1450 different proteins. In some cases, a panel size of 12 particle types is capable of identifying 1100 to 1500 different proteins. The particle types may include nanoparticle types.

A particle panel may comprise a combination of particles with silica and polymer surfaces. For example, a particle panel may comprise a SPION coated with a thin layer of silica, a SPION coated with poly(dimethyl aminopropyl methacrylamide) (PDMAPMA), and a SPION coated with poly(ethylene glycol) (PEG). A particle panel consistent with the present disclosure could also comprise two or more particles selected from the group consisting of silica coated SPION, an N-(3-Trimethoxysilylpropyl) diethylenetriamine coated SPION, a PDMAPMA coated SPION, a carboxyl-functionalized polyacrylic acid coated SPION, an amino surface functionalized SPION, a polystyrene carboxyl functionalized SPION, a silica particle, and a dextran coated SPION. A particle panel consistent with the present disclosure may also comprise two or more particles selected from the group consisting of a surfactant free carboxylate microparticle, a carboxyl functionalized polystyrene particle, a silica coated particle, a silica particle, a dextran coated particle, an oleic acid coated particle, a boronated nanopowder coated particle, a PDMAPMA coated particle, a Poly(glycidyl methacrylate-benzylamine) coated particle, and a Poly(N-[3-(Dimethylamino)propyllmethacrylamide-co42-(methacryloyloxy)ethylldimethyl-(3-sulfopropyl)ammonium hydroxide, P(DMAPMA-co-SBMA) coated particle. A particle panel consistent with the present disclosure may comprise silica-coated particles, N-(3-Trimethoxysilylpropyl)diethylenetriamine coated particles, poly(N-(3-(dimethylamino)propyl) methacrylamide) (PDMAPMA)-coated particles, phosphate-sugar functionalized polystyrene particles, amine functionalized polystyrene particles, polystyrene carboxyl functionalized particles, ubiquitin functionalized polystyrene particles, dextran coated particles, or any combination thereof.

A particle panel consistent with the present disclosure may comprise a silica functionalized particle, an amine functionalized particle, a silicon alkoxide functionalized particle, a carboxylate functionalized particle, and a benzyl or phenyl functionalized particle. A particle panel consistent with the present disclosure may comprise a silica functionalized particle, an amine functionalized particle, a silicon alkoxide functionalized particle, a polystyrene functionalized particle, and a saccharide functionalized particle. A particle panel consistent with the present disclosure may comprise a silica functionalized particle, an N-(3-Trimethoxysilylpropyl)diethylenetriamine functionalized particle, a PDMAPMA functionalized particle, a dextran functionalized particle, and a polystyrene carboxyl functionalized particle. A particle panel consistent with the present disclosure may comprise 5 particles including a silica functionalized particle, an amine functionalized particle, a silicon alkoxide functionalized particle.

Protein Corona Analysis in Biological Samples

The particles and methods of use thereof disclosed herein can bind a large number of unique proteins in a biological sample (e.g., a biofluid). Non-limiting examples of biological samples that may be analyzed using the protein corona analysis methods described herein include biofluid samples (e.g., cerebral spinal fluid (CSF), synovial fluid (SF), urine, plasma, serum, tears, semen, whole blood, milk, nipple aspirate, ductal lavage, vaginal fluid, nasal fluid, ear fluid, gastric fluid, pancreatic fluid, trabecular fluid, lung lavage, prostatic fluid, sputum, fecal matter, bronchial lavage, fluid from swabbings, bronchial aspirants, sweat or saliva), fluidized solids (e.g., a tissue homogenate), or samples derived from cell culture. For example, a particle disclosed herein can be incubated with any biological sample disclosed herein to form a protein corona comprising at least 100 unique proteins, at least 120 unique proteins, at least 140 unique proteins, at least 160 unique proteins, at least 180 unique proteins, at least 200 unique proteins, at least 220 unique proteins, at least 240 unique proteins, at least 260 unique proteins, at least 280 unique proteins, at least 300 unique proteins, at least 320 unique proteins, at least 340 unique proteins, at least 360 unique proteins, at least 380 unique proteins, at least 400 unique proteins, at least 420 unique proteins, at least 440 unique proteins, at least 460 unique proteins, at least 480 unique proteins, at least 500 unique proteins, at least 520 unique proteins, at least 540 unique proteins, at least 560 unique proteins, at least 580 unique proteins, at least 600 unique proteins, at least 620 unique proteins, at least 640 unique proteins, at least 660 unique proteins, at least 680 unique proteins, at least 700 unique proteins, at least 720 unique proteins, at least 740 unique proteins, at least 760 unique proteins, at least 780 unique proteins, at least 800 unique proteins, at least 820 unique proteins, at least 840 unique proteins, at least 860 unique proteins, at least 880 unique proteins, at least 900 unique proteins, at least 920 unique proteins, at least 940 unique proteins, at least 960 unique proteins, at least 980 unique proteins, at least 1000 unique proteins, from 100 to 1000 unique proteins, from 150 to 950 unique proteins, from 200 to 900 unique proteins, from 250 to 850 unique proteins, from 300 to 800 unique proteins, from 350 to 750 unique proteins, from 400 to 700 unique proteins, from 450 to 650 unique proteins, from 500 to 600 unique proteins, from 200 to 250 unique proteins, from 250 to 300 unique proteins, from 300 to 350 unique proteins, from 350 to 400 unique proteins, from 400 to 450 unique proteins, from 450 to 500 unique proteins, from 500 to 550 unique proteins, from 550 to 600 unique proteins, from 600 to 650 unique proteins, from 650 to 700 unique proteins, from 700 to 750 unique proteins, from 750 to 800 unique proteins, from 800 to 850 unique proteins, from 850 to 900 unique proteins, from 900 to 950 unique proteins, from 950 to 1000 unique proteins. In some cases, several different types of particles can be used, separately or in combination, to identify large numbers of proteins in a particular biological sample. In other words, particles can be multiplexed in order to bind and identify large numbers of proteins in a biological sample. Protein corona analysis of the biomolecule corona may compress the dynamic range of the analysis compared to a total protein analysis method.

The compositions and methods disclosed herein can be used to identify various biological states in a particular biological sample. For example, a biological state can refer to an elevated or low level of a particular protein or a set of proteins. In other examples, a biological state can refer to identification of a disease, such as cancer. The compositions and methods disclosed herein may be used to identify the presence or absence of a protein-protein interaction in a biological sample (e.g., a biofluid). The presence or absence of the protein-protein interaction may be indicative of a biological state. One or more particle types can be incubated with CSF, allowing for formation of a protein corona. Said protein corona can then be analyzed by gel electrophoresis or mass spectrometry in order to identify a pattern of proteins (e.g., protein-protein interactions). Analysis of protein corona (e.g., by mass spectrometry or gel electrophoresis) may be referred to as corona analysis. The pattern of proteins can be compared to the same methods carried out on a control sample. Upon comparison of the patterns of proteins, it may be identified that the first CSF sample comprises an elevated level of markers corresponding to a particular type of brain cancer. The particles and methods of use thereof, can thus be used to diagnose a particular disease state.

The particles and methods of us thereof can be used to distinguish between two biological states. The two biological states may be related diseases states (e.g., two HRAS mutant colon cancers or different stages of a type of a cancer). The two biological states may be different phases of a disease, such as pre-Alzheimer's and mild Alzheimer's. The two biological states may be distinguished with a high degree of accuracy (e.g., the percentage of accurately identified biological states among a population of samples). For example, the compositions and methods of the present disclosure may distinguish two biological states with at least 60% accuracy, at least 70% accuracy, at least 75% accuracy at least 80% accuracy, at least 85% accuracy, at least 90% accuracy, at least 95% accuracy, at least 98% accuracy, or at least 99% accuracy. The two biological states may be distinguished with a high degree of specificity (e.g., the rate at which negative results are correctly identified among a population of samples). For example, the compositions and methods of the present disclosure may distinguish two biological states with at least 60% specificity, at least 70% specificity, at least 75% specificity at least 80% specificity, at least 85% specificity, at least 90% specificity, at least 95% specificity, at least 98% specificity, or at least 99% specificity.

Protein corona analysis may comprise an automated component. For example, an automated instrument may contact a sample with a particle or particle panel, identify proteins on the particle or particle panel (e.g., digest the proteins on the particle or particle panel and perform mass spectrometric analysis), and generate data for identifying a protein-protein interaction. The automated instrument may divide a sample into a plurality of volumes, and perform analysis on each volume. The automated instrument may analyze multiple separate samples, for example by disposing multiple samples within multiple wells in a well plate, and performing parallel analysis on each sample.

Protein Corona Analysis Methods

The methods disclosed herein include isolating one or more particle types from one or more than one sample (e.g., a biological sample or a serially interrogated sample). The particle types can be rapidly isolated or separated from the sample using a magnetic. Moreover, multiple samples that are spatially isolated can be processed in parallel. Thus, the methods disclosed herein provide for isolating or separating a particle type from unbound protein in a sample. A particle type may be separated by a variety of means, including but not limited to magnetic separation, centrifugation, filtration, or gravitational separation. Particle panels may be incubated with a plurality of spatially isolated samples, wherein each spatially isolated sample is in a well in a well plate (e.g., a 96-well plate). After incubation, the particle types in each of the wells of the well plate can be separated from unbound protein present in the spatially isolated samples by placing the entire plate on a magnet. This simultaneously pulls down the superparamagnetic particles in the particle panel. The supernatant in each sample can be removed to remove the unbound protein. These steps (incubate, pull down) can be repeated to effectively wash the particles, thus removing residual background unbound protein that may be present in a sample. This is one example, but one of skill in the art could envision numerous other scenarios in which superparamagnetic particles are rapidly isolated from one or more than one spatially isolated samples at the same time.

The methods and compositions of the present disclosure provide identification and measurement of particular proteins in the biological samples by processing of the proteomic data via digestion of coronas formed on the surface of particles. Examples of proteins that can be identified and measured include highly abundant proteins, proteins of medium abundance, and low-abundance proteins. A low abundance protein may be present in a sample at concentrations at or below about 10 ng/mL. A high abundance protein may be present in a sample at concentrations at or above about 10 μg/mL. A protein of moderate abundance may be present in a sample at concentrations between about 10 ng/mL and about 10 μg/mL. Examples of proteins that are highly abundant proteins include albumin, IgG, and the top 14 proteins in abundance that contribute 95% of the mass in plasma. Additionally, any proteins that may be purified using a conventional depletion column may be directly detected in a sample using the particle panels disclosed herein. Examples of proteins may be any protein listed in published databases such as Keshishian et al. (Mol Cell Proteomics. 2015 September; 14(9):2375-93. doi: 10.1074/mcp.M114.046813. Epub 2015 Feb. 27.), Farr et al. (J Proteome Res. 2014 Jan. 3; 13(1):60-75. doi: 10.1021/pr4010037. Epub 2013 Dec. 6.), or Pernemalm et al. (Expert Rev Proteomics. 2014 August; 11(4):431-48. doi: 10.1586/14789450.2014.901157. Epub 2014 Mar. 24.).

Examples of proteins that can be measured and identified using the methods and compositions disclosed herein include albumin, IgG, lysozyme, CEA, HER-2/neu, bladder tumor antigen, thyroglobulin, alpha-fetoprotein, PSA, CA125, CA19.9, CA 15.3, leptin, prolactin, osteopontin, IGF-II, CD98, fascin, sPigR, 14-3-3 eta, troponin I, B-type natriuretic peptide, BRCA1, c-Myc, IL-6, fibrinogen. EGFR, gastrin, PH, G-CSF, desmin. NSE, FSH, VEGF, P21, PCNA, calcitonin, PR, CA125, LH, somatostatin. S100, insulin. alpha-prolactin, ACTH, Bcl-2, ER alpha, Ki-67, p53, cathepsin D, beta catenin. VWF, CD15, k-ras, caspase 3, EPN, CD10, FAS, BRCA2. CD3OL, CD30, CGA, CRP, prothrombin, CD44, APEX, transferrin, GM-CSF, E-cadherin, IL-2, Bax, IFN-gamma, beta-2-MG, TNF alpha, c-erbB-2, trypsin, cyclin D1, MG B, XBP-1, HG-1, YKL-40, S-gamma, NESP-55, netrin-1, geminin, GADD45A, CDK-6, CCL21, BrMS1, 17betaHDI, PDGFRA, Pcaf, CCLS, MMP3, claudin-4, and claudin-3. In some cases, other examples of proteins that can be measured and identified using the particle panels disclosed herein are any proteins or protein groups listed in the open targets database for a particular disease indication of interest (e.g., prostate cancer, lung cancer, or Alzheimer's disease).

The methods and compositions disclosed herein may also elucidate protein classes or interactions of the protein classes. A protein class may comprise a set of proteins that share a common function (e.g., amine oxidases or proteins involved in angiogenesis); proteins that share common physiological, cellular, or subcellular localization (e.g., peroxisomal proteins or membrane proteins); proteins that share a common cofactor (e.g., heme or flavin proteins); proteins that correspond to a particular biological state (e.g., hypoxia related proteins); proteins containing a particular structural motif (e.g., a cupin fold); or proteins bearing a post-translational modification (e.g., ubiquitinated or citrullinated proteins). A protein class may contain at least 2 proteins, 5 proteins, 10 proteins, 20 proteins, 40 proteins, 60 proteins, 80 proteins, 100 proteins, 150 proteins, 200 proteins, or more.

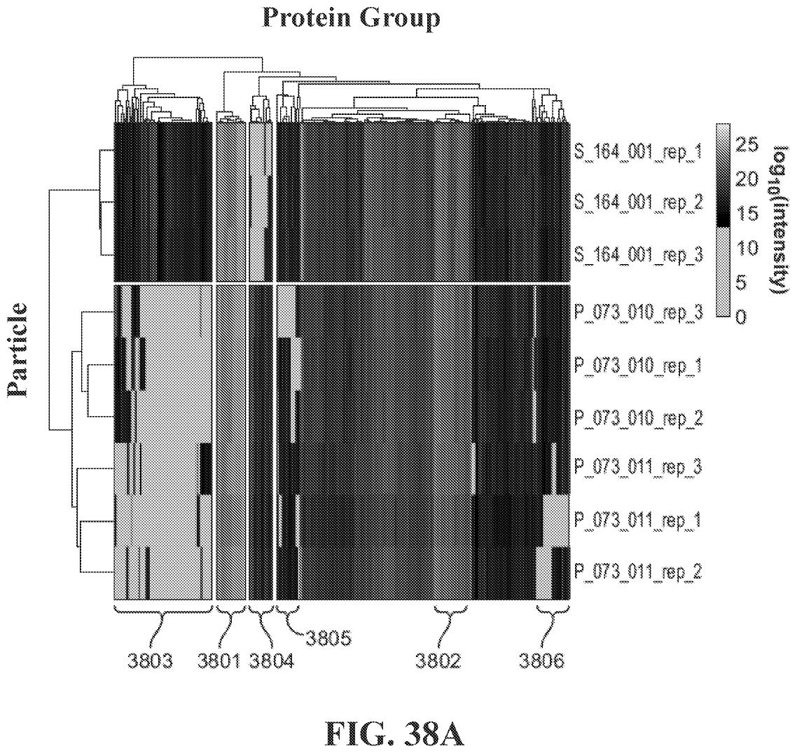

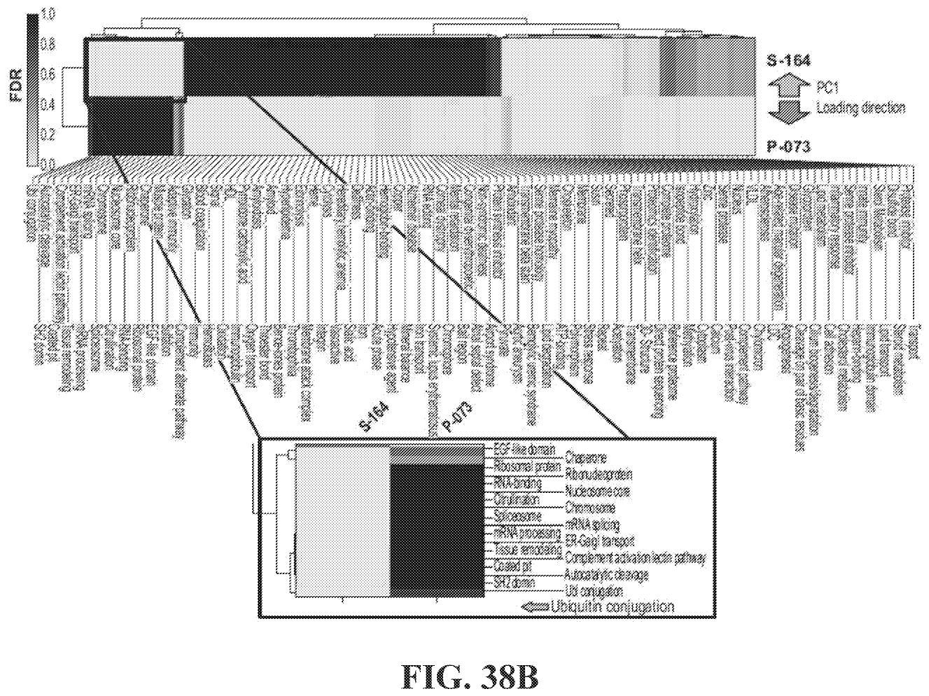

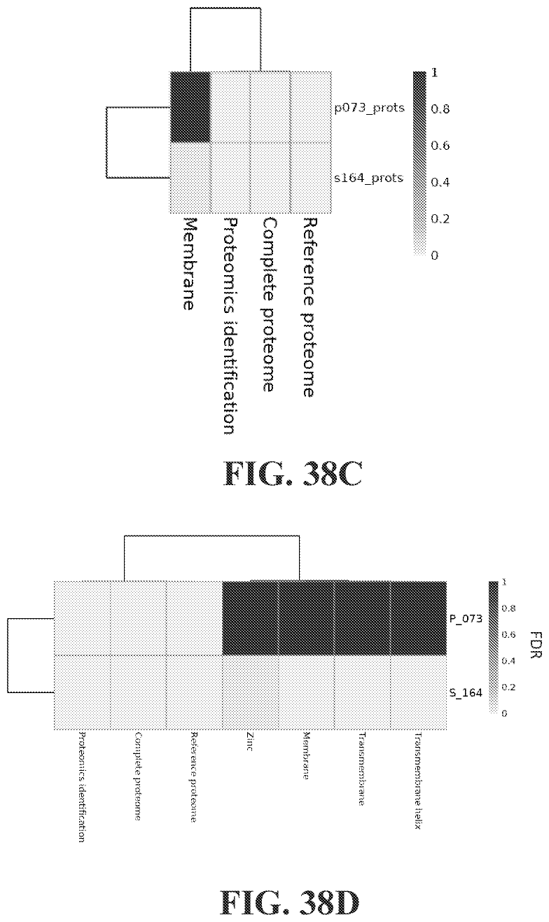

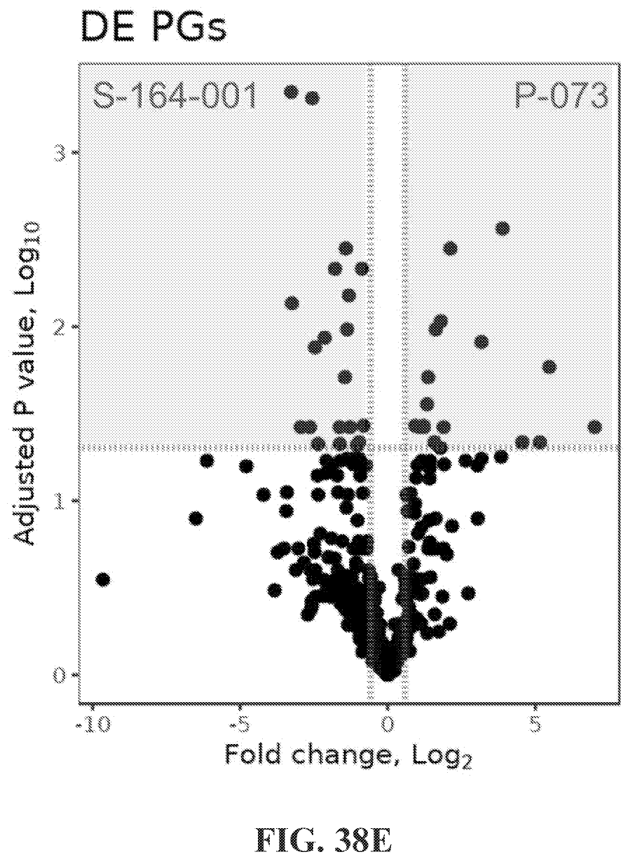

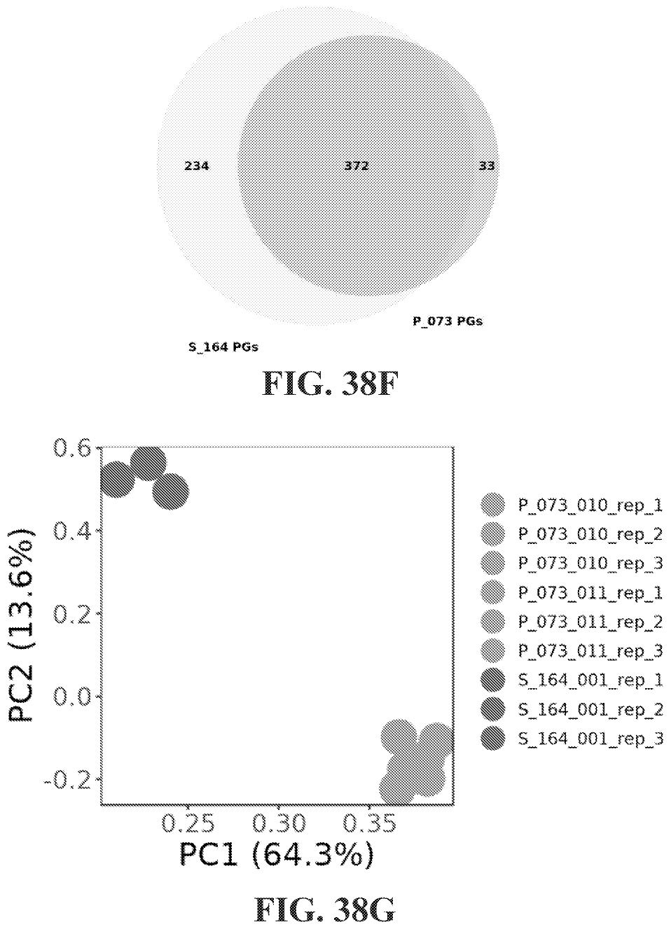

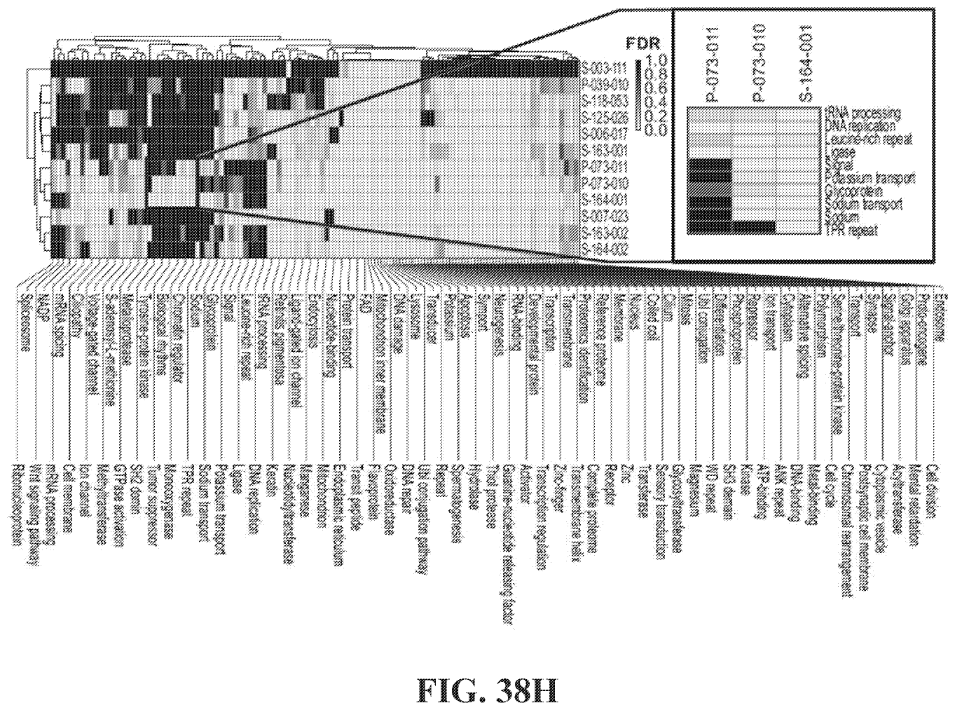

A protein class may be identified by observing a feature common to the class, such as a portion of a heme binding motif to elucidate the presence of heme proteins in a sample, or crosslinked tyrosine residues to indicate the presence of copper proteins. Protein class identification is illustrated in FIG. 38B, which provides confidence (FDR-adjusted p-values) for different protein class identifications. Protein class elucidation may be enhanced by particle functionalization. For example, functionalizing a particle surface with ubiquitin can enhance the collection of proteins bearing ubiquitin and ubiquitin-like post-translational modifications, as is shown in the blown up portion of the plot below the figure.

Protein class identifications may also aid in the identification of protein-protein interactions. For example, the identification or quantification of a protein class associated with a protein-protein interaction may confirm the presence of that protein-protein interaction, such as in cases where low quantities of the protein-protein interaction pair are recovered for analysis. For example, identification of elevated mTOR signaling or autophagy regulatory proteins may be used to confirm protein-protein interactions implicated in and indicative of Huntington's disease, such as transcription factor (e.g., CREB-binding and TATA-binding proteins) binding with huntingtin protein. Protein class identifications may be used to negatively scan for protein-protein interactions. Such an identification may be determined by identifying a protein class that indicates the presence of two proteins, along with an absence of signals or signal intensities corresponding to those proteins, thus indicating that the two proteins may be interacting in solution.

The proteomic data of the biological sample can be identified, measured, and quantified using a number of different analytical techniques. For example, proteomic data can be generated using SDS-PAGE or any gel-based separation technique. Peptides and proteins can also be identified, measured, and quantified using an immunoassay, such as ELISA. Alternatively, proteomic data can be identified, measured, and quantified using mass spectrometry, high performance liquid chromatography, LC-MS/MS, Edman Degradation, immunoaffinity techniques, methods disclosed in EP3548652, WO2019083856, WO2019133892, each of which is incorporated herein by reference in its entirety, and other protein separation techniques.

An assay may comprise protein collection of particles, protein digestion, and mass spectrometric analysis (e.g., MS, LC-MS, LC-MS/MS). The digestion may comprise chemical digestion, such as by cyanogen bromide or 2-Nitro-5-thiocyanatobenzoic acid (NTCB). The digestion may comprise enzymatic digestion, such as by trypsin or pepsin. The digestion may comprise enzymatic digestion by a plurality of proteases. The digestion may comprise a protease selected from among the group consisting of trypsin, chymotrypsin, Glu C, Lys C, elastase, subtilisin, proteinase K, thrombin, factor X, Arg C, papaine, Asp N, thermolysine, pepsin, aspartyl protease, cathepsin D, zinc mealloprotease, glycoprotein endopeptidase, proline, aminopeptidase, prenyl protease, caspase, kex2 endoprotease, or any combination thereof. The digestion may cleave peptides at random positions. The digestion may cleave peptides at a specific position (e.g., at methionines) or sequence (e.g., glutamate-histidine-glutamate). The digestion may enable similar proteins to be distinguished. For example, an assay may resolve 8 distinct proteins as a single protein group with a first digestion method, and as 8 separate proteins with distinct signals with a second digestion method. The digestion may generate an average peptide fragment length of 8 to 15 amino acids. The digestion may generate an average peptide fragment length of 12 to 18 amino acids. The digestion may generate an average peptide fragment length of 15 to 25 amino acids. The digestion may generate an average peptide fragment length of 20 to 30 amino acids. The digestion may generate an average peptide fragment length of 30 to 50 amino acids.

An assay may rapidly generate and analyze proteomic data. Beginning with an input biological sample (e.g., a buccal or nasal smear, plasma, or tissue), an assay of the present disclosure may generate and analyze proteomic data in less than 7 hours. Beginning with an input biological sample, an assay of the present disclosure may generate and analyze proteomic data in 5-7 hours. Beginning with an input biological sample, an assay of the present disclosure may generate and analyze proteomic data in less than 5 hours. Beginning with an input biological sample, an assay of the present disclosure may generate and analyze proteomic data in 3-5 hours. Beginning with an input biological sample, an assay of the present disclosure may generate and analyze proteomic data in 2-4 hours. Beginning with an input biological sample, an assay of the present disclosure may generate and analyze proteomic data in 2-3 hours. Beginning with an input biological sample, an assay of the present disclosure may generate and analyze proteomic data in less than 3 hours. Beginning with an input biological sample, an assay of the present disclosure may generate and analyze proteomic data in less than 2 hours. The analyzing may comprise identifying a protein-protein interaction. The analyzing may comprise identifying a protein group. The analyzing may comprise identifying a protein class. The analyzing may comprise quantifying an abundance of a protein-protein interaction, a protein group, or a protein class. The analyzing may comprise identifying a biological state.

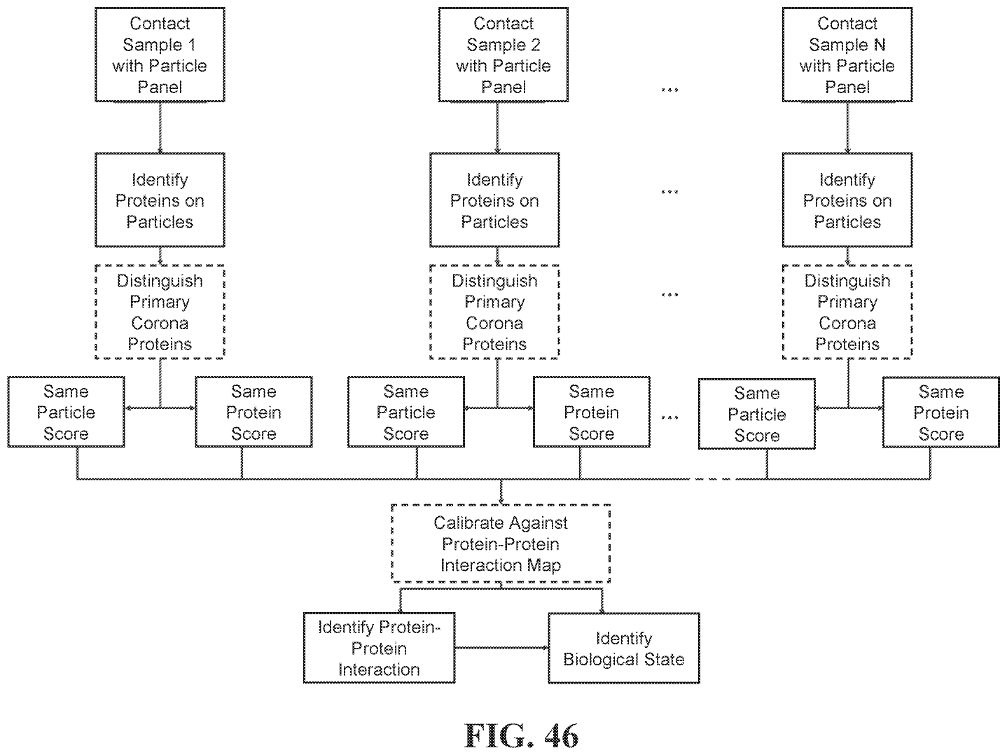

FIG. 46 illustrates a method for identifying a protein-protein interaction. A plurality of samples may each be contacted with a particle panel. The particles from the particle panel may be contacted to the sample collectively (e.g., as a mixture), or separately, for example by contacting one particle type from the particle panel to separate sample aliquots. The particle panel may be incubated with the samples. Each sample may then be separately analyzed to identify proteins bound to each particle. Optionally, the identifying can comprise determining, in vitro, whether a protein was present in the primary corona of a particle. Alternatively, protein corona data may be provided for analysis.

The data may be analyzed to determine same particle and same protein scores for proteins identified on the particle panel. The same particle scores provide information on the associations between pairs of proteins in the sample, while the same protein scores identify the affinities between that individual proteins have for particular particles. The same particle and same protein scores may optionally be calibrated against a protein-protein interaction map. The protein-protein interaction map may raise or lower a same particle or same protein score based on the structure, native localization, biological function, or known protein-protein interactions for a protein identified in the assay.

The same particle and same protein scores may be used to identify a protein-protein interaction. In some cases, a same particle score that is greater than the same protein scores for a pair of proteins may indicate a protein-protein interaction. In some cases, a same protein score above a designated threshold may distinguish a protein-protein interaction. In some cases, a positive same protein score and negative same particle score may indicate a protein-protein interaction.

The data may also be used to identify a biological state of the sample. The identification of the biological state may be based on the identified protein data. The identification may also comprise an identified protein-protein interaction, which may constitute a datapoint for identifying the biological state, or may be used to cluster or recalibrate (e.g., weight) the identified protein data.

Kits

Provided herein are kits comprising compositions of the present disclosure that may be used to perform the methods of the present disclosure. A kit may comprise one or more particle types to interrogate a sample to identify the presence or absence of a protein-protein interaction. In some cases, a kit may comprise a particle type provided in TABLES 1, 7, 9, 10, 11, or 17. In some cases, a kit may comprise a particle type comprising a bait molecule. The kit may be pre-packaged in discrete aliquots. In some cases, the kit can comprise a plurality of different particle types that can be used to interrogate a sample. The plurality of particle types can be pre-packaged where each particle type of the plurality is packaged separately. Alternately, the plurality of particle types can be packaged together to contain combination of particle types in a single package. A particle may be provided in dried (e.g., lyophilized) form, or may be provided in a suspension or solution. The particles may be provided in a well plate. For example, a kit may contain a 24-384 well plate with the particles sealed within the wells. Two wells in such a well plate may contain different particles or concentrations of particles. Two wells may comprise different buffers or chemical conditions. For example, a well plate may be provided with different particles in each row of wells and different buffers in each column of rows. A well may be sealed by a removable covering. For example, a kit may comprise a well plate comprising a plastic slip covering a plurality of wells. A well may be sealed by a pierceable covering. For example, a well may be covered by a septum that a needle can pierce to facilitate sample movement into and out of the well.

Computer Control Systems

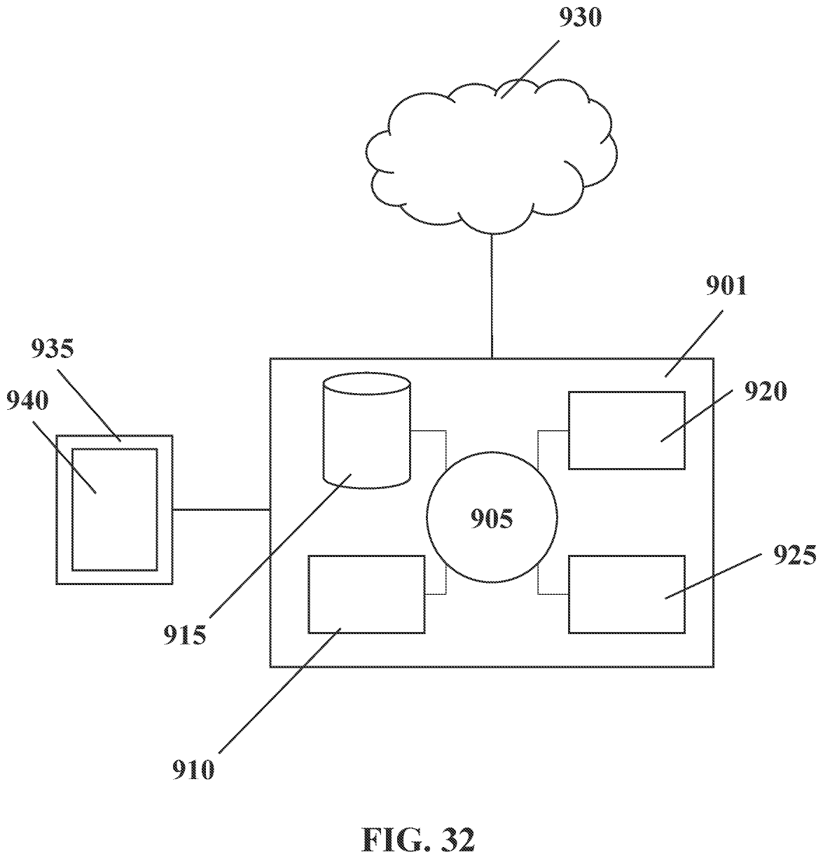

The present disclosure provides computer control systems that are programmed to implement methods of the disclosure. FIG. 32 shows a computer system that is programmed or otherwise configured to implement methods provided herein. The computer system 901 can regulate various aspects of the assays disclosed herein, which are capable of being automated (e.g., movement of any of the reagents disclosed herein on a substrate). The computer system 901 can be an electronic device of a user or a computer system that is remotely located with respect to the electronic device. The electronic device can be a mobile electronic device.

The computer system 901 includes a central processing unit (CPU, also “processor” and “computer processor” herein) 905, which can be a single core or multi core processor, or a plurality of processors for parallel processing. The computer system 901 also includes memory or memory location 910 (e.g., random-access memory, read-only memory, flash memory), electronic storage unit 915 (e.g., hard disk), communication interface 920 (e.g., network adapter) for communicating with one or more other systems, and peripheral devices 925, such as cache, other memory, data storage and/or electronic display adapters. The memory 910, storage unit 915, interface 920 and peripheral devices 925 are in communication with the CPU 905 through a communication bus (solid lines), such as a motherboard. The storage unit 915 can be a data storage unit (or data repository) for storing data. The computer system 901 can be operatively coupled to a computer network (“network”) 930 with the aid of the communication interface 920. The network 930 can be the Internet, an internet and/or extranet, or an intranet and/or extranet that is in communication with the Internet. The network 930 in some cases is a telecommunication and/or data network. The network 930 can include one or more computer servers, which can enable distributed computing, such as cloud computing. The network 930, in some cases with the aid of the computer system 901, can implement a peer-to-peer network, which may enable devices coupled to the computer system 901 to behave as a client or a server.

The CPU 905 can execute a sequence of machine-readable instructions, which can be embodied in a program or software. The instructions may be stored in a memory location, such as the memory 910. The instructions can be directed to the CPU 905, which can subsequently program or otherwise configure the CPU 905 to implement methods of the present disclosure. Examples of operations performed by the CPU 905 can include fetch, decode, execute, and writeback.

The CPU 905 can be part of a circuit, such as an integrated circuit. One or more other components of the system 901 can be included in the circuit. In some cases, the circuit is an application specific integrated circuit (ASIC).

The storage unit 915 can store files, such as drivers, libraries and saved programs. The storage unit 915 can store user data, e.g., user preferences and user programs. The computer system 901 in some cases can include one or more additional data storage units that are external to the computer system 901, such as located on a remote server that is in communication with the computer system 901 through an intranet or the Internet.

The computer system 901 can communicate with one or more remote computer systems through the network 930. For instance, the computer system 901 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 901 via the network 930.

Methods as described herein can be implemented by way of machine (e.g., computer processor) executable code stored on an electronic storage location of the computer system 901, such as, for example, on the memory 910 or electronic storage unit 915. The machine executable or machine readable code can be provided in the form of software. During use, the code can be executed by the processor 905. In some cases, the code can be retrieved from the storage unit 915 and stored on the memory 910 for ready access by the processor 905. In some situations, the electronic storage unit 915 can be precluded, and machine-executable instructions are stored on memory 910.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 901, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 901 can include or be in communication with an electronic display 935 that comprises a user interface (UI) 940 for providing, for example a readout of the proteins identified using the methods disclosed herein. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 905.

Determination, analysis or statistical classification is done by methods known in the art, including, but not limited to, for example, a wide variety of supervised and unsupervised data analysis and clustering approaches such as hierarchical cluster analysis (HCA), principal component analysis (PCA), Partial least squares Discriminant Analysis (PLSDA), machine learning (also known as random forest), logistic regression, decision trees, support vector machine (SVM), k-nearest neighbors, naive bayes, linear regression, polynomial regression, SVM for regression, K-means clustering, and hidden Markov models, among others. The computer system can perform various aspects of analyzing the protein sets or protein corona of the present disclosure, such as, for example, comparing/analyzing the biomolecule corona of several samples to determine with statistical significance what patterns are common between the individual biomolecule coronas to determine a protein set that is associated with the biological state. The computer system can be used to develop classifiers to detect and discriminate different protein sets or protein corona (e.g., characteristic of the composition of a protein corona). Data collected from the presently disclosed sensor array can be used to train a machine learning algorithm, specifically an algorithm that receives array measurements from a patient and outputs specific biomolecule corona compositions from each patient. Before training the algorithm, raw data from the array can be first denoised to reduce variability in individual variables. Machine learning can be generalized as the ability of a learning machine to perform accurately on new, unseen examples/tasks after having experienced a learning data set. Machine learning may include the following concepts and methods. Supervised learning concepts may include AODE; Artificial neural network, such as Backpropagation, Autoencoders, Hopfield networks, Boltzmann machines, Restricted Boltzmann Machines, and Spiking neural networks; Bayesian statistics, such as Bayesian network and Bayesian knowledge base; Case-based reasoning; Gaussian process regression; Gene expression programming; Group method of data handling (GMDH); Inductive logic programming; Instance-based learning; Lazy learning; Learning Automata; Learning Vector Quantization; Logistic Model Tree; Minimum message length (decision trees, decision graphs, etc.), such as Nearest Neighbor Algorithm and Analogical modeling; Probably approximately correct learning (PAC) learning; Ripple down rules, a knowledge acquisition methodology; Symbolic machine learning algorithms; Support vector machines; Random Forests; Ensembles of classifiers, such as Bootstrap aggregating (bagging) and Boosting (meta-algorithm); Ordinal classification; Information fuzzy networks (IFN); Conditional Random Field; ANOVA; Linear classifiers, such as Fisher's linear discriminant, Linear regression, Logistic regression, Multinomial logistic regression, Naive Bayes classifier, Perceptron, Support vector machines; Quadratic classifiers; k-nearest neighbor; Boosting; Decision trees, such as C4.5, Random forests, ID3, CART, SLIQ SPRINT; Bayesian networks, such as Naive Bayes; and Hidden Markov models. Unsupervised learning concepts may include; Expectation-maximization algorithm; Vector Quantization; Generative topographic map; Information bottleneck method; Artificial neural network, such as Self-organizing map; Association rule learning, such as, Apriori algorithm, Eclat algorithm, and FPgrowth algorithm; Hierarchical clustering, such as Singlelinkage clustering and Conceptual clustering; Cluster analysis, such as, K-means algorithm, Fuzzy clustering, DBSCAN, and OPTICS algorithm; and Outlier Detection, such as Local Outlier Factor. Semi-supervised learning concepts may include; Generative models; Low-density separation; Graph-based methods; and Co-training.

Reinforcement learning concepts may include; Temporal difference learning; Q-learning; Learning Automata; and SARSA. Deep learning concepts may include; Deep belief networks; Deep Boltzmann machines; Deep Convolutional neural networks; Deep Recurrent neural networks; and Hierarchical temporal memory. A computer system may be adapted to implement a method described herein. The system includes a central computer server that is programmed to implement the methods described herein. The server includes a central processing unit (CPU, also “processor”) which can be a single core processor, a multi core processor, or plurality of processors for parallel processing. The server also includes memory (e.g., random access memory, read-only memory, flash memory); electronic storage unit (e.g. hard disk); communications interface (e.g., network adaptor) for communicating with one or more other systems; and peripheral devices which may include cache, other memory, data storage, and/or electronic display adaptors. The memory, storage unit, interface, and peripheral devices are in communication with the processor through a communications bus (solid lines), such as a motherboard. The storage unit can be a data storage unit for storing data. The server is operatively coupled to a computer network (“network”) with the aid of the communications interface. The network can be the Internet, an intranet and/or an extranet, an intranet and/or extranet that is in communication with the Internet, a telecommunication or data network. The network in some cases, with the aid of the server, can implement a peer-to-peer network, which may enable devices coupled to the server to behave as a client or a server.

The storage unit can store files, such as subject reports, and/or communications with the data about individuals, or any aspect of data associated with the present disclosure.

The computer server can communicate with one or more remote computer systems through the network. The one or more remote computer systems may be, for example, personal computers, laptops, tablets, telephones, Smart phones, or personal digital assistants.

In some applications the computer system includes a single server. In other situations, the system includes multiple servers in communication with one another through an intranet, extranet and/or the internet.

The server can be adapted to store measurement data or a database as provided herein, patient information from the subject, such as, for example, medical history, family history, demographic data and/or other clinical or personal information of potential relevance to a particular application. Such information can be stored on the storage unit or the server and such data can be transmitted through a network.

Methods as described herein can be implemented by way of machine (or computer processor) executable code (or software) stored on an electronic storage location of the server, such as, for example, on the memory, or electronic storage unit. During use, the code can be executed by the processor. In some cases, the code can be retrieved from the storage unit and stored on the memory for ready access by the processor. In some situations, the electronic storage unit can be precluded, and machine-executable instructions are stored on memory. Alternatively, the code can be executed on a second computer system.

Aspects of the systems and methods provided herein, such as the server, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical, and electromagnetic waves, such as used across physical interfaces between local devices, through wired and optical landline networks and over various air-links. The physical elements that carry such waves, such as wired or wireless likes, optical links, or the like, also may be considered as media bearing the software. As used herein, unless restricted to non-transitory, tangible “storage” media, terms such as computer or machine “readable medium” can refer to any medium that participates in providing instructions to a processor for execution.

The computer systems described herein may comprise computer-executable code for performing any of the algorithms or algorithms-based methods described herein. In some applications the algorithms described herein will make use of a memory unit that is comprised of at least one database.

Data relating to the present disclosure can be transmitted over a network or connections for reception and/or review by a receiver. The receiver can be but is not limited to the subject to whom the report pertains; or to a caregiver thereof, e.g., a health care provider, manager, other health care professional, or other caretaker; a person or entity that performed and/or ordered the analysis. The receiver can also be a local or remote system for storing such reports (e.g. servers or other systems of a “cloud computing” architecture). In one embodiment, a computer-readable medium includes a medium suitable for transmission of a result of an analysis of a biological sample using the methods described herein.

Aspects of the systems and methods provided herein can be embodied in programming. Various aspects of the technology may be thought of as “products” or “articles of manufacture” typically in the form of machine (or processor) executable code and/or associated data that is carried on or embodied in a type of machine readable medium. Machine executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

### Classification of Protein Corona(s) Using Machine Learning

The method of determining protein-protein interaction candidates include the analysis of the corona of the at least two samples. This determination, analysis or statistical classification is done by methods known in the art, including, but not limited to, for example, a wide variety of supervised and unsupervised data analysis, machine learning, deep learning, and clustering approaches including hierarchical cluster analysis (HCA), principal component analysis (PCA), Partial least squares Discriminant Analysis (PLS-DA), random forest, logistic regression, decision trees, support vector machine (SVM), k-nearest neighbors, naive Bayes, linear regression, polynomial regression, SVM for regression, K-means clustering, and hidden Markov models, among others. In other words, the proteins in the corona of each sample are compared/analyzed with each other to determine with statistical significance what patterns are common between the individual corona to determine a set of protein pairs that form potential protein-protein interactions.

Generally, machine learning algorithms are used to construct models that accurately assign class labels to examples based on the input features that describe the example. In some case it may be advantageous to employ machine learning and/or deep learning approaches for the methods described herein. For example, machine learning can be used to identify potential protein-protein interactions (e.g. two or more proteins that may directly or indirectly interact with each other). For example, in some cases, one or more machine learning algorithms are employed in connection with a method of the invention to analyze data detected and obtained by the protein corona and sets of proteins derived therefrom. In some cases, protein-protein interactions may depend on a sample type or biological state. For example, in one embodiment, machine learning can be coupled with the sensor array described herein to identify protein-protein interactions in a biological sample corresponding to a first biological state (e.g., cancer) and in a biological sample corresponding to a second biological state (e.g., no cancer). Protein-protein interactions that differ between the first biological state and the second biological state may be used to identify a biological state in an unknown biological sample. For example, a protein-protein interaction may be present in a cancer sample but not in a non-cancer sample.

A method of the present disclosure may comprise a machine learning algorithm for identifying protein-protein interactions. Such a method may comprise obtaining data corresponding to a plurality of proteins collected on a plurality of particles, indicating known protein-protein interactions from among the data, and training an algorithm to identify protein-protein interactions based on the provided data. A trained algorithm may recalibrate a same particle or same protein score for a first protein and a second protein based on an identified third protein, or based on a pattern of identified proteins. A trained algorithm may factorize or transform protein data.

Unless otherwise defined, all technical terms used herein have the same meaning as commonly understood by one of ordinary skill in the art to which this invention belongs. As used in this specification and the appended claims, the singular forms “a,” “an,” and “the” include plural references unless the context clearly dictates otherwise. Any reference to “or” herein is intended to encompass “and/or” unless otherwise stated.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” “less than or equal to,” or “at most” precedes the first numerical value in a series of two or more numerical values, the term “no more than,” “less than” or “less than or equal to,” or “at most” applies to each of the numerical values in that series of numerical values. For example, less than or equal to 3, 2, or 1 is equivalent to less than or equal to 3, less than or equal to 2, or less than or equal to 1.

Where values are described as ranges, it will be understood that such disclosure includes the disclosure of all possible sub-ranges within such ranges, as well as specific numerical values that fall within such ranges irrespective of whether a specific numerical value or specific sub-range is expressly stated.

### EXAMPLES

The following examples are illustrative and non-limiting to the scope of the compositions, devices, systems, kits, and methods described herein.

### Example 1

### 1D-Enrichment Analysis Between Protein Annotations and Particle Biophysicochemical Properties

This example describes 1D-enrichment analysis between protein annotations and particle biophysicochemical properties. The depth of plasma proteome coverage for a 10 nanoparticle (NP) panel using a pooled plasma sample was determined by comparison of the NP-detected proteins to published MS intensities and spanned nearly the entire reported range. Examining protein annotations (e.g., GO Cellular Compartment and Biological Process, KEGG and Pfam) within each NP corona revealed correlations by 1D-enrichment analysis between protein annotations and NP biophysicochemical properties suggesting specific relationships at the nano-bio surface.

Selection and optimization of a panel of 10 NPs for plasma proteome profiling were demonstrated. The breadth and depth of this panel's ability to accurately and precisely quantify proteins from plasma was determined. The Proteograph platform may enable population-scale deep and unbiased proteomics analysis previously not feasible using existing workflows. The Proteograph platform may enable identification of protein-protein interactions in protein corona.

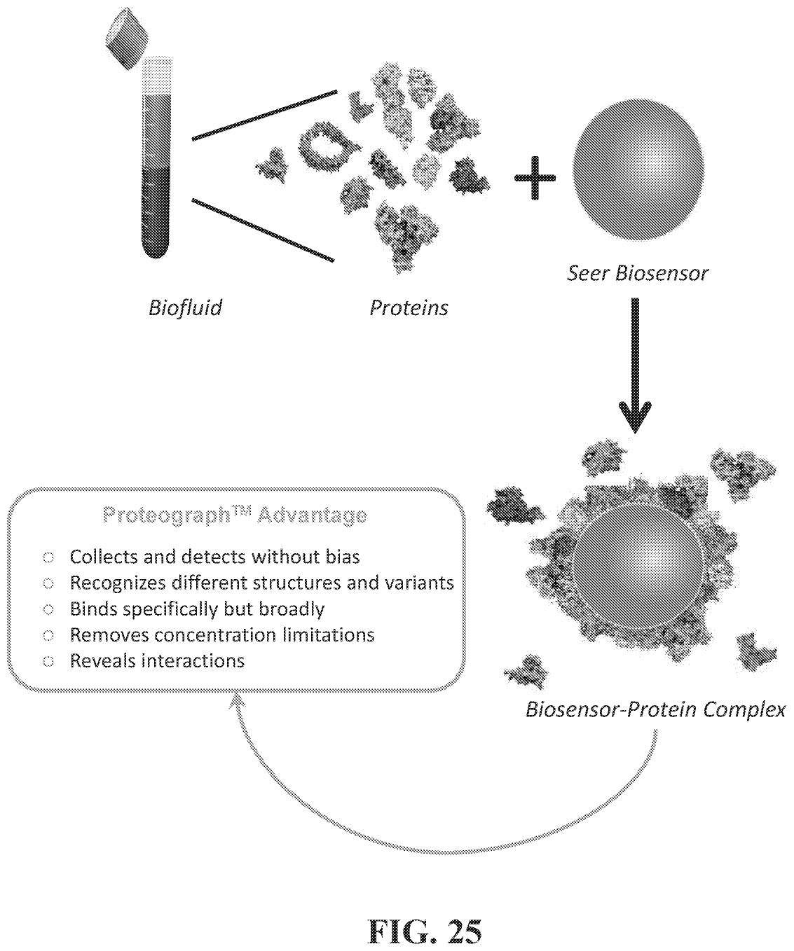

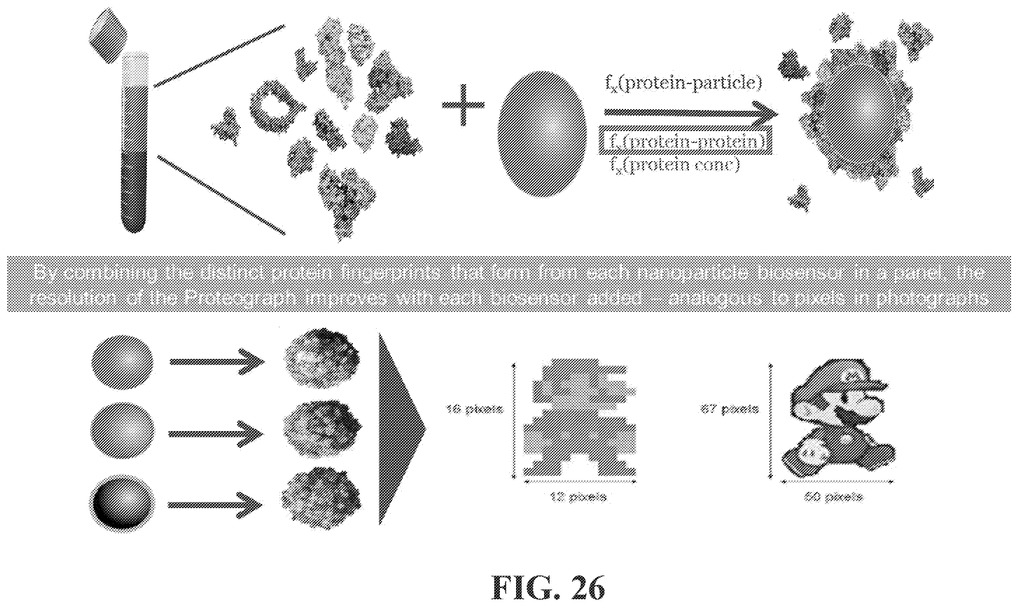

FIG. 25 shows a schematic of a protein corona analysis assay, also referred to as a Proteograph assay, performed on a biofluid. FIG. 26 shows a schematic of a protein corona analysis assay, also referred to as a Proteograph assay, to identify protein fingerprints on multiple particle types (“biosensors”).

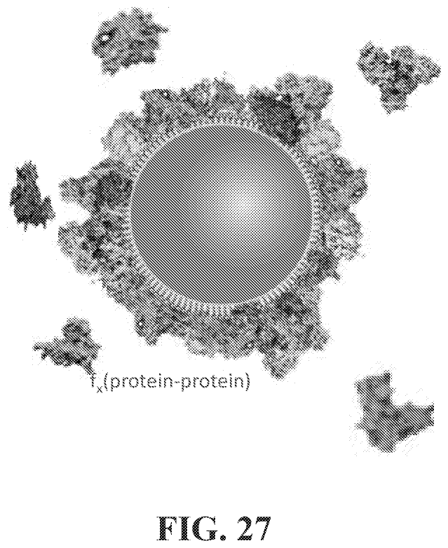

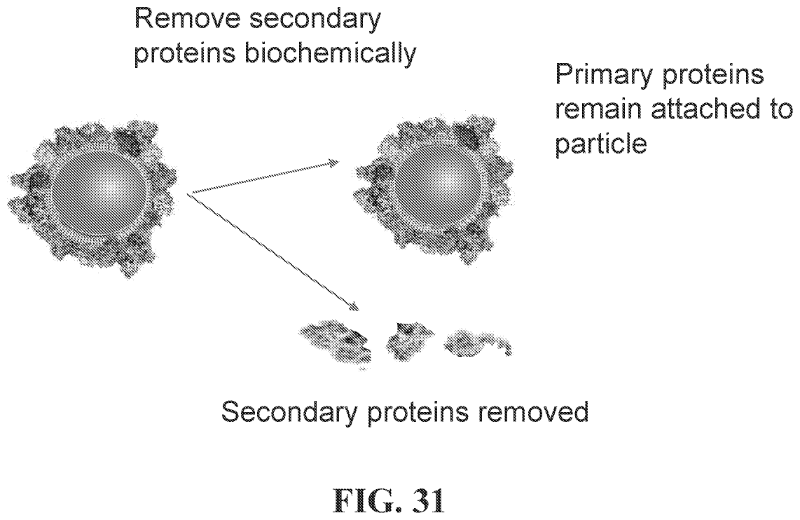

FIG. 27 shows a schematic of primary proteins, secondary proteins, tertiary proteins, and so on, interacting with a particle. Primary proteins are proteins which are aggregated primarily through their direct interactions with the particle surface. Secondary proteins are proteins which are aggregated primarily through their interactions with primary proteins. Tertiary proteins are proteins which are aggregated primarily through their interactions with secondary proteins. Additional protein layers may also form. FIG. 31 shows a schematic illustrating a method to determine both primary and secondary proteins using protein corona analysis. Secondary proteins in a protein corona may be removed biochemically while primary proteins remain attached to the particle. With a diverse set of particles and a sufficient number of protein coronas, protein-protein interactions may be identified. If protein B is only observed as a secondary protein when protein A is present as a primary protein (or vice-versa), then a protein-protein interaction between protein A and protein B is identified. Protein-protein interactions may also be identified in protein corona without biochemical removal of secondary proteins.

### Example 2

### Plasma Protein-Protein Interactome (PPI) Maps Derived from the Protein Corona Captured at the Nano-Bio Interface of Nanoparticles Reveal Differential Networks for Non-Small Cell Lung Cancer (NSCLC) and Control Subjects

This example describes plasma protein-protein interaction (PPI) maps derived from the protein corona captured at the nano-bio interface of nanoparticles reveal differential networks for non-small cell lung cancer (NSCLC) and control subjects.

Understanding changes in PPI maps from a healthy and diseased state can illuminate the understanding of biological changes and disease processes. PPI maps enable a higher order of information than a simple listing of components by providing functional context, yet existing maps grossly underrepresent the total biological information potential of PPIs. Proteograph is a novel platform that leverages the nano-bio interactions of nanoparticles (NPs) for deep and unbiased proteomic sampling that can provide insights on PPI across biological samples. Proteograph leverages the protein corona that forms on the surface of NPs as a function of their distinct biophysicochemical properties. NPs reproducibly bind subsets of proteins from biofluids as a function of protein concentration, protein-NP affinity, and protein-protein interactions to form a corona on the NP surface. Proteograph was employed to quantify known PPIs using a panel of 3 distinct NPs to capture plasma proteins and derive maps of NSCLC and control subjects in order to identify biological changes in interactions, potentially indicative of health and disease.

Method and Results: Plasma samples were collected from 288 subjects: healthy (n=82), comorbid (n=81) and NSCLC stages I-IV (n=125). In this initial study, three NPs were used with distinct properties and evaluated the protein corona of plasma samples by mass spectrometry (MS) to quantify 1,235 protein groups (1% FDR). A fully automated assay workflow enabled preparation of 3 NPs' corona for MS analysis across 288 subjects in approximately 6 days. The protein groups were mapped to a PPI map derived from the STRING database. Partitioning the network into clusters identified 9 interaction clusters with greater than 10 protein members. These clusters enabled investigation of differences in the PPI networks between NSCLC patients vs. controls. Evaluating the expression of proteins in these groups, interaction clusters were identified that had significant differences between cancer vs. control (t-test, p<0.01 Bonferroni corrected). Six of the clusters show differential behavior between NSCLC vs. healthy controls (p <0.01). Two of these clusters show differential behavior between NSCLC vs. healthy and comorbid (p<0.01). Investigation of these differentially expressed clusters reveals links to known cancer biology with proteins related to the immune system and endocytosis pathways.

Discussion: The Proteograph platform was used to identify PPI clusters that are differentiated between NSCLC and control individuals. The efficiency of the Proteograph platform applied to sufficiently powered studies may enable comprehensive understanding of known PPIs, and potentially infer and confirm new PPIs, in health and disease.

### Example 3

### Synthesis and Characterization of Iron Oxide NPs with Distinct Surface Chemistries

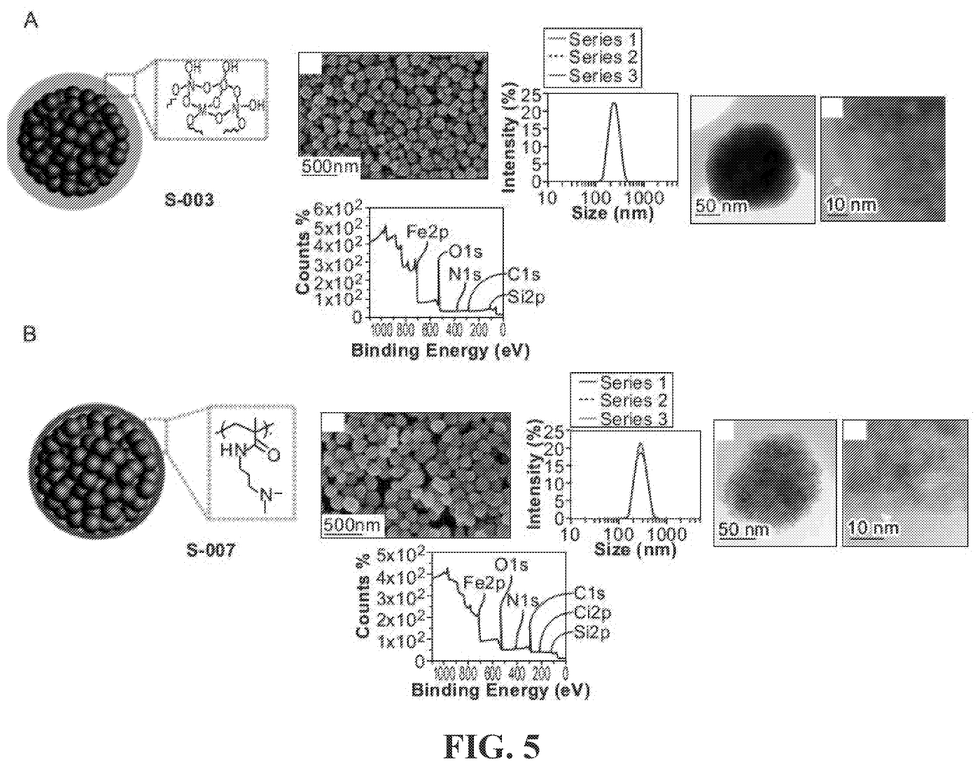

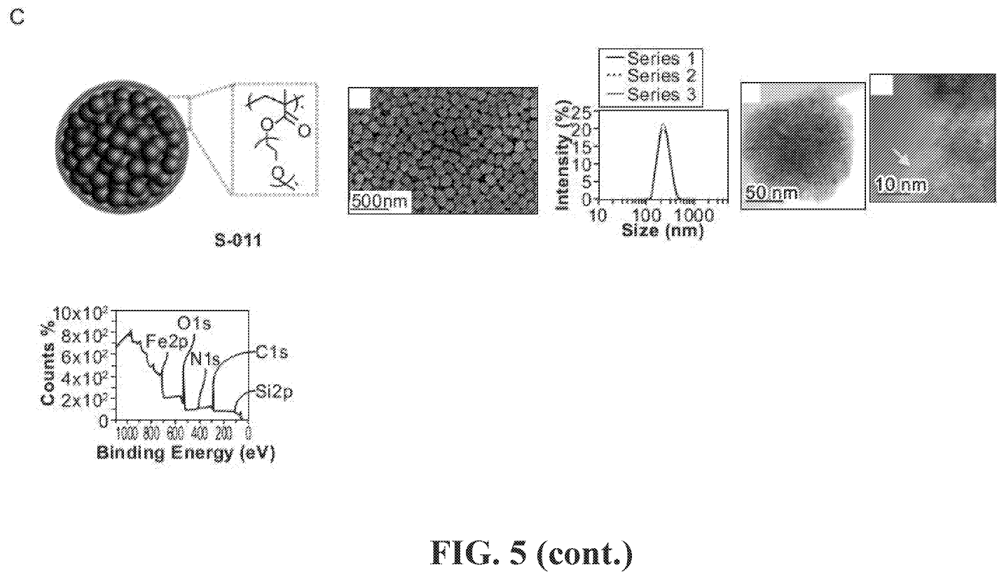

This example describes synthesis and characterization of iron oxide NPs with distinct surface chemistries. To address the need for robust particles that can be easily separated, without the need for, but which is also capable of withstanding, repeated centrifugation or membrane filtration to separate particle protein corona from free plasma proteins and to wash away loosely attached proteins from the particles, superparamagnetic iron oxide NPs (SPIONs) were developed (FIG. 5, at top) for protein corona formation. The iron oxide particle core facilitated rapid separation of the particles from plasma solution in <30 sec using a magnet (FIG. 2). This drastically reduced the time needed for extraction of particle protein corona for LC-MS/MS analysis. Moreover, SPIONs were robustly modified with different surface chemistries, which facilitated the generation of distinct patterns of protein corona for more broadly interrogating the proteome.

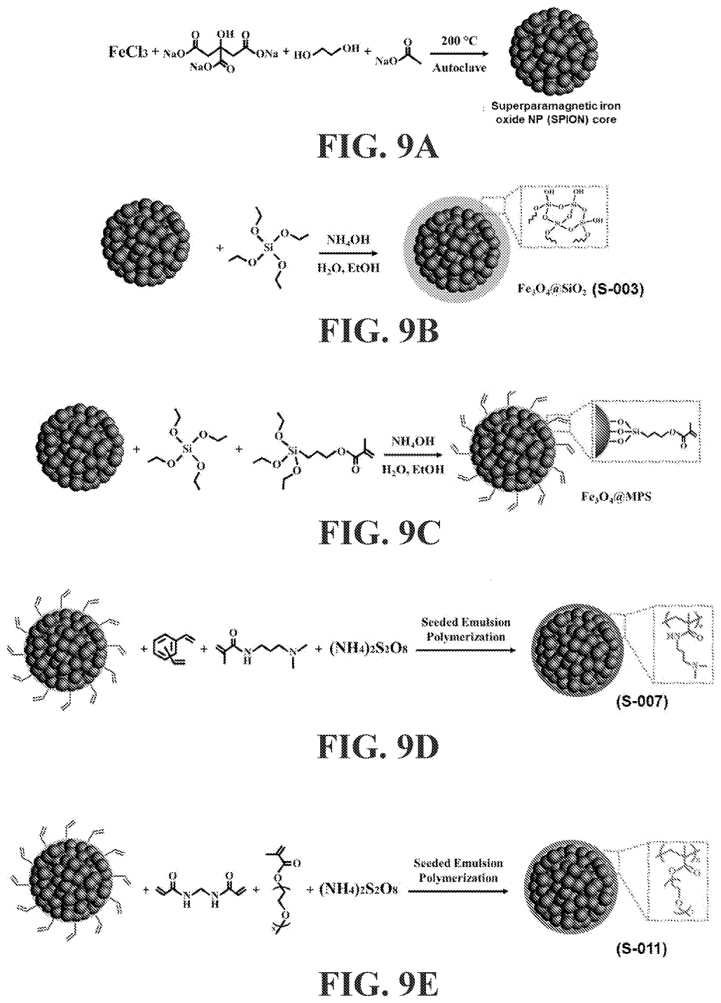

Three SPIONs (SP-003, SP-007, and SP-011) with different surface functionalization were synthesized (FIG. 9). SP-003 was coated with a thin layer of silica by a modified Stober process using tetraethyl orthosilicate (TEOS). For synthesis of poly(dimethyl aminopropyl methacrylamide) (PDMAPMA)-coated SPIONs (SP-007) and poly(ethylene glycol) (PEG)-coated SPIONs (SP-011), w the iron oxide particle core was first modified with vinyl groups by a modified Stober process using TEOS and 3-(trimethoxysilyl)propyl methacrylate. Next, the vinyl group-functionalized SPIONs were surface modified by free radical polymerization with N[3-(dimethylamino)propyl]methacrylamide and poly(ethylene glycol) methyl ether methacrylate, respectively, to prepare SP-007 and SP-011.

The three SPIONs were characterized using various techniques, including scanning electron microscopy (SEM), dynamic light scattering (DLS), transmission electron microscopy (TEM), high-resolution TEM (HRTEM), and X-ray photoelectron spectroscopy (XPS), to evaluate the size, morphology, and surface properties of SPIONs (FIG. 5). The results of DLS measurements showed that SP-003, SP-007, and SP-011 had average sizes of ˜233 nm, ˜283 nm, and ˜238 nm, respectively. This was consistent with SEM measurements, which showed that all three SPIONs had spherical and semi-spherical morphologies with sizes ranging from 200 nm to 300 nm. The surface charge of SPIONs was evaluated by zeta potential analysis, which showed ζ-potential values of −36.9 mV, +25.8 mV, and −0.4 mV for SP-003, SP-007, and SP-011, respectively, at pH 7.4 (TABLE 2-4).

TABLE 2
Particle diameter and zeta potential
of SP-003 SPION, as measured by DLS

| | | | Zeta potential |
|---|---|---|---|
| Measurement # | Z-average size | PDI | (mV) |
| 1 | 233.8 | 0.053 | −36.4 |
| 2 | 235.3 | 0.039 | −36.8 |
| 3 | 230.4 | 0.055 | −37.4 |
| Average | 233.2 nm | 0.05 | −36.9 mV |

TABLE 3
Particle diameter and zeta potential
of SP-007 SPION, as measured by DLS

| | | | Zeta potential |
|---|---|---|---|
| Measurement # | Z-average size | PDI | (mV) |
| 1 | 284.4 | 0.049 | 25.7 |
| 2 | 286.1 | 0.119 | 25.9 |
| 3 | 279.7 | 0.113 | 25.9 |
| Average | 283.4 nm | 0.09 | +25.8 mV |

TABLE 4
Particle diameter and zeta potential
of SP-011 SPION, as measured by DLS

| | | | Zeta potential |
|---|---|---|---|
| Measurement # | Z-average size | PDI | (mV) |
| 1 | 236.5 | 0.207 | 0.08 |
| 2 | 238.9 | 0.198 | −0.67 |
| 3 | 237.6 | 0.201 | −0.74 |
| Average | 237.7 nm | 0.2 | −0.4 mV |

This indicated that the SP-003, SP-007, and SP-011 had negative, positive, and neutral surfaces, which was consistent with the charge of coating functionalities used to modify the surface of each particle as shown in the schematics of FIG. 5. The thickness of the coatings was evaluated using HRTEM. For SP-003, a complete amorphous shell was observed around the iron oxide core with a thickness greater than 10 nm (FIG. 5, column 5 at top). For SP-007 and SP-011, a relatively thin (<10 nm) amorphous feature was observed at the surface of particles (arrows in FIG. 5; column 5, at middle and bottom). In addition, XPS was performed for surface analysis, which, along with HRTEM images, confirmed the successful coating of the particles with respective functional groups. The analytical results described above confirm that these three SPIONs constitute a diverse test set of NPs, which were further evaluated for protein detection coverage, precision, and linearity of response.

### Example 4

### Rapid and Deep Proteomic Analysis by the Corona Analysis Workflow

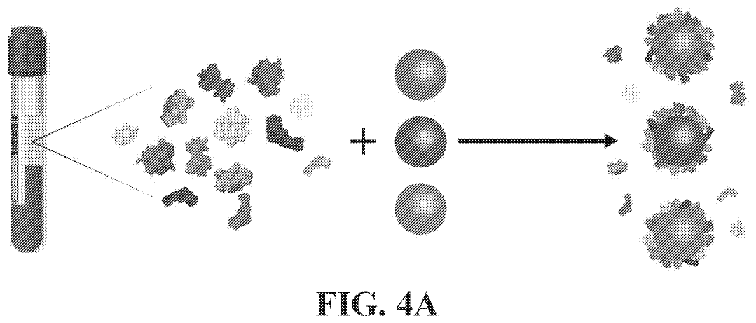

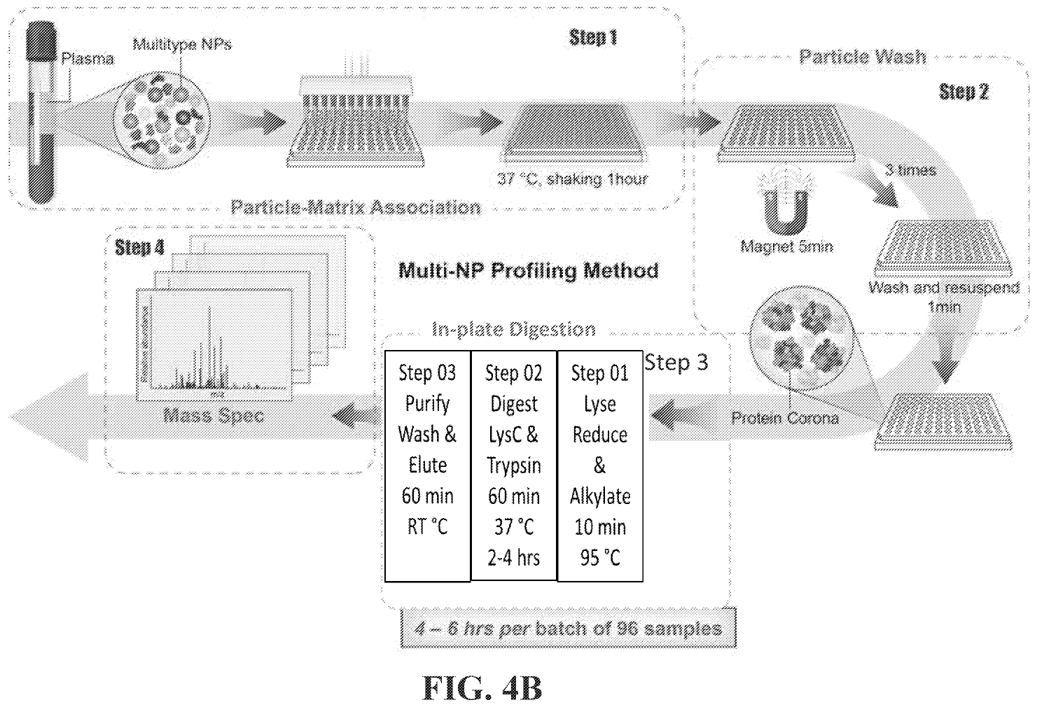

This example describes rapid and deep proteomic analysis by the corona analysis workflow. To evaluate the multi-particle type protein corona analysis platform (FIG. 4B) for analysis of plasma proteome, SPIONs were tested with a pooled plasma sample combined from eight colorectal cancer (CRC) cancer subjects. Each of these three particle types was first incubated with the plasma sample for about 1 hour at about 37° C. for protein corona formation, followed by a magnet-based purification of particles from unbound proteins (6 min per cycle for 3 times). The proteins bound onto particle were then lysed, digested, purified and eluted; these steps taking ˜2-4 hours combined, before MS analysis. Notably, this preparation workflow required only ˜4-6 hours in total for a batch of 96 corona samples.

The peptides from the NP-bound corona were analyzed by LC-MS and subsequent MS2 peptide-spectrum matching and protein group assembly (MaxQuant 1% protein 1% peptide FDR). The results, summarized in TABLE 5, show the counts of protein groups and their individual proteins were reproducibly detected in each of the three assay replicates performed in the experiment, a robust test for reproducibility. The three SPIONs detected a total of 589 protein groups (1% protein false detection rate; MaxQuant output Supplemental File proteinGroups_InitialPanel.txt). The protein groups included 196 that were common to all three SPIONs, 168 that were detected on two of three SPIONs, and 225 (38% of the 589 detected protein groups) were unique to just one of the three diverse SPIONs in this initial evaluation set. TABLE 5. Protein group and individual protein count from the NP corona of the three initial SPIONs, S-003, S-007, and 5-011 as determined by DDA LC-MS and MaxQuant (1% protein FDR). The totals represent the proteins detected in each of three replicates.

TABLE 5
Initial Evaluation Set

| | Total | Unique | Total | Unique |
|---|---|---|---|---|
| | Protein | Protein | Individual | Individual |
| Nanoparticle | Groups | Groups | Proteins | Proteins |
| S-003 | 427 | 89 | 1259 | 292 |
| S-007 | 344 | 99 | 985 | 329 |
| S-011 | 378 | 37 | 1097 | 118 |
| Panel | 589 | | 1782 | |

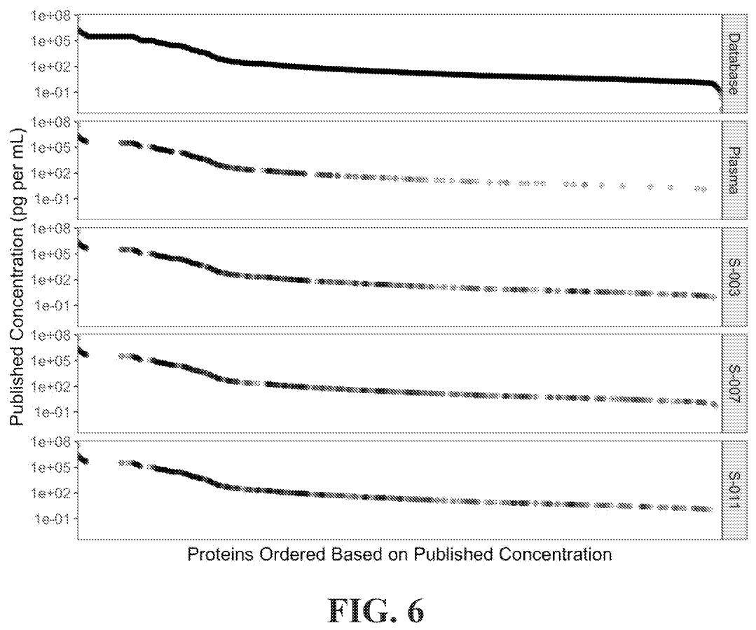

After MS analysis and data processing, the resulting MS2 peptide-spectral matches (PSM) were used to identify proteins present in each particle type corona. In parallel, proteins were also detected from a neat plasma sample directly, without particle corona formation. Comparing the identified proteins from the samples to a compiled database of MS measured or inferred plasma protein concentrations, the depth and extent of coverage by particle corona or plasma was examined by plotting observed proteins versus the database values of published protein concentrations (FIG. 6). First, the 1,255 proteins from the database covering almost 11-orders of magnitude in order from most abundant to least abundant protein were plotted. For each of the experimentally evaluated samples (neat plasma vs. SP-003/SP-007/SP-011 particle corona), the proteins matching the database were similarly plotted. As can be seen in FIG. 6, the measured plasma proteome's dynamic range as defined by the range of concentrations for database-matching proteins was 2-fold greater for particle corona (e.g., from 40 mg/mL to 0.54 ng/mL for SP-007) than it was for neat plasma (from 40 mg/mL to 1.2 ng/mL) with a 10-fold increase in the number of low abundant proteins present below 100 ng/mL (842 for particles and 84 for neat plasma). There were only 12 proteins annotated in the database with a lower concentration than the lowest protein detected on the particles. In addition, the total number of unique proteins for each of the particle type corona (1,000) is greater (>2-fold) than that observed for neat plasma (<500), as clearly demonstrated in TABLE 6.

TABLE 6
Coverage of proteins identified by SP-003, SP-
007, and SP-011 particles versus neat plasma

| Group | Total Proteins | Match to Database | Fraction in Database |
|---|---|---|---|
| Plasma | 492 | 272 | 0.55 |
| SP-003 | 1062 | 387 | 0.36 |
| SP-007 | 991 | 383 | 0.39 |
| SP-011 | 1062 | 393 | 0.37 |

In addition, the fraction of proteins that were previously unobserved by comparison to the literature MS compilation was greater (61-64%) for particles as compared to neat plasma (45%). In other words, more proteins unannotated with a prior MS concentration in the published database were identified in particle corona than were observed in neat plasma. The plot of the particle protein identifications which overlap the database confirm that different particle types select differential subsets of the plasma proteins. This could be attributable to the different surface properties of the three SPION particle types, which largely determine the protein composition of corona.

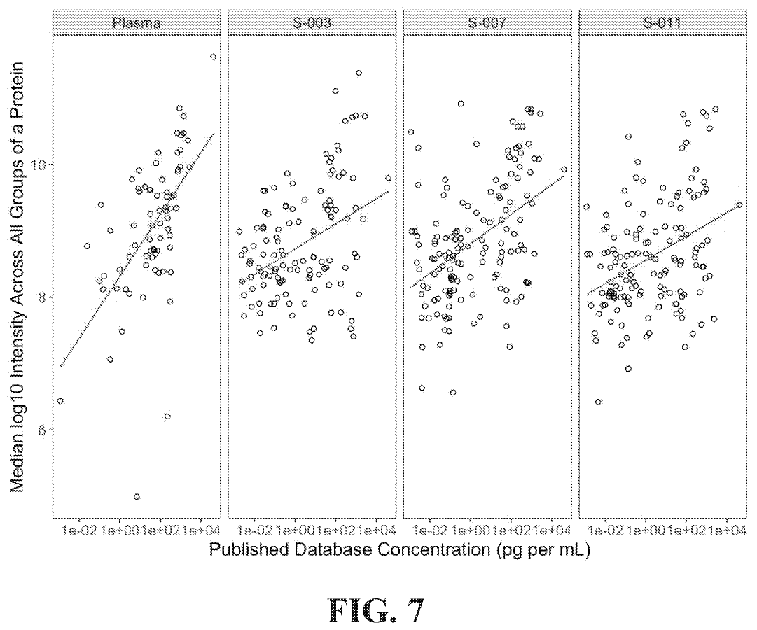

In order to evaluate the ability of particles to compress the measured dynamic range, measured and identified protein feature intensities were compared to the published values for the concentration of the same protein. First, the resulting peptide features for each protein (as presented in FIG. 6) was selected with the maximum MS-determined intensity of all possible features for a protein (using the OpenMS MS data processing tools to extract monoisotopic peak values), and then the intensities were modeled against the published abundance levels for those same proteins (FIG. 7). By comparing the regression model slopes and the intensity span of the measured data, the particle coronas contain more proteins at lower abundances (measured or reported) than does plasma, similar to FIG. 6. The dynamic range of those measured values was compressed (the slope of the regression model is reduced) for particle measurements as compared to plasma measurements. This was consistent with previous observations that particle can effectively compress the measured dynamic range for abundances in the resulting corona as compared to the original dynamic range in plasma, which could be attributable to the combination of absolute concentration of a protein, its binding affinity to particles, and its interactions with neighboring proteins. All the above results indicate that the multi-particle type protein corona strategy facilitated the identification of a broad spectrum of plasma proteins, particularly those in the low abundance that are challenging for rapid detection by conventional proteomic techniques.

To evaluate the robustness of protein identification using the particle corona MS assay, full-assay triplicates were performed using the three particle type panel to create individual protein corona samples from the same pooled CRC plasma sample. For each combination of particle types ranging from any one, to all groups of two, to the single group of three, the number of unique proteins enumerated by the combination is shown in TABLE 7.

TABLE 7
Summary of the protein coverage from the combinations
of SP-003, SP-007 and SP-011 Particles

| Particle Type Combination | Only One | Any One | All Three |
|---|---|---|---|
| SP-003 | 1058 ± 27.8 | 1313 | 844 |
| SP-007 | 961 ± 87.4 | 1277 | 660 |
| SP-011 | 1022 ± 37.8 | 1249 | 821 |
| SP-003:SP-007 | 1412 ± 18.9 | 1816 | 1052 |
| SP-003:SP-011 | 1272 ± 22.5 | 1595 | 973 |
| SP-007:SP-011 | 1372 ± 27.3 | 1746 | 1026 |
| SP-003:SP-007:SP-011 | 1576 ± 14.5 | 2030 | 1150 |

In the ‘Only One’ column, the protein counts were developed using each of the three replicates independently and then finding the mean and standard deviation for all of the combination counts. As can be seen, more proteins were discovered when increasing the number of particle types in the particle panel, with >1,500 unique proteins by the group of three particle types (65 of which are FDA-cleared/approved biomarkers, as listed in TABLE 8, below). In the ‘Any One’ replicate column, the protein counts were developed using the union of a particle type replicate protein lists. In the ‘All Three’ replicates column, the protein counts were developed using the intersection of a particle type replicate protein lists. As an additional measure of particle replicate overlap of identified proteins, the Jaccard Index, a metric for set similarity, was calculated for each pairwise-comparison. The values for SP-003, SP-007, and SP-011 were 0.74±0.018, 0.65±0.078, and 0.76±0.019 (mean±sd), respectively. Enumeration of protein content in a given MS sample is subject to the stochastic nature of MS2 data collection and may represent an undercount of the proteins represented within a sample or shared in common between samples. PSM mapping to shared MS1 features represents one approach that may alleviate this issue and will be developed for future analysis.

TABLE 8
FDA-Cleared/Approved Biomarkers

| UP_Accession | UP_Name | Class |
|---|---|---|
| P02647 | APOA1_HUMAN | Particles |
| P00747 | PLMN_HUMAN | Particles |
| P02671 | FIBA_HUMAN | Particles |
| P02675 | FIBB_HUMAN | Particles |
| P04114 | APOB_HUMAN | Particles |
| P02775 | CXCL7_HUMAN | Particles |
| P02768 | ALBU_HUMAN | Particles |
| P02679 | FIBG_HUMAN | Particles |
| P0C0L4 | CO4A_HUMAN | Particles |
| P0C0L5 | CO4B_HUMAN | Particles |
| P61626 | LYSC_HUMAN | Particles |
| P0DOY2 | IGLC2_HUMAN | Particles |
| P01024 | CO3_HUMAN | Particles |
| P08519 | APOA_HUMAN | Particles |
| P04075 | ALDOA_HUMAN | Particles |
| P00738 | HPT_HUMAN | Particles |
| P00736 | C1R_HUMAN | Particles |
| P00488 | F13A_HUMAN | Particles |
| P02765 | FETUA_HUMAN | Particles |
| P01023 | A2MG_HUMAN | Particles |
| P61769 | B2MG_HUMAN | Particles |
| P01009 | A1AT_HUMAN | Particles |
| P01834 | IGKC_HUMAN | Particles |
| P00751 | CFAB_HUMAN | Particles |
| P02746 | C1QB_HUMAN | Particles |
| P07225 | PROS_HUMAN | Particles |
| P02751 | FINC_HUMAN | Particles |
| P00450 | CERU_HUMAN | Particles |
| P02747 | C1QC_HUMAN | Particles |
| P01031 | CO5_HUMAN | Particles |
| P05155 | IC1_HUMAN | Particles |
| P09871 | C1S_HUMAN | Particles |
| P02790 | HEMO_HUMAN | Particles |
| P02745 | C1QA_HUMAN | Particles |
| P01034 | CYTC_HUMAN | Particles |
| P08697 | A2AP_HUMAN | Particles |
| P02741 | CRP_HUMAN | Particles |
| P17936 | IBP3_HUMAN | Particles |
| P01008 | ANT3_HUMAN | Particles |
| P04278 | SHBG_HUMAN | Particles |
| P19652 | A1AG2_HUMAN | Particles |
| P02787 | TRFE_HUMAN | Particles |
| P02786 | TFR1_HUMAN | Particles |
| P02763 | A1AG1_HUMAN | Particles |
| P04275 | VWF_HUMAN | Particles |
| P07195 | LDHB_HUMAN | Particles |
| P00338 | LDHA_HUMAN | Particles |
| P30613 | KPYR_HUMAN | Particles |
| P02766 | TTHY_HUMAN | Particles |
| P09972 | ALDOC_HUMAN | Particles |
| O75874 | IDHC_HUMAN | Particles |
| P06858 | LIPL_HUMAN | Particles |
| P05164 | PERM_HUMAN | Particles |
| P05121 | PAI1_HUMAN | Particles |
| P00740 | FA9_HUMAN | Particles |
| P05543 | THBG_HUMAN | Particles |
| P04070 | PROC_HUMAN | Particles |
| P08833 | IBP1_HUMAN | Particles |
| P00742 | FA10_HUMAN | Particles |
| P07477 | TRY1_HUMAN | Particles |
| P07478 | TRY2_HUMAN | Particles |
| P02753 | RET4_HUMAN | Particles |
| P43251 | BTD_HUMAN | Particles |
| P24666 | PPAC_HUMAN | Particles |
| P05160 | F13B_HUMAN | Particles |
| P01023 | A2MG_HUMAN | Plasma |
| P02768 | ALBU_HUMAN | Plasma |
| P02671 | FIBA_HUMAN | Plasma |
| P01008 | ANT3_HUMAN | Plasma |
| P01024 | CO3_HUMAN | Plasma |
| P00450 | CERU_HUMAN | Plasma |
| P02775 | CXCL7_HUMAN | Plasma |
| P02787 | TRFE_HUMAN | Plasma |
| P08697 | A2AP_HUMAN | Plasma |
| P01031 | CO5_HUMAN | Plasma |
| P0C0L4 | CO4A_HUMAN | Plasma |
| P0C0L5 | CO4B_HUMAN | Plasma |
| P01009 | A1AT_HUMAN | Plasma |
| P00736 | C1R_HUMAN | Plasma |
| P02647 | APOA1_HUMAN | Plasma |
| P02751 | FINC_HUMAN | Plasma |
| P09871 | C1S_HUMAN | Plasma |
| P00738 | HPT_HUMAN | Plasma |
| P04114 | APOB_HUMAN | Plasma |
| P00740 | FA9_HUMAN | Plasma |
| P0DOY2 | IGLC2_HUMAN | Plasma |
| P02675 | FIBB_HUMAN | Plasma |
| P00751 | CFAB_HUMAN | Plasma |
| P05543 | THBG_HUMAN | Plasma |
| P02679 | FIBG_HUMAN | Plasma |
| P02790 | HEMO_HUMAN | Plasma |
| P05155 | IC1_HUMAN | Plasma |
| P02765 | FETUA_HUMAN | Plasma |
| P61769 | B2MG_HUMAN | Plasma |
| P01834 | IGKC_HUMAN | Plasma |
| P07225 | PROS_HUMAN | Plasma |
| P00338 | LDHA_HUMAN | Plasma |
| P07195 | LDHB_HUMAN | Plasma |
| P00488 | F13A_HUMAN | Plasma |
| P19652 | A1AG2_HUMAN | Plasma |
| P00747 | PLMN_HUMAN | Plasma |
| P02747 | C1QC_HUMAN | Plasma |
| P08519 | APOA_HUMAN | Plasma |
| P43251 | BTD_HUMAN | Plasma |
| P02763 | A1AG1_HUMAN | Plasma |
| P02741 | CRP_HUMAN | Plasma |
| P04275 | VWF_HUMAN | Plasma |
| P02746 | C1QB_HUMAN | Plasma |
| P17936 | IBP3_HUMAN | Plasma |
| P02745 | C1QA_HUMAN | Plasma |
| P00742 | FA10_HUMAN | Plasma |
| P04075 | ALDOA_HUMAN | Plasma |
| P01034 | CYTC_HUMAN | Plasma |
| P05160 | F13B_HUMAN | Plasma |
| P02753 | RET4_HUMAN | Plasma |
| P04070 | PROC_HUMAN | Plasma |
| P06744 | G6PI_HUMAN | Plasma |
| P02766 | TTHY_HUMAN | Plasma |
| P61626 | LYSC_HUMAN | Plasma |
| P05062 | ALDOB_HUMAN | Plasma |
| P06276 | CHLE_HUMAN | Plasma |
| P04278 | SHBG_HUMAN | Plasma |
| P02786 | TFR1_HUMAN | Plasma |

Dynamic Range. The three-particle type panel was assessed for its ability to assay proteins in a sample across a wide dynamic range of protein concentrations. Feature intensities corresponding to proteins that were identified by mass spectrometry were compared to the values determined by other assays for the same protein at the same concentration. After mass spectrometry analysis and data processing, MS2 peptide-spectral matches (PSM) were used to identify peptides and associated proteins present in the corona of the distinct particles types in the particle panel. In parallel, peptides were also directly detected in a plasma sample, without the use of the three-particle type panel for corona analysis via the Proteograph workflow. Resulting peptide features having the maximum MS-determined intensity of all observed features, as determined using the OpenMS MS data processing tools to extract monoisotopic peak values, was selected for each protein. The MS-determined intensities were then modeled against comparable published abundance levels for the same proteins. FIG. 7 shows a correlation between the maximum intensities of proteins in distinct coronas from the distinct nanoparticle types for each particle type in the three-particle type panel relative to plasma proteins and concentration of the same proteins determined using other methods. As shown by the regression model slopes and the intensity span of the measured data, the particle coronas contained more protein hits at lower abundances than does plasma. Additionally, the dynamic range of those measured values was compressed, as shown by a reduced slope of the regression models, for particle measurements as compared to plasma measurements, showing that particles effectively compressed the measured dynamic range of protein abundance in the corona as compared to in plasma. This may be attributed to a combination of absolute protein concentration, protein binding affinity to particles, and protein interactions with neighboring proteins. These results indicate that the methods disclosed herein of using a multi-particle type panel for enrichment of proteins in distinct coronas corresponding to the distinct particle types facilitated the identification of a broad spectrum of plasma proteins, particularly those in the low abundance that are challenging for rapid detection by conventional proteomic techniques.

### Example 5

### Particle Panel for Assaying Proteins in a Sample

This example illustrates a 10-particle type particle panel for assaying proteins in a sample. This particle panel shown in TABLE 9 includes 10 distinct particle types, which differ in size, charge, and polymer coating. All particle types in this particle panel are superparamagnetic. The panel shown in below was used to assay proteins in samples.

TABLE 9
10 Particle Type Particle Panel

| Particle | Particle | Mean DLS | Mean Zeta |
|---|---|---|---|
| ID | Description | diameter (nm) | Potential (mV) |
| SP-003 | Thick silica coated | 262 | −36.9 |
| | SPION | | |
| SP-006 | N-(3-Trimethoxysilylpropyl) | 232 | 20.9 |
| | diethylenetriamine | | |
| | coated | | |
| SP-007 | PDMAPMA-coated | 259 | 25.8 |
| | SPION | | |
| SP-010 | Carboxylated, PAA | 366 | −47.9 |
| SP-353 | Amino surface | 606 | 27.2 |
| | microparticle, 0.4-0.6 μm | | |
| SP-333 | Carboxylate | 1300 | −28.5 |
| | microparticle, surfactant | | |
| | free | | |
| SP-339 | Polystyrene carboxyl | 410 | −31.4 |
| | functionalized | | |
| SP-347 | Silica coated, 200 nm | 281 | −21.8 |
| SP-365 | Silica | 231 | −39.0 |
| SP-373 | Dextran based coating, | 169 | −0.5 |
| | 0.13 μm | | |

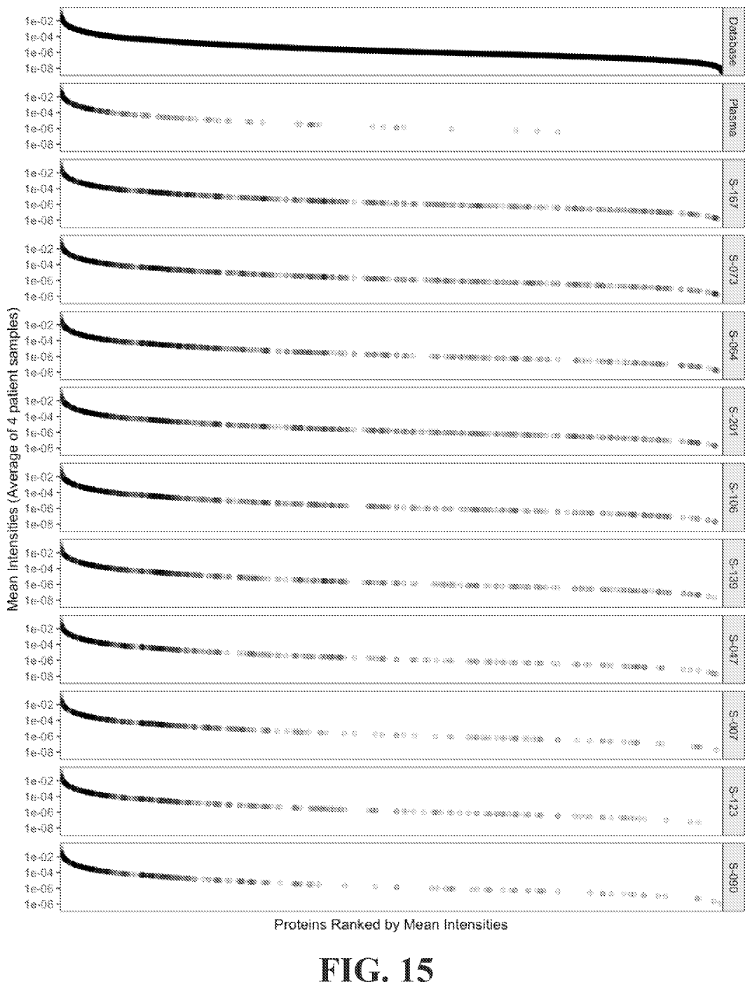

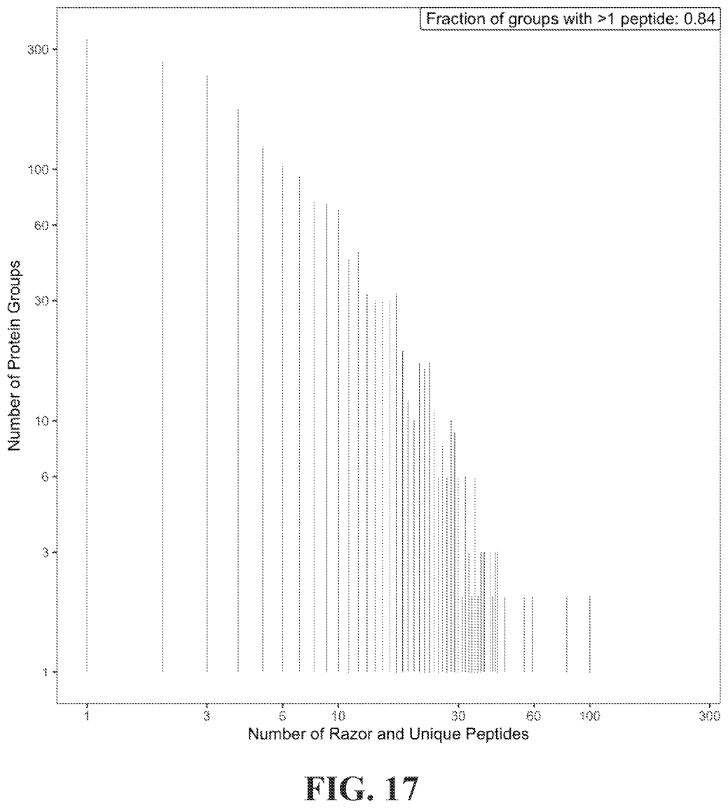

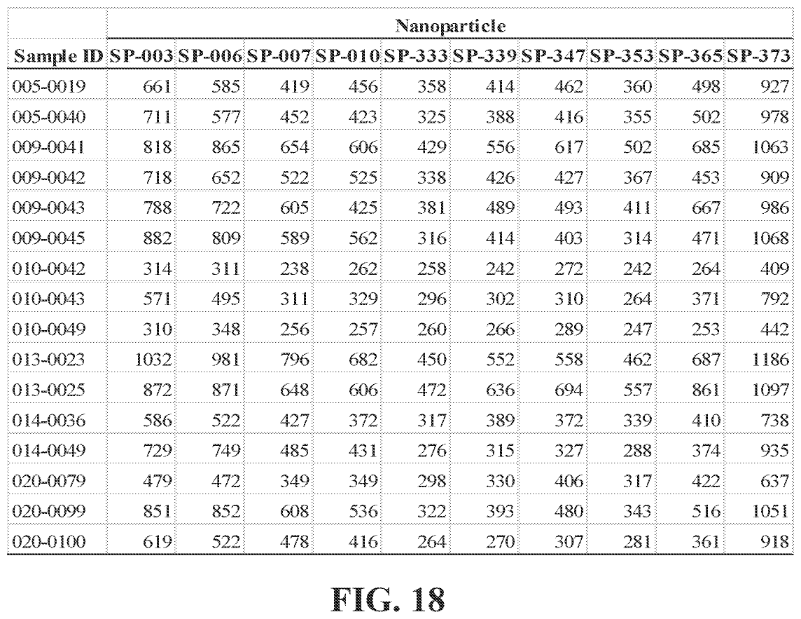

Protein coverage of V1 panel. Using the two-tiered screening approach, an optimized panel of 10 NPs was selected. To evaluate the total protein group coverage seen across multiple samples in a clinical sample set, plasma samples from 16 individuals were evaluated for a panel of ten distinct particle types shown in TABLE 9 and referred to as the V1 panel. using the sample preparation, MS data acquisition and MS data analysis methods described herein. A mix of non-small-cell lung carcinoma (NSCLC) patients and healthy individuals (n=8 for each) was used to provide a diverse set of proteins and protein groups, present in both healthy and cancer cells, for analysis and identification using the methods described herein. At the 1% FDR (protein and peptide) rate, a total of 2,009 protein groups were were identified, 84% of which (1,688) were defined by more than one peptide. A summary of the number of peptides used to define protein groups is plotted in summary FIG. 17. The full data for protein group detection by MaxQuant across the samples is provided as Supplemental File proteinGroups_10NPpanel.txt), and the results are summarized in FIG. 18 by protein group count per NP per subject. For comparison, in the previously mentioned published study, 4,500 protein groups were detected across 16 individual plasma samples in a complex workflow comprised by more than 70 steps and more than 30 MS fractions per sample, likely taking weeks to complete. Using the MS-derived plasma protein group intensities from that published study, the coverage of each of the NPs in the 10-particle, optimized panel was compared both to that full database as well as to the coverage obtained by MS analysis of neat, digested plasma (no depletion or enrichment). The 10 NPs together detected substantially more proteins than observed in neat plasma (plasma, 162; NPs range (216-479; FIG. 18). In addition, plasma proteins matching the database were skewed towards higher-abundance proteins found in the full plasma protein database, whereas the protein constituents of the protein coronas from all 10 NPs extended throughout nearly the full database's dynamic range (FIG. 15). Only 21 proteins in the database had intensities lower than the lowest protein group matched from a NP.

### Example 6

### Linearity of the Corona Analysis Assay

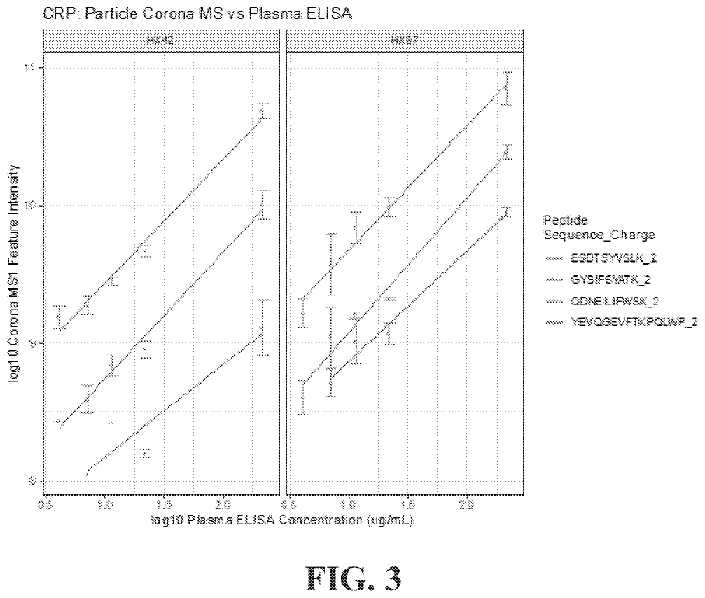

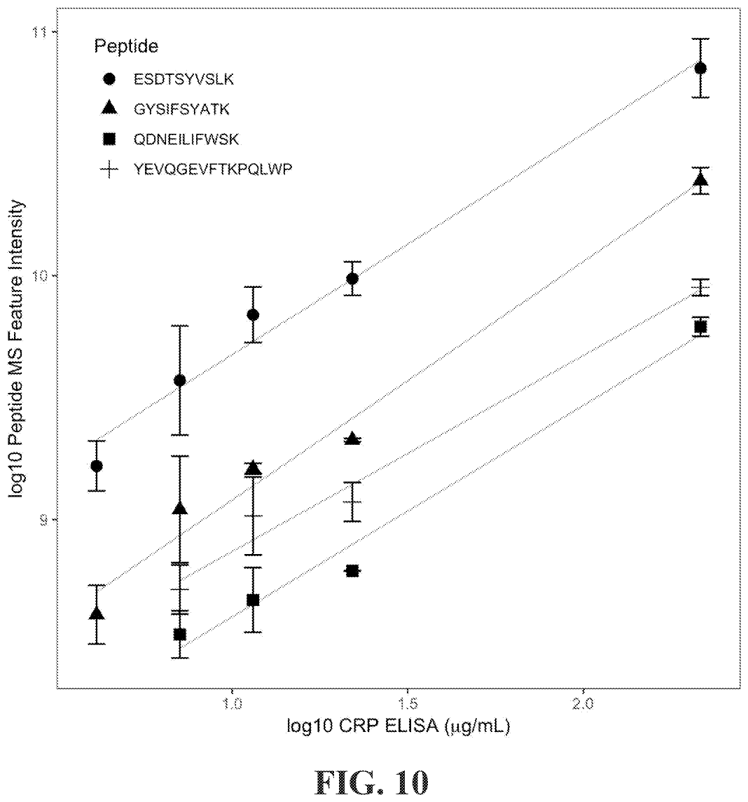

This example describes the linearity of the corona analysis assay. The linearity of a method should be sufficiently robust for detecting a true difference between groups of samples in biomarker discovery and validation studies. Linearity of the corona analysis assay was determined by comparing corona analysis assay results to those obtained by other methods. To evaluate the corona analysis assay's linearity, a spike recovery study was performed using the SP-007 nanoparticles. C-reactive protein (CRP) was selected for analysis based on the measurement of its endogenous levels. Using the enzyme-linked immunosorbent assay (ELISA)-determined endogenous plasma levels for CRP, known amounts of the purified protein (see Methods) were spiked to achieve testable multiples of the endogenous levels. The CRP levels after spiking were determined empirically by ELISA to be 4.11, 7.10, 11.5, 22.0, and 215.0 pg/mL for the 1× (unspiked), 2×, 5×, 10×, and 100× samples, respectively. The extracted MS1 feature intensities were plotted for the four indicated CRP tryptic peptides detected by MS on the SP-007 particles versus the CRP concentrations (FIG. 3). FIG. 10 also shows the linearity of measurements for CRP proteins on the SP-007 particles in a spike-recovery experiment for four different peptides. The MS1 feature intensity cannot be detected for two of the peptides at the unspiked 1× concentration of CRP. The fitted lines were linear models using the given feature's spike intensities.

Fitting a regression model to all 4 of the CRP tryptic peptides resulted in a slope of 0.9 (95% CI 0.81-0.98) for the response of corona MS signal intensity versus ELISA plasma level, which is close to a slope of 1 that would be considered to be perfect analytical performance. In contrast, a similar regression model fitted to 1,308 other (non-spiked) MS features identified in at least 4 of the 5 plasma samples, for whom the signals from associated MS features should not vary across the samples, had a slope of −0.086 (95% CI −0.1-−0.068). These results indicated the ability of that particle type to accurately describe differences between samples will provide a useful tool to quantify potential markers in comparative studies. If a protein level changes in a sample due to some factor, the methods disclosed herein will detect a similar level change of protein bound to particle types of the particle panel, which is a critical property of the present particle type to be effective in any given assay. Moreover, the response of the spiked-protein peptide features also suggests that with appropriate calibration, the particle protein corona method could be used to determined absolute analyte levels as opposed to just relative quantitation.

Linearity of response was explored in greater depth with the addition of two other spiked proteins, Angiogenin and Calprotection (S100a8/9) comprising three additional polypeptides and three additional NPs. The intensity data for these additional proteins and NPs (MaxQuant output Supplemental File proteinGroups_Accuracy.txt) was modeled against the measured ELISA values by linear regression, and a summary of the fits for the models is shown in TABLE 13. The mean slope across all proteins and NPs is 1.06, indicating a linear response across the two orders of magnitude used in the spiked sample preparation (1× to 100× endogenous levels). The adjusted-r² correlation for the intensities is also high (mean 0.95). These results confirm the linearity of response and indicate the ability of the NP platform to measure relative changes in peptide/protein levels across a broad range of concentrations with high precision.

### Example 7

### 10-Particle Type Particle Panel for Protein Assaying

This example illustrates the development of a 10-particle type particle panel for methods of assaying proteins using biomolecule corona analysis, as described herein.

Particle Screen. To demonstrate the ability of the corona analysis platform to expand its coverage through guided particle addition, biomolecule coronas from 43 particles types with distinct physicochemical properties and screened in a similar manner to the three-particle type particle panel disclosed herein.

TABLE 10
Particles for Screening

| | | DLS | | Zeta |
|---|---|---|---|---|
| Particle | | Diameter | DLS | potential |
| type | Particle Description | (nm) | PDI | (mV) |
| SP-001 | Carboxylated citrate coated | 374 | 0.23 | −34.0 |
| SP-002 | Phenol-formaldehyde resin coated | 335 | 0.39 | −29.0 |
| SP-003 | Silica coated SPION | 233 | 0.05 | −36.9 |
| SP-004 | Polystyrene coated | 411 | 0.32 | −45.7 |
| SP-005 | Carboxylate Poly(styrene-co-methacrylic acid) | 247 | 0.19 | −36.5 |
| SP-006 | N-(3-Trimethoxysilylpropyl)diethylenetriamine | 232 | 0.30 | 20.9 |
| | coated | | | |
| SP-007 | PDMAPMA-coated SPION | 283 | 0.09 | 25.8 |
| SP-008 | 1,2,4,5-Benzenetetracarboxylic acid coated | 426 | 0.43 | −34.5 |
| SP-009 | PVBTMAC coated | 229 | 0.11 | 35.9 |
| SP-010 | Carboxylated, Polyacrylic acid | 366 | 0.23 | −47.9 |
| SP-016 | Titanium(IV) oxide coated | 1623 | 0.92 | −32.1 |
| SP-019 | Phenylboronic acid coated | 305 | 0.44 | −36.4 |
| SP-047 | Poly(glycidyl methacrylate-benzylamine) coated | 1255 | 0.54 | 18.1 |
| SP-060 | Maleimide base surface | 302 | 0.15 | −40.8 |
| SP-064 | Poly(N-[3-(Dimethylamino)propyl]methacrylamide- | 302 | 0.25 | 27.7 |
| | co-[2-(methacryloyloxy)ethyl]dimethyl-(3- | | | |
| | sulfopropyl)ammonium hydroxide, P(DMAPMA- | | | |
| | co-SBMA) coated | | | |
| SP-065 | Modified Random 30 nt ssDNA | 364 | 0.21 | −43.5 |
| SP-066 | Smaller size carboxylated citrate coated | 210 | 0.25 | −35.3 |
| SP-369 | Carboxylated, Original coating, 50 nm | 104 | 0.15 | −31.5 |
| SP-373 | Dextran based coating, 0.13 μm | 169 | 0.07 | −0.6 |
| SP-374 | Silica Silanol coated with lower acidity | 225 | 0.11 | −25.6 |
| SP-389 | BioMag ®Plus Wheat Germ Agglutinin coated | 3514 | 0.97 | −21.6 |
| | microparticle | | | |
| SP-390 | Oleic acid- Hydrophilic/hydrophobe surface | 98 | 0.10 | −38.0 |
| SP-391 | Rare earth doped phosphor particles | 130 | 0.15 | −16.0 |
| SP-392 | Gadolinium oxide nanopowder coated | 1199 | 0.82 | −4.9 |
| SP-393 | Oligonucleotide-philic Apostle MiniMaxTM | 614 | 0.23 | −41.9 |
| | Magnetic Nanoparticles | | | |
| SP-394 | Iron Oxide Nanoparticles with Azide Groups | 64 | 0.11 | −17.0 |
| | coating, 30 nm | | | |
| SP-397 | DEAE starch coated | 99 | 0.18 | 13.6 |
| SP-398 | Poly(maleic acid-co-olefin) amphiphilic coating | 393 | 0.31 | −27.8 |
| SP-399 | Polyvinyl alcohol coated | 163 | 0.10 | −8.9 |
| SP-300 | Poly(4-vinylpyridine) (P4VP) coated | 177 | 0.21 | −19.3 |
| SP-301 | Poly-diallyldimethylamine coated, strong anion | 114 | 0.14 | 24.6 |
| | exchanger | | | |
| SP-305 | Amine small clusters | 75 | 0.17 | −9.5 |
| SP-406 | Boronated nanopowder surface | 491 | 0.45 | −40.7 |
| SP-413 | Nanotrap Blue VSA CS Magnetic Porous surface | 3500 | 0.77 | −3.0 |
| SP-333 | Carboxylate microparticle, surfactant free | 1348 | 0.66 | −28.5 |
| SP-339 | Polystyrene carboxyl functionalized | 410 | 0.03 | −31.4 |
| SP-341 | Carboxylic acid, 150 nm | 154 | 0.10 | −26.0 |
| SP-347 | Silica coated, 200 nm | 281 | 0.18 | −21.8 |
| SP-353 | Amino surface microparticle, 0.4-0.6 μm | 1723 | 0.75 | 31.4 |
| SP-356 | Silica amino functionalized microparticle, 0.1-0.39 | 2634 | 0.62 | 19.8 |
| | μm | | | |
| SP-363 | Jeffamine, 0.1-0.39 μm | 253 | 0.13 | −35.4 |
| SP-364 | Polystyrene microparticle, 2.0-2.9 μm | 3176 | 0.96 | −55.9 |
| SP-365 | Silica | 231 | 0.02 | −39.0 |

The 43 particle types were evaluated using 6 conditions, as described in the methods sections, and the most optimal conditions were used in a secondary analysis to select the best combination based on total identified protein number. The 43-particle type screen was conducted using a plasma pool of healthy and lung cancer patients, different from the CRC pool used for the three-particle type particle panel, to demonstrate platform validation across biological samples. A pooled sample was used to increase protein diversity. Strict criteria were used to identify potential proteins for panel selection and optimization. For maximum potential evaluation, a protein had to be represented by at least one peptide-spectral-match (PSM; 1% false discovery rate (FDR)) in each of three full assay replicates to be counted as “identified.” The panel with the largest number of individual unique Uniprot identifiers was selected for the 10-particle type particle panel. This approach avoids any differential protein grouping effects possible across different combinations of evaluated NPs, since protein groups are based on the empirical data contained within any given analysis and might be confounded by so many diverse NP corona subsets.

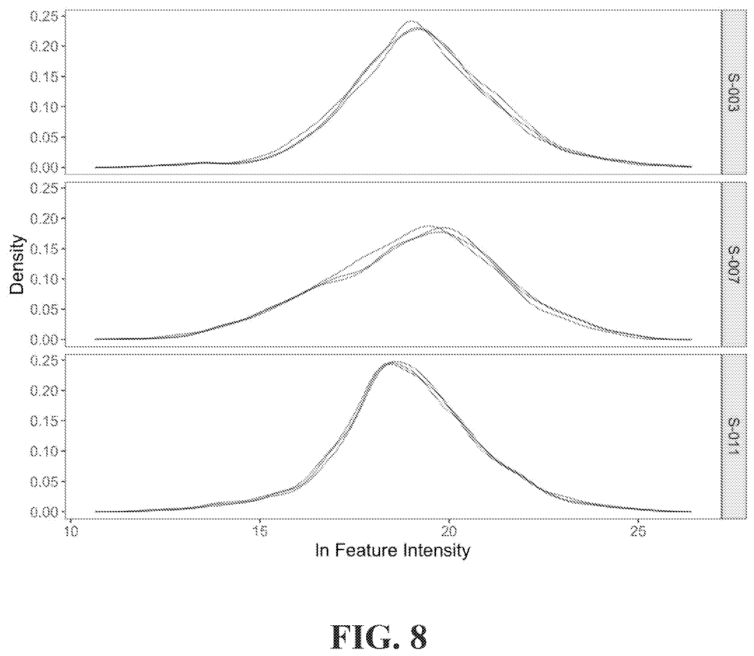

Protein Coverage of 10-Particle Type Particle Panel. Data disclosed herein confirms that the particle panels provided can be used to determine changes in proteomic content across many biological samples. The particle panels disclosed herein have high precision and accuracy and provide methods that take an unbiased approach that doesn't require specific ligands to known proteins. Thus, these panels are particularly well suited to biomarker discovery. The breadth and depth of plasma protein coverage using the 10-particle type panel was investigated. Using a database (n=5,304) of MS-derived plasma protein intensities (a close correlate to concentration), the coverage of the 10-particle type panel was compared against the full extent of the database as well as against the coverage obtained by MS evaluation of simple plasma (direct MS analysis of the same plasma sample without particle-based sampling). FIG. 15 shows matching and coverage of a particle panel of the 10 distinct particle types to a 5,304 plasma protein database of MS intensities. The ranked intensities for the database proteins are shown in the top panel (“Database”), the intensities for proteins from simple plasma MS evaluation are shown in the second panel (“Plasma”) and the intensities for the optimal 10-particle panel are shown in the remaining panels. The plasma protein intensities database is from Keshishian et al. (2015). Multiplexed, Quantitative Workflow for Sensitive Biomarker Discovery in Plasma Yields Novel Candidates for Early Myocardial Injury. Molecular & Cellular Proteomics, 14(9), 2375-2393. The results, shown in FIG. 15, confirmed and extended the results shown for the particle panel of 3 distinct particle types described above, which were used in precision experiments shown FIG. 8. The particle panel of 10 distinct particle types identified 1,598 proteins vs. 268 proteins for simple plasma. Furthermore, each individual particle type detected substantially more proteins than direct MS analysis of simple plasma. Unlike MS analysis on simple plasma, the particle panel of 10 distinct particle types interrogated the entire spectrum of the concentration of plasma proteins. Said differently, while the proteins identified from the simple plasma sample were skewed toward the higher intensity proteins (that is, higher abundance proteins), the proteins identified from the particle panel of 10 distinct particle types extended over 8 orders of magnitude in dynamic range of the concentrations in the database. Only 21 proteins in the database had intensities lower than the lowest protein matched from the particle panel of 10 distinct particle types. As demonstrate in FIG. 15, the particle panel of 10 distinct particle types demonstrated high precision, accuracy, and broad coverage across a wide range of protein concentrations in plasma and enables broad-scale, unbiased proteomic analyses in parallel across large numbers of biological samples, and can match the cost and speed of what is possible in genomic data acquisition today.

Precision of a Particle Panel Including 10 Distinct Particle Types. This example describes reproducibility of particle corona for a particle panel including 10 distinct nanoparticle types. Particles were analyzed to determine the coefficient of variation (CV) of each feature group between the replicate runs for each particle type of the particle panel including 10 distinct nanoparticle types. A low CV indicated high precision and reproducibility between replicate runs. The data was processed using the software program OpenMS and retained feature groups which contained an observed precursor feature from each of three replicates. The bottom 5% of the data was removed to eliminate statistical outliers based on a quality score of the clustering algorithm. Group feature intensities were median normalized, and the overall precision of the coronas of each particle type was estimated. Normalization was performed such that the overall median intensity for each injection remained the same, and intensities were adjusted for each compared distribution to account for intensity shifts due to, for example, overall differences in instrument response. Differences in instrument response may arise in a variety of analysis methods, including X-ray photoelectron spectroscopy, high-resolution transmission electron microscopy, and other analytical methods. The normalized values of the coefficients of variation (CVs) of each feature group were then evaluated for each particle type of the particle panel including 10 distinct nanoparticle types. TABLE 11 shows the optimized panel of 10 distinct particle types.

TABLE 11
10 Particle Type Particle Panel

| Particle Type | Particle Description |
|---|---|
| SP-333 | Carboxylate microparticle, surfactant free |
| SP-339 | Polystyrene carboxyl functionalized |
| SP-347 | Silica coated, 200 nm |
| SP-365 | Silica |
| SP-373 | Dextran based coating, 0.13 μm |
| SP-390 | Oleic acid- Hydrophilic/hydrophobe surface |
| SP-406 | Boronated nanopowder surface |
| SP-007 | PDMAPMA-coated SPION |
| SP-047 | Poly(glycidyl methacrylate-benzylamine) coated |
| SP-064 | Poly(N-[3-(Dimethylamino)propyl]methacrylamide-co- |
| | [2-(methacryloyloxy)ethyl] dimethyl-(3- |
| | sulfopropyl)ammonium hydroxide, P(DMAPMA-co- |
| | SBMA) coated |

TABLE 12 shows the median percent of quantile normalized CV (QNCV %) for precision evaluation of the protein corona-based Proteograph workflow for plasma and a particle panel including 10 distinct particle types for features, peptides and proteins. A 1% peptide and 1% protein false discovery rate (FDR) was applied. Using the NP screening data for the 10-particle panel comprising three full-assay replicates, interrogating a common pooled plasma sample for each particle, the median CVs were determined for protein group quantification using MaxQuant (See Methods). The results ranged from 16.4% to 30.8% (TABLE 12). Data was processed using MaxLFQ analysis software, applying the condition that each protein group have at least one peptide ratio-count and detection in all replicates, which reduced the number of groups used for the precision analysis. For each particle type of the particle panel including 10 distinct nanoparticle types, the median CVs, including percent of quantile normalized CV or QNCV %, are shown in TABLE 12. A similar analysis was performed at a peptide and protein level using MaxQuant to align identifiable feature groups to features, peptides, and proteins (TABLE 12). The number of identifiable features decreases from features to peptides to proteins, as peptides can comprise multiple features and proteins can comprise multiple peptides. This nanoparticle panel detected 1,184 protein groups with a 1% false discovery rate (FDR).

TABLE 12
Median QNCV % for a particle panel including
10 distinct nanoparticle types

| | Features | | Peptides | | Proteins | |
|---|---|---|---|---|---|---|
| | (OpenMS) | | (MaxQuant) | | (MaxQuant) | |
| | # | Median | # | Median | # | Median |
| Particle | Features | CV | Peptides | CV | Proteins | CV |
| Plasma | 2141 | 22.5 | 976 | 22.7 | 162 | 17.1 |
| SP-333 | 2163 | 17.2 | 1192 | 20.5 | 250 | 18.2 |
| SP-339 | 2330 | 19.4 | 1406 | 20.8 | 296 | 17.9 |
| SP-347 | 2792 | 15.4 | 2105 | 19.9 | 469 | 16.4 |
| SP-365 | 2322 | 17.9 | 1867 | 22.4 | 447 | 18.4 |
| SP-373 | 2796 | 27.1 | 2091 | 30.3 | 479 | 25.5 |
| SP-390 | 2267 | 29.3 | 1265 | 25.8 | 216 | 19.1 |
| SP-406 | 3823 | 28.7 | 1947 | 30.8 | 410 | 28.3 |
| SP-007 | 2351 | 21.1 | 1292 | 21.5 | 250 | 17.1 |
| SP-047 | 2233 | 36.5 | 1176 | 35.7 | 279 | 30.8 |
| SP-064 | 2984 | 20.2 | 2112 | 23.3 | 433 | 19.3 |

Coefficients of variation (CVs) were examined at the level of features, peptides and proteins independently. Analysis of feature, peptide, and protein CVs provide complementary views of assay precision. OpenMS and MaxQuant software engines were used for feature, peptide, and protein matching. MaxQuant was used to for protein grouping with FDR. OpenMS was used to perform peptide-spectrum-matching (PSM) using the X!Tandem matching tool. MaxQuant was configured to use the Andromeda algorithm. Peptide CVs and protein CVs were used to assess precision of the platform for use with biological variables. The mean CV decreased with increasing peptide size, such that the mean CV was lower for peptides than for proteins. The particles maintain a CV similar to plasma, while particles have higher occurrences of features, peptides, and proteins than plasma. In particular, the number of proteins on particles of any given particle type is higher than plasma (average: 218% higher, range: 133% — 296% higher) while maintaining a comparable CV (21.1% vs 17.1% for particles and plasma, respectively). Furthermore, the panel of the particle types identified 1,184 proteins while only identifying 162 proteins for plasma alone.

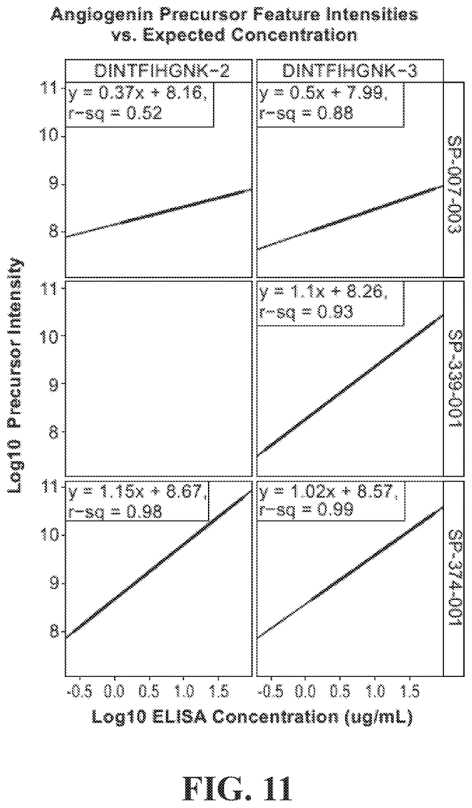

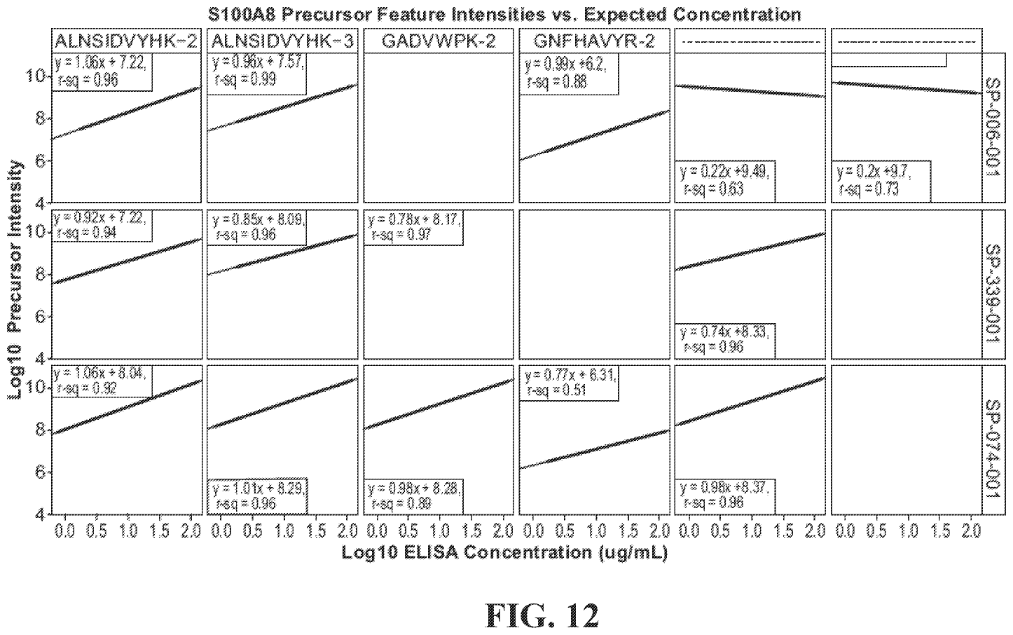

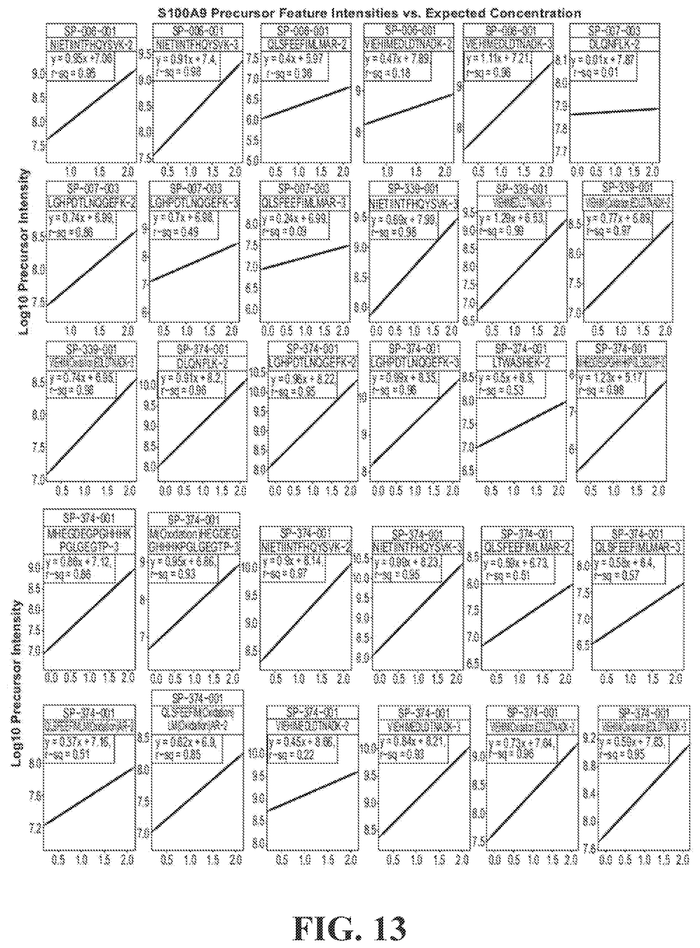

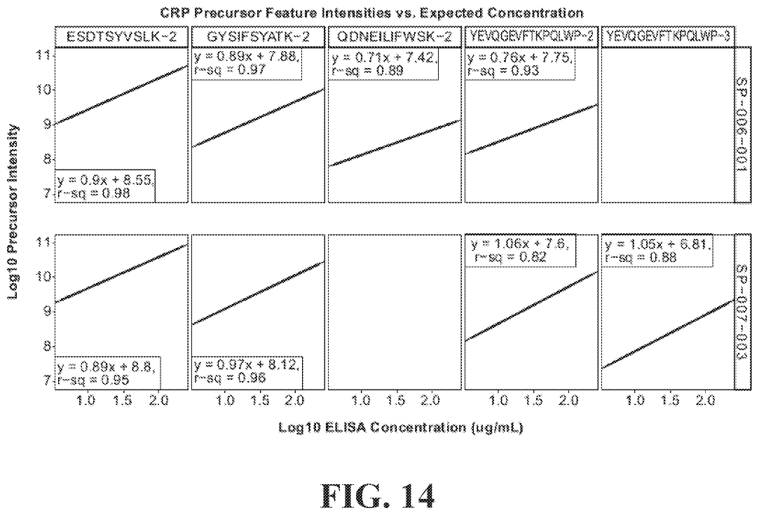

Linearity of a Particle Panel Including 10 Distinct Nanoparticle Types. The linearity of for the particle panel including 10 distinct nanoparticle types to detect a real difference between groups of samples in biomarker discovery and validation studies was assessed. Linearity was determined by measuring spike recovery data in the presence a nanoparticle types SP-007, and C-reactive protein (CRP). Spike recovery data was further measured in the presence of one three additional polypeptides (S100A8/9, and Angiogenin) in combination with each of three particle types (SP-006, SP-339, SP-374). Known amounts of each polypeptide were spiked in at different concentrations, increasing by factors of 10 (e.g., 1×, 2×, 5×, 10×, and 100λ). The level of each polypeptide was measured by ELISA. Derived peptide and protein intensities were plotted against the ELISA protein concentration. Peptide intensities were derived using OpenMS MS1/MS2 pipeline to find clustered feature groups that have a target protein MS2 ID assigned to at least one feature within the cluster. Only cluster groups with representation in at least one replicate for the top spike levels were used for the analysis. Protein intensities were derived using the MaxQuant software. Intensity values for each protein were summarized. and the data was scaled such that the maximal concentration was 2. MS datasets were performed in triplicate for each spike concentration (e.g., 1×, 2×, 5×, 10×, and 100×), providing 15 individual protein or peptide measurements. Not all peptides were detected in all particle types or particle type replicates. Results of the MS datasets are shown in FIG. 11-14. FIG. 11 shows the linearity of peptide feature measurements of Angiogenin in a spike-recovery experiment. FIG. 12 shows the linearity of peptide feature measurements of S10A8 in a spike-recovery experiment. FIG. 13 shows the linearity of peptide feature measurements of S10A9 in a spike-recovery experiment. FIG. 14 shows the linearity of peptide feature measurements of CRP in a spike-recovery experiment. The fitted lines are linear fits to the spike intensities of each feature.

FIG. 11-14 illustrate the results of three spike recovery experiments to determine the linearity of peptide feature measurements of Angiogenin, S10A8, S10A9, and CRP, respectively. The data demonstrated high degrees of correlation between individual measurements for peptides (mean r² is 0.81) and proteins (mean r² is 0.97). The mean slope across all proteins is 1.06. TABLE 13 showed the r² correlation per comparison and also the mean r² correlation per protein. Out of 20 peptides, only two showed no correlation between ELISA assays on two different particles types, in which one peptide presented in two charge states. The aberrations decreased with increasing peptide size, such that the frequency of aberrations was lower for peptides than for proteins. The two peptides that showed now correlation with the ELISA on two different particles showed a high degree of correlation to ELISA in the other particle types. The offending peptide may be co-eluting with another peptide that masks its signal, for example through charge stealing.

TABLE 13 provides a summary of regression fits to protein intensity as measured by corona analysis or ELISA. Values are shown for individual particle types and averaged between four repeats per particle type. The protein concentrations, as measured by corona analysis, were consistent across a range of conditions and a range of particle types. As shown in TABLE 13, protein measurements were well correlated, as shown by high r² values (mean 0.97, range across individual particles 0.92-1.0; range averaged across particles 0.94-0.99). This consistent behavior across the four proteins as measured by an ELISA illustrates the linearity of the corona analysis assay. TABLE 13 shows a summary of regression fit of protein intensity as measured by MaxQuant protein group intensity versus measurement by ELISA. Values for individual particles and the average values over the four particles are shown. The proteins are Angiogenin, ANG; C Reactive-Protein, CRP; and Calprotectin, S100A8/9.

TABLE 13
Summary of protein intensity regression fit.

| | Particle | | | | | | | | |
|---|---|---|---|---|---|---|---|---|---|
| Protein | Type | intercept | slope | r_sq | adj_r_sq | intercept | slope | r_sq | adj_r_sq |
| ANG | SP-006 | 0.16 | 1.05 | 0.94 | 0.91 | 0.30 | 0.96 | 0.97 | 0.95 |
| ANG | SP-007 | 0.75 | 0.78 | 0.93 | 0.90 | | | | |
| ANG | SP-339 | 0.05 | 1.05 | 1.00 | 1.00 | | | | |
| ANG | SP-374 | 0.23 | 0.98 | 0.99 | 0.99 | | | | |
| CRP | SP-006 | −0.24 | 0.96 | 1.00 | 1.00 | −0.85 | 1.22 | 0.99 | 0.99 |
| CRP | SP-007 | −0.48 | 1.07 | 0.99 | 0.99 | | | | |
| CRP | SP-339 | −1.08 | 1.31 | 0.99 | 0.98 | | | | |
| CRP | SP-374 | −1.60 | 1.54 | NA | NA | | | | |
| S100A8 | SP-006 | −0.12 | 1.02 | 1.00 | 1.00 | 0.03 | 0.98 | 0.97 | 0.95 |
| S100A8 | SP-007 | −0.20 | 1.12 | 0.92 | 0.89 | | | | |
| S100A8 | SP-339 | 0.34 | 0.81 | 0.99 | 0.98 | | | | |
| S100A8 | SP-374 | 0.10 | 0.96 | 0.96 | 0.95 | | | | |
| S100A9 | SP-006 | −0.56 | 1.34 | 0.90 | 0.87 | −0.09 | 1.06 | 0.94 | 0.92 |
| S100A9 | SP-007 | −0.44 | 1.27 | 0.93 | 0.91 | | | | |
| S100A9 | SP-339 | 0.51 | 0.68 | 0.98 | 0.97 | | | | |
| S100A9 | SP-374 | 0.11 | 0.96 | 0.95 | 0.93 | | | | |

Comparison to other platforms. The methods disclosed herein using multi-particle types panels to enrich proteins in distinct coronas corresponding to each protein type in the panel (e.g., corona analysis using the Proteograph workflow) provides wide and unbiased coverage of protein identification in the proteome. Other methods that attempt broad coverage of the proteome require multiple fractionation steps, complex workflows, and are slow in comparison to the methods presented herein. Other methods lack the breadth and impartiality of the methods disclosed herein and are compared herein to the presently disclosed methods of assaying proteins.

Geyer et al (Cell Systems 2016) utilized a rapid shotgun proteomics approach and yielded an average of 284 protein groups per assay and 321 protein groups across all replicates. The assessment utilized a slower, multi-day protocol with fractionation that yielded approximately 1,000 protein groups. No replicates were performed, likely due to prohibitive costs and time requirements, and so no variance could be determined.

Geyer used a short run to generate 321 protein groups, and the CV of each protein was determined. The 321 groups assessed by Geyer and the 1,184 protein groups identified by the 10 particle type panel comprised 88 protein groups in common between the two methods. As protein groups may comprise multiple related proteins which may be differentially combined based on the detected peptides, identification of 88 common protein groups is unexpectedly high.

For the 88 common protein groups, the data from Geyer et al. was analyzed, and a median CV of 12.1% was determined. In contrast, the same 88 common protein groups, as analyzed by Proteograph, had a lower CV of only 7.2%. Thus, the instant methods of corona analysis using multi-particle type panels and the Proteograph workflow provided improved precision over the methods of Geyer et al. Additionally, Geyer et al.'s assessment showed an r², indicative of assay linearity, of 0.99 for 4 proteins. Similarly, the Proteograph assay showed an r² of 0.97.

Geyer et al. further assessed the number of protein groups with CVs <20%, the commonly used cutoff for in vitro diagnostic assays. The particle panel methods detected 761 protein groups with CV<20% which was 3.7 times greater than the number identified by Geyer et al. A further assessment by Dr. Mann (Niu et al, 2019) identified 272 protein groups with CV <20%, 2.8-fold lower than the number identified by the multi particle type panels and methods of use thereof disclosed herein.

Bruderer et al. assessed protein group CV's using data generated by a Biognosys platform (Bruderer et al, 2019). This assessment identified 465 proteins, wherein those 465 proteins had a median CV of 5.2% and 404 of those proteins had CVs <20%. In contrast, the best 465 proteins from the 1,184 proteins identified using the methods disclosed herein had a median CV of 4.7% and 761 of the 1,184 proteins identified by Proteograph had CV's<20%.

In comparison to the assessments of Geyer et al., Niu et al, and Bruderer et al., the instant particle panels provided improved CVs for an equivalent number of proteins as well as number of proteins meeting a CV threshold, over other identification methods. The methods disclosed herein additionally have reduced bias relative to other methods, such as targeted mass spectrometry and other analyte specific reagents (e.g., Olink). Such approaches measure a small number of pre-selected proteins, thereby introducing bias during the protein panel selection process. As a result, these approaches have low CVs and high r² for the proteins on their panel as compared to the proteins identified by Proteograph and are limited to detecting proteins on the panel.

### Example 8

### Materials and Methods for Particle Synthesis

This example describes materials and methods for particle synthesis.

Materials. Iron (III) chloride hexahydrate ACS, sodium acetate (anhydrous ACS), ethylene glycol, ammonium hydroxide 28˜30%, ammonium persulfate (APS) (≥98%, Pro-Pure, Proteomics Grade), ethanol (reagent alcohol ACS) and methanol (≥99.8% ACS) were purchased from VWR. N,N′-Methylenebisacrylamide (99%) was purchased from EMD Millipore. Trisodium citrate dihydrate (ACS reagent, ≥99.0%), tetraethyl orthosilicate (TEOS) (reagent grade, 98%), 3-(trimethoxysilyl)propyl methacrylate (MPS) (98%) and poly(ethylene glycol) methyl ether methacrylate (OEGMA, average Mn 500, contains 100 ppm MEHQ as inhibitor, 200 ppm BHT as inhibitor) were purchased from Sigma-Aldrich. 4,4′-Azobis(4-cyanovaleric acid) (ACVA, 98%, cont. ca 18% water) and divinylbenzene (DVB, 80%, mixture of isomers) were purchased from Alfa Aesar and purified by passing a short silica column to remove the inhibitor. N-(3-Dimethylaminopropyl)methacrylamide (DMAPMA) was purchased from TCI and purified by passing a short silica column to remove the inhibitor. The ELISA kit to measure human C-reactive protein (CRP) was purchased from R&D Systems (Minneapolis, Minn.). Human CRP protein purified from human serum was from Sigma Aldrich.

Synthesis of superparamagnetic iron oxide nanoparticle (SPION)-based SP-003, SP-007, and SP-011. The iron oxide core was synthesized via solvothermal reaction (FIG. 9A-E, at top (FIG. 9A)) (Liu, J., et al. Highly water-dispersible biocompatible magnetite particles with low cytotoxicity stabilized by citrate groups. Angew Chem Int Ed Engl 48, 5875-5879 (2009); Xu, S., et al. Toward designer magnetite/polystyrene colloidal composite microspheres with controllable nanostructures and desirable surface functionalities. Langmuir 28, 3271-3278 (2012)). Typically, about 26.4 g of iron (III) chloride hexahydrate was dissolved in about 220 mL of ethylene glycol at about 160° C. for ˜10 min under mixing. Then about 8.5 g of trisodium citrate dihydrate and about 29.6 g sodium acetate anhydrous were added and fully dissolved by mixing for about an additional 15 min at about 160° C. The solution was then sealed in a Teflon-lined stainless-steel autoclave (300 mL capacity) and heated to about 200° C. for about 12h. After cooling down to room temperature, the black paramagnetic product was isolated by a magnet and washed with DI water 3-5 times. The final product was freeze-dried to a black powder for further use.

The silica-coated iron oxide nanoparticles (SP-003) were prepared through a modified Stober process as reported before (FIG. 9B)(Deng, Y., Qi, D., Deng, C., Zhang, X. & Zhao, D. Superparamagnetic high-magnetization microspheres with an Fe3O4@SiO2 core and perpendicularly aligned mesoporous SiO2 shell for removal of microcystins. J Am Chem Soc 130, 28-29 (2008); Teng, Z. G., et al. Superparamagnetic high-magnetization composite spheres with highly aminated ordered mesoporous silica shell for biomedical applications. J Mater Chem B 1, 4684-4691 (2013)). Typically, about 1 g of the SPIONs were homogeneously dispersed in the mixture of ethanol (about 400 mL), DI water (about 10 mL), and concentrated ammonia aqueous solution (about 10 mL, 28˜30 wt %), followed by the addition of TEOS (about 2 mL). After stirring at about 70° C. for about 6 h, amorphous silica coated SPIONs (denoted as Fe₃O₄@SiO₂) were obtained and washed 3 times with methanol and additional 3 times with water and the final product was freeze-dried to a powder.

To prepare SP-007 (PDMAPMA-modified SPION) and SP-011 (PEG-modified SPION), vinyl group functionalized SPIONs (denoted as Fe3O4@MPS) were first prepared through a modified Stober process as previously reported (FIG. 9C) (Crutchfield, C. A., Thomas, S. N., Sokoll, L. J. & Chan, D. W. Advances in mass spectrometry-based clinical biomarker discovery. Clin Proteomics 13, 1 (2016)). Briefly, about 1 g of the SPIONs was homogeneously dispersed under the aid of vortexing (or sonication) in the mixture of ethanol (about 400 mL), DI water (about 10 mL), and concentrated ammonia aqueous solution (about 10 mL, 28˜30 wt %), followed by the addition of TEOS (about 2 mL). After stirring at about 70° C. for about 6 h, about 2 mL of 3-(trimethoxysilyl)propyl methacrylate was added into the reaction mixture and stirred at about 70° C. overnight. Vinyl functionalized SPIONs were obtained and washed 3 times with methanol and additional 3 times with water and the final product was freeze-dried to a powder. Next, for synthesis of poly(dimethyl aminopropyl methacrylamide) (PDMAPMA)-coated SPIONs (denoted as Fe3O4@PDMAPMA, SP-007 in FIG. 9D), about 100 mg of Fe3O4@MPS were homogeneously dispersed in about 125 mL of DI water. After bubbling with N2 for about 30 min, about 2 g of N[3-(dimethylamino)propyl]methacrylamide (DMAPMA) and about 0.2 g of divinylbenzene (DVB) were added into the Fe3O4@MPS suspension under N2 protection. After the resulting mixture was heated to about 75° C., about 40 mg of ammonium persulfate (APS) in about 5 mL DI water was added and stirred at about 75° C. overnight. After cooling down, Fe3O4@PDMAPMA were isolated with a magnet and washed 3-5 times with water. The final product was freeze-dried to a dark brown powder. For synthesis of poly(ethylene glycol) (PEG)-coated SPIONs (denoted as Fe3O4@PEGOMA, SP-011 in FIG. 9E), about 100 mg of Fe3O4@MPS were homogeneously dispersed in about 125 mL of DI water. After bubbling with N2 for about 30 min, about 2 g of poly(ethylene glycol) methyl ether methacrylate (OEGMA, average Mn 500) and about 50 mg of N,N′-Methylenebisacrylamide (MBA) were added into the Fe3O4@MPS suspension under N2 protection. After the resulting mixture was heated to about 75° C., about 50 mg of 4,4′-azobis(4-cyanovaleric acid) (ACVA) in about 5 mL ethanol was added and stirred at about 75° C. overnight. After cooling down, Fe3O4@POEGMA were isolated with a magnet and washed 3-5 times with water. The final product was freeze-dried to a dark brown powder.

### Example 9

### Patient Samples

This example describes patient samples used in the present disclosure. A set of 8 colorectal cancer (CRC) plasma samples with 8 age- and gender-matched controls was purchased from BioIVT (Westbury, N.Y.). A set of 28 non-small cell lung cancer (NSCLC) serum samples with 28 controls matched by age and gender was also obtained from BioIVT. The detailed information regarding the CRC/NSCLC patient samples and controls are shown in TABLE 14 and TABLE 15.

TABLE 14
NSCLC and Controls

| Class | Age | Gender | Diagnosis | Medications |
|---|---|---|---|---|
| Diseased | 53 | Female | Non Small Cell Lung | Alimta 800 mg/Carboplatin 760 mg, Advil |
| | | | Cancer (NSCLC) | 200 mg, Compazine 10 mg, Dexamethasone |
| | | | | 4 mg, Diclofenac Sodium 50 mg, |
| | | | | Dicyclomine 10 mg, Folic Acid 1 mg, |
| | | | | Lactulose 10 g/15 ml, Lansoprazole 30 mg, |
| | | | | Multivitamin, Oxycodone 5 mg, Reglan |
| | | | | 10 mg, Vitamin C 1000 mg, Vitamin D2 |
| | | | | 50000 iu |
| Diseased | 64 | Female | Non Small Cell Lung | Opdivo, Alendronate Sodium 10 mg, |
| | | | Cancer (NSCLC), | Allegra Allergy 180 mg, Anoro Ellipta |
| | | | Vitamin B Deficiency, | 62.5 mcg-25 mcg, Aspirin 81 mg, Bystolic |
| | | | Hypertension (HTN), | 5 mg, Calcium and D 500 mg-200 iu, |
| | | | Hyperlipidemia | Compazine 10 mg, Crestor 40 mg, Dilaudid |
| | | | | 1200 mg, Emla 2.5%-2.5%, Erythromycin |
| | | | | 5 mg, Fish Oil 340 mg-1000 mg, Flonase |
| | | | | Allergy Relief 50 mcg, Hydromorphone |
| | | | | 4 mg, Isosorbide Mononitrate 60 mg, |
| | | | | Levothyroxine 75 mcg, Lisinopril 20 mg, |
| | | | | Medical Marijuana, Multivitamin 9 mg-Iron |
| | | | | 15 ml, Neurontin 300 mg, Nitro, Oxycodone |
| | | | | 5 mg, Plavix 75 mg, Protonix 20 mg, Unisom |
| | | | | 25 mg, Ventolin 90 mcg, Vitamin D3 |
| | | | | 5000 iu, Xanax 1 mg |
| Diseased | 73 | Female | Non Small Cell Lung | Carboplatin/Paclitaxel, Acetaminophen |
| | | | Cancer (NSCLC), | 325 mg, Multivitamin 1000 mg, Cymbalta |
| | | | Impaired Fasting | 60 mg, Eliquis 5 mg, Guaifenesin |
| | | | Glucose (IFG), | 100 mg/5 ml, Neurontin 300 mg, Synthroid |
| | | | Pulmonary Nodule | 100 mcg, Zofran 8 mg |
| Diseased | 75 | Female | Non Small Cell Lung | Osimertinib, Colace 100 mg, Flonase |
| | | | Cancer, Pneumothorax | 50 mcg, Zofran 8 mg, Restasis 0.05%, Norco |
| | | | | 5 mg-325 mg, Megace 400 mg/10 ml, |
| | | | | Tagrisso 80 mg |
| Diseased | 65 | Female | Non Small Cell Lung | Ceritinib 150 mg, Cipro 500 mg, Excedrin |
| | | | Cancer (NSCLC), | 500 mg, Lasix 40 mg, Glimepiride 4 mg, |
| | | | Type 2 Diabetes, | Lamotrigine 200 mg, Metformin 1000 mg, |
| | | | Multiple Sclerosis | Naproxen 500 mg, Zofran 8 mg, Slow |
| | | | | Release Iron 142 mg |
| Diseased | 94 | Male | Non Small Cell Lung | Keytruda 100 mg/4 ml, Betamethasone |
| | | | Cancer (NSCLC), | Dipropionate 0.05%, Eliquis 2.5 mg, |
| | | | Anemia (CKD), | Fludrocortisone 0.1 mg, Folic Acid 1 mg, |
| | | | Chronic Kidney | Lomotil 2.5 mg-0.025 mg, Midodrine 10 mg, |
| | | | Disease (CKD), | Omega Q, Prednisone 5 mg, Ranitidine |
| | | | Hyperlipidemia | 150 mg, Simvastatin 40 mg |
| | | | (HLD), Prostate | |
| | | | Cancer | |
| Diseased | 65 | Female | Non Small Cell Lung | Atenolol 50 mg, Biotin 2500 mcg, Melatonin |
| | | | Cancer (NSCLC), | 3 mg, Mometasone 0.1%, Vitamin D3 |
| | | | Hypertension (HTN) | 1000 iu, Zofran 8 mg |
| Diseased | 79 | Female | Non Small Cell Lung | Amlodipine 5 mg, Amoxicillin 875 mg, |
| | | | Cancer (NSCLC), | Estradiol 0.01%, Folic Acid 1 mg, Januvia |
| | | | Type 2 Diabetes, | 100 mg, Lidocaine/Prilocaine 2.5%, |
| | | | Hypercholesterolemia, | Losartan HCL 50 mg, Nitrofurantoin |
| | | | Emphysema | 100 mg, Pantoprazole 20 mg, Simvastatin |
| | | | | 40 mg, Urinary Pain Relief 95 mg, Zofran |
| | | | | 8 mg, Gemcitabine, Carboplatin |
| Diseased | 57 | Male | Non Small Cell Lung | Carboplatin/Etoposide, Norvasc 10 mg, |
| | | | Cancer (NSCLC), | Lotensin HCT 20 mg-12.5 mg, Celexa |
| | | | Lung Mass, Primary | 20 mg, Mycelex 10 mg, Lasix 20 mg, Norco |
| | | | Adenocarcinoma of | 7.5 mg-325 mg, Magnesium 400 mg, |
| | | | Lower Lobe of Right | Melatonin Gummies 2.5 mg, Metformin |
| | | | Lung, Brain | 750 mg, Mycostatin 100,000 iu/mL, Zofran |
| | | | Metastases, | 8 mg, Potassium Chloride 20 mEq, |
| | | | Hypokalemia | Compazine 10 mg |
| Diseased | 63 | Female | Non Small Cell Lung | Alimta, Carboplatin, Calcium-Vitamin D, |
| | | | Cancer (NSCLC), | Folvite 1 mg, Keppra 500 mg, Synthroid |
| | | | Hypertension (HTN), | 125 mcg, Prilosec 20 mg, Zofran 8 mg, |
| | | | Hypercholesterolemia, | Compazine 10 mg, Zocor 40 mg |
| | | | Gastroesophageal | |
| | | | Reflux Disease | |
| | | | (GERD), | |
| | | | Diverticulitis, Disease | |
| | | | of Thyroid, Arhropathy, Actinic | |
| | | | Keratosis | |
| Diseased | 77 | Male | Non Small Cell Lung | Singulair 10 mg, Meclizine HCL 25 mg, |
| | | | Cancer (NSCLC), | Xarelto 20 mg, Synthroid 125 mcg, Miralax |
| | | | Hypertension (HTN), | 17 g, Lidocaine 5%, Arnuity Ellipta |
| | | | Myelodysplastic | 100 mcg, Medipro Vegan Chocolate |
| | | | Syndromes, Anemia | 23.28 oz, Exos Catalyte, Zinc Picolinate |
| | | | (Iron), | 15 mg, Albuterol Sulfate 90 mcg, Vitamin |
| | | | Hypothyroidism, | B12/Folic Acid 500 mcg/400 mcg |
| | | | Prostate Cancer, | |
| | | | Bradycardia | |
| Diseased | 70 | Female | Non Small Cell Lung | Alimta/Carboplatin/Keytruda/Neulasta, |
| | | | Cancer (NSCLC), | Decadron 4 mg, Breo Ellipta |
| | | | Hypertension (HTN), | 200 mcg/25 mcg, Folvite 1 mg, Lasix 40 mg, |
| | | | Polycythemia, Left | Neurontin 300 mg, Emla, Zestril 5 mg, |
| | | | Lower Extremity | Magic Mouthwash, Glucophage 500 mg, |
| | | | Edema, Cellulitis of | Aleve 220 mg, Zofran 8 mg, Potassium |
| | | | Left Lower Extremity | Chloride 10 meq, ProAir HFA 108 mcg, |
| | | | | Spiriva 18 mcg, Valtrex 1000 mg |
| Diseased | 70 | Female | Non Small Cell Lung | Taxotere 75 mg-Cyramza 10 mg-Nuelasta, |
| | | | Cancer (NSCLC), | Ativan 0.5 mg, Basaglar 100 iu/mL, |
| | | | Hypertension (HTN), | Dexamethasone 4 mg, Eliquis 5 mg, |
| | | | Type 2 Diabetes | Fentanyl 25 mcg, Folic Acid 1 mg, |
| | | | | Glimepiride 2 mg, Hydrochlorothiazide |
| | | | | 12.5 mg, Lorazepam 0.5 mg, Magnesium |
| | | | | 300 mg, Metformin 1000 mg, Multivitamin |
| | | | | 9 mg Iron/15 mL, Oxycodone 5 mg, |
| | | | | Tramadol 50 mg, Trazodone 50 mg, Vitamin |
| | | | | D3 2000 iu |
| Diseased | 78 | Female | Non Small Cell Lung | Folic Acid 1 mg, Lasix 20 mg, Atarax 25 mg, |
| | | | Cancer (NSCLC), | Hydroxyzine 25 mg, Klor Con 10 meq, |
| | | | Hypothyroidism | Emla, Methylprednisolone 4 mg, Zofran |
| | | | | 8 mg, Synthroid 100 mcg, Kenalog 0.025% |
| Diseased | 68 | Female | Non Small Cell Lung | Abraxane 100 mg, Procrit 40000 iu, Aspirin |
| | | | Cancer (NSCLC), | 325 mg, Benadryl 25 mg, Calcium 500 mg, |
| | | | Leukocystosis, | Clopidogrel 75 mg, Codeine- Guaifenesin |
| | | | Hypercalcemia, | 10 mg-100 mg/5 ml, Imodium 2 mg, Iron |
| | | | Asthma, Major | 325 mg, Klor-Con 20 meq, Lasix 20 mg, |
| | | | Depressive Disorder, | Levothyroxine 50 mcg, Metformin 500 mg, |
| | | | Hypothyroidism, | Niacin 500 mg, Ondansetron 8 mg, Proventil |
| | | | Hyperlipidemia, Type | 90 mcg, Spiriva 18 mcg, Symbicort 160 mcg- |
| | | | 2 Diabetes | 4.5 mcg, Tylenol 500 mg, Xanax 0.5 mg, |
| | | | | Zolpidem 10 mg |
| Diseased | 78 | Male | Non Small Cell Lung | Xgeva 120 mg, Keytruda, Atorvastatin |
| | | | Cancer (NSCLC), | 40 mg, Digoxin 125 mcg, Furosemide 40 mg, |
| | | | Lymphadenitis, | Lexapro 20 mg, Medrol 4 mg, Metoprolol |
| | | | Hypertension (HTN), | Tartrate 100 mg, Namzaric 21 mg-10 mg, |
| | | | Hyperlipidemia, Atrial | Noxylane 500 mg, Vitamin D2 50000 iu, |
| | | | Fibrillation (AF), Malignant Neoplasm | Warfarin 2 mg |
| | | | of Left Main Bronchus | |
| Diseased | 79 | Female | Non Small Cell Lung | Feraheme Non-ESRD, Amlodipine 5 mg, |
| | | | Cancer (NSCLC), | Aspirin 81 mg, Atenolol 50 mg, Bayer |
| | | | Pancytopenia, Liver | Aspirin 325 mg, Calcium/Vitamin D3 |
| | | | Cirrhosis, Zoster, | 1250 mg, Caltrate/Vitamin D3 1500 mg, |
| | | | Neuralgia, Neuritis, | Cartia XT 180 mg, Crestor 20 mg, Duragesic |
| | | | Essential Primary | 25 mcg, Eliquis 5 mg, Folic Acid 1 mg, |
| | | | Hypertension | Gabapentin 300 mg, |
| | | | | Oxycodone/Acetaminophen 5 mg/325 mg, |
| | | | | Percocet 5 mg/325 mg, Prednisone 1 mg, |
| | | | | Procrit 40000 iu/ml, Tessalon Perles 100 mg, |
| | | | | Vitamin D2 50000 iu |
| Diseased | 79 | Male | Non Small Cell Lung | Octagam Liquid 10%, Calcium 600 mg, |
| | | | Cancer (NSCLC), | Digoxin 125 mcg, Folic Acid 400 mcg, |
| | | | Prostate Cancer, | Metoprolol Tartrate 25 mg, Probiotic, |
| | | | Immune | Rosuvastatin 10 mg |
| | | | Thrombocytopenic | |
| | | | Purpura, HTN, | |
| | | | Hyperlipidemia | |
| Diseased | 54 | Male | Non Small Cell Lung | Pembrolizumab, Dexamethasone 4 mg, |
| | | | Cancer (NSCLC), | Glipizide 5 mg, Hydrocodone- |
| | | | Type 2 Diabetes, | Acetaminophen 10 mg-325 mg, Ipratropium |
| | | | Hypertension (HTN) | Bromide 17 mcg, Lantus, Lisinopril 10 mg, |
| | | | | Metformin 500 mg, Pravastatin Sodium |
| | | | | 40 mg |
| Diseased | 69 | Male | Non Small Cell Lung | Aloxi 0.25 mg/5 ml, Cardizem 120 mg, |
| | | | Cancer (NSCLC), | Crestor 20 mg, Digoxin 250 mcg, Eliquis |
| | | | Unilateral Primary | 5 mg, Furosemide 20 mg, Glucosamine |
| | | | Osteoarthritis of Left | Chondroitin PLUS |
| | | | Knee, Essential | 375 mg/100 mg/36 mg/54 mg, Metformin ER |
| | | | Primary Hypertension, | 750 mg, Potassium Chloride ER 10 meq, |
| | | | Type 2 Diabetes | Zofran 8 mg |
| Diseased | 81 | Male | Non Small Cell Lung | Ipratropium Albuterol 0.5 mg/3 mg, |
| | | | Cancer (NSCLC), | Metoprolol 50 mg, Coumadin 7.5 mg, |
| | | | Vascular Dementia, | Atorvastatin 80 mg, Lovenox 80 mg/0.8 ml, |
| | | | Hypertension (HTN), | Magace ES 625 mg/5 ml, Lexapro 10 mg |
| | | | Lipid Disease | |
| Diseased | 70 | Female | Non Small Cell Lung | Nplate, Procrit 40,000 iu, Alimta 500 mg, |
| | | | Cancer (NSCLC), | Ferrous Sulfate 325 mg, Folic Acid 1 mg, |
| | | | Anemia | Medrol 4 mg, Metformin 500 mg, |
| | | | (Antineoplastic | Simvastatin 40 mg, Tudorza Pressair |
| | | | Chemotherapy), | 400 mcg |
| | | | Idiopathic | |
| | | | Thrombocytopenia | |
| | | | (ITP), Malignant | |
| | | | Neoplasm of Upper | |
| | | | Left Lobe, Vitamin | |
| | | | B12 Deficiency, Folic | |
| | | | Acid Deficiency, Asthma, Type 2 | |
| | | | Diabetes, | |
| | | | Hyperlipidemia | |
| | | | (HLD), Hypertension | |
| | | | (HTN), | |
| | | | Hypercholesterolemia | |
| Diseased | 76 | Female | Non Small Cell Lung | Biotin 300 mcg, Cleocin 1%, Fenofibrate |
| | | | Cancer (NSCLC), | 160 mg, Flonase 50 mcg, Medrol 4 mg, |
| | | | Anemia (Iron | Ranitidine 150 mg, Tagrisso 80 mg, Tarceva |
| | | | Deficiency), , Impaired | 150 mg, Ventolin HFA 90 mcg, Vitamin D2 |
| | | | Fasting Glucose (IFG) | 50000 iu |
| Diseased | 91 | Male | Non Small Cell Lung | Clotrimazole 1%, Dextran, Digoxin |
| | | | Cancer (NSCLC), | 125 mcg, Furosemide 20 mg, Hydrocodone- |
| | | | Anemia (Iron | Homatropine 5 mg-1.5 mg/5 ml, Lasix 20 mg, |
| | | | Deficiency), | Levothyroxine 25 mcg, Metoprolol |
| | | | Hypothyroidism, HTN | Succinate 25 mg, Miracle Mouthwash, |
| | | | | Mucinex 30 mg-600 mg, Nystatin 100000 iu, |
| | | | | Omeprazole 20 mg, Oxycodone 20 mg, |
| | | | | Pravastatin 10 mg, Prednisone 10 mg, Proair |
| | | | | 90 mcg, Procto-Med 2.5%, Relistor 150 mg, |
| | | | | Sodium Chloride 1 g |
| Diseased | 69 | Female | Non Small Cell Lung | Ativan 0.5 mg, Trazodone 50 mg |
| | | | Cancer (NSCLC), | |
| | | | Anemia (Iron | |
| | | | Deficiency), Vitamin | |
| | | | B12 Deficiency, T- | |
| | | | Cell Prolymphocytic | |
| | | | Leukemia, | |
| | | | Hypertension (HTN) | |
| Diseased | 65 | Female | Non Small Cell Lung | Dexamethasone 4 mg, Emla 2.5%, |
| | | | Cancer (NSCLC), | Loperamide 2 mg, Lorazepam 1 mg, |
| | | | Chronic Obstructive | Nystatin 100000 iu/ml, Ondansetron 4 mg, |
| | | | Pulmonary Disease | Oravig 50 mg, Symbicort 160 mcg/4.5 mcg, |
| | | | (Emphysema), | Ventolin HFA 90 mcg, Navelbine 30 mg |
| | | | Cardiovascular | |
| | | | Disease, Osteoporosis | |
| Diseased | 77 | Female | Non Small Cell Lung | Avastin 15 mg/kg, Alimta |
| | | | Cancer (NSCLC) | 500 mg/Carboplatin/Neulasta then Alimta |
| | | | | 500 mg, Aspirin 81 mg, Chantix 1 mg, |
| | | | | Dexamethasone 4 mg, Folic Acid 1 mg, |
| | | | | Instaflex, Metformin 500 mg, Quinapril |
| | | | | 40 mg, Simvastatin 20 mg, Vitamin D3 |
| | | | | 2000 iu, Zofran 8 mg |
| Diseased | 85 | Female | Non Small Cell Lung | Aletinib |
| | | | Cancer (NSCLC) | |
| Control | 53 | Female | Normal Donor | None |
| Control | 64 | Female | Normal Donor | None |
| Control | 67 | Female | Normal Donor, | Atorvastatin 40 mg |
| | | | Hyopercholesterolemia | |
| Control | 72 | Female | Normal Donor | None |
| Control | 73 | Female | Normal Donor, | Prolia, Aciphex 20 mg |
| | | | Osteoarthritis (OA) | |
| Control | 87 | Male | Normal Donor, Donor | Vitamin B Complex, Zinc 50 ml, Alka |
| | | | with Fever | Seltzer, Vitamin B6, Vitamin B1, Vitamin |
| | | | | B12, Pepsin 40 mg |
| Control | 65 | Female | Normal Donor, | None |
| | | | Hypercholesterolemia | |
| Control | 80 | Female | Normal Donor | None |
| Control | 57 | Male | Normal Donor | Multivitamin |
| Control | 63 | Female | Normal Donor | None |
| Control | 75 | Male | Normal Donor, | Lisinopril 2.5 mg |
| | | | Hypertension (HTN) | |
| Control | 70 | Female | Normal Donor | None |
| Control | 70 | Female | Normal Donor | Multivitamin 1000 mg |
| Control | 77 | Female | Normal Donor | Lipitor 20 mg, Prevacid 20 mg |
| Control | 68 | Female | Normal Donor, | Lisinopril 10 mg |
| | | | Hypertension (HTN) | |
| Control | 73 | Male | Normal Donor | Tamsulosin HCL, Finasteride 5 mg |
| Control | 81 | Female | Normal Donor | Norvasc, Ditropan |
| Control | 77 | Male | Normal Donor, | Lipitor 20 mg, Tricor 145 mg, Metoprolol |
| | | | Cataract, Progressive | 50 mg, Omeprazole 20 mg, Aspirin 80 mg |
| | | | Hearing Loss, | |
| | | | Hypertension (HTN), | |
| | | | Hypercholesterolemia | |
| Control | 56 | Male | Normal Donor | Nexium 60 mg, Zocor 40 mg |
| Control | 64 | Male | Normal Donor | Protonix 40 mg, Asiprin 325 mg |
| Control | 80 | Male | Normal Donor | Vitamin D, Lipitor, Aspirin 81 mg |
| Control | 73 | Female | Normal Donor, | Allegra 180 mg |
| | | | Allergic Rhinitis | |
| Control | 77 | Female | Normal Donor | None |
| Control | 83 | Male | Normal Donor | None |
| Control | 68 | Female | Normal Donor | Losartan 50 mg, Lipitor 20 mg |
| Control | 66 | Female | Normal Donor, | Lisinopril 10 mg |
| | | | Hypertension (HTN) | |
| Control | 78 | Female | Normal Donor | Lipitor 10 mg, Toprol 50 mg, Ambien 10 mg |
| Control | 86 | Female | Normal Donor, | Amlodipine 2.5 mg, Vitamin B |
| | | | Hypertension (HTN) | |

TABLE 15
CRC and Controls

| Class | Age | Gender | Diagnosis |
|---|---|---|---|
| Diseased | 74 | Female | Colorectal Cancer |
| Diseased | 41 | Male | Colorectal Cancer |
| Diseased | 57 | Male | Colorectal Cancer, Anemia (Iron Deficiency), Type 2 Diabetes |
| Diseased | 78 | Male | Colorectal Cancer, Chronic Lymphocytic Leukemia (CLL), |
| | | | Hypertension (HTN), Type 2 Diabetes, Arthritis |
| Diseased | 60 | Female | Colorectal Cancer, CKD, Iron Deficiency, RLS, Carcinoma of |
| | | | Colon, Carcinoma of Right Ovary, Anxiety |
| Diseased | 37 | Male | Colorectal Cancer, Erectile Dysfunction, Thrombocytopenia |
| Diseased | 68 | Female | Colorectal Cancer, Major Depressive Disorder (MDD), Type 2 |
| | | | Diabetes, Hernia with Obstruction, Ventral Hernia, Dysphonia, |
| | | | Hypercholesterolemia, Hyperlipidemia, Hypertension (HTN), |
| | | | Migraine, Obesity, Diabetic Polyneuropathy, Reflux Esophagitis, |
| | | | Edema, Asthma, Chronic Obstructive Pulmonary Disease |
| | | | (COPD), E Coil Bacteremia, Subdural Hematoma, Hepatic |
| | | | Abscess |
| Diseased | 60 | Female | Colorectal Cancer, Rectal Cancer, Type 1 Diabetes, |
| | | | Hypertension (HTN), Hypothyroidism |
| Control | 75 | Female | Normal Donor |
| Control | 42 | Male | Normal Donor |
| Control | 56 | Male | Normal Donor |
| Control | 78 | Male | Normal Donor |
| Control | 58 | Female | Normal Donor |
| Control | 36 | Male | Normal Donor |
| Control | 68 | Female | Normal Donor |
| Control | 59 | Female | Normal Donor |

### Example 10

### Characterization of Physicochemical Properties of Particle Types

This example describes characterization of particle physicochemical properties by various techniques. Dynamic light scattering (DLS) and zeta potential were performed on a Zetasizer Nano ZS (Malvern Instruments, Worcestershire, UK). Particles were suspended at 10 mg/mL in water with about 10 min of bath sonication prior to testing. Samples were then diluted to approximately 0.02 wt % for both DLS and zeta potential measurements in respective buffers. DLS was performed in water at about 25° C. in disposable polystyrene semi-micro cuvettes (VWR, Randor, Pa., USA) with a about 1 min temperature equilibration time and consisted of the average from 3 runs of about 1 min, with a 633 nm laser in 173° backscatter mode. DLS results were analyzed using the cumulants method. Zeta potential was measured in 5% pH 7.4 PBS (Gibco, PN 10010-023, USA) in disposable folded capillary cells (Malvern Instruments, PN DTS1070) at about 25° C. with an about 1 min equilibration time. 3 measurements were performed with automatic measurement duration with a minimum of 10 runs and a maximum of 100 runs, and a 1 min hold between measurements. The Smoluchowski model was used to determine the zeta potential from the electrophoretic mobility.

Scanning electron microscopy (SEM) was performed by using a FEI Helios 600 Dual-Beam FIB-SEM. Aqueous dispersions of particles were prepared to a concentration of about 10 mg/mL from weighted particle powders re-dispersed in DI water by about 10 min sonication. Then, the samples were 4× diluted by methanol (from Fisher) to make a dispersion in water/methanol that was directly used for electron microscopy. The SEM substrates were prepared by drop-casting about 6 μL of particle samples on the Si wafer from Ted Pella, and then the droplet was completely dried in a vacuum desiccator for about 24 hours prior to measurements.

A Titan 80-300 transmission electron microscope (TEM) with an accelerating voltage of 300 kV was used for both low- and high-resolution TEM measurements. The TEM grids were prepared by drop-casting about 2 μL of the particle dispersions in water-methanol mixture (25-75 v/v %) with a final concentration of about 0.25 mg/mL and dried in a vacuum desiccator for about 24 hours prior to the TEM analysis. All measurements were performed on the lacey holey TEM grids from Ted Pella.

X-Ray Photoelectron Spectroscopy (XPS) was performed by using a PHI VersaProbe and a ThermoScientific ESCALAB 250e III. XPS analysis was performed on the particle fine powders kept sealed and stored under desiccation prior to the measurements. Materials were mounted on a carbon tape to achieve a uniform surface for analysis. A monochromatic Al K-alpha X-ray source (50 W and 15 kV) was used over a 200 μm² scan area with a pass energy of 140 eV, and all binding energies were referenced to the C-C peak at 284.8 eV. Both survey scans and high-resolution scans were performed to assess in detail elements of interest. The atomic concentration of each element was determined from integrated intensity of elemental photoemission features corrected by relative atomic sensitivity factors by averaging the results from two different locations on the sample. In some cases, four or more locations were averaged to assess uniformity.

### Example 11

### Protein Corona Preparation and Proteomic Analysis

This example describes protein corona preparation and proteomic analysis. Plasma and serum samples were diluted 1:5 in a dilution buffer composed of TE buffer (10 mM Tris, 1 mM disodium EDTA, 150 mM KCl) with 0.05% CHAPS. Particle powder was reconstituted by sonicating for about 10 min in DI water followed by vortexing for about 2-3 sec. To make a protein corona, about 100 μL of particle suspension (SP-003, 5 mg/ml; SP-007, 2.5 mg/ml; SP-011, 10 mg/ml) was mixed with about 100 μL of diluted biological samples in microtiter plates. The plates were sealed and incubated at 37° C. for about 1 hour with shaking at 300 rpm. After incubation, the plate was placed on top of magnetic collection for about 5 mins to pellet down the nanoparticles. Unbound proteins in supernatant were pipetted out. The protein corona was further washed with about 200 μL of dilution buffer for three times with magnetic separation. For the 10 particle type particle panel screen, the five additional assay conditions that were evaluated were identical to the description above with one of the following exceptions. First, a low concentration of particles was evaluated that was 50% the concentration of the original particle concentration (ranging from 2.5-15 mg/ml for each particle, depending on expected peptide yield). For the second and third assay variations, both low and high particle concentrations were run using an undiluted, neat plasma rather than diluting the plasma in buffer. For the fourth and fifth assay variations, both low and high particle concentrations were run using a pH 5 citrate buffer for both dilution and rinse.

To digest the proteins bound onto nanoparticles, a trypsin digestion kit (iST 96X, PreOmics, Germany) was used according to protocols provided. Briefly, about 50 μL of Lyse buffer was added to each well and heated at about 95° C. for about 10 min with agitation. After cooling down the plates to room temperature, trypsin digest buffer was added and the plate was incubated at about 37° C. for about 3 hours with shaking. The digestion process was stopped with a stop buffer. The supernatant was separated from the nanoparticles by a magnetic collector and further cleaned up by a peptide cleanup cartridge included in the kit. The peptide was eluted with about 75 μL of elution buffer twice and combined. Peptide concentration was measured by a quantitative colorimetric peptide assay kit from Thermo Fisher Scientific (Waltham, Mass.).

Next, the peptide eluates were lyophilized and reconstituted in 0.1% TFA. A 2 μg aliquot from each sample was analyzed by nano LC-MS/MS with a Waters NanoAcquity HPLC system interfaced to an Orbitrap Fusion Lumos Tribrid Mass Spectrometer from Thermo Fisher Scientific. Peptides were loaded on a trapping column and eluted over a 75 pm analytical column at 350 nL/min; (NanoAcquity HPLC) or 250 nL/min (UltiMate 3000 RSLCnano system) using a gradient of 2-35% acetonitrile over 44 minutes, for a total time between injections of 64 (UltiMate 3000 RSLCnano system) or 66 minutes (NanoAcquity HPLC). The mass spectrometer was operated in a data-dependent mode, with MS and MS/MS performed in the Orbitrap at 60,000 FWHM resolution and 15,000 FWHM resolution, respectively. The instrument was run with a 3 sec cycle for MS and MS/MS.

### Example 12

### Mass Spectrometry Data Analysis

This example describes mass spectrometry data analysis methods. The acquired MS data files were processed using the OpenMS suite of tools. These tools include modules and pipeline scripts for the conversion of vendor instrument raw files to mzML files, for MS1 feature identification and intensity extraction, for MS dataset run-time alignment and feature-group clustering, and for MS2 spectrum database matching with the X! Tandem search engine. During spectrum-database searching the precursor ion and fragment ion matching tolerances were set to 10 and 30 ppm, respectively. Default settings for fixed, Carbamidomethyl (C), and variable, Acetyl (N-term) and Oxidation (M), modifications were enabled. The UniProtKB/Swiss-Prot protein sequence database (accession date Jan. 27, 2019) was used for searches and peptide spectral matches (PSMs) were scored using a standard reverse-sequence decoy database strategy at 1% FDR. Using the PSMs, protein lists for each particle type replicate were compiled using a single PSM as sufficient evidence to add a protein to a given particle type replicate's enumerated protein list. In addition, a PSM that matched more than one protein added all of the possible proteins to the given particle type replicate's enumerated protein list. Although this threshold for protein enumeration is permissive, and possibly includes false-positives (higher sensitivity, lower specificity), the more stringent test of requiring 2 or more peptides (including at least one unique peptide) suffers from the opposite problem of having false-negatives (lower sensitivity, higher specificity). For quantitative analysis of known peptides, a custom R script was used to assign MS2 PSMs to MS1 feature groups based on positional overlap with 1 da and 30 sec tolerances for mz and retention time, respectively. In the event that more than one PSM initially mapped to an MS1 feature within the tolerances previously specified, the PSM which was closest to the MS1 feature (within MS datasets) or to the center of the MS1 feature cluster (between MS datasets) was used. It should be noted that not all MS2s have been assigned to MS1 feature group clusters, and not all MS1 feature group clusters have an assigned MS2; work continues in this area to improve mapping and subsequent peptide feature identification.

### Example 13

### Identification of Protein Groups

This example describes methods for identification of protein groups by mass spectrometry. For protein group-level analysis, the MS data at the protein group level was performed as follows. MS raw files were processed with MaxQuant (v. 1.6.7) and Andromeda, searching MS/MS spectra against the UniProtKB human FASTA database (UP000005640, 74,349 forward entries; version from August 2019) employing standard settings. Enzyme digestion specificity was set to trypsin allowing cleavage N-terminal to proline and up to 2 miscleavages. Minimum peptide length was set to 7 amino acids and maximum peptide mass was set to 4,600 Da. Methionine oxidation and protein N-terminus acetylation were configurated as a variable modification, carbamidomethylation of cysteines was set as fixed modification. MaxQuant improves precursor ion mass accuracy by time-dependent recalibration algorithms and defines individual mass tolerances for each peptide. Initial maximum precursor mass tolerances allowed were 20 ppm during the first search and 4.5 ppm in the main search. The MS/MS mass tolerance was set to 20 ppm. For analysis, a false discovery rate (FDR) cutoff of 1% was applied at the peptide and protein level (in the proteinGroups.t×t table, all protein groups are reported with their corresponding q-value). “Match between runs,” was disabled. Number of identifications where counted based on protein intensities (counting only proteins with q-value lower than 1%) requiring at least one razor peptide. MaxLFQ normalized protein intensities (requiring at least 1 peptide ratio count) are reported in the raw output and were used only for the CV precision analysis. Peptides that could be distinguished were sorted into their own protein groups and proteins that could not be discriminated based on unique peptides were assembled in protein groups. Furthermore, proteins were filtered for a list of common contaminants included in MaxQuant. Proteins identified only by site modification were strictly excluded from analysis.

### Example 14

### Spike Recovery

This example describes methods for spike recovery experiments of C-reactive protein (CRP). Baseline concentration of CRP in a pooled healthy plasma sample was measured with the ELISA kit as described in EXAMPLE 7 according to the manufacturer-suggested protocols. A stock solution and appropriate dilutions of CRP were prepared and spiked into the identical pooled plasma samples to make final concentrations that were 2×, 5×, lox, and 100× of baseline, endogenous concentrations for CRP. The volume of additions to the pooled plasma was 10% of the total sample volume. A spike control was made by adding same volume of buffer to the pooled plasma sample. Concentrations of spiked samples were measured again by ELISA to confirm the CRP levels in each spiking level. The samples were used to evaluate particle corona measurement linearity as described in the Results above.

### Example 15

### Proteomic Analysis of NSCLC Samples and Healthy Controls

This example describes proteomic analysis of NSCLC samples and health controls.

Serum samples from 56 subjects, 28 with Stage IV NSCLC and 28 age- and gender-matched controls were purchased commercially and evaluated with SP-007 nanoparticle corona formation. Sample acquisition is described in EXAMPLE 9 and corona formation and processing are described in EXAMPLE 11. MS spectral data for each corona were collected as described and the raw data were processed as described in EXAMPLE 12. 19,214 groups of features were identified, as described in EXAMPLE 13, and extracted across the 56 subject samples with group sizes ranging from one (singleton features in just one sample, n=6,249 or 0.29% of the data) to 56 (features present in all samples, n=450 or 12% of the data). The clustering algorithm calculates a ‘group_quality’ metric which is related to the spatial uniformity of grouping of features with groups between datasets. The bottom quartile of groups, partitioned by group size, was then removed from consideration due to the skewed nature of the distribution of low-quality scores leaving 15,967 groups. As an additional filter prior to analysis, only those groups with features present in at least 50% of at least one of the classes, diseased or control, were carried forward leaving a set of 2,507 feature groups for analysis.

Peptide and protein identities were assigned to the feature groups as follows. MS2 PSMs and MS1 feature groups were assigned together as described above (MS data analysis). 25% of the 19,249 original feature groups were associated with a peptide sequence using this approach. All feature groups, with or without assigned peptide sequence, were carried through the univariate statistical comparison between the groups.

### Example 16

### Statistical Analysis

This example describes statistical analysis of the data disclosed herein. Statistical analysis and visualization were performed using R (v3.5.2) with appropriate packages (R: A language and environment for statistical computing. R Foundation for Statistical Computing, Vienna, Austria. URL https://www.R-project.org/).

### Example 17

### Precision of the Corona Analysis Assay

This example describes precision of the corona analysis assay. To investigate the reproducibility of the platform, the peptide MS feature intensities were extracted and compared from the three full-assay replicates for all three NPs. All quantifiable MS1 features were used in order to fully explore the precision possible in future studies, regardless of whether a given MS feature is currently identified. The raw MS files for each replicate were converted to mzML, a standard, interchangeable MS file format, using the msconvert.exe utility from the openMS suite of programs. Also using the openMS processing pipeline, MS1 features (monoisotopic peaks) were extracted from the raw data and aligned into groups by overlapping retention time and mass-charge ratio (mz) values. Groups selected contained a feature from each of the three replicates and were filtered to remove the bottom decile based on the clustering algorithm's quality score (90% of feature groups retained for subsequent precision analysis). For S-003, S-007, and 5-011 NPs, a total of 2,744, 2,785, and 3,209 clustered MS1 feature groups (respectively) were used for analysis. Overall precision was then estimated by normalizing the group feature intensities using quantile normalization, assuming that all compared distributions are identical and adjusting the intensities for each compared distribution appropriately. With the normalized values, the standard deviations were evaluated and the coefficients of variation (CVs) determined using the appropriate transformation of log-treated data. The median CVs (percent of quantile normalized CV or QNCV %) for each NP are shown in TABLE 16; the average precision was CV 24%. The NP-measured protein MS feature intensities have sufficient precision (across thousands of intensities observed) to detect relatively small differences in reasonably small studies. For example, a study with just 25 samples and assuming 2,000 features, would have 85% power to detect differences of 50% in protein concentrations between groups with a Bonferroni-corrected alpha=0.05/2000.

TABLE 16
Median quantile normalized CV % for precision evaluation
of the NP protein corona-based Proteograph workflow

| Particle | Median QNCV % | Count |
|---|---|---|
| S-003 | 23 | 2744 |
| S-007 | 29 | 2785 |
| S-011 | 20 | 3209 |

### Example 18

### Diversity of Information Across the 10-NP Panel

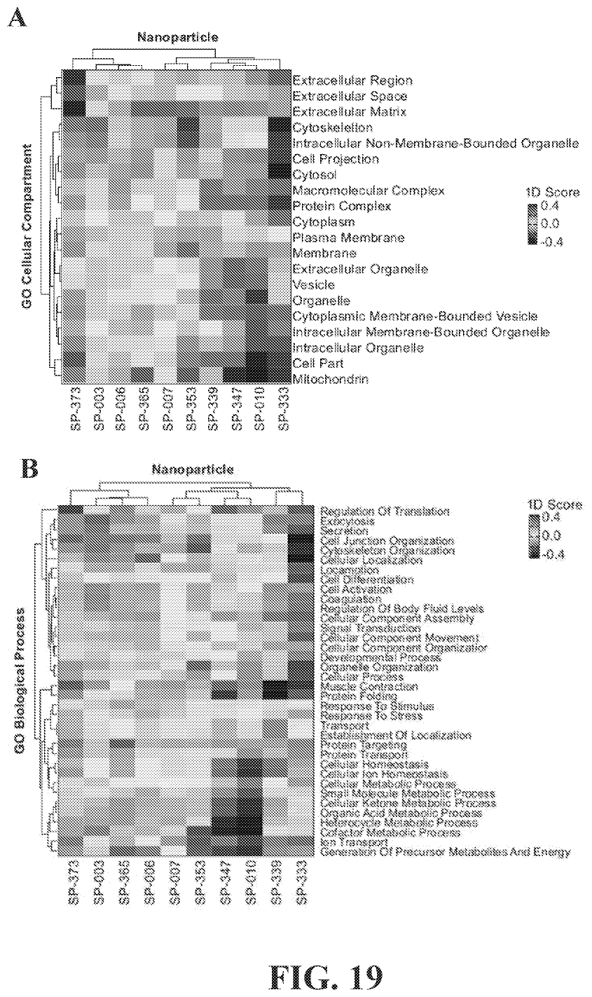

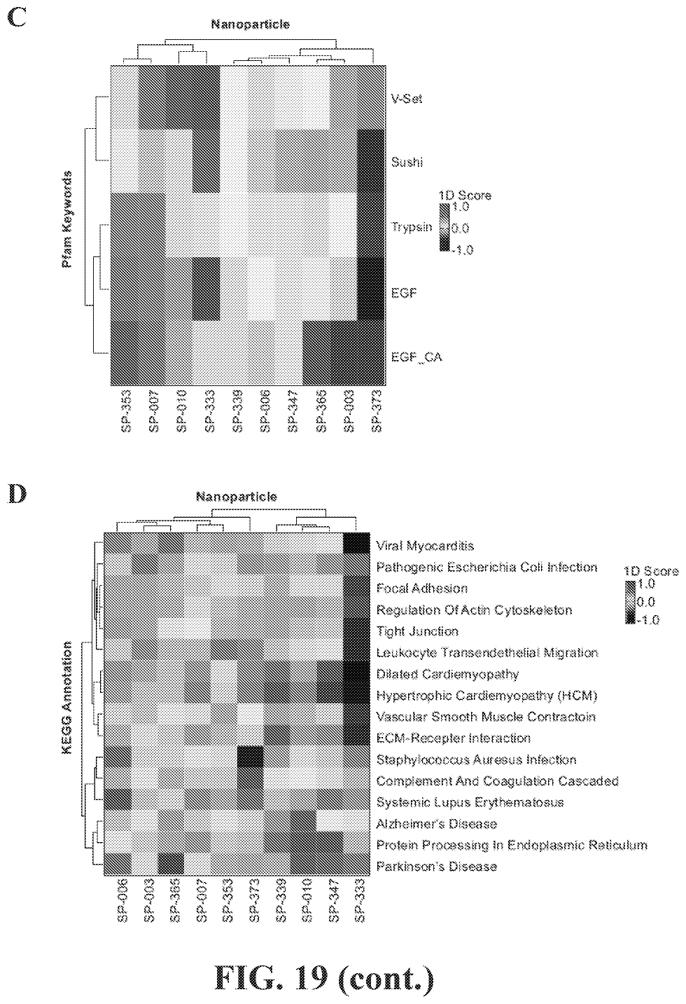

This example describes diversity of information across the 10-NP panel. While it is certainly of interest to compare the individual protein IDs that make up the 2,009 protein groups detected by MaxQuant across the subjects at the 1% FDR protein level, it is also of interest to determine if any differentiation between the particles exists at one or more levels of annotation. To investigate the degree to which NP coronas are enriched or depleted for proteins associated with specific biochemical and biological pathways, NP-specific enrichment and depletion were analyzed within the cancer subset of the 16 samples used for coverage described above (10 NPs for each of eight subjects). The high sensitivity and wide dynamic range (more than three orders of magnitude per sample) achieved with modern mass spectrometers limits the applicability of a categorical enrichment analysis that evaluates only the dichotomous presence or absence of a feature (e.g. hypergeometric distribution tests like Fisher's Exact test). For this reason a 1D annotation enrichment was employed to compare protein coronas on a functional level. As shown in FIG. 19, clustering based on 1D enrichment analysis shows distinct and differential patterns of enrichment and depletion across the 10-NP panel. For example, the GO Cellular Compartment annotation space characterizes protein location. In that category, NPs cluster into two major branches (Cluster 1 with SP-373, SP-003, SP-006, and SP-365 versus Cluster 2 with S-007, SP-353, SP-339, SP-347, SP-010, and SP-333). The second cluster shows depletion of most annotations and an enrichment of proteins associated with the extracellular region. SP-373 (cluster 1) shows a particularly strong enrichment for intracellular proteins and strong depletion for extracellular proteins. Many high-abundance proteins in plasma including immune globulins and albumin are annotated as extracellular, illustrating the capacity of NPs to sample a large dynamic range. This is consistent with the profile observed for protein families (Pfam), in a particular V-set, which includes antibody variable domains. Moreover, in Pfam, SP-373 is depleted for EGF-associated categories (EGF and EGF-CA), while these annotations are particularly enriched in SP-353 and SP-007. With respect to the associated gene ontology biological processes (GOBP), NP coronas cluster quite similarly to GOCC; but SP-373 shows a more distinct separation, being particularly enriched for proteins associated with metabolic processes. Some enriched disease- and inflammation-associated signatures are suggested by the KEGG results. In particular, SP-006 shows a strong enrichment for lupus and S. aureus infection. In summary, annotation enrichments show that NP coronas can be categorized not only on the level of individual proteins but also based on the functional groups of proteins. In principle, an experiment could take advantage of different subsets of particles focusing on specific protein group IDs or enriched annotations, which might be more relevant to the question at hand. Moreover, the capacity to interrogate different functional classes of proteins (extracellular region, membrane, or cytosol) illustrates how NP coronas are capable of sampling a wide dynamic range in complex proteomes.

### Example 19

### Coverage of the Interactome

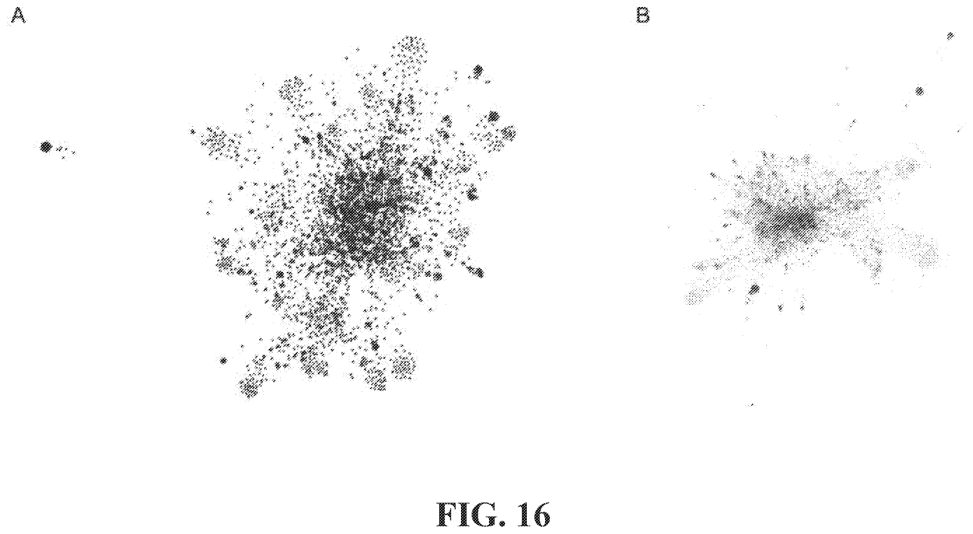

This example describes coverage of the interactome. To determine how broadly the 2,009 identified protein groups identified by the 10-NP panel across the 16 individual plasma samples covered the known protein-protein interactome, the constituents of each protein group were mapped to genes, discarding groups that mapped to multiple loci, reducing the 2,009 protein groups to 1,829 gene loci. Coverage was then evaluated against whole-genome and plasma proteome—specific interactome maps (Methods). The whole-genome interactome contains 12,746 members, of which the 10-NP panel covers 9,057 (71%) either directly or through a direct interaction. For the plasma proteome—specific interactome, the panel covers 3,053 out of 3,482 (88%), also either directly or through a direct interaction. Thus, the proteins covered by the panel span the whole interactome and can be used to interrogate a wide range of samples FIG. 16.

### Example 20

### Annotation Diversity Analysis

This example describes annotation diversity analysis. Continuous enrichment analysis (e.g., 1D annotation enrichment) was used to compare NPs at the annotation level which has the advantage of using quantitative comparison as a more powerful evaluation tool instead of requiring a binary input (e.g., presence/absence, threshold counting, etc.). This method was used to interrogate annotations enriched in the protein coronas by computing the 1D enrichment scores for each nanoparticle in the panel. In summary, log 2-transformed MaxQuant intensities for each protein group in each sample were normalized by median subtraction. Protein groups that were not quantified in at least 4 of the 8 biological replicates used in the analysis on at least one NP were removed. Only the 8 cancer samples from the 16 samples for overall profiling were used for this analysis to avoid any enrichment between NPs being confounded by any differences between healthy subjects and those with cancer. A difference score was calculated for each protein group between the medians on one NP versus the average for that group across all of the other NPs. Annotations from five different spaces, GO Cellular Compartment (GOCC), GO Biological Process (GOBP), Uniprot Keywords, Protein families (Pfam), and Kyoto Encyclopedia of Genes and Genomes (KEGG), were matched to the protein groups based on the Uniprot identifiers reported in the MaxQuant output for each group as Majority Protein IDs. To match identifier format in the annotation reference, the isoform extensions were removed. The annotation references were retrieved from Uniprot on Nov. 25, 2019 using the Persueus/MaxQuant framework. The 1D annotation enrichment was calculated using R scripts adapted from. The results were filtered requiring 1) an annotation group size (ie., number of protein groups with that annotation) greater than 10, and 2) a Benjamini-Hochberg-adjusted p-value (FDR) less than a 5% for enrichment or depletion for at least one NP. The 1D enrichment score was visualized as a heatmap after hierarchical clustering as shown in FIG. 4A) Gene Ontology Cellular Component (GOCC), B) Gene Ontology Biological Process (GOBP), C) Uniprot Keywords, D) Protein families (Pfam), E) Kyoto Encyclopedia of Genes and Genomes (KEGG).

### Example 21

### Interactome Analysis

This example describes interactome analysis. Protein-protein interactions were downloaded from the STRING database version 11.0 (available at string-db.org). Interactions with a score <700 were removed. The plasma proteome interactome was derived by including only those interactions in which both proteins of an interacting pair were present in the plasma proteome. The list of proteins in the plasma proteome comprised the union of proteins identified as shown in EXAMPLE 5, and the proteins identified in Niu L et al. (2019) Mol Syst Biol 15:e8793, Zhou W et al. (2019) Nature 569:663-671, Geyer P E et al. (2016) Mol Syst Biol 12:901, and Bruderer et al. (2019) Molecular & Cellular Proteomics 18(6):1242-1254. The interactome was plotted using Gephi.

### Example 22

### Identifying Protein-Protein Interactions Using Protein Corona Analysis

This example describes identifying protein-protein interactions (PPIs) using protein corona analysis. Protein-protein interaction candidates were identified by correlating protein intensities identified in protein corona across samples from 288 subjects. Correlations of intensities of a single protein were compared between two different particles (“same protein” correlation), and correlations of protein intensities were compared between two different proteins on the same particle type (“same particle” correlation). If a protein-protein interaction was present between the two proteins, the correlation of protein intensities between the two proteins on the same particle was expected to be high, while the correlation of protein intensity for one of the proteins between the two particle types was expected to be low.

FIG. 20A and FIG. 20B show schematics illustrating a method to identify protein-protein interactions present in biomolecule corona. FIG. 20A shows a first protein (dark gray small ovals 2005) that binds directly to two particle types with distinct physicochemical properties (“P1” and “P2”). Because the first protein binds directly to both particle types, the measured protein intensity is well correlated on both particle types across multiple samples. Protein intensity across different samples (e.g., a protein intensity pattern) for each particle type is depicted by the jagged line to the right of each particle. FIG. 20B shows a first protein (dark gray small ovals 2005) that binds directly to a first particle type (“P1”) and binds indirectly to a second particle type (“P2”). The first protein binds to the second particle type through protein-protein interactions with a second protein (lighter gray small oval 2010). Because the first protein 2005 binds to the second particle type through the second protein 2010, the protein intensities of the first protein and the second protein on the second particle type are well correlated across multiple samples. Since the first protein binds directly to the first particle type but indirectly to the second particle type, the first protein intensity is not well correlated on the first particle type and the second particle type across multiple samples. Protein intensity across different samples for each protein on particle type is depicted by the jagged line to the right of each protein and particle type.

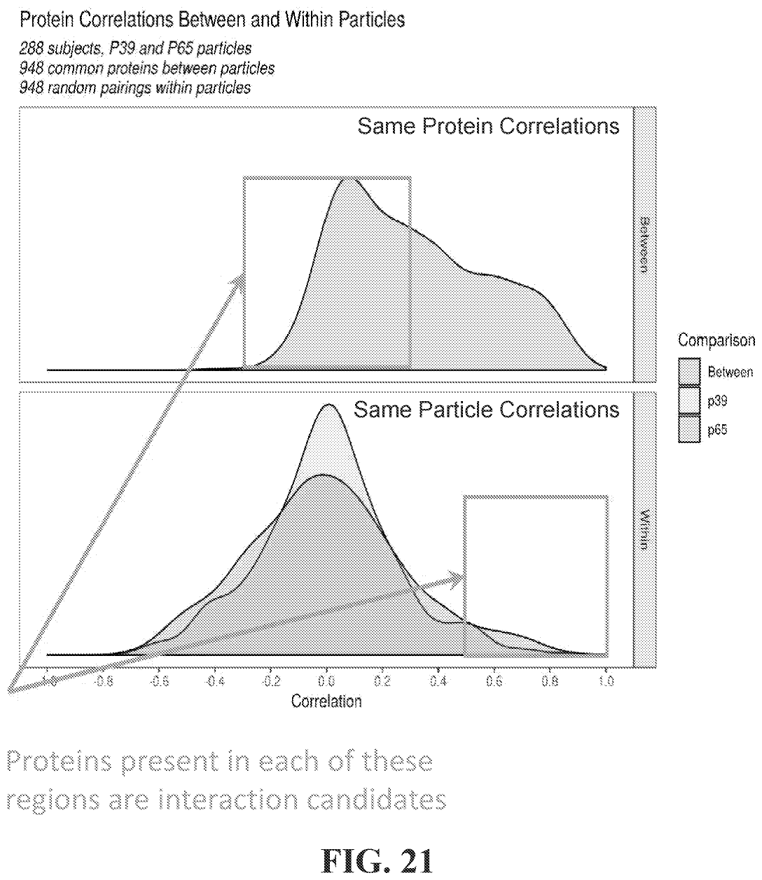

A protein corona analysis assay was performed on samples from 288 subjects using two particle types, P39 (polystyrene carboxyl functionalized particles) and P65 (silica particles). 948 proteins were identified that were common between protein corona formed on the two particle types. 948 random protein pairings were tested within each particle type. FIG. 21 shows distributions of protein correlations across multiple subject samples for two different particle types (P39 and P65). The top plot shows correlations of identified proteins across 288 samples between the two particle types. The bottom plot shows pairwise correlations of random protein parings within each of the two particle types. Protein pairings which showed high correlation within the two particle types (indicated by the box on the right side of the bottom plot) and where one of protein of the pair showed low correlation between the two particle types (indicated by the box on the left side of the top plot) were identified as protein-protein interaction candidates.

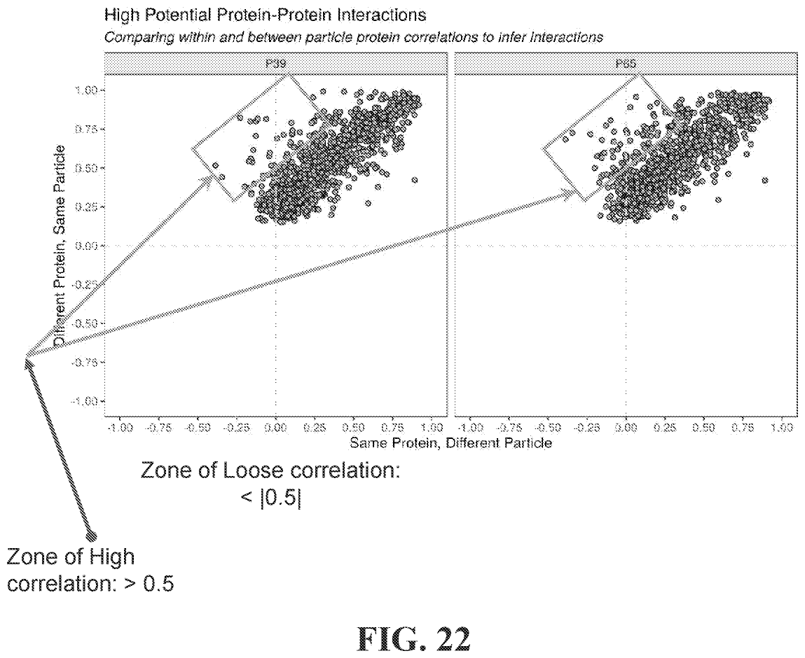

FIG. 22 shows a plot of the protein-protein interaction candidates identified in FIG. 21. The x-axis of each plot shows the correlation of the identified proteins between the two particle types (as plotted in the top panel of FIG. 21), and the y-axis of each plot shows the pairwise correlation between the protein-protein interaction candidates (as plotted in the bottom panel of FIG. 21) on either the P39 particle type (left plot) or the P65 particle type (right plot). Interactions falling in the zone of high correlation (≥0.5) on the y-axis and the zone of loose correlation on the x-axis (<|0.5|), identified by the boxed regions, correspond to potential protein-protein interactions.

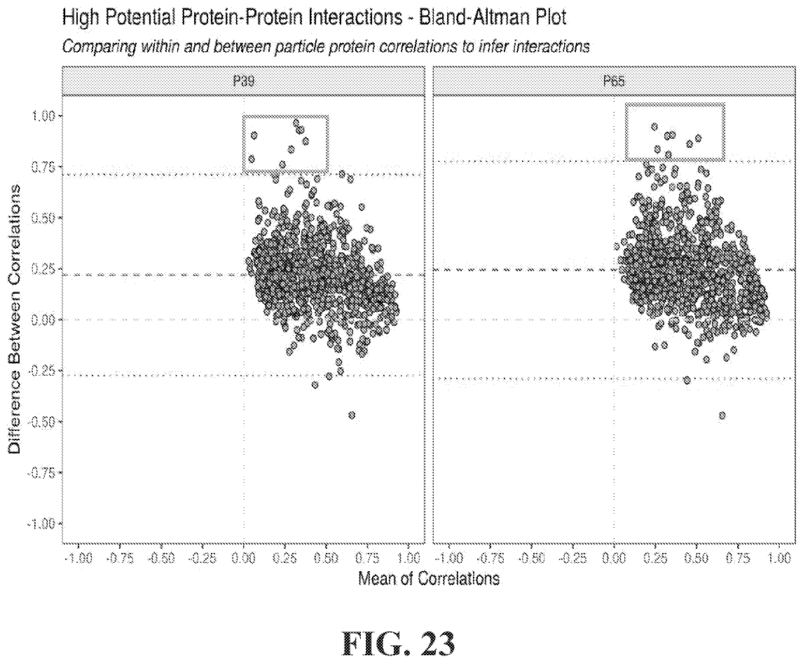

FIG. 23 shows a plot of the protein-protein interaction candidates identified in FIG. 21 and plotted in FIG. 22. The x-axis of each plot shows the average of the correlation of a protein between two particles and the pairwise correlation of two proteins interaction candidates on the same particle type (p39, left plot, or P65, right plot). The y-axis shows the different between the pairwise correlation of two proteins interaction candidates on the same particle type and the correlation of a protein between two particles. Protein pairs with high difference between correlations, denoted by boxes, represent protein pairs with high potential for protein-protein interactions.

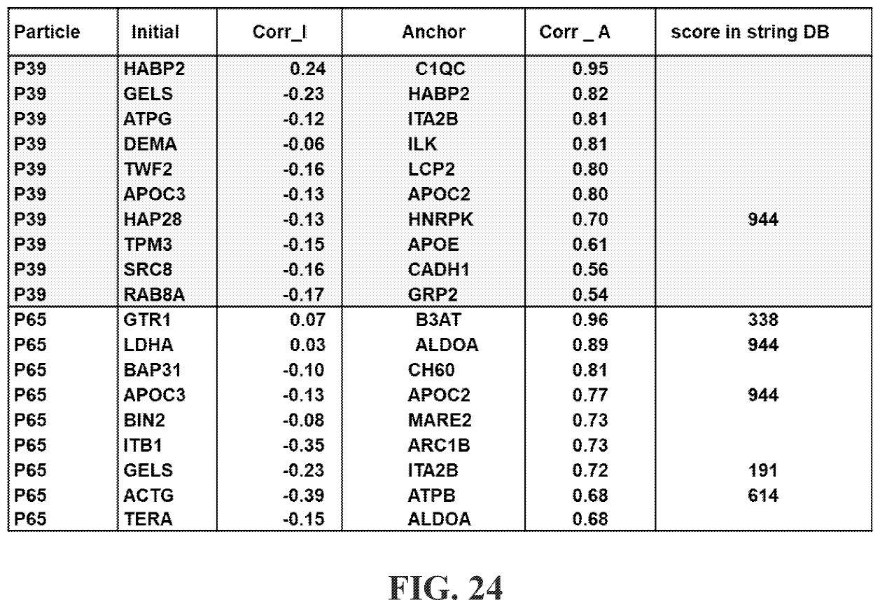

FIG. 24 shows a table of correlation values for potential protein-protein interaction pairs identified from the data plotted in FIG. 21-FIG. 23. Initial correlation values (“Corr_I”) indicate the correlation between the protein intensity of the initial protein (“Initial”) on the P39 and P65 particle types. Anchor correlation values (“Corr_A”) indicate the correlation between the protein intensity of the initial protein and the anchor protein (“Anchor”) on the same particle type (“Particle”). The protein-protein interaction score from the STRING database is provided where applicable, with a higher score indicating a greater likelihood of a protein-protein interaction for a protein pair (high confidence scores are greater than 700).

The following protein pairs were identified as protein-protein interaction candidates: HABP2 and C1QC, GELS and HABP2, ATPG and ITA2B, DEMA and ILK, TWF2 and LCP2, APOC3 and APOC2, HAP28 and HNRPK, TPM3 and APOE, SRC8 and CADH1, RAB8A and GRP2, GTR1 and B3AT, LDHA and ALDOA, BAP31 and CH60, BIN2 and MARE2, ITB1 and ARC1B, GELS and ITA2B, ACTG and ATPB, and TERA and ALDOA. As can be seen from FIG. 24, the majority of protein-protein interactions identified in the particle data from 288 subjects were previously unknown, highlighting the power of the present methods for discerning protein-protein interactions from particle data. As shown in this non-limiting example, the method can find new pairs and recapitulate existing pairs.

### Example 23

### Protein Cluster Representation in Protein Corona

This example describes protein cluster representation in protein corona. Protein populations captured in protein corona on different particle types were compared to biological protein-protein interaction maps of known protein interactions. Interaction maps, in which nodes represent proteins and connections represent interactions, were generated such that proteins that interact together and are more closely related were positioned closer together. Biological protein-interactions were taken from the STRING public database and were identified using yeast-hybrid assays to identify in vivo protein-protein interactions.

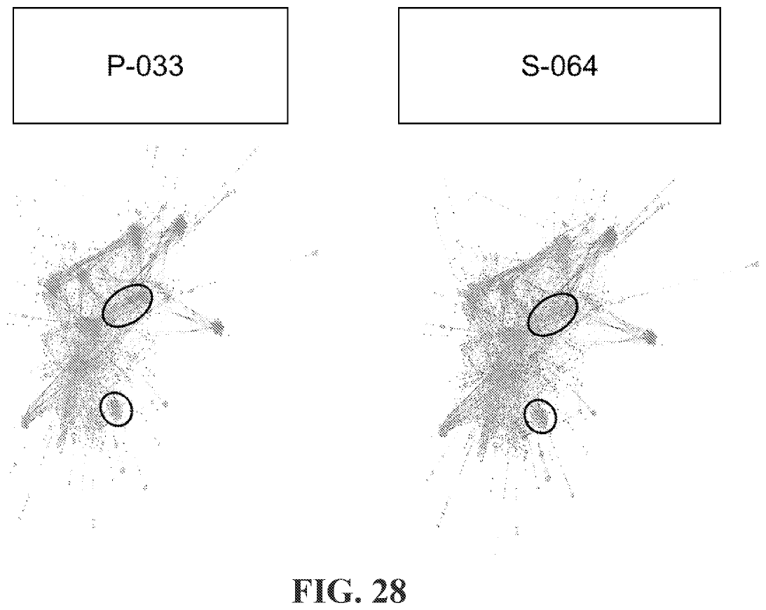

FIG. 28 shows construct maps of biological physical protein-protein interactions from the STRING public database. Protein-protein interaction maps were colored by whether or not a protein is identified in a corona of either a P-033 particle type (surfactant free carboxylate microparticles, left plot) or a S-064 particle type (2.0-2.9 μm polystyrene microparticles, right plot). Proteins that were identified in the particle corona are lightly shaded, and proteins that were not identified in the particle corona are shown in darkly shaded. Patterns present in each interaction map indicated that the patterns are different for each particle type and that the patterns are non-random, suggesting that there is a relationship between the proteins present in the protein corona and the underlying biology represented by the interaction map. Two examples of regions with differences in identified protein abundances are circled.

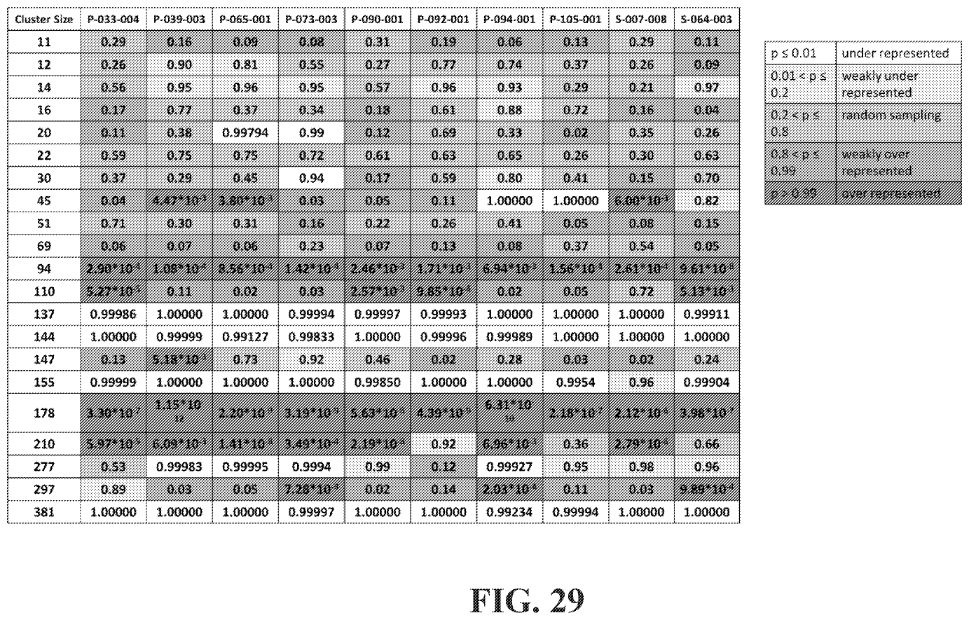

FIG. 29 shows a table of probabilities that a particle sampled the observed number of proteins from that group based on particle type, shown in columns, and protein cluster, shown in rows. Cell shading depicts whether the protein cluster is over represented or under represented on the given particle type. Light shading indicates that the protein cluster was underrepresented. Dark shading indicates that the protein cluster was over represented. Moderate shading can indicate that the identification of the protein cluster was commensurate with random sampling..

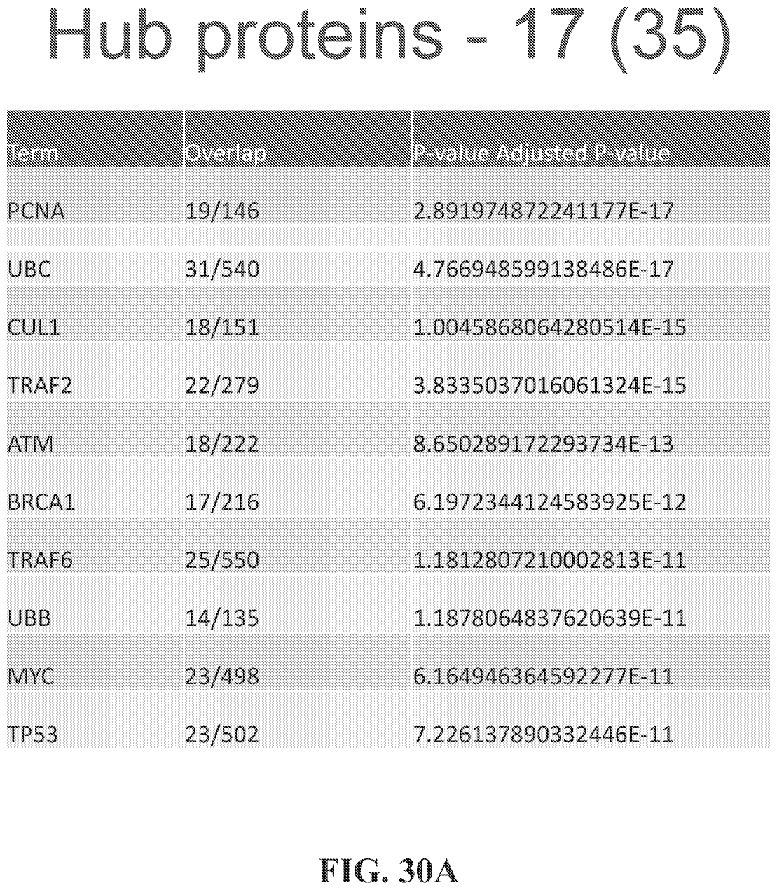

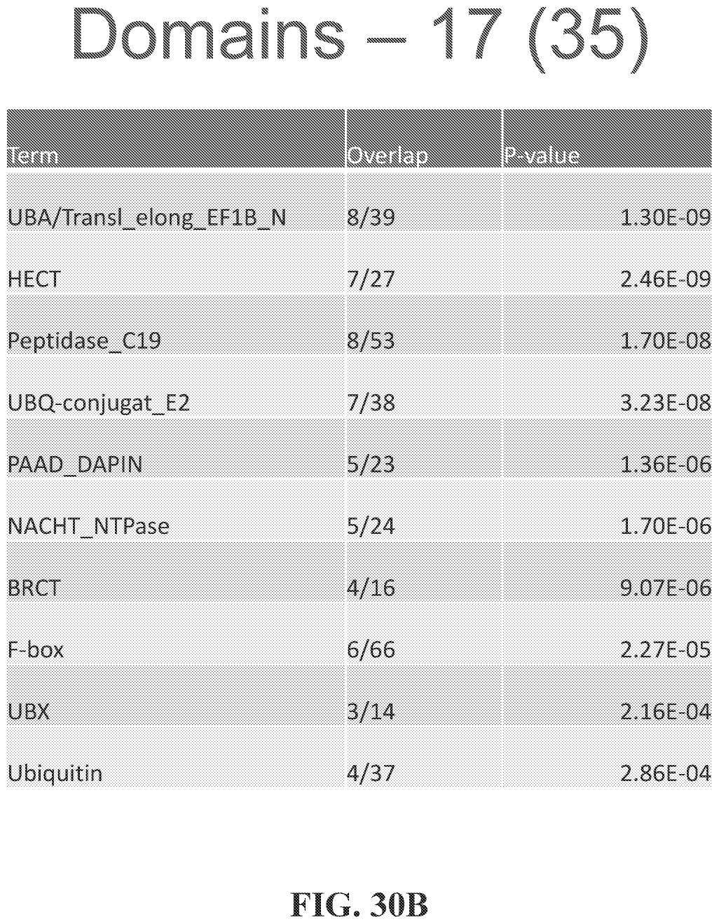

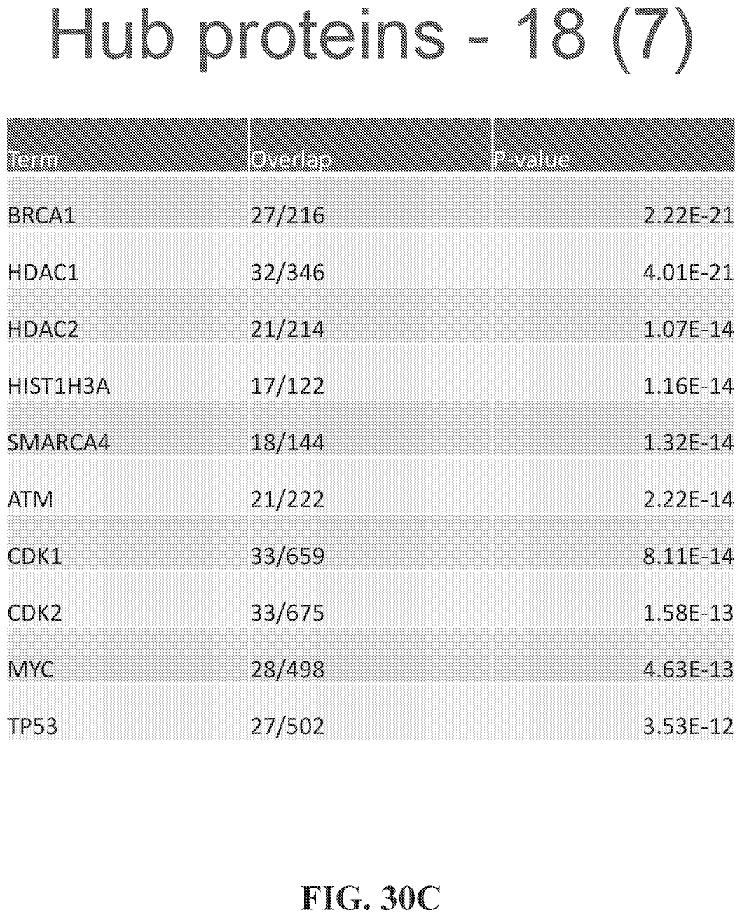

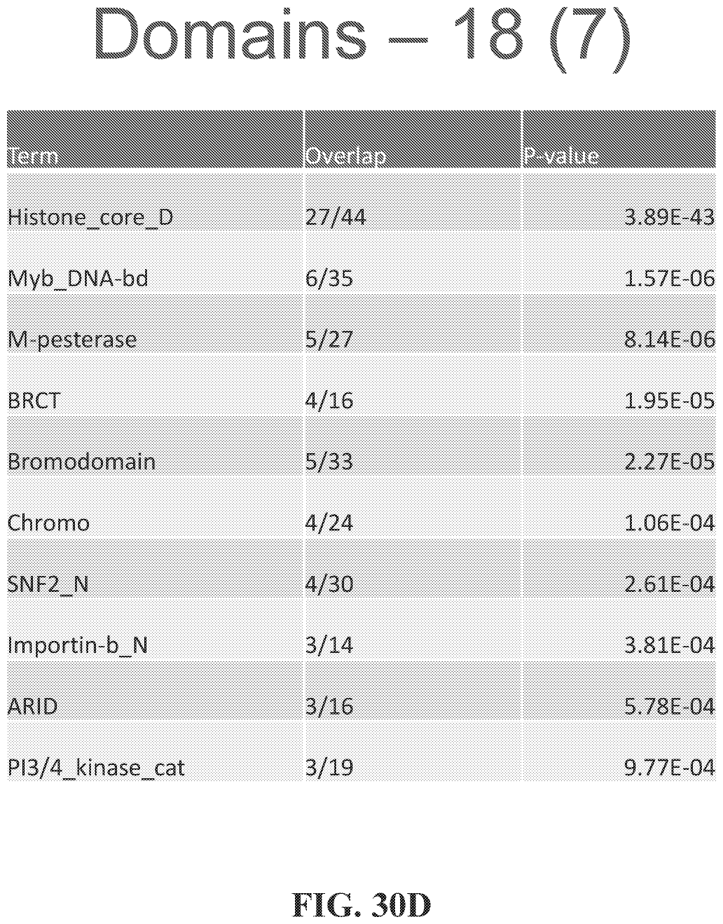

FIG. 30A-D shows the top 10 hub proteins (FIG. 30A and FIG. 30C) and top 10 protein domains (FIG. 30B and FIG. 30D) common to many proteins in each of two under represented protein clusters, cluster 17 (FIG. 30A and FIG. 30B) and cluster 18 (FIG. 30C and FIG. 30D). Hubs represent clusters of proteins.

### Example 24

### Protein Collection on Ubiquitin Functionalized Particles

This example describes a protein collection assay with a high degree of profiling depth. The assay compared protein group counts for ‘macromolecular functionalized’ particles and ‘small molecule functionalized’ particles (with silica, amine, phosphate sugar (glucose-6-phosphate), and carboxyl surface functionalities)

The assay identified nearly 2000 distinct protein groups from human plasma Achieving such a high level of profiling depth required the collection of more than a thousand sub ng/ml proteins with highly varied physical properties. While the present disclosure provides particles capable of collecting hundreds of protein groups from plasma, collecting greater than 1000, 1500, or 2000 types of proteins from a single sample required optimization of protein collection-complementarity in a multi-particle panel. Macromolecular functionalized particles not only provided high protein group counts, but also collected large numbers of different proteins not identified on the small molecule functionalized particles.

A plasma sample was contacted to three types of macromolecular functionalized particles and 6 types of small molecule functionalized particles, listed in TABLE 17. The macromolecular functionalized particles included one dextran coated particle and two types of ubiquitin functionalized particles, one with ubiquitin conjugated through a genetically engineered single cysteine residue at the N-terminus by a heterobifunctional crosslinker, and therefore with ubiquitin identically oriented relative to the particle surface(cis-ubiquitin functionalized, S-163-001 & S-163-002), and one with amine group linked, and therefore randomly oriented, ubiquitin (S-164-001 & S-164-002). Plasma samples were diluted 1:5 in a dilution buffer composed of TE buffer (10 mM Tris, 1 mM disodium EDTA, 150 mM KCl) with 0.05% CHAPS, and then apportioned in 100 μl aliquots between microplate wells, and then mixed 1:1 (v:v) with solutions containing 2.5-15 mg/ml of a single type of particle. The plates were sealed and incubated at 37° C. for about 1 hour with shaking at 300 rpm, after which point the particles were pelleted and separated from the supernatant, thereby removing unbound protein. The resulting protein coronas were further washed with about 200 μL of dilution buffer for three times, digested, and then analyzed by tandem mass spectrometry. Each particle preparation was tested in triplicate.

TABLE 17
Small molecule functionalized and macromolecule functionalized particles

| Batch No. | Functionalization | Description |
|---|---|---|
| S-003-111 | SMALL MOLECULE | Silica-coated superparamagnetic iron oxide NPs |
| | FUNCTIONALIZED | (SPION) |
| S-006-017 | | N-(3-Trimethoxysilylpropyl)diethylenetriamine |
| | | coated SPION |
| S-007-023 | | Poly(N-(3-(dimethylamino)propyl) |
| | | methacrylamide) (PDMAPMA)-coated SPION |
| S-118-053 | | glucose-6-phosphate-functionalized |
| S-125-026 | | 3-aminopropyltriethoxysilane-functionalized |
| P-039-010 | | Polystyrene carboxyl functionalized |
| S-163-001 | MACROMOLECULE | Cis-ubiquitin-functionalized |
| S-163-002 | FUNCTIONALIZED | Cis-ubiquitin-functionalized |
| S-164-001 | | Ubiquitin-functionalized |
| S-164-002 | | Ubiquitin-functionalized |
| P-073-010 | | Dextran based coating, 0.13 μm |
| P-073-011 | | Dextran based coating, 0.13 μm |

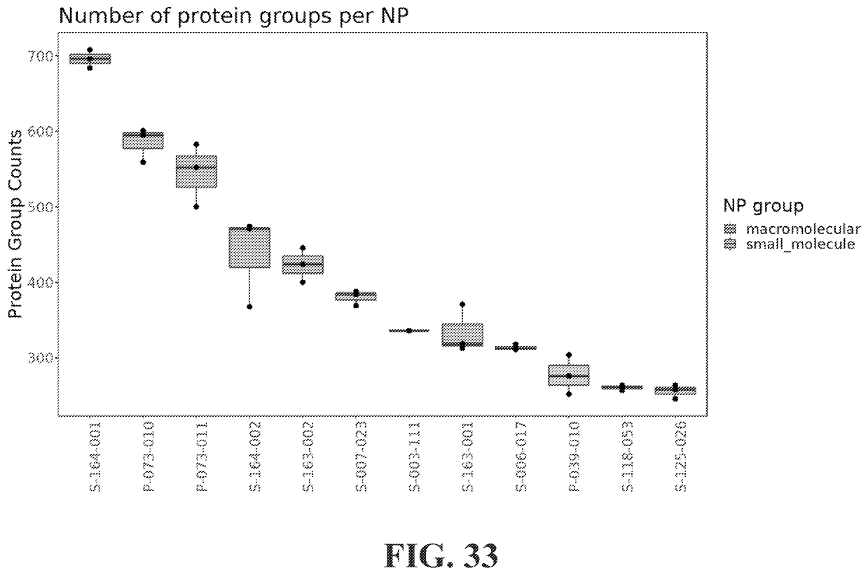

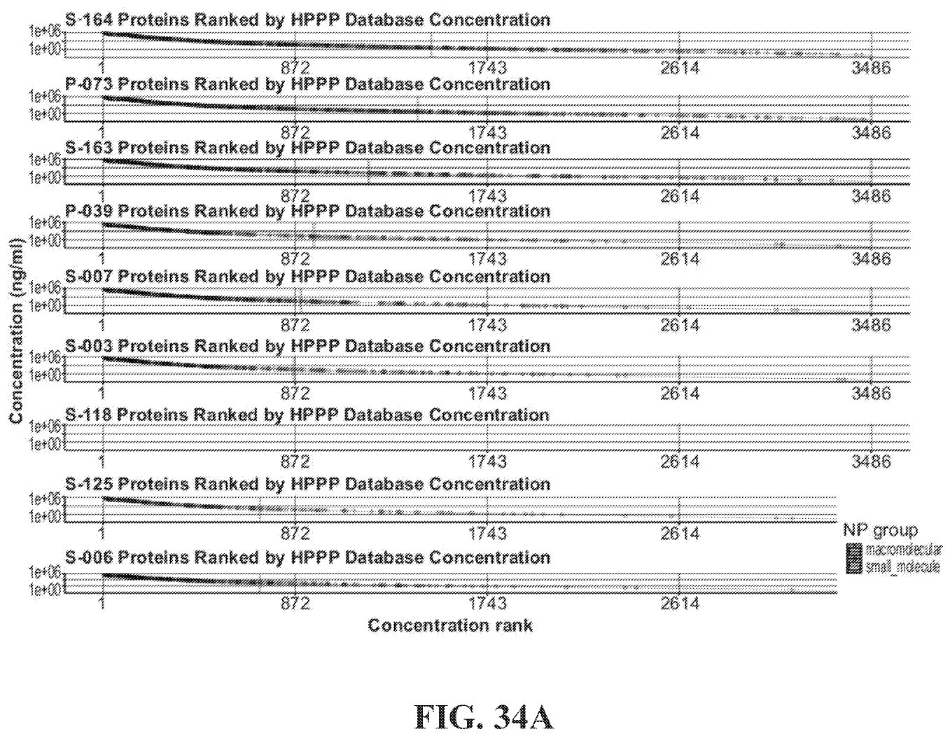

FIG. 33 shows the number of protein groups collected on each particle preparation. The greatest protein group counts were observed for the three macromolecule functionalized particles, with the S-164-001 particle preparation yielding the greatest protein group count of nearly 700. The small molecule functionalized particles provided protein group counts of between 350 and 250, with the S-125-026 and S-118-053 particle preparations yielding the lowest protein group counts of around 260.

FIG. 34A illustrates the plasma concentrations of protein groups collected on each type of particle. Each circle on the plots represents a protein group collected on the corresponding particle type, with the degree of shading indicating the relative amount of the protein collected on the particle (darker corresponds to a greater amount of protein collected), the y-axes provide solution concentrations, and the x-axes provide a rank for solution concentration. For example, albumin, the most abundant plasma protein, has an x-axis value of 1. The vertical line on each plot represents the 50^{th} percentile solution abundance for the protein groups identified on a particle. FIG. 34B-34J provide blown up versions of the plots provided in FIG. 34A. Each plot provides 25^{th}, 50^{th}, and 75^{th} percentile lines for protein groups identified on the particle, and the number of protein groups identified from each plasma protein quartile. The horizontal lines on each plot depict the plasma concentrations of the 25th, 50th, and 75^{th} percentile protein group identified on each particle type.

FIG. 34K provides the protein group numbers for the particles illustrated in FIG. 34A-J. For each type of particle, the total number of protein groups (‘Overall N match’); the relative plasma abundances of the 25^{th} percentile, 50^{th} percentile, and 75^{th} percentile protein groups; and the percent of protein groups identified on each particle from the top quartile (Q1), second quartile (Q2), third quartile (Q3), and bottom quartile (Q4) of human plasma proteins based on mean plasma concentration.

As can be seen in FIG. 34, the macromolecular functionalized particles collected greater numbers of protein groups and a greater proportion of low concentration protein groups. Of the 729 protein groups identified on the ubiquitin (S-164) particles, 119 were from the lower two quartiles in terms of plasma concentration (less than about 40 ng/ml), nearly 10-times higher than the average number collected on the small molecule functionalized particles. In fact, more bottom quartile proteins were individually identified on the ubiquitin (S-164) and on the dextran (P-073) particles (36 and 27, respectively) than on all small molecule particles, combined (17). 729 and 490 protein groups were identified from the ubiquitin and cis-ubiquitin particles, respectively. The dextran particles collected 662 distinct protein groups. Comparatively, the small molecule functionalized particles collected between 276 and 329 protein groups. Thus, macromolecular functionalization enhanced collection of high and low concentration proteins, including proteins at concentrations of less than 10 ng/ml.

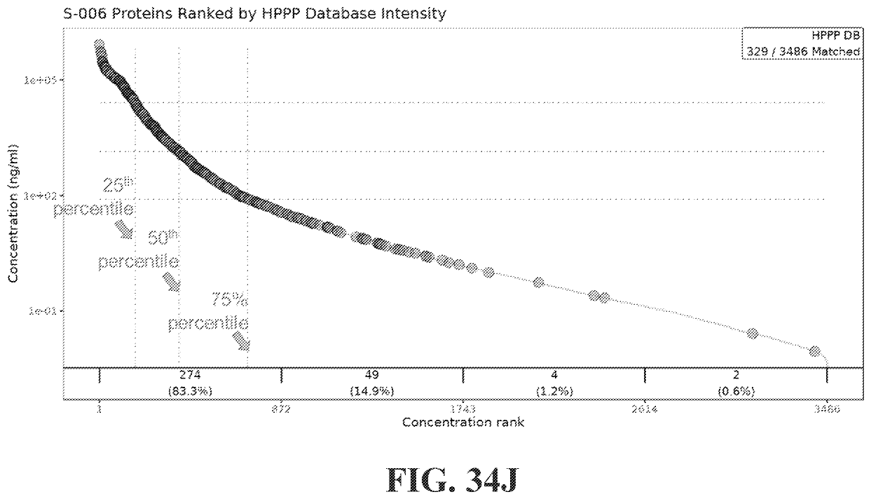

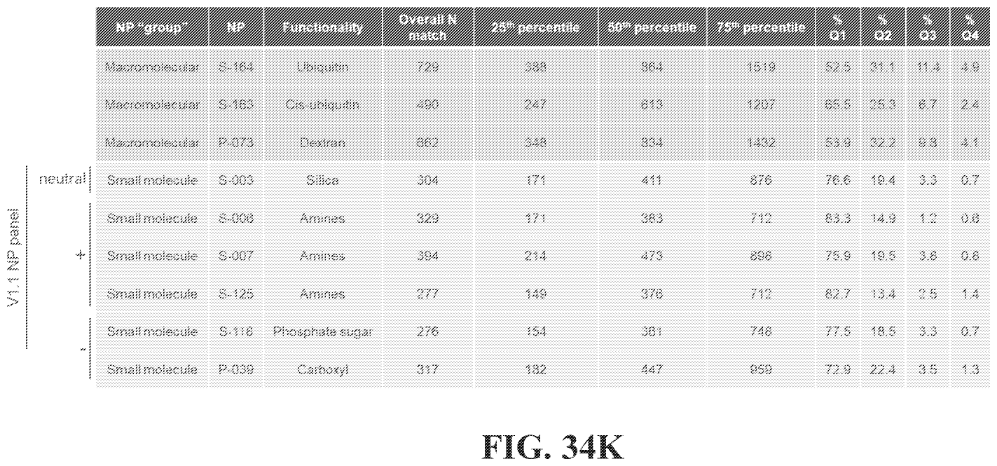

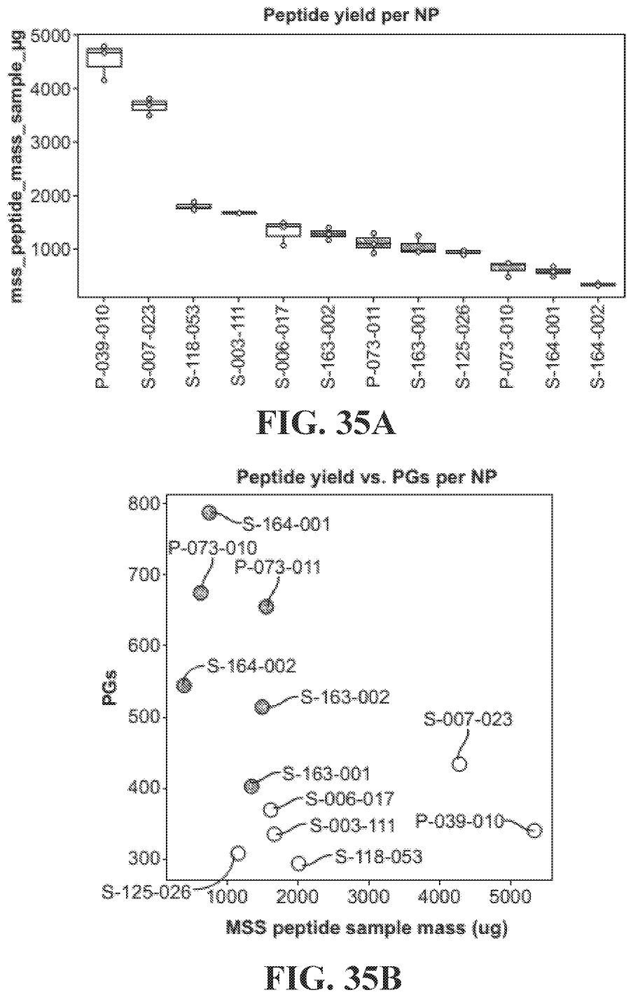

FIG. 35A shows the mean collected peptide mass by particle preparation and type. Contrasting their high protein group counts, the macromolecular functionalized particles displayed relatively low protein yields of about 300 to 1500 μg protein per contacted sample. While four of the six small molecular functionalized particles provided similar yields, two types of particles, (S-007 and P-039) yielded multi-fold higher protein yields. Given that these two particle types are oppositely charged (positive for S-007, negative for P-039), it is unlikely that a positive or negative charge alone favors higher protein yield.

FIG. 35B plots the protein group (y-axis) versus protein mass (x-axis) yields for the twelve particle preparations. The macromolecular functionalized particles nearly uniformly provided lower protein mass yields and higher protein group counts than the small molecule functionalized particles, indicating that their higher protein group counts are due to increased collection diversity, and not simply increased protein collection quantity.

Quantitative depiction of the protein group overlap between particles is shown in the FIG. 36A UpSet plot, in which the bottom left horizontal bar graph shows the total number of protein groups collected on each particle type, the top vertical bar graph shows the number of protein groups within different clusters, and the bottom right dot-plot indicates which clusters were present on each particle type. Multiple dots under a protein group cluster indicates that the cluster was observed on multiple particle types, while a single dot indicates the cluster was only observed on a single type of particle. As can be seen in column 3 of the plot, the S-164-001 (ubiquitin functionalized) particles collected 50 unique protein groups, the most of any of the 12 particle preparations. The small molecule functionalized particles S-006, S-007, and P-039 all collected at least 20 unique protein groups, demonstrating that a number of protein types are specifically attracted to small molecule functionalities. Similarly, many of the protein group clusters were specific to the macromolecular functionalized particles, including those depicted in columns 2-6, 9, 11-14, and 16, and representing a total of 312 protein groups.

FIG. 36B provides a Venn diagram comparing the protein groups collected on the ubiquitin functionalized particles (S-164-001) and the dextran functionalized particles (P-073). Of the 787 protein groups identified on the ubiquitin functionalized particles and 741 protein groups identified on the dextran functionalized particles, 137 were unique to the ubiquitin functionalized particles, while 91 were unique to the dextran functionalized particles. 650 protein groups were common between the two particle types, suggesting that many protein groups have a general affinity for macromolecular biomolecules, and are not discriminated between ubiquitin and polysaccharide functionalizations.

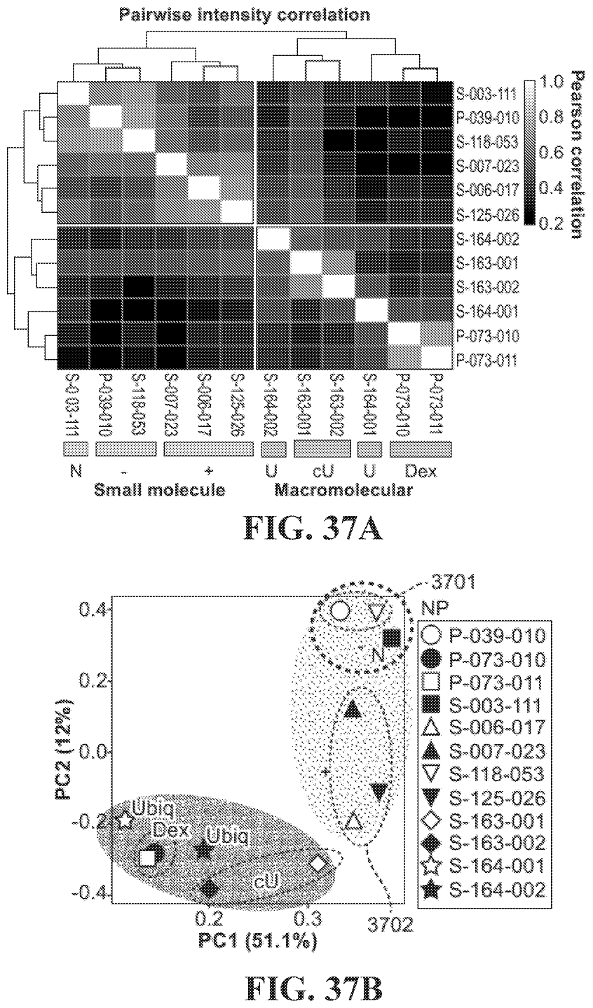

FIG. 37A shows the Pearson correlations for protein groups collected on each of the 12 particle preparations, with higher values indicating greater overlap between collected protein groups. By definition, each diagonal entry has a value of 1. The top left quadrant, which corresponds to pairs of small molecule functionalized particles, has the highest correlation values, illustrating that a large percentage of the protein groups collected on the small molecule functionalized particles are common to all 6 particle types. The upper right and lower left quadrants, representing pairings between macromolecular functionalized and small molecule functionalized particles, has low correlation between particle types, indicating that the protein groups collected on macromolecular functionalized and on small molecule functionalized particles have low overlap, and suggesting that combinations of the two classes of particles could be used to generate complementary protein group profiles from biological samples to yield high profiling depths.

FIG. 37B provides a principle component analysis plot for the protein groups collected on the 12 particle preparations. As can be seen from the chart, the macromolecular functionalized particles (bottom left highlighted area) cluster separately from the small molecule functionalized particles (top right highlighted area). The small molecule functionalized particle cluster contains two sub-clusters, with the negatively charged particles (P-039 and 5-118) and neutral particle (S-003) constituting a first sub-cluster 3701, and the positively charged particles (S-006, S-007, and S-125) constituting a second sub-cluster 3702. Thus, for small molecule functionalized particles, it appears that surface charge has a large impact on protein group collection. Among the macromolecular functionalized particles, the two dextran coated particles share the highest degree of similarity, while both pairs of ubiquitin functionalized particles exhibit a moderate degree of dissimilarity, possibly reflecting different degrees of ubiquitin surface coverage achieved across the separate particle preparations. Across clusters, a cis-ubiquitin particle (S-163-001) appears to be highly similar to one of the amine functionalized particles (S-006), indicating that a small molecule functionalized particle may be tailored to mimic the protein collection properties of a macromolecular functionalized particle.

FIG. 38A shows the Pearson correlations between particle types and collected protein groups for the ubiquitin functionalized particles (S-164-001) and dextran functionalized particles (P-073-010 and P-073-011), with three replicates shown for each particle preparation. The intensity of each spot indicates the abundance of a particular protein group on the indicated particle type, with high values indicating a large amount of the protein group collected on the particle type. Large portions of the plot show little variance between particle preparations. Of note, two bands 3801 and 3802 represent families of protein groups collected in high abundance on all 3 particle preparations. A number of regions depict proteins specific to a particular particle preparation 3803-3806. Collectively, these results indicate that the majority of protein groups are conserved across the two macromolecular functionalized particle types, but that a number of protein groups are specific to each particle type and preparation.

FIG. 38B provides FDR adjusted p-values for 100 plasma protein classes observed on the ubiquitin functionalized and dextran coated particles, with lower values indicating a higher degree of confidence in the indicated group's enrichment. This data representation accounts for false positives and discoveries that can occur during multi-assay comparisons. A large number of protein classes are observed for only one of the two particle types. As is indicated in the blown up portion of the plot, the ubiquitin functionalized particles actively enriched for ubiquitin- and ubiquitin-like protein (e.g., neural precursor cell-expressed developmentally down-regulated protein 4 (NEDD4), small ubiquitin-like modifier (SUMO)) functionalized proteins. The ubiquitin functionalized particles also selectively collected proteins associated with nucleic acid splicing and synthesis. Among the many classes selectively enriched on the dextran functionalized particles were a number of metalloenzyme classes, including iron, heme, copper, and zinc proteins. FIGS. 38C & D provide highlight regions of FIG. 38B, highlighting the selectivity of the ubiquitin functionalized particles for membrane (including transmembrane) proteins, and of both particle types for the complete human proteome (‘complete proteome’). These results show that particles can be tailored to not only collect individual biomarkers, but also to collect particular classes of biomolecules and proteins.

FIG. 38E shows the p-values for the identification of protein classes collected on the dextran and ubiquitin functionalized particles as a function of the ratio of mass spectrometric intensities of the protein classes between the two particle types. Each data point on the plot represents a protein group identified on the two particle types. As can be seen in the plot, a number of protein types were strongly associated with one of the ubiquitin or the dextran functionalized particles, and could be identified with a high degree of confidence.

FIG. 38F illustrates the numbers of protein groups identified on the ubiquitin functionalized and dextran coated particles. Of the 639 protein groups collected on the two particles, 372 were common to both particle types and 234 were unique to the ubiquitin functionalized particles, while only 33 were unique to the dextran coated particles.

FIG. 38G provides a principle component analysis plot for the three replicates of the particle preparations shown in FIG. 38A. The replicates for the ubiquitin functionalized particles (top left) and dextran coated particles (bottom right) form separate clusters, with the two preparations of the dextran coated particles being nearly identical. Collectively, these results show that the variation between particle types is considerably larger than the variation between assays, and thus demonstrate a high degree of repeatability for assays using a defined particle type.

FIG. 38H provides FDR adjusted p-values for about 100 plasma protein classes observed on the studied particle types. Only protein classes with at least one term with p<0.05 are shown. A number of protein classes are identified with high degrees of confidence across all of the particle types, including membrane, zinc, and receptor proteins. Few protein classes are specific to a single particle type. Some protein classes were specific to macromolecular functionalized particles, such as ion channel and RNA processing proteins, while others, such as lysosome proteins, were specific to small molecule functionalized particles.

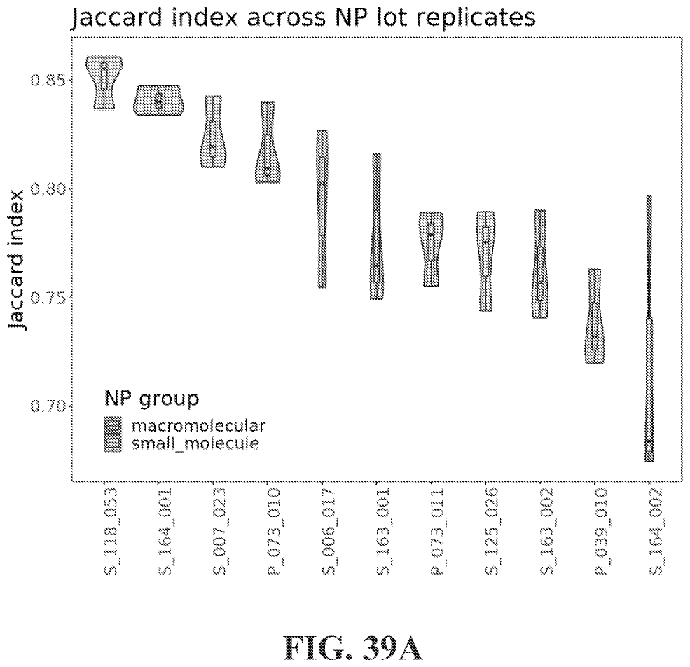

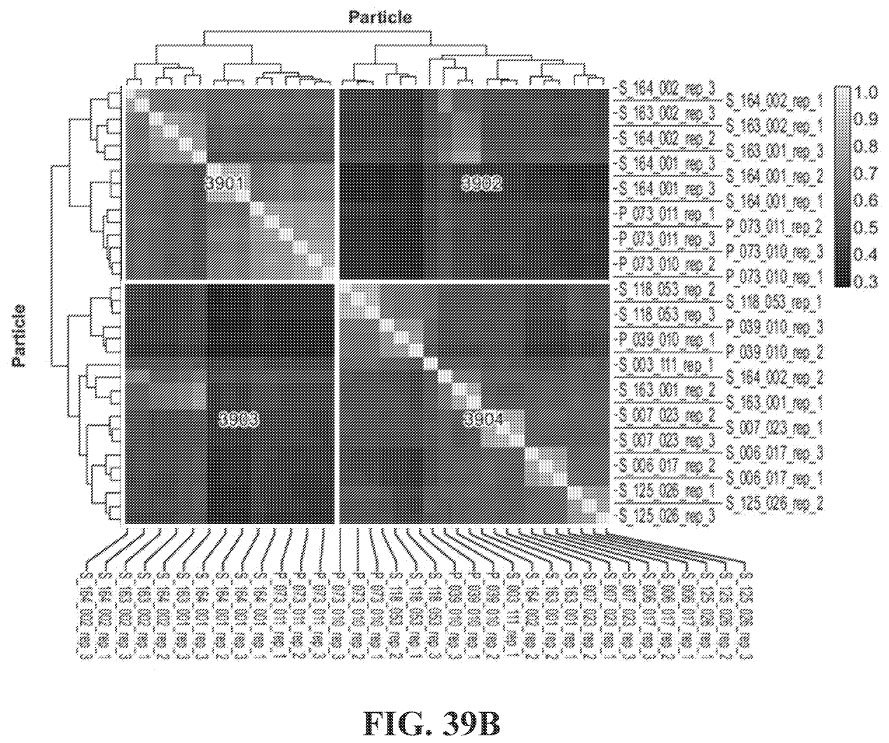

FIG. 39A shows Jaccard indices for the proteins identified on the different particle preparations across multiple assays. The high Jaccard indices indicate low variation in the number and types of identified proteins across replicates, thus revealing a high degree of repeatability for the protein corona assays. Some variability was observed between different preparations for the same type of particle. For example, the two preparations of ubiquitin functionalized particles (S-164-001 and S-164-002) yielded Jaccard indices of about 0.83 and 0.68, while the two preparations of the dextran functionalized particles (P-073-010 and P-073-011) yield Jaccard indices of 0.82 and 0.76. It is likely that the different preparations of these particles yielded slightly different properties, manifesting in different protein corona formation behaviors.

FIG. 39B shows the Jaccard indices for the proteins collected in separate assays on the various particle types tested, with 1 indicating identical protein collections and values close to zero representing disparate results. The greatest similarities are observed for replicates with the same particle type and preparation (the boxes 1 or 2 spaces from the diagonal). Additionally, the data appear to group into 4 quadrants, corresponding to high Jaccard indices among the pairs of macromolecular functionalized particle replicates (3901), low Jaccard indices for pairings between a replicate with a macromolecular functionalized particle and a replicate with a small molecule functionalized particle (3902 and 3903), and high Jaccard indices among the pairs of small molecule functionalized particle replicates (3904). These results indicate that the small molecular functionalized particles and macromolecular functionalized particles collect distinct sets of plasma proteins.

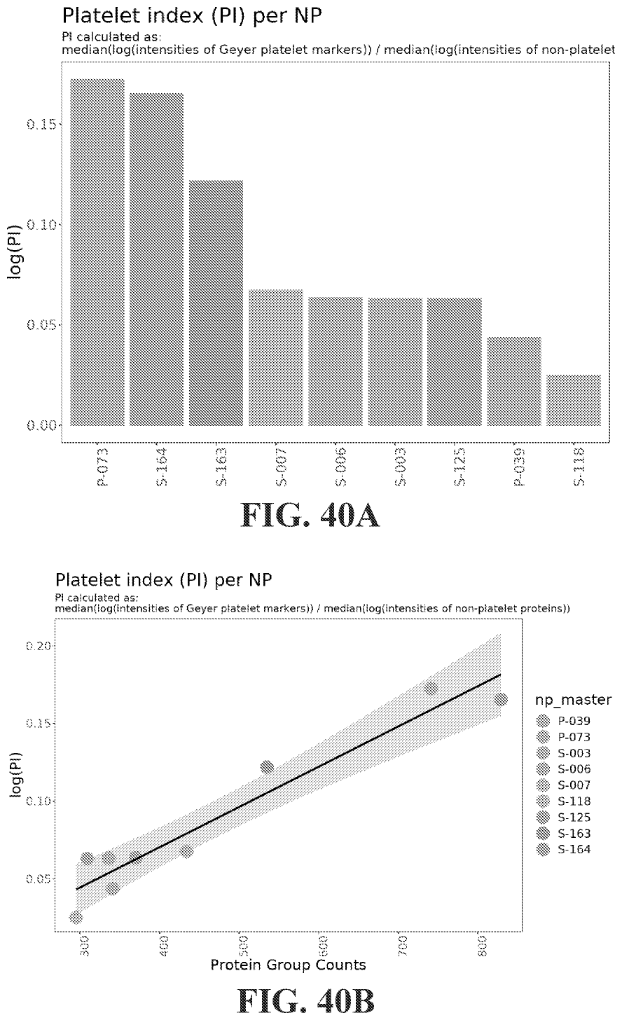

Platelet Marker Collection. FIG. 40A shows the platelet indices for each of the studied particle types, measured as the ratios of the relative intensities of platelet marker proteins and non-platelet marker proteins identified on each particle. The three macromolecular functionalized particles have the highest platelet indices, spanning from 0.17 for the dextran functionalized particles to 0.12 for the cis-ubiquitin particles. The phosphate sugar (small molecule) functionalized particles (S-118) had the lowest index of around 0.025.

FIG. 40B compares the platelet indices to the number of protein groups identified (‘protein group counts’) on each particle type. As can be seen from the best fit line in the chart, platelet index correlates with protein group count. Furthermore, the three macromolecular functionalized particles have the highest protein group counts and platelet indices. Thus, particles can not only be optimized to collect a large number of proteins, but also to collect large quantities of a particular class of proteins, such as platelet markers.

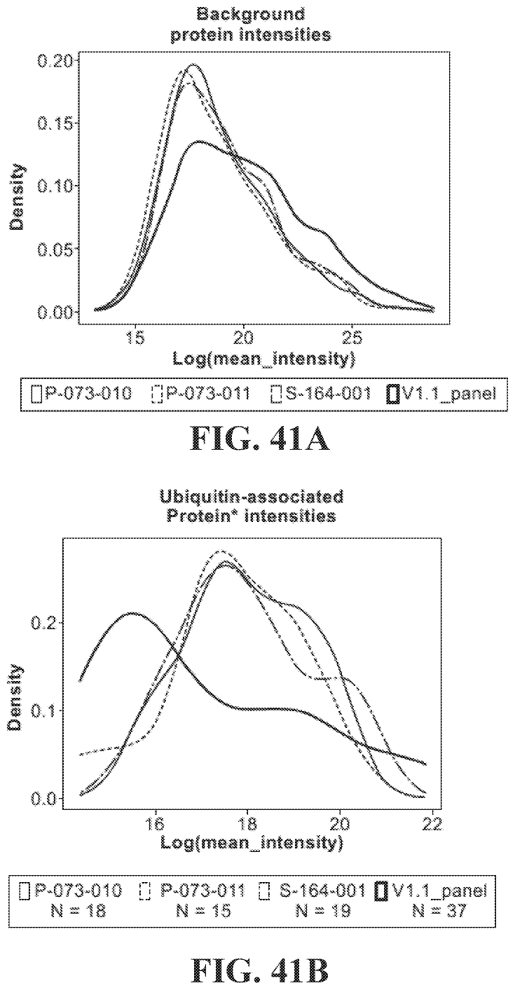

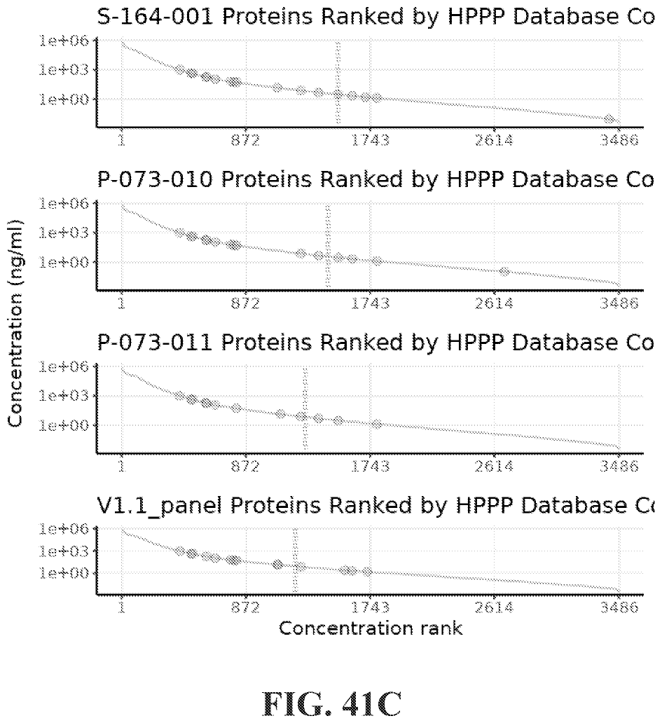

Ubiquitin-Associated Protein Collection. The small molecule functionalized proteins collected greater proportions and amounts of ubiquitin-associated proteins than the small molecule functionalized particles. FIG. 41A shows the distribution of mass spectrometric signal intensities for non-ubiquitin associated (‘Background’) proteins collected on the dextran functionalized particles, the ubiquitin functionalized particles, and on a particle panel comprising the 6 small molecule functionalized particles. The small molecule particle panel generated a higher total mass spectrometric intensity than the macromolecular functionalized particles, with a nearly three orders of magnitude higher mean intensity, and a greater number of very high intensities peaks (10²⁴ or greater). As is shown in FIG. 41B, the intensity of features corresponding to with ubiquitin-associated proteins is considerably higher for the macromolecular functionalized proteins. While the intensity distributions are similar for the macromolecular functionalized particles between the background and ubiquitin-associated protein intensity plots, the small molecule functionalized panel displays predominantly low (sub 10¹⁶) intensity features for ubiquitin associated proteins.

FIG. 41C provides the plasma concentrations (in ng/ml) of ubiquitin associated proteins identified on the macromolecular functionalized particles and on the small molecule functionalized particle panel, with darker circles representing greater mass spectrometric intensities for an identified protein. The greatest number of ubiquitin-associated proteins were identified on the ubiquitin functionalized particles, while the fewest were identified on the small molecule particle panel. Furthermore, the macromolecular functionalized particles each identified a sub ng/ml concentration ubiquitin-associated protein, which was not achieved by the small molecule functionalized particle panel.

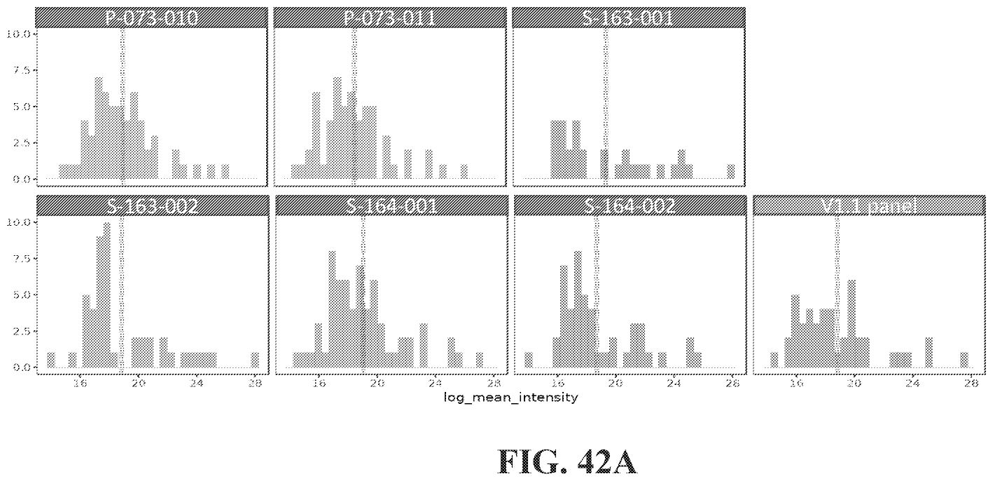

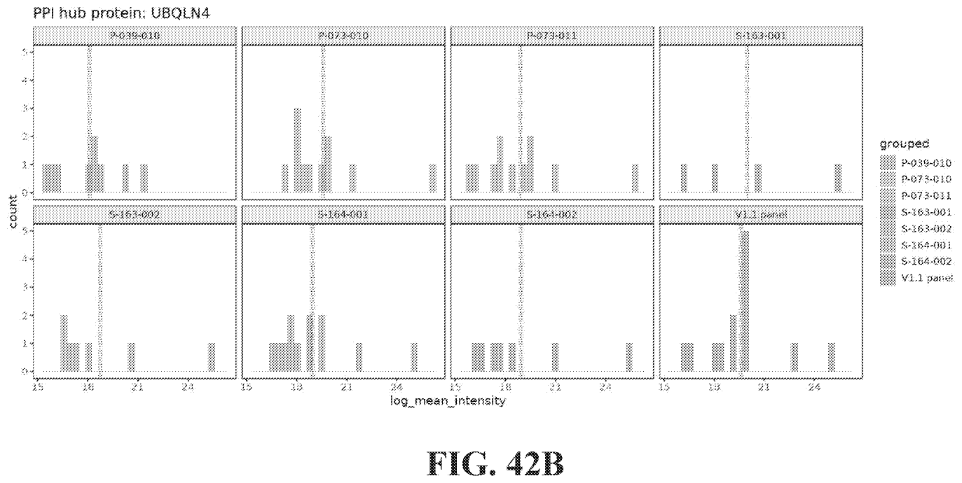

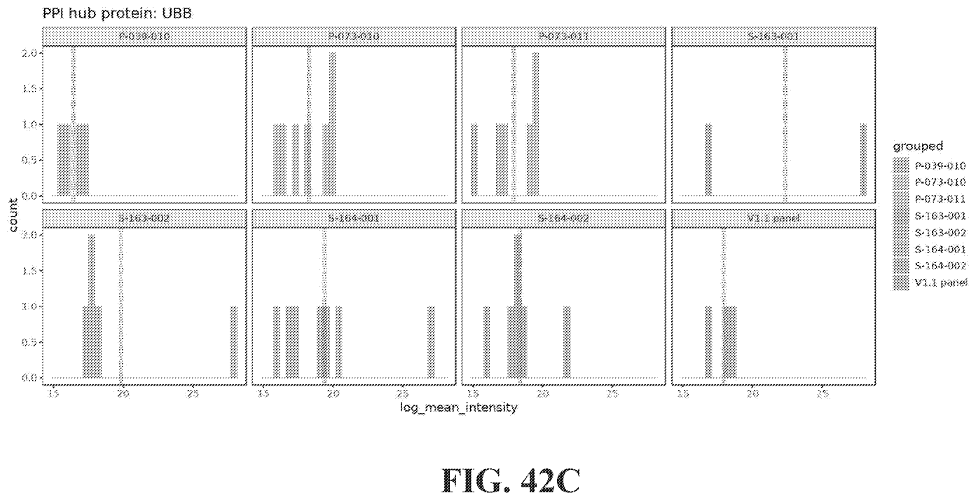

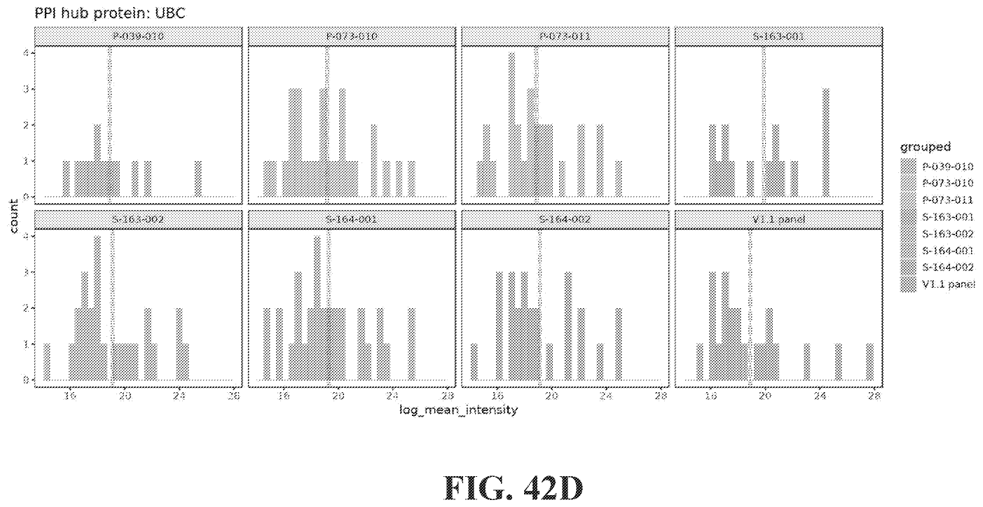

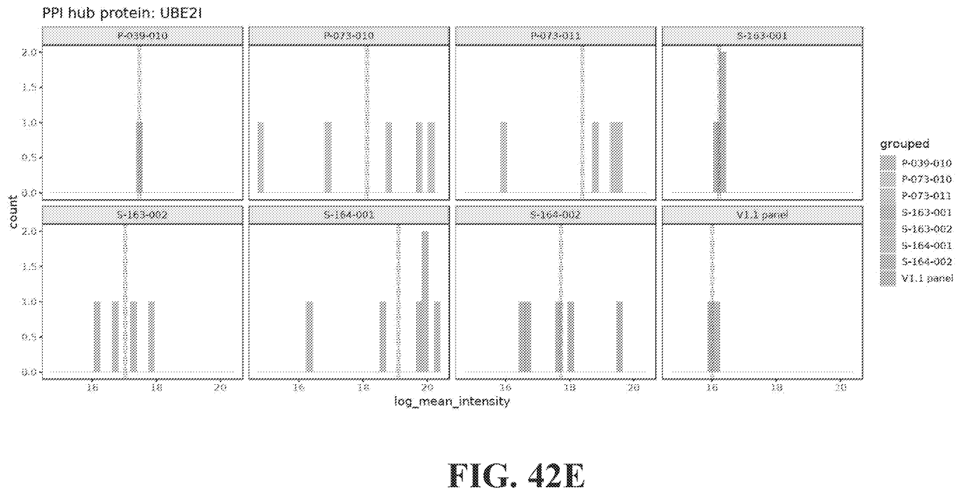

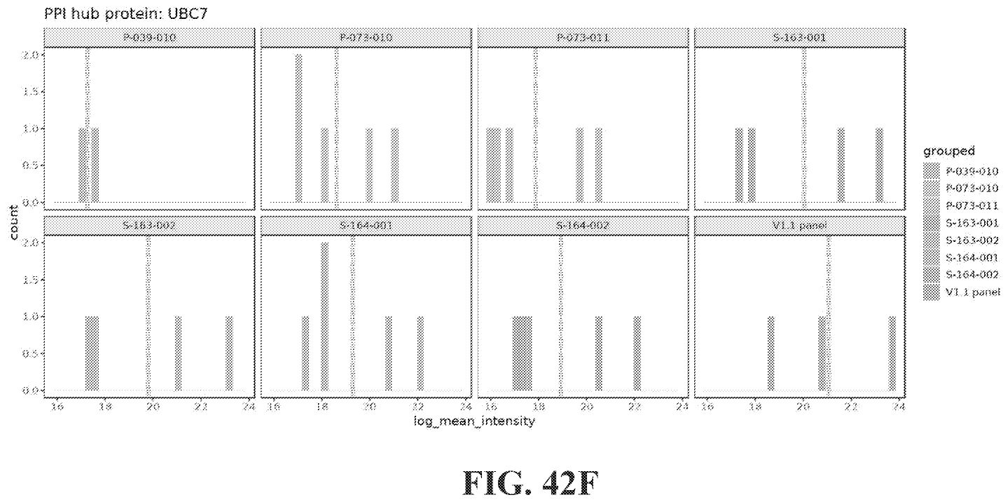

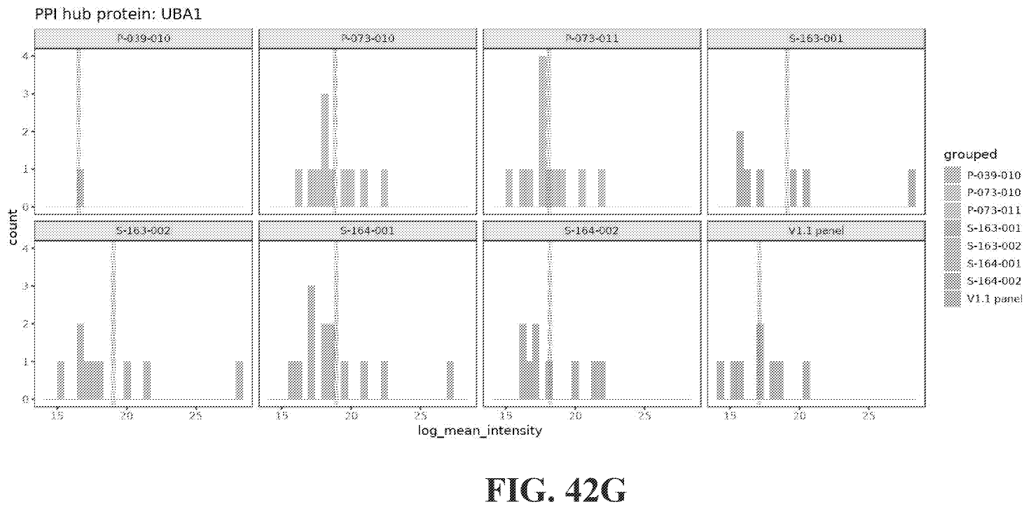

FIG. 42A-F display the intensities of mass spectrometric features corresponding to six separate ubiquitin hub proteins collected from plasma samples on the dextran, ubiquitin, and cis-ubiquitin functionalized particles and on the small molecule functionalized particle panel. FIG. 42A shows the aggregate distributions for all six ubiquitin hub proteins. As can be seen from these plots, the small molecule functionalized particle panel and macromolecular functionalized particles generate similar intensities for the ubiquitin hub proteins. FIG. 42B-G display feature intensities distributions for the individual hub proteins. For the six hub proteins analyzed, three displayed the highest intensity mass spectrometric features on the small molecule functionalized particle panel, one displayed the highest intensity mass spectrometric features on the ubiquitin functionalized particles, one displayed the highest intensity mass spectrometric features on the cis-ubiquitin functionalized particles, and one displayed the highest intensity mass spectrometric features on the dextran functionalized particle. Collectively, these results suggest that mixed particle panels can be the most effective means for assaying certain protein classes.

Particle Panel Optimization. The performance of the macromolecular functionalized particles motivated the creation of a particle panel containing a mixture of macromolecular functionalized particles and small molecule functionalized particles. While the macromolecular functionalized particles collected more protein groups than any of the individual small molecule functionalized particles, each type of particle collected unique types of protein groups, suggesting that a combination of particle types could enhance protein collection, and thus sample profiling depth.

FIG. 43A depicts the scheme used to generate mixed particle panels with small molecule functionalized and macromolecular functionalized particles. 20 distinct particle panels were generated by substituting either ubiquitin (S-164-001 and S-164-002) or cis-ubiquitin (S-163-001 and S-163-002) functionalized particles for one of the five particle types in a control particle panel containing S-003, S-006, S-007, S-118, and S-125 particles. The particle panels were contacted to plasma solutions to form biomolecule coronas, which were assayed for protein groups as described above.

FIG. 43B depicts the total number of protein groups collected on each particle panel. As can be seen on the right of the figure, the control particle panel (containing only small molecule functionalized particles) collected the fewest number of protein groups, approximately 600 in total. Substituting any one of the 5 control panel particles for a macromolecular functionalized particle increased the number of protein groups collected, maximizing at around 1000 protein groups upon substitution for an S-164-001 particle. Individually, this particle type collected 729 protein groups from plasma (FIG. 34K), meaning that nearly a third of its protein groups were unique as compared to the protein groups collected on the small molecule functionalized particles.

### Example 25

### Protein-Protein Interaction Maps For NSCLC

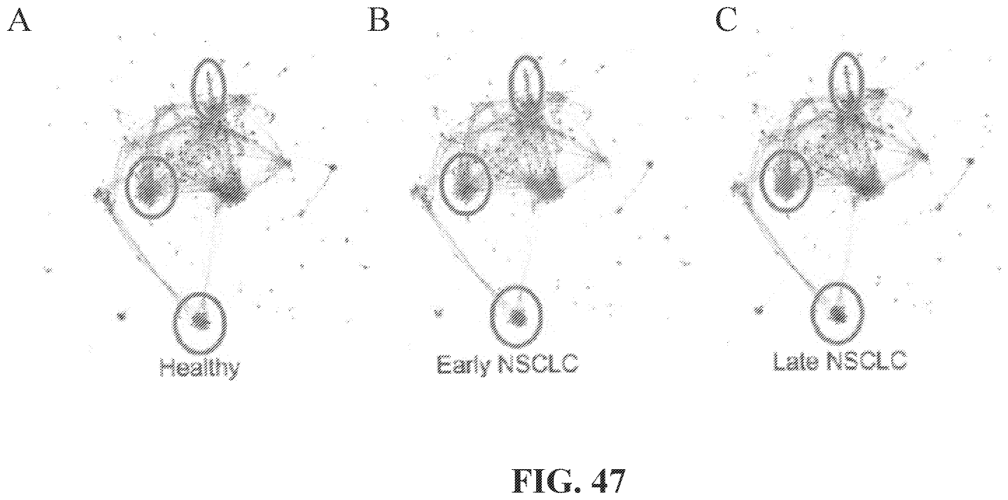

This example overviews the generation of a protein-protein interaction map relevant to non-small cell lung cancer (NSCLC). Plasma samples from a total of 276 were analyzed using a particle panel, and the proteins identified from the samples were used to generate the protein-protein interaction map shown in FIG. 47 using the STRING database. In the map, dots represent proteins, with lighter shading indicating higher abundances. A cluster of proteins may represent hubs of proteins with related functions or biological pathways, with lines indicating a potential relationship between two proteins. FIG. 47 panel A shows a map for healthy patients.

Panel B shows a map for early stage NSCLC patients. Panel C shows a map for late stage NSCLC patients.

FIG. 47 contains multiple nodes, including three which are circled for emphasis. Across the patient types, the abundances of proteins in (as identified on the particle panels) these hubs differ considerably, with the highest occupancy of the middle and bottom circled hubs observed for late stage NSCLC patients, and the highest occupancy of the top circled hub observed for healthy patients. Of note, the middle hub contains Golgi vesicle transport proteins, which is putatively linked to NSCLC. Thus, the NSCLC map is not only able to distinguish healthy subjects from NSCLC subjects, but is able to identify proteins that may be pertinent to NSCLC.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

1. A method of assaying a protein-protein interaction in a sample, the method comprising:
(a) obtaining data comprising biomolecule information for a plurality of distinct biomolecule coronas from the sample, wherein the plurality of distinct biomolecule coronas correspond to a plurality of distinct particle types, wherein the plurality of distinct particle types comprises a first particle type;
(b) detecting at least a primary protein and a secondary protein in a biomolecule corona of a first particle type from the data, and
(c) identifying the protein-protein interaction by measuring the primary protein associated with the first particle type and the secondary protein associated with the first particle type, wherein the secondary protein is more strongly associated with the primary protein than the first particle type, thereby indicating a presence of the protein-protein interaction between the primary protein and secondary protein.

2. The method of claim 1, wherein the measuring comprises detecting associations of at least (i) the primary protein and the first particle type, (ii) the secondary protein and the first particle type, and (iii) the primary protein and the secondary protein, wherein the secondary protein has a greater association with the first protein than with the first particle type.

3. (canceled)

4. The method of claim 1, wherein said measuring comprises quantifying the primary protein associated with the first particle type and the second protein associated with the first particle type.

5. The method of claim 1, wherein the data further comprises biomolecule information from a plurality of samples assayed using the plurality of distinct particle types.

6.-19. (canceled)

20. The method of claim 1, wherein the identifying further comprises measuring the primary protein and the secondary protein associated with a second particle type.

21. The method of claim 1, the assaying further comprising:
determining a between-particle score based on a first signal detected upon binding of the primary protein to the particle type of the plurality of distinct particle types and a second signal detected upon binding of the first protein to a second particle type of the plurality of distinct particle types, and
determining a same-particle score based on the first signal detected upon binding of the primary protein to the particle type and a third signal detected upon binding of the secondary protein to the particle type.

22. (canceled)

23. The method of claim 21, wherein the first signal, the second signal, and the third signal, the between-particle score, the same-particle score, or any combination thereof are used as training data for a machine learning algorithm.

24.-70. (canceled)

71. The method of claim 1, wherein the plurality of distinct particle types comprises one or more positively charged particle and one or more negatively charged particle.

72.-79. (canceled)

80. The method of claim 1, wherein the identifying the protein-protein interaction comprises identifying a biological state.

81.-90. (canceled)

91. The method of claim 90, wherein the assay comprises a mass spectrometric assay.

92.-93. (canceled)

94. A kit for assaying protein-protein interactions, the kit comprising: a first particle type and a second particle type, wherein the first particle type and the second particle type are one or more particle types selected from the group consisting of poly(acrylamide) particles, polyethylene glycol particles, carboxylate (Citrate) superparamagnetic iron oxide nanoparticle (SPION), a phenol-formaldehyde coated SPION, a silica-coated SPION, a polystyrene coated SPION, a carboxylated poly(styrene-co-methacrylic acid) coated SPION, a N-(3-Trimethoxysilylpropyl)diethylenetriamine coated SPION, a poly(N-(3-(dimethylamino)propyl) methacrylamide) (PDMAPMA)-coated SPION, a 1,2,4,5-Benzenetetracarboxylic acid coated SPION, a poly(Vinylbenzyltrimethylammonium chloride) (PVBTMAC) coated SPION, a carboxylate, PAA coated SPION, a poly(oligo(ethylene glycol) methyl ether methacrylate) (POEGMA)-coated SPION, a carboxylate microparticle, a polystyrene carboxyl functionalized particle, a carboxylic acid coated particle, a silica particle, a carboxylic acid particle, an amino surface particle, a silica amino functionalized particle, a Jeffamine surface particle, a polystyrene particle, a particle coated with a dextran based coating of about 0.13 μm in diameter, or a silica silanol coated particle.

95. (canceled)

96. The kit of claim 94, wherein one or more of the particles comprises a paramagnetic or superparamagnetic core material.

97.-169. (canceled)

170. A system comprising:
computer memory comprising data comprising biomolecule information for a plurality of distinct biomolecule coronas from a sample, wherein the plurality of distinct biomolecule coronas corresponds to a plurality of distinct particle types, wherein the plurality of distinct particle types comprises a first particle type;
a computer in communication with the computer memory, wherein the computer comprises a computer processor and computer readable medium comprising machine-executable code that, upon execution by the one or more computer processors, implements a method comprising:
(i) receiving the data from the computer memory;
(ii) from the data, detecting at least a primary protein and a secondary protein in a biomolecule corona of a first particle type; and
(iii) identifying the protein-protein interaction by measuring the association of the primary protein with the first particle type, the association of the secondary protein with the first particle type, and the association of the primary protein with the secondary protein,
wherein the association of the primary protein with the secondary protein is greater than the association of the secondary protein with the first particle type, thereby indicating a presence of the protein-protein interaction between the primary protein and secondary protein.

171.-172. (canceled)

173. The system of claim 170, wherein the measuring comprises identifying a variance in an association of (iii) across said at least said subset of distinct biomolecule coronas.

174. The system of claim 170, wherein (ii) and (iii) are repeated for a plurality of distinct pairs of primary and secondary proteins.

175. (canceled)

176. The system of claim 170, wherein the associations in (iii) comprise scores, wherein the scores are based on correlations.

177.-179. (canceled)

180. The system of claim 176, wherein the score is calculated based on Pearson value or correlation.

181. (canceled)

182. The system of claim 170, wherein (iii) further comprises calibrating an association of (iii) with a weighted algorithm or a machine learning algorithm.

183.-187. (canceled)

188. The system of claim 170, further comprising detecting a biological state based on the protein-protein interaction between the primary protein and the secondary protein.

189. The system of claim 170, wherein the data is transmitted to the computer memory over a communication network.

190.-203. (canceled)
